(19) United States
(12) Patent Application Publication
(10) Pub. No.: US 2017/0156335 A1
(43) Pub. Date: Jun. 8, 2017

(54) SYSTEMS AND METHODS FOR RELEASING CHLORINE DIOXIDE

(71) Applicants: Adva Bar-on, Azar (IL); Omri Mazar, Hadera (IL); Alon Polakewicz, Pardes Hanna Karkur (IL); Amir Shapira, Herzeliya (IL); Avi Shani, Kfar Haoranim (IL); Amos Golan, Tel Aviv (IL)

(72) Inventors: Adva Bar-on, Azar (IL); Omri Mazar, Hadera (IL); Alon Polakewicz, Pardes Hanna Karkur (IL); Amir Shapira, Herzeliya (IL); Avi Shani, Kfar Haoranim (IL); Amos Golan, Tel Aviv (IL)

(21) Appl. No.: 15/322,049

(22) PCT Filed: Jul. 1, 2015
(86) PCT No.: PCT/IB15/01680

§ 371 Date: Dec. 23, 2016

Related U.S. Application Data
(60) Provisional application No. 62/019,678, filed on Jul. 1, 2014.
(60) Provisional application No. 62/142,290, filed on Apr. 2, 2015.

(51) Int. Cl. A01N 59/00; A61L 15/44; C02F 1/76; A61L 15/42; B65D 81/28; A01N 25/34; A61L 15/18

(52) U.S. Cl. CPC A01N 59/00; A01N 25/34; A61L 15/44; A61L 15/18; A61L 15/425; B65D 81/28; C02F 1/76; A61L 2300/106; C02F 2303/04

(57) ABSTRACT

The present invention provides for a composition, including: a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the composition is contacted with an aqueous liquid, the sufficient first amount of the first active agent dispersion and the sufficient second amount of the at least one chlorite salt dispersion results in a generation of chlorine dioxide radicals.

### RELATED APPLICATIONS

This application claims the priority of U.S. provisional application U.S. Patent Appln. No. 62/019,678; filed Jul. 1, 2014; entitled “SYSTEMS AND METHODS FOR RELEASING CHLORINE DIOXIDE,” and U.S. provisional application U.S. Patent Appln. No. 62/142,290; filed Apr. 2, 2015; entitled “ADHESIVE COMPOSITIONS AND METHODS OF USE THEREOF,” which are incorporated herein by reference in their entireties for all purposes.

### TECHNICAL FIELD

In some embodiments, the present instant invention is related to compositions including at least one active agent dispersion and at least one chlorite salt dispersion and methods of use thereof.

### BACKGROUND

Chlorine dioxide radicals can be used to reduce a population of microorganisms. Microorganisms include, but are not limited to, bacteria, archea, fungi, and protists.

### SUMMARY OF INVENTION

In some embodiments, the present invention provides for a composition, including: a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the composition is contacted with an aqueous liquid, the sufficient first amount of the first active agent dispersion and the sufficient second amount of the at least one chlorite salt dispersion results in a generation of chlorine dioxide radicals at a rate ranging from 0.001 mg/min-0.02 mg/min. In some embodiments, the at least one chlorite salt dispersion is selected from the group consisting of: sodium chlorite, potassium chlorite, barium chlorite, calcium chlorite, magnesium chlorite, and any combination thereof. In some embodiments, the sufficient first amount of the first active agent dispersion is in a first layer, and the sufficient second amount of the at least one chlorite salt dispersion is in a second layer. In some embodiments, the active agent dispersion and the at least one chlorite salt dispersion are configured in the composition to define a plurality of cavities. In some embodiments, each cavity of the plurality of cavities measures between 0.5-50 micrometers in length. In some embodiments, the first active agent dispersion has a pKa of 0.1-1.5. In some embodiments, the composition is configured to allow for a water uptake measurement ranging from 10-90% over 1 hour. In some embodiments, the composition further includes: a substrate component in contact with of the first active agent dispersion or the at least one chlorite salt dispersion, where the substrate component includes polyethylene terephthalate, high-density polyethylene, low-density polyethylene, polypropylene, polystyrene, polyamide, polyvinylchloride, or any combination thereof. In some embodiments, the composition further includes: a protection component configured to reduce a reaction between the first active agent dispersion and the at least one chlorite salt dispersion, where the protection component includes an acrylic dispersion, a styrene acrylate dispersion, a polyurathene, an epoxy co-polymer, a cellulose, a polymer or copolymer dispersion, or any combination thereof, and where the protection component is in contact with at least the first active agent dispersion or the at least one chlorite salt dispersion. In some embodiments, the composition further includes: a neutralizing agent selected from the group consisting of: sodium thiosulfate, ferrous chloride, ferrous sulfate, vitamin E, and any combination thereof. In some embodiments, the composition further includes: a second active agent dispersion having a pKa of 0.1-2.0, where the at least one chlorite salt dispersion is in contact with the first active agent dispersion and the second active agent dispersion. In some embodiments, the sufficient second amount of the second active agent dispersion has a pKa of 0.1-1.5. In some embodiments, the sufficient first amount of the first active agent dispersion is in a first layer, where the sufficient second amount of the at least one chlorite salt dispersion is in a second layer, where the sufficient second amount of the second active agent dispersion is in a third layer, and where the second layer is positioned between the first layer and the third layer. In some embodiments, the composition further includes: a stabilizing agent selected from the group consisting of: ammonia, methylamine, sodium hydroxide, sodium bicarbonate, Purolite A200-MBOH, Dow FPA-55, a basic zeolite, and any combination thereof.

In some embodiments, the first active agent dispersion has a pKa of 0.1-1.0. In some embodiments, the first active agent dispersion has a pKa of 0.1-0.5. In some embodiments, the first active agent dispersion has a pKa of 0.5-2.0. In some embodiments, the first active agent dispersion has a pKa of 1.0-2.0. In some embodiments, the first active agent dispersion has a pKa of 1.5-2.0. In some embodiments, the first active agent dispersion has a pKa of 1.0-1.5.

In some embodiments, the plurality of particles has a median diameter of between 1-1000 micrometers. In some embodiments, the plurality of particles has a median diameter of between 10-1000 micrometers. In some embodiments, the plurality of particles has a median diameter of between 100-1000 micrometers. In some embodiments, the plurality of particles has a median diameter of between 500-1000 micrometers. In some embodiments, the plurality of particles has a median diameter of between 0.1-500 micrometers. In some embodiments, the plurality of particles has a median diameter of between 0.1-100 micrometers. In some embodiments, the plurality of particles has a median diameter of between 0.1-10 micrometers. In some embodiments, the plurality of particles has a median diameter of between 0.1-1 micrometers. In some embodiments, the plurality of particles has a median diameter of between 1-500 micrometers. In some embodiments, the plurality of particles has a median diameter of between 10-100 micrometers.

In some embodiments, the present invention provides for a composition, including: a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the sufficient second amount of the at least one chlorite salt dispersion is tested in the composition with the sufficient first amount of the first active agent dispersion, the sufficient first amount of the first active agent dispersion and the sufficient second amount of the at least one chlorite salt dispersion are contacted with an aqueous liquid, and chlorine dioxide radicals are generated at a rate ranging from 0.001 mg/min-0.02 mg/min so as to result in a microbial reduction of between 2 log CFU/mL and 10 log CFU/mL from between 1 and 60 minutes.

In some embodiments, the present invention provides for a composition including a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the composition is contacted with an aqueous liquid, the sufficient first amount of the first active agent dispersion and the sufficient second amount of the at least one chlorite salt dispersion results in a generation of chlorine dioxide radicals at a Cmax ranging from 15 ppm-25 ppm from between 4 hours-6 hours.

In some embodiments, the present invention provides for a composition, including: a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the composition is contacted with an aqueous liquid, the sufficient first amount of the first active agent dispersion and the sufficient second amount of the at least one chlorite salt dispersion results in a generation of chlorine dioxide radicals at a Cmax ranging from 5 ppm-15 ppm from between 10 hours-20 hours. In some embodiments, the present invention is a product, including an absorbant pad, where the absorbent pad includes the composition of claim 1. In some embodiments, the present invention is a product, including a package insert, where the package insert includes the composition of claim 1.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular component

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

FIG. 1 is an example of an embodiment of the composition of the present invention, showing a scanning electron microscope micrograph of the cross section of the two coating polymer matrix layers.

FIG. 2 is an example of an embodiment of the composition of the present invention, showing a scanning electron microscope micrograph of the surface of the polymer system showing clear surface defects.

FIG. 3 is an example of an embodiment of the composition of the present invention, showing a scanning electron microscope micrograph, the micrograph illustrating the cross section of the two coating layers.

FIGS. 4-7 are graphs showing chlorine dioxide radical release kinetics of exemplary embodiments of the compositions of the present invention.

FIGS. 8 and 9 are illustrations of exemplary embodiments of the compositions of the present invention, showing polymer matrix antimicrobial systems.

FIG. 10 is an illustrative exemplary embodiment of the composition of the present invention, showing a cross-section of a polymer matrix antimicrobial system.

FIG. 11 is an illustrative exemplary embodiment of the composition of a structure of an active coating of the present invention, a “sandwiched” configuration, on a milk carton.

FIG. 12 shows the coating is located on the top, bottom, middle, or a combination thereof, of the container.

FIG. 13 is an illustrative example of an embodiment of the composition of the present invention, showing an active chlorine dioxide radical solution and system scheme.

FIGS. 14A-14I are graphs of water uptake of exemplary embodiments of the compositions of the present invention.

FIG. 15 is a photograph showing embodiments of the compositions of the present invention.

FIGS. 16A and 16B are photographs showing assemblies of some embodiments of the compositions of the present invention.

FIGS. 17A and 17B show graphs of Clostridium perfringens viable counts after contact with some embodiments of the compositions of the present invention.

FIGS. 18 and 19 show graphs of Legionella viable counts after contact with some embodiments of the compositions of the present invention.

FIG. 20 shows some embodiments of reversed assemblies of the compositions of the present invention.

FIG. 21 shows viability counts of microorganisms after subjected to some embodiments of the compositions of the present invention.

FIGS. 22A-D show chlorine dioxide radical accumulation time derivative results of some embodiments of the compositions of the present invention.

FIG. 23 illustrates a chlorine dioxide radical measurement array, including a CDO display, a sheet, a humidity sensor, a chlorine dioxide radical sensor, and/or a water reservoir for use to assess some embodiments of the compositions of the present invention.

FIG. 24 illustrates some embodiments of the compositions of the present invention assemblies after 4 hours of immersion.

FIG. 25 shows some embodiments of the models of the present invention tested for CDO release over time.

FIGS. 26A and 26B show some embodiments of the apparatus used to record CDO release of the composition of the present invention, showing the measurement apparatus without fruit (FIG. 26A) and with fruit (FIG. 26B). FIGS. 27A-C show release kinetics, measuring CDO (ppm) over time (hours).

FIGS. 28A-28C show the action of an embodiment of the floating device.

FIGS. 29A and 29B show an embodiment of the floating device.

### DESCRIPTION OF EXEMPLARY EMBODIMENTS

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

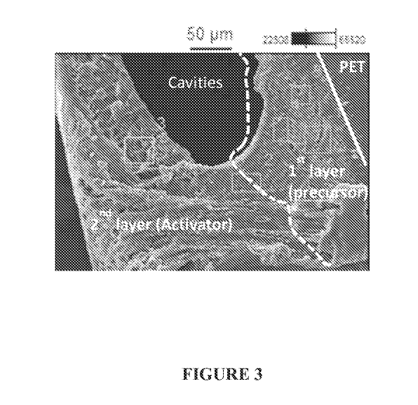

Some embodiments of the present invention are directed to a multi-layered antimicrobial coating structure that creates a singular system designed to generate an effective amount of chlorine dioxide radical (CDO) in a period of time and not to effect the surrounding medium. In some embodiments of the present invention, the period of time is between about 2 minutes to more than 4 hours, (e.g., a burst) that allows for a microbial killing ability. In some embodiments, the period of time is between 2 minutes and 3 hours. In some embodiments, the period of time is between 2 minutes and 2 hours. In some embodiments, the period of time is between 2 minutes and 1 hour. In some embodiments, the period of time is between 2 minutes and 30 minutes. For example, FIGS. 3-7 show various periods of time for CDO release kinetics with a minimum amount of active material and produces CDO. In some embodiments, the period of time is between 1 minute and 150 hours. In some embodiments of the present invention, the active material is a combination of acid and salt. In some embodiments, a minimum amount is measured up to a 6 log CFU/mL reduction in 30 minutes, e.g., AWT042, AWT047, and AWT048. In some embodiments, the minimum measures 5 ppm. In some embodiments, evaluation measurements are made according to EPA method 4500 CDO E, which is incorporated by reference in its entirety.

In some embodiments, CDO is released from the composition of the present invention for between 1 minute and 150 hours, where the CDO is released after an aqueous liquid contacts the composition of the present invention. In some embodiments, CDO is released for between 1 minute and 100 hours. In some embodiments, CDO is released for between 1 minute and 75 hours. In some embodiments, CDO is released for between 1 minute and 50 hours. In some embodiments, CDO is released for between 1 minute and 25 hours. In some embodiments, CDO is released for between 1 minute and 10 hours. In some embodiments, CDO is released for between 1 minute and 5 hours. In some embodiments, CDO is released for between 1 minute and 1 hour. In some embodiments, CDO is released for between 1 minute and 0.5 hours. In some embodiments, CDO is released for between 1 minute and 0.25 hours. In some embodiments, CDO is released for between 1 minute and 0.1 hours.

In some embodiments, CDO is released for between 0.1 hours and 150 hours. In some embodiments, CDO is released for between 0.25 hours and 150 hours. In some embodiments, CDO is released for between 0.5 hours and 150 hours. In some embodiments, CDO is released for between 1 hour and 150 hours. In some embodiments, CDO is released for between 5 hours and 150 hours. In some embodiments, CDO is released for between 10 hours and 150 hours. In some embodiments, CDO is released for between 25 hours and 150 hours. In some embodiments, CDO is released for between 50 hours and 150 hours. In some embodiments, CDO is released for between 75 hours and 150 hours. In some embodiments, CDO is released for between 100 hours and 150 hours.

In some embodiments of the present invention, a multi-layered structure contains, in close proximity, i) an acid in a solid state and ii) a salt and iii) polymers. In some embodiments of the present invention, the salt is sodium chlorite. In some embodiments of the present invention, the multi-layered structure includes i) a separating polymer, where the separating polymer physically separates the acid from the salt, and ii) a top layer of polymer, wherein the polymer prevents the entry of humidity into the structure. In some embodiments of the present invention, the multi-layered structure contains physical cracks. In some embodiments of the present invention, the multi-layered structure contains hydrophilic, expanding materials (e.g., solid acid) close to its surface. In some embodiments of the present invention, when the multi-layered structure is exposed to water, the multi-layered structure allows for the absorption of water from the bulk/target environment. In some embodiments, the absorption of water occurs between 5-15 minutes. In some embodiments of the present invention, the multi-layered structure allows for water entry through engineered cracks to generate CDO. In some embodiments of the present invention, the multi-layered structure is a singular system, where quantities of the present invention are sufficient for generating the CDO, and where the bulk/target environment is not significantly affected by the present invention. In one embodiment, “not significantly affected” means a measurement up to two pH units from an initial medium pH.

In some embodiments of the present invention, a polymer separates an acid and a salt by two or more layers, and an additional layer of polymer could be added on top of the top layer to separate the system from the environment liquid.

In some embodiments of the present invention, a chlorite salt is used to generate CDO. In some embodiments of the present invention, a chlorite salt may be any commercially available alkali metal or alkaline earth metal chlorite. In some embodiments, suitable metal chlorites include sodium chlorite, potassium chlorite, barium chlorite, calcium chlorite, magnesium chlorite, etc.

In some embodiments of the present invention, the multi-layered structure is designed such that: (i) the CDO generation is inhibited and not activated by moisture for a period of time (see, e.g., AWT081 for two weeks inhibition in 50% humidity and AWT082 for 70% humidity); (ii) the CDO is generated only when the structure is in contact with aqueous liquid; (iii) once in contact with liquid there is an engineered reaction between the structure and the aqueous liquid producing a burst of CDO (e.g., seconds to hours); (iv) the structure complies with industry requirements, where the amount, the multi-layered structure and the level of flexibility (e.g., no chipping or cracking when manual bending is applied to the product) and adhesion (e.g., cross cut and 3M scotch tape testing) allow for application as a coating for packaging, where the polymer binder, and where the singularity (i.e., describing the situation of small confined volume which has substantially different chemical properties (i.e., concentrations of specific ions and chemical species) then its surroundings) allows for substantially no effect on the product (e.g., up to 2 pH units from initial medium pH); (v) the yield of CDO generated by the reaction between the structure and the liquid is high (e.g., 100%, 90%, 80%) resulting in a high killing ability (see, e.g., AWT034 and AWT036, showing higher killing ability when the same quantity of sodium chlorite is activated by the present invention compared to other commercial solutions such as bulk addition of phosphoric acid) without the need for a large amount of acid (e.g., the volume of the multilayer coating is substantially less than the volume of the substrate) and chlorite salt; (vi) the chlorite residuals are limited because the generation of the CDO is from an engineered, closed system that facilitates a nearly complete conversion of chlorite from the salt to CDO (see, e.g., method 4500-CDO is the method used in Table 2); (vii) the structure generates a potent level (e.g., a potent level meaning a minimum ppm of chlorite converted to CDO that enables total eradicating; see, e.g., AWT042) of CDO without requiring additional biocides and/or chemicals for the activation of generation of CDO, such as chlorate and sodium dichloroisocyanurate and hypochloric acid; (viii) there is no need to affect (e.g., acidify) the liquid environment to generate the CDO to achieve a high killing efficiency; (ix) the structure does not produce a significant influence (i.e. change in pH level) in the liquid environment (singular); and (x) the structure does not dissolve into the environment and does not leave components, such as clay, solid and liquid acids (HCl, citric acid, and/or phosphoric acid) in the target environment/liquids.

In some embodiments of the present invention, the concentration of CDO in the target environment/liquid has a measurement that exceeds the required threshold of CDO necessary to kill a target microorganism population. In some embodiments of the present invention, the release provides an advantage of CDO chemical reaction end products that are constituents normally found in beverages (water and table salt) so the consumer is not exposed to any CDO material.

In an embodiment, the present invention is a solvent based coating system. In an embodiment, a first layer of the present invention is generated using three steps: i) dissolving a solid polymer in a solvent; ii) adding a powder form or water solution form of sodium chlorite to the suspension of step i); and iii) applying the system to a substrate (e.g. PET) and drying the substrate to form a film. In an embodiment, drying speed can be altered to change the porosity of the film and the time for the CDO burst to occur. In an embodiment, drying speed can be altered to modify surface cracks. In an embodiment, modification of surface cracks affects the water uptake kinetics of the film. In an embodiment, a second layer of the present invention is added to the first layer, which adds a cation exchange capability.

### Example 1: An Engineering Structure of the System

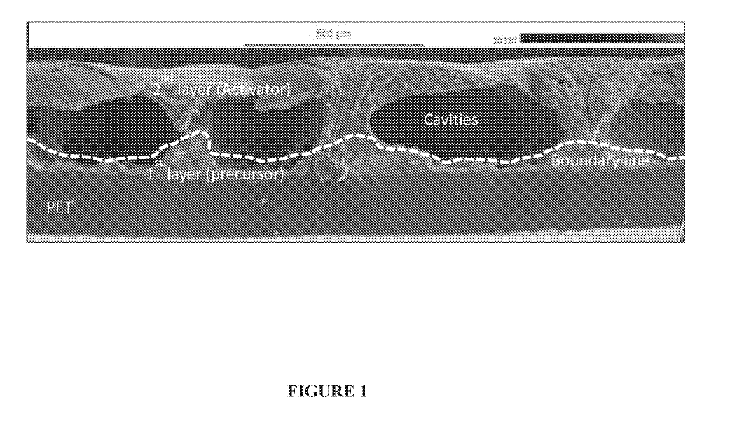

Some embodiments of the present invention are directed to an engineered system, singular and continuous, based on one or more polymeric coatings, hydrophilic and hydrophobic, anhydrous, where the engineered system allows the combination in close and substantially no contact proximity of the hygroscopic active raw materials (precursor and activator: sodium chlorite and strong cation exchanger), not to be adjacent, in at least 100 nm distance between them but no more than 500 μm. In one example, the sodium chlorite and cation exchanger are included in the film so that they are physically separated one from the other but permit water uptake upon immersion, or under exposure to humid environment (e.g., a humidity of less than 100%), thus creating water “bridge” between them. In some embodiments of the present invention, the hygroscopic active raw materials are separated by a distance between 100 nm and 100 μm. In some embodiments of the present invention, the hygroscopic active raw materials are separated by a distance between 500 nm and 100 μm. In some embodiments of the present invention, the hygroscopic active raw materials are separated by a distance between 1.0 μm and 500 μm. In some embodiments of the present invention, the hygroscopic active raw materials are separated by a distance between 10 μm and 500 μm. In some embodiments of the present invention, the hygroscopic active raw materials are separated by a distance between 200 μm and 500 μm. In some embodiments of the present invention, the hygroscopic active raw materials are separated by a distance between 100 nm and 1.0 μm. In some embodiments of the present invention, the hygroscopic active raw materials are separated by a distance between 1 μm and 200 μm. In some embodiments of the present invention, the hygroscopic active raw materials are separated by a distance between 1 μm and 100 μm. In some embodiments of the present invention, the hygroscopic active raw materials are separated by a distance between 1 μm and 10 μm. Some embodiments of the present invention are directed to a system with the reactive components are controlled and primed activated under different conditions. In an embodiment of the present invention, the system depicted in FIG. 1 describes the application of a double layer/coating (i.e., the first layer and the second layer). In some embodiments of the present invention, the first layer/coating consists of a precursor (e.g., NaClO₂) and has an irregular surface morphology (e.g., FIG. 1) and surface energy (post drying) higher (e.g., measured by “Dyne test kit” provided by Dyne Technologies) than the surface tension of the second layer coating that consists of the activator, including a cation exchanger, that is applied directly on the first dried coating/layer. In some embodiments of the present invention, the second layer/coating exhibits limited wetting on the first layer/coating and an interface is formed with hollow cavities. In some embodiments of the present invention, both layers/coatings go through a separate fast drying process that creates two effects. In some embodiments, the two effects are i) an immediate drying, i.e. evaporation of the carrier solvent, and ii) an increase in viscosity. In some embodiments of the present invention, the drying comprises evaporation of the carrier solvent, where an increase in viscosity occurs that generates an unleveled coating(s), both the first lower layer/coating and the second upper layer/coating. In some embodiments of the present invention, the process conditions are such that evaporation of a carrier solvent is non-uniform across the thickness of the layer/coating, evaporating first on the upper exposed side of the layer/coating, which forms a crust, where the crust traps the remaining solvent vapor inside the layer/coating. In some embodiments of the present invention, the solvent vapor accumulates in the cavities, and increases the pressure so that the cavities expand to substantial dimensions (e.g., up to hundreds of microns), allowing the vapors to escape by bursting through the capillary cracks of the layer/coating. In some embodiments of the present invention, the mechanism is an engineering structure and composition that facilitates a clear and distinct separation between the two reactive hygroscopic raw materials with almost no contact, and structured cavities that enable high efficiency reaction zone (see, e.g., FIG. 1 showing porous cavities that contain and react the salt and acid), only upon aqueous fluid exposure, that dissolves the protons from the activator and the chlorite ion to an acidic environment activity zone with low pH. In some embodiments, the low pH is <3. In some embodiments, the low pH is <2. In some embodiments, the low pH is <1.5. In some embodiments, “almost no contact” means between 0.1-200 μm. In some embodiments, “almost no contact” means between 0.1-300 μm. In some embodiments, “almost no contact” means between 0.1-400 μm. In some embodiments, “almost no contact” means between 0.1-500 μm. In some embodiments, “almost no contact” means between 1-500 μm. In some embodiments, “almost no contact” means between 10-500 μm. In some embodiments, “almost no contact” means between 100-500 μm. In an embodiment of the present invention, the reaction between the two hygroscopic raw materials produces the CDO inside the cavities, identified herein as “lacunar accelerated-activation sites.” In some embodiments of the present invention, liquid water penetrates a polymer matrix through structural defects on the layer/coating surface and throughout the matrix, down towards the cavities in a self-accelerating process. In some embodiments, after the chemical reaction and the generation of the antimicrobial agent, CDO, a chemical potential gradient is obtained between the system and the target fluid. In some embodiments of the present invention, the acceleration of water penetration and the chemical potential gradient increases the rate of release, where a burst of CDO is created from within the multi-layered system to the target liquid that is in direct contact with the multi-layered system.

In some embodiments of the present invention, the pH changes only inside a matrix. In some embodiments, the pH is <3. In some embodiments, the pH is <2. In some embodiments, the pH is <1.5.

Diverse Polymeric Matrixes Systems

In some embodiments of the present invention, diverse polymeric matrixes systems comprise at least one layer/coating. In some embodiments of the present invention, the layer/coating consists of a hydrophobic polymer, solvent soluble, with low surface energy (between 20-60 dyne/cm), that inhibits the diffusion and penetration of water in liquid state, and does not condense or absorb water vapor. In some embodiments of the present invention, the layer/coating prevents penetration of water into the matrix. Although the coating comprises hygroscopic materials, there is no condensation and water absorption and thus no undesired activation at an unwanted timing occurs even at significant humidity (e.g., 80%) (see, e.g., AWT081 and AWT082) unless a substantial condensation occurs on the surface of the coating.

In some embodiments of the present invention, the system comprises multiple coatings/layers, where the first coatings/layers implemented on the substrate are made of a formulation comprising an emulsion water based polymer or a solvent based polymer solution/emulsion/dispersion and the active precursor. In some embodiments, the number of coatings/layers is between 2-10. In some embodiments, the number of coatings/layers is between 2-8. In some embodiments, the number of coatings/layers is between 2-6. In some embodiments, the number of coatings/layers is between 2-4. In some embodiments, the coating includes at least one hydrophobic layer (e.g., but not limited to, 1 layer, 2 layers, 3 layers, 4 layers, 5 layers, etc.) and at least one hydrophilic layer (e.g., but not limited to, 1 layer, 2 layers, 3 layers, 4 layers, 5 layers, etc.). In some embodiments, the number of coatings/layers is one (i.e., a single-layer coating). In some embodiments of the present invention, after drying of the first layer/coating, the second layer/coating is applied which is formulated of a polymer dissolved in a solvent and a strong cation exchanger. In some embodiments of the present invention, the solvent is water free (e.g., no water was intentionally added). In some embodiments of the present invention, the strong cation exchanger is acidic. In some embodiments of the present invention, post drying the system comprises of hydrophobic polymers containing hygroscopic reactive materials. In some embodiments of the present invention, post drying the system further comprises the use of reinforcing agents that facilitate a structural modification. In some embodiments of the present invention, the structural modification comprises: i) at least one precursor, ii) at least one activator, and iii) at least one filler. In some embodiments of the present invention, the at least one precursor is an active material comprising chlorite metal salt. In some embodiments of the present invention, the at least one activator is a strong fixed proton donor with pKa<3. In some embodiments, the pKa is <2. In some embodiments, the pKa is <1.5. In some embodiments of the present invention, the at least one filler comprises at least one reinforcing structural agent.

TABLE 1
In some embodiments of the present invention, Table 1 illustrates a Hach measurement of the chlorite
and CDO content at 1 h and 4 h post water exposure of models, consisting of a solvent based coating
in accelerated aging conditions tested at extreme temperature and humidity for 4 weeks.

| | | 1 h | | | | 4 h | | | |
|---|---|---|---|---|---|---|---|---|---|
| Model | | | | total | | | | total | |
| # | model description | CDO | ClO2— | ClOx's | pH | CDO | ClO2— | ClOx's | pH |
| 1 | Fresh film | 5.104 | Below | 5.104 | 4.68 | 3.964 | Below | 3.964 | 4.19 |
| | Layer 1 - Hydrophobic | | detection | | | | detection | | |
| | binder + CG8-H | | limit | | | | limit | | |
| | Layer 2 - Hydrophobic | | | | | | | | |
| | binder + NaClO₂ | | | | | | | | |
| | Layer 3 - Hydrophobic | | | | | | | | |
| | binder + CG8-H | | | | | | | | |
| 2 | Aged film @ 40° C. & 80% | 1.936 | Below | 1.936 | 4.42 | 2.892 | Below | 2.892 | 4.26 |
| | humidity oven | | detection | | | | detection | | |
| | Layer 1 - Hydrophobic | | limit | | | | limit | | |
| | binder + CG8-H | | | | | | | | |
| | Layer 2 - Hydrophobic | | | | | | | | |
| | binder + NaClO₂ | | | | | | | | |
| | Layer 3 - Hydrophobic | | | | | | | | |
| | binder + CG8-H | | | | | | | | |

Some embodiments of the present invention are presented in Table 1, illustrating that the system is potent after accelerating aging condition model, and produces substantially the same amount of CDO without any chlorite residue when compared to an as prepared sample. In some embodiments, the present invention preserves and sustains the active system for a period of at least 4 weeks under severe conditions.

In some embodiments of the present invention, the system components comprising an activator and a precursor, are inside the polymeric coating layer. In some embodiments of the present invention, the system components create a singular well-defined and continuous system. In some embodiments of the present invention, an active salt, wherein the active salt is comprised of a chlorite ion, that is the initiating factor of the reaction. In some embodiments of the present invention, an excess of the proton donor, namely the cation exchanger, is present (see, e.g., AWT024). In some embodiments of the present invention, the active materials are separated to a point of substantially no contact between them yet maintain a closely adjacent distance. In some embodiments of the present invention, although the activator and the precursor consist of a residual amount of water, the system maintains activity of the activator and the precursor for a period of time (see, e.g., AWT086, e.g., 1 year).

In some embodiments of the present invention, the present invention comprises at least one structural discontinuum defect of the polymer coating surface volume (i.e., bulk). In some embodiments of the present invention, structural defects facilitate capillary flow of water into the coating depth (see, e.g., FIG. 1). In some embodiments of the present invention, at least one structural characteristic, where the structural characteristic is the structural discontinuum defect, is a controlled parameter and determined by the system method of preparation. In some embodiments of the present invention, the system method of preparation comprises: i) drying temperature, ii) drying time, iii) time between application and the drying process, iv) layer thickness, v) layer order (see, e.g., AWT048: active salt is the upper layer in contact with the medium resulting in reduced efficacy and shelf life), vi) volume fraction of the different components in the formulation, wherein the volume fraction include additives, vii) polymer matrix (binder) selection, and viii) solvent used in the system. In some embodiments, the specific property of the polymer that will determine film morphology is glass transition temperature (T_{g}). In some embodiments, T_{g} of the binders determines the flexibility of the coating. In some embodiments of the present invention, the system characteristics comprise a method of application, where the method of application is selected from the group consisting of: spraying, drawdown, flexo, screen printing, coating heads slot di, and a combination thereof.

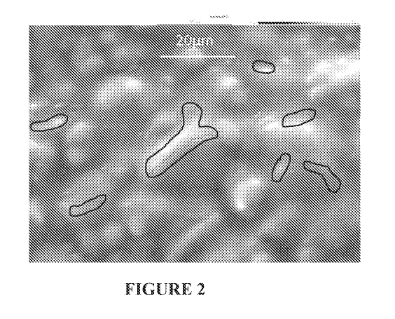

In some embodiments of the present invention, defects dimensions are characterized by: 1) length, wherein length is measured between 1-100 μm, 2) width, wherein width is measured between 0.1-10 μm, 3) depth, where depth is measured between 0.1-50 μm, and 4) defects surface density measuring between 1 discontinuum/25 μm²-1 discontinuum/2500 μm² (see, e.g., FIG. 2).

In some embodiments of the present invention, water molecules penetrate the system and immediately absorbed by a proton donor, where the proton donor is a strong cation exchanger, where the proton donor is physically present in the first contact layer in connection with the outer target product/environment. In some embodiments of the present invention, two progressions are present: 1) the swelling of the cation exchanger and 2) the release of the protons to the cavities. In some embodiments of the present invention, the swelling of the cation exchanger causes a distortion/deformation of a system matrix, where the defects expand and allow faster water penetration, where faster water penetration accelerates the activation phase. In some embodiments of the present invention, a release of the protons induces a pH reduction of the absorbed water in close proximity and only in the confined system matrix to a low pH value, and facilitates a reaction of a chlorite species to CDO. In some embodiments, the low pH measures approximately 3. In some embodiments, the low pH measures approximately 2. In some embodiments, the low pH measures approximately 1.5.

In some embodiments of the present invention, particle size distribution and of a proton donor particles, where the proton donor is a strong cation exchanger, in a matrix constitutes a parameter that defines the optimal particle package that enables the conditions for the activation of the chlorite. In some embodiments of the present invention, a particle size has a median diameter (D₅₀) of 10 μm. In some embodiments of the present invention, a particle size has a D₅₀ of 0.5 μm. In some embodiments of the present invention, a particle size has a D₅₀ of 100 μm.

In some embodiments of the present invention, a flow of acidified water penetrates through the expended defects and the discontinuums, wets and dissolves the hygroscopic chlorite salt and immediately initiates a chemical reaction between a chlorite ion and a proton, wherein the chemical reaction generates CDO. In some embodiments of the present invention, the water penetration and CDO release kinetics is strongly dependent on the layer/coating thickness. In some embodiments of the present invention, coating thicknesses of 1-100 μm, the kinetic dependency and the release of CDO is substantially dominant at low layer/coating thickness. In some embodiments of the present invention, a CDO burst is achieved in a short period of time. In some embodiments of the present invention, the specific time of burst is calibrated to a specific need, with a length and release intensity calibrated by adjusting the layer/coating thickness and polymer choice.

In some embodiments of the present invention, to obtain a CDO burst at the target bulk medium, water must penetrate a system polymer matrix, where a minimal 5% weight increase occurs (system dependent) within approximately 30 minutes of exposure of the system to the target bulk medium. In some embodiments of the present invention, the water penetrating the system must contact i) a proton donor (e.g., a strong cation exchanger) and iii) a chlorite salt, where chemical reaction produces CDO.

In some embodiments of the present invention, prevention of water penetration to the matrix halts the chemical reaction that produces CDO. In some embodiments of the present invention, prevention of water penetration to the matrix maintains shelf life and potency, a temporal inert top coating or a constant inert top coating, where the temporal inert top coating or the constant inert top coating seals the multilayered structure from water and protects the system from an early activation. In an embodiment of the present invention, the top coat is removable or is inert and loses sealing capabilities over a predetermined period of time or under predefined conditions, such as temperature, pH etc., where water is capable of penetrating the system and initiating the required reaction at a desired onset time. In some embodiments, the top coat is an adhered sealing top coat, where the adhered sealing top coat is removed at a required activation time. In some embodiments of the present invention, the top coat is a humidity coating sealer that swells at 100% humidity and becomes a water permeable layer.

In some embodiments, the present invention includes no top barrier layer. In some embodiments, the coating with no barrier layer are configured such that when stored in a closed bag or equivalent, humid air alone provides insufficient quantity of water or hydrostatic pressure to adequately penetrate the coating to initiate CDO generation via the proton donor and the chlorite salt.

In some embodiments of the present invention, processes occurring inside the system matrix develop a singular distinct environment from the surrounding bulk target medium. In some embodiments of the present invention, the distinct environment is generated by the polymer being physically separated from the target medium and is characterized with compositions and features essentially different and unique from the target medium. In some embodiments, the system uses a low mass of the activator (cation exchanger), having no or little effect on the target medium bulk pH (having low pH, below 2, inside the system that is optimal to initiate the CDO generation).

In some embodiments of the present invention, the CDO concentration increases inside the system coating, where the system coating comprises the matrix, and the chemical potential gradient of the CDO increases versus the target medium bulk). In some embodiments of the present invention, a minimal value to be implemented in the system is approximately 0.1 J as calculated in an example system below:
Assumptions—CDO concentration is 10 ppm, liquid target volume is 500 cc, and temperature is 25° C. (298K)

$$Δμ = {RTlnC}_{CDO} = 8.314 [ \frac{J}{mol ⋆ K} ] ⋆ 298 [ K ] ⋆ ln 10 = 5704.8 [ \frac{J}{mol} ]$$

$$Δμ = 5704.8 [ \frac{J}{mol} ] * 10 [ \frac{mg}{L} ] * \frac{1}{67 \text{,} 452} [ \frac{mol}{mg} ] * 0.5 [ L ] = 0.423 J$$

In some embodiments of the present invention, the concentration gradient (i.e., potential delta) between the system matrix and the target medium bulk is the driving force for the CDO burst to the target medium bulk where the potential is low. In some embodiments of the present invention, CDO then reacts in the target medium bulk, (for example, see FIGS. 3-7) and consumed, maintaining the gradient.

In some embodiments, as long as the CDO source is not completely depleted, the CDO concentration surrounding the source is still at higher than in the bulk even if the bulk concentration is increasing.

In some embodiments of the present invention, a spatial effect occurs. In some embodiments of the present invention, the spatial effect occurs in the location of the singular system, where the spatial effect releases CDO in the target medium bulk. In some embodiments of the present invention, the spatial location in the target medium bulk determines the dispersion rate and the dose of the active CDO in a medium volume. In some embodiments of the present invention, the chemical potential and/or concentration gradient control the rate for dispersing the CDO across the entire target medium bulk. In some embodiments of the present invention, the effective and controlled release of the ingredient considering the 3-dimensional characteristics of the target medium bulk, both peripheral and volume, promotes antimicrobial activity when compared with a one dimensional/narrow space (i.e. bottom only, side only and top only engineered solution). In some embodiments of the present invention, the distances and the relative singular weight and position of the CDO generated in the coating system is compared to concentration of CDO in the liquid/gas volume of the target product. In some embodiments of the present invention, additional factors that influence the spatial effect are selected from a group consisting of: viscosity, temperature, solid/gas particles, and type of medium.

In some embodiments of the present invention, specific conditions are required to initiate a CDO burst from the polymer matrix. In some embodiments of the present invention, the specific conditions are: 1) presenting a proton donor comprising a pKa<2, 2) having at least one structural defect of the polymer matrix, 3) having proximity conditioning, and 4) presenting a singular system. In some embodiments, the method further includes 5) having an active system post-applying pre-wash, 6) applying stirring and homogenization in an aqueous system, and/or 7) containing a neutralizing phase. In some embodiments of the present invention, the proton donor is a cation exchanger (e.g., CG8-H by ResinTech). In some embodiments of the present invention, a pKa of sulfonic acid groups of poly(styrene sulfonate) (PSS) is 1.

TABLE 2
In some embodiments of the present invention, Table 2 illustrates the pKa of the
proton donor and the amount of CDO generated and released within 1 hour and 4 hours
of exposure to water under the same conditions and in the same matrix system.

| | | 1 h | | | | 4 h | | | |
|---|---|---|---|---|---|---|---|---|---|
| Model | | | | pH | Water | | | pH | Water |
| # | Model | CDO | ClO2— | (bulk) | uptake | CDO | ClO2— | (bulk) | uptake |
| 1 | Hydrophobic | 4.675 | 0 | 4.49 | 80.7% | 4.635 | 0 | 4.35 | 90.5% |
| | binder\NaClO2\Proton | | | | | | | | |
| | donor pKa = 1 | | | | | | | | |
| 2 | Hydrophobic | <DL | 8.588 | 6.65 | ND | ND | ND | ND | ND |
| | binder\NaClO2\Proton | | | | | | | | |
| | donor pKa = 4.6 (WAC-G) | | | | | | | | |
| 3 | Hydrophobic | <DL | 10.44 | 6.65 | ND | ND | ND | ND | ND |
| | binder\NaClO2\Proton | | | | | | | | |
| | donor pKa = 6 (Kamin) | | | | | | | | |
| 4 | Aquamira pKa | 0.304 | 8.172 | 3.55 | ND | 1.032 | 3.464 | 3.55 | ND |
| | phosphoric acid = 2.12 | | | | | | | | |

In some embodiments of the present invention, as presented on Table 2, the use of a strong fixated acid donor with pKa<2 (i.e. cation exchanger) in model 1, inside a singular system, facilitates the conversion of the chlorite ion in the early stages of the reaction and gives superior results with only CDO detected by the Hach measurement with no chlorite residuals or significant change to the target medium pH. In some embodiments of the present invention, e.g., model 4, which uses a free acid and is therefore, not a singular system, has a significant influence on the pH of the medium and does not exploit the system to the full capacity/capability, only generates a fraction of CDO and mostly releases chlorite residuals. In some embodiments of the present invention, e.g., models 2 and 3, a weak acid donor does not significantly convert chlorite to CDO within the timed parameters of testing. In some embodiments of the present invention, a strong acid proton donor, where the strong acid proton donor is fixated in a singular system, increases efficiency and produces a CDO burst.

In some embodiments of the present invention, a structural defect and a discontinuum of the polymer, promote water penetration into a hydrophobic polymer matrix system. In some embodiments of the present invention, according to the Young-Dupré equation, the contact angle of water with surface tension of 72.9 dyne/cm on a polymer hydrophobic surface with surface energy below 40 dyne/cm is θ>90°, promoting de-wetting of the surface and substantially hydrophobic. In some embodiments of the present invention, when a structural defect is present, the hygroscopic/hydrophilic raw materials are exposed and allow wetting of the crevasses and liquid water to penetrate to the cavities.

In some embodiments of the present invention, proximity conditioning is the presence of active raw materials, where the active raw materials are i) chlorite salt and ii) cation exchanger, in contiguity, yet separated with almost/substantially no contact, by a hydrophobic polymer. In some embodiments of the present invention, FIG. 3 illustrates two layers: i) a first layer containing the chlorite ion, and ii) a second layer containing the cation exchanger, where the first and second layers are one on top of the other. In some embodiments of the present invention, combining the Energy Dispersive Spectroscopy (EDS) data in Table 3 and analyzing the elements of the layers, the 1^{st} layer close to the poly ethylene tertphthalate (PET), referred to as “pt1” and “pt5” in Table 3, has no sulfur indication, a key element in the cation exchanger. In some embodiments of the present invention, the 2^{nd} layer, referred to as “pt3” in Table 3, illustrates sulfur only at sharp peak measured by EDS, which corresponds with the presence of the cation exchanger and no chlorite. In some embodiments of the present invention, points 2 and 4 in FIG. 3 correspond to a boundary layer (point 4 being close to the cavity boundary zone) where both the cation exchanger and the chlorite salt (represented by the sodium counter part of the chlorite ion) are present, and as seen in Table 3, “pt2” and “pt4” contain both elements sodium and sulfur.

TABLE 3
Table 3 illustrates some embodiments of the present invention, presenting
EDS analysis results of Figure 3. In some embodiments of the present
invention, sulfur represents the presence of the cation exchanger, and
sodium (Na⁺) represents the counter ion with a chlorite ion. In some embodiments
of the present invention, a Cl element is present in the polymer is not significant.

| | C-K | O-K | Na-K | S-K | Cl-K |
|---|---|---|---|---|---|
| pt1 | 11.05 | 19.79 | 18.21 | 0.00 | 50.96 |
| pt2 | 27.60 | 25.89 | 0.73 | 12.19 | 33.57 |
| pt3 | 35.21 | 30.71 | | 12.06 | 22.01 |
| pt4 | 22.04 | 25.62 | 7.12 | 6.04 | 39.18 |
| pt5 | 15.46 | 18.23 | 9.38 | 0.00 | 56.93 |

In some embodiments of the present invention, a singular system is a well-defined separate and different system compared to the characteristics of the medium bulk or surrounding where the CDO generation occurs. In some embodiments of the present invention, a parameter is the pH, where the pH is measured for the surface of the singular system and for the target bulk medium and the results are presented in the following paragraph:

pH_{surface}=1.04<<4.66=pH_{bulk}.

In some embodiments of the present invention, the present invention comprises an active system post-applying pre-wash. In some embodiments of the present invention, the active system is regulated to activate at a calculated time, where activation comprises a CDO burst, a pre-wash procedure using an aqueous wash i.e. sterile water, hydrogen peroxide (H₂O₂), acidified sodium chlorite, acidic solution etc. to produce a controlled pre-activation mechanism, allowing for the sterilization of the target active container prior to filling the container with the target medium. In some embodiments of the present invention, the process can also be applied to alter calculated CDO burst.

In some embodiments of the present invention, an aqueous target system is stirred and homogenized. In some embodiments of the present invention, a physical and/or a mechanical stirring of the liquid medium containing the produced CDO, is applied in an effective period (using, e.g., shakers/mixers/stirrers/ultrasonic, etc.) (see, e.g., AWT052). In some embodiments of the present invention, the effective period is 10 min after system activation. In some embodiments of the present invention, the effective period is 1 hr after system activation. In some embodiments of the present invention, the effective period is 3 hrs after system activation. In some embodiments of the present invention, the effective period is 24 hrs after system activation. In some embodiments of the present invention, the effective period is between 10 min and 3 hr after system activation. In some embodiments of the present invention, the effective period is between 10 min and 24 hrs after activation. In some embodiments of the present invention, the effective period is between 10 min and 150 hrs after activation.

In some embodiments of the present invention, a neutralizing phase is an engineered control release system. In some embodiments of the present invention, the neutralizing phase controls and reduces the chlorite and CDO chemical moieties, after a calculated activation phase (upon completion of the sterilization phase). In an embodiment of the present invention, the following steps comprise: 1) adding the neutralizer agent in a formulation with a binder and a controlled release and/or controlled exposure is set to a time when the system finished the required CDO release phase, where the neutralizer is selected from materials that react with CDO such as a group consisting of: a) Sodium thiosulfate, Na₂S₂O₃, b) Ferrous chloride, FeCl₂, c) Ferrous sulfate, FeSO₄, d) Vitamin E, and e) any combination thereof; and 2) adding a fixated coating on the surface of the container and neutralizing the species CDO/chlorite by surface contact, where the fixated coating is a polymer matrix comprised of neutralizing agent, e.g. FPA-55. In some embodiments of the present invention, the initial NaClO₂ concentration is equal in all models and measures 10 ppm.

In some embodiments of the present invention, the total quantitative release in each system varies and is dependent on additional variables such as: matrix impermeability, detector sensitivity and sampling time. In some embodiments of the present invention, a series of experiments that measure, monitor and control all variables is required to quantitate the total sum.

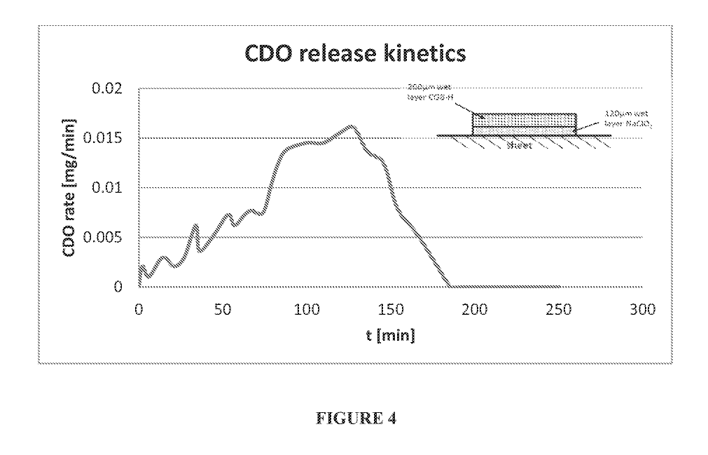

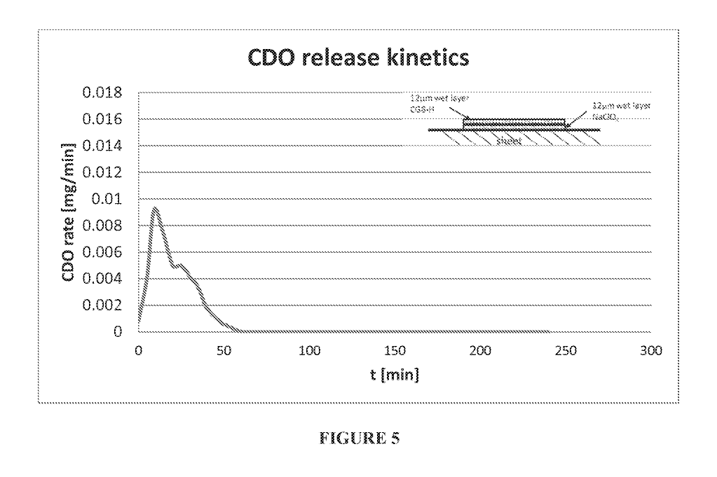

In some embodiments of the present invention, a comparison of FIGS. 4 and 5 illustrates that a layer thickness of 12 μm consisting of the precursor chlorite salt, a peak of ˜0.01 [mg/min] CDO is measured at 5 minutes. In some embodiments of the present invention, a thickness layer of 120 μm consisting of the precursor chlorite salt registers a similar peak ˜0.01 [mg/min], yet is measured at 140 minutes. In some embodiments of the present invention, an increase in the thickness of the salt layer inhibits the CDO release. In some embodiments of the present invention, the water penetration time (i.e., diffusion) is extended with a thicker layer, thus only delaying the burst and not suppressing it.

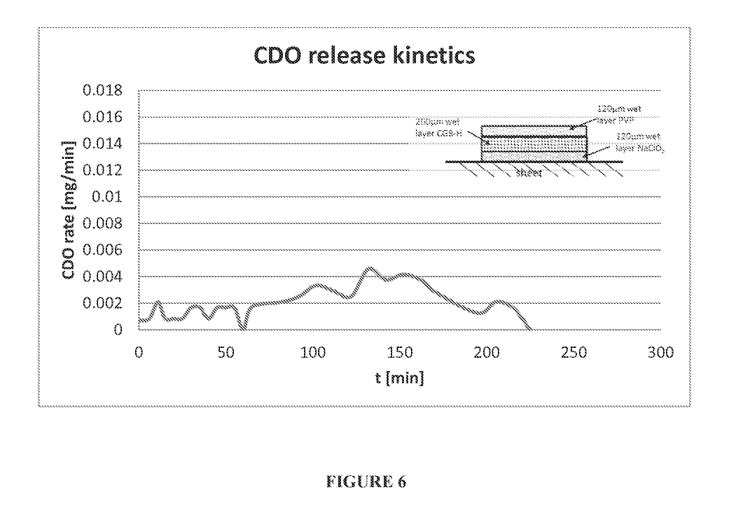

In some embodiments of the present invention, a comparison of FIGS. 4 and 6 illustrates: when coating the outer layer with an additional inhibiting polymer, a 3^{rd} layer is generated. In some embodiments of the present invention, the 3^{rd} layer provides an extension in the release time, measuring approximately 20% added time, and suppresses a CDO peak of a third of its original measurement. In some embodiments of the present invention, this system blocks i) the CDO rate of generation and ii) the rate of release.

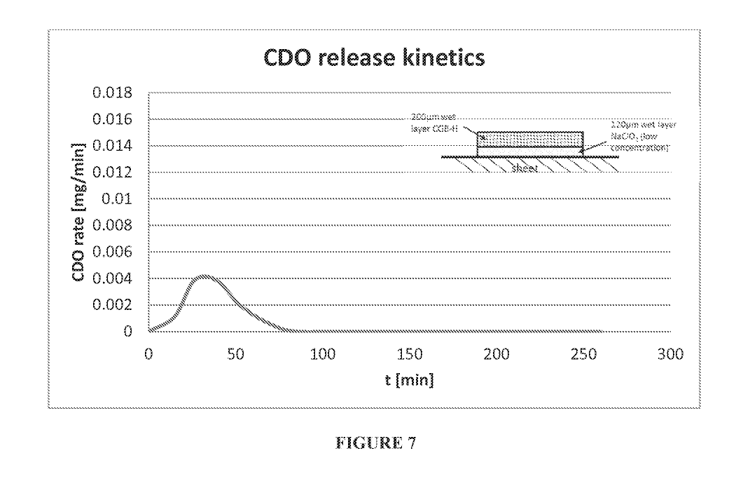

In some embodiments of the present invention, a comparison of FIGS. 4 and 7 illustrates that reducing the volume concentration of the chlorite salt results in a reduction of the total CDO released by an order of magnitude. As used herein, pigment volume concentration (i.e., “PVC”) means the total volume fraction (concentration) of fillers in the polymer matrix, including active salt cation exchange and any other filler in the polymer matrix. In some embodiments of the present invention, the blocking generated by the relative increase of the hydrophobic polymer binder used in the formulation causes a reduction in CDO rate of generation and release.

In some embodiments of the present invention, a chemical reaction related with the generation of CDO occurring inside the polymer system comprises: i) initial exposure of the strong acid, proton donor, to water causes complete dissociation of the acid and the release of the proton to the water inside the polymer matrix system, expressed in the reaction:

HA [symbol] H⁺+A⁻ pKₐ<2

dropping the pH of the water inside the polymer matrix system to the pKa of the acid; ii) the penetration of the acidified water to the 1^{st} layer containing the chlorite salt dissolving the salt, expressed in the reaction:

NaClO₂_{_{(aq)}}→Na⁺_{(aq)}+ClO₂⁻_{(aq)}

iii) The reaction of the protons in the acidified water with the dissolved chlorite ion to produce chlorous acid, expressed in the reaction:

ClO₂⁻+H⁺ [symbol] HClO₂ pKₐ=1.86

the low pH of the water drives the reaction towards the chlorous product; and iv) reaction of the chlorous acid generates and releases the CDO radical:

HClO₂→ClO*₂

In some embodiments of the present invention, the rate of CDO generation increases with elevated temperatures and lower pH values (acidity). In some embodiments of the present invention, a summary reaction is: 5HClO₂→4ClO*₂+Cl⁻+H⁺+2H₂O.

In some embodiments, the CDO radical decomposition reaction rate is rapid, at about 10̂9 M̂−1 ŝ−1.

In an embodiment of the present invention, FIG. 1 is an illustrative example of a scanning electron microscope micrograph of the cross section of the two coating polymer matrix layers. In an embodiment of the present invention, a 1^{st} layer (on PET substrate) consists of the precursor (NaClO₂) and the 2^{nd} layer consists of the cation exchanger. In an embodiment of the present invention, a boundary layer comprising cavities as long as 500 μm in length and 200 μm in height is present.

In an embodiment of the present invention, FIG. 2 is an illustrative example of a scanning electron microscope micrograph of the surface of the polymer system showing clear surface defects.

In this patent the following alternative designs are engineered layers that show the possible structures of the polymer matrix layers (non-limiting):

| Model | First layer | | |
|---|---|---|---|
| name | (on top of | | |
| material | the PET substrate) | Second layer | Third layer |
| Regular | Activator (CG8-H, | Precursor (sodium | None or inert |
| | cation exchanger) | chlorite salt) | |
| Reverse | Precursor (sodium | Activator (CG8-H, | None or inert |
| | chlorite salt) | cation exchanger) | |
| Sandwich | Activator (CG8-H, | Precursor (sodium | Activator (CG8-H, |
| | cation exchanger) | chlorite salt) | cation exchanger) |

In an embodiment of the present invention, FIG. 3 is an illustrative example of a scanning electron microscope micrograph, the micrograph illustrating the cross section of the two coating layers. In an embodiment of the present invention, a 1^{st} layer (on PET substrate) consists of the precursor (NaClO₂) and the 2^{nd} layer consists of the cation exchanger. In an embodiment of the present invention, the micrograph was analyzed with EDS for elemental analysis.

In an embodiment of the present invention, FIG. 4 is an illustrative example of a reverse 200 μm wet layer CG8-H placed on top of a 120 μm wet layer NaClO₂ at relative humidity 75%, 25° C. In an embodiment of the present invention, water up-take measured 32%.

In an embodiment of the present invention, FIG. 5 is an illustrative example of a reverse 12 μm wet layer CG8-H placed on top of a 12 μm wet layer NaClO₂.

In an embodiment of the present invention, FIG. 6 is an illustrative example of a reverse 200 μm wet layer CG8-H placed on top of a 120 μm wet layer NaClO₂ where the top cover comprises PVP polymer 120 μm.

In an embodiment of the present invention, FIG. 7 is an illustrative example of a reverse 200 μm wet layer CG8-H placed on top of a 120 μm wet layer NaClO₂ (low Poly vinyl chloride).

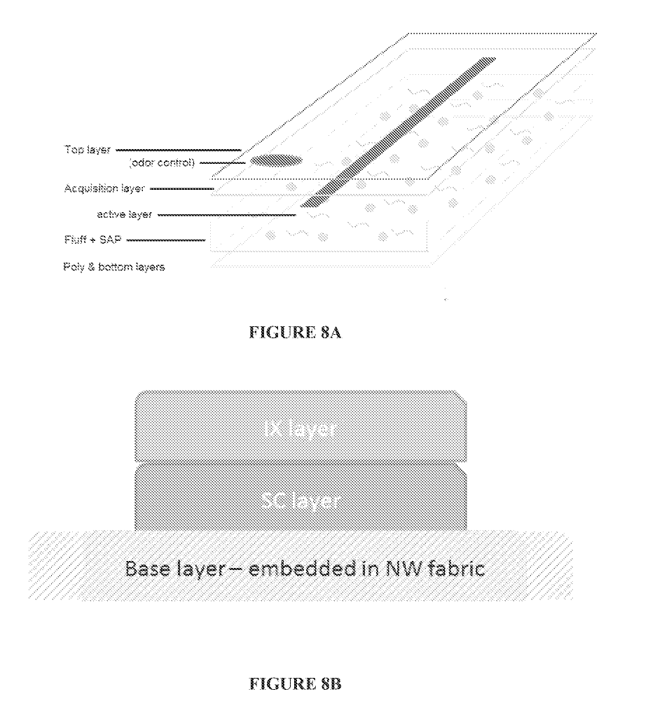

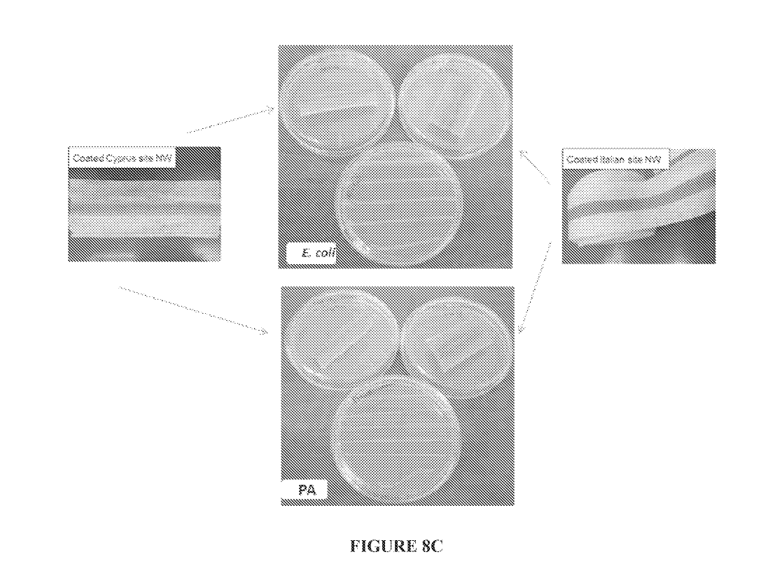

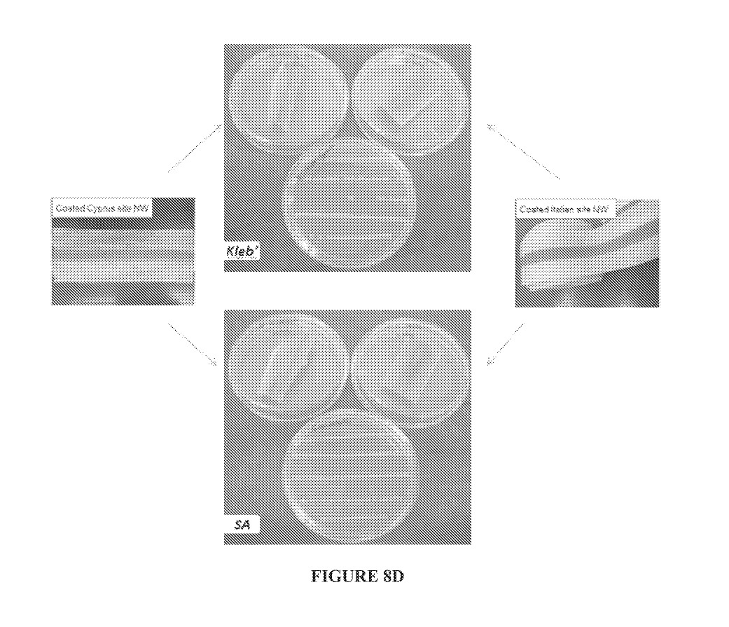

In an embodiment of the present invention, FIGS. 8A and 8B are illustrative exemplary embodiments of the composition incorporated into a diaper. FIGS. 8C and 8D illustrate the anti-microbial effect of using an embodiment of the composition of the present invention.

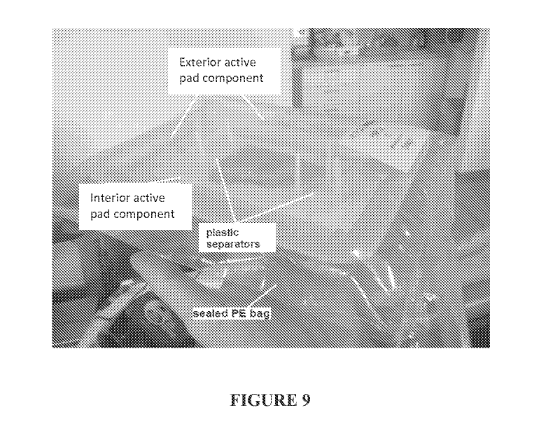

In an embodiment of the present invention, FIG. 9 is an illustrative example of the polymer matrix antimicrobial system inserted into a meat wrap.

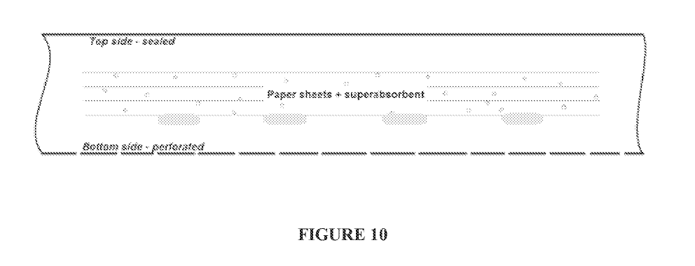

In an embodiment of the present invention, FIG. 10 is an illustrative example of the polymer matrix antimicrobial system inserted into an active pad (cross section).

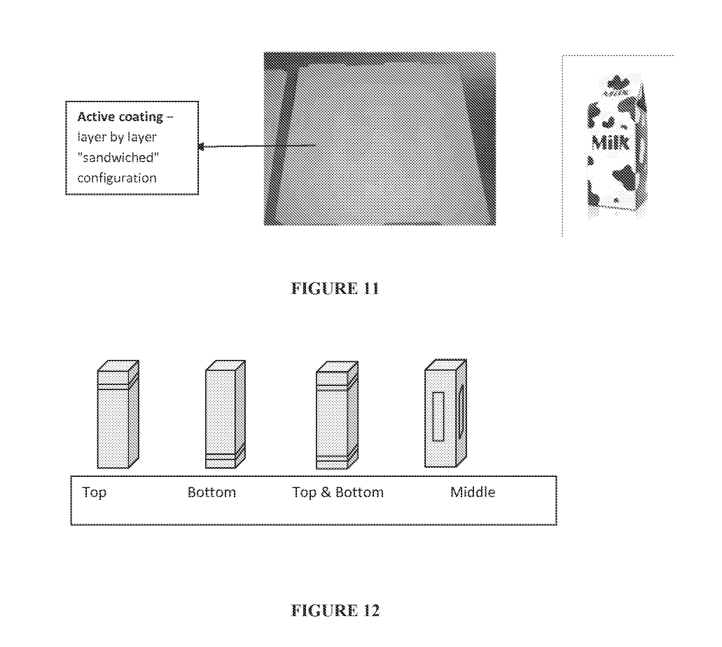

In an embodiment of the present invention, FIG. 11 is an illustrative example of a structure of an active coating, a “sandwiched” configuration, on a milk carton. In some embodiments, FIG. 12 shows the coating is located on the top, bottom, middle, or a combination thereof, of the container.

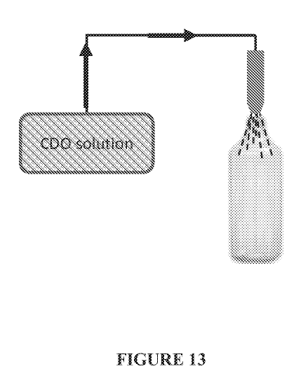

In an embodiment of the present invention, FIG. 13 is an illustrative example of an active CDO solution and system scheme. Large and rapid water uptake profile is essential for obtaining rapid CDO activation and release kinetics. In some embodiments, CDO is pumped through a system that delivers CDO into bottles/containers. The active CDO solution is prepared by activating the polymeric matrix system in water. The Experimental procedure is as follows: (1) a model specimen is sliced to a known area (3 triplicates), (2) the dry specimens are weighed using an analytical balance, (3) the samples are submerged in a beaker filled with 50 ml of DDW, (4) after 0.5, 1, 2, 3, 5, 10, 15, 30, 60, and 240 min, the specimens are removed from the water, wiped from excess water using a clean wipe, and weighed using an analytical balance, and (5) the water uptake is calculated using the acquired data.

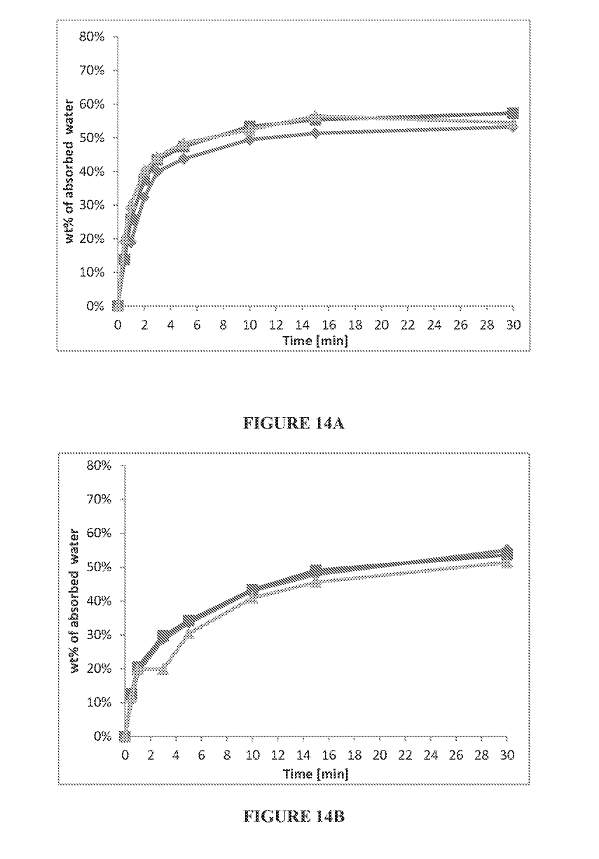

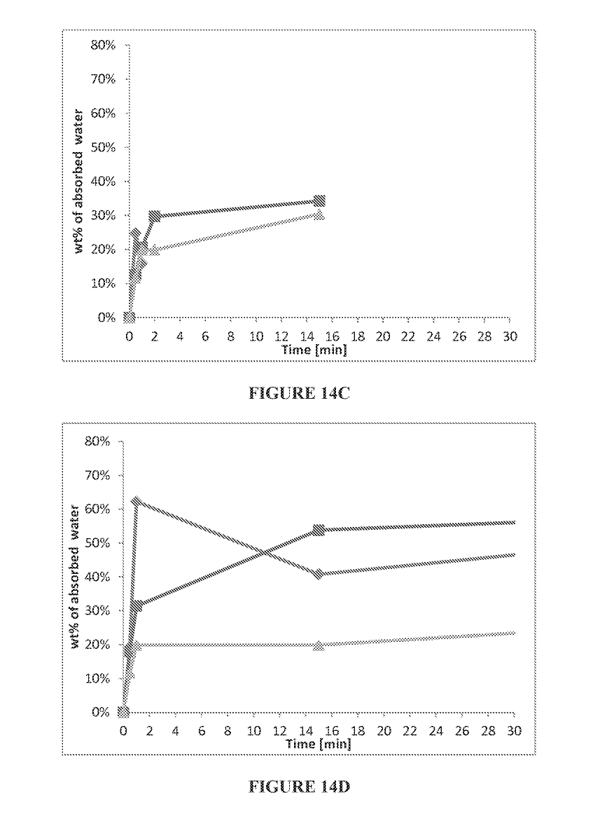

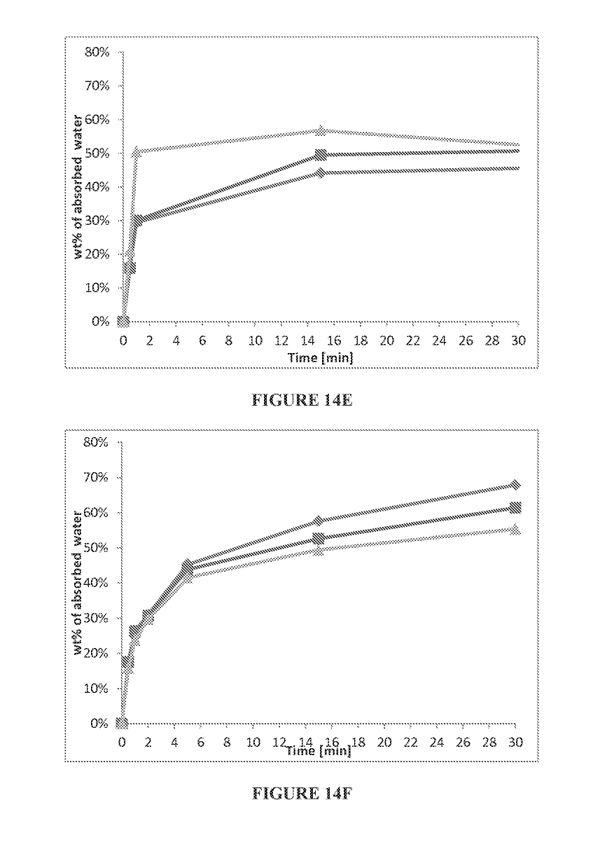

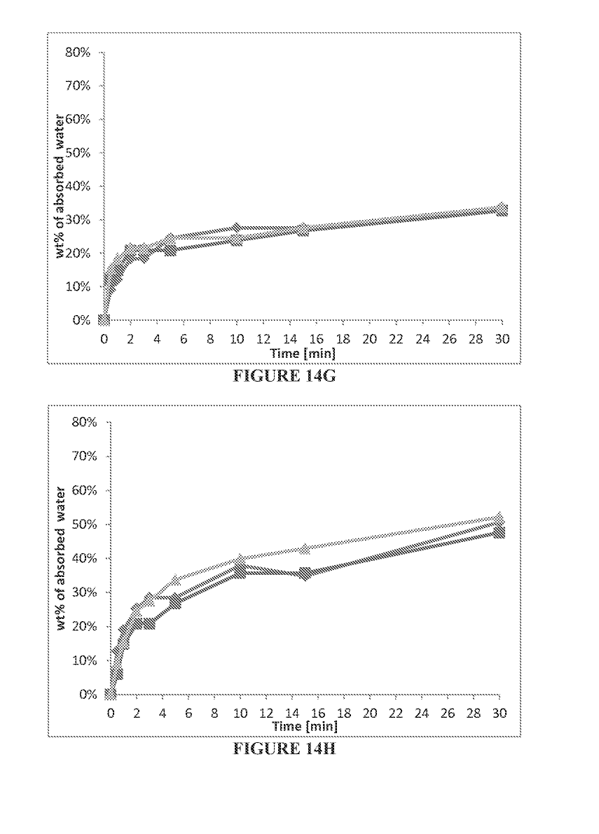

In an embodiment of the present invention, FIG. 14A shows water uptake of a reversed assembly prepared with Vinnol/EtOAc (20 wt %), 120 μm/200 μm, 40 wt % SC(s), 50 wt % CG8-H.

In an embodiment of the present invention, FIG. 14B shows water uptake of a sandwiched assembly prepared with Vinnol/EtOAc (20 wt %), 200 μm/120 μm/200 μm, 40 wt % SC(s), 50 wt % CG8-H.

In an embodiment of the present invention, FIG. 14C shows water uptake of a reversed assembly prepared with Elvacite/EtOAc (30 wt %), 120 μm/200 μm, 20 wt % SC(s), 50 wt % CG8-H.

In an embodiment of the present invention, FIG. 14D shows water uptake of a reversed assembly prepared with Elvacite/EtOAc (30 wt %), 120 μm/200 μm, 20 wt % SC(aq), 50 wt % CG8-H.

In an embodiment of the present invention, FIG. 14E shows water uptake of a reversed assembly prepared with Elvacite/EtOAc (30 wt %), 120 μm/200 μm, 40 wt % SC(aq), 50 wt % CG8-H.

In an embodiment of the present invention, FIG. 14F shows water uptake of a reversed assembly prepared with Elvacite/EtOAc (30 wt %), 120 μm/200 μm, 20 wt % SC(aq)+30 wt % KaMin 70C, 50 wt % CG8-H.

In an embodiment of the present invention, FIG. 14G shows water uptake of a reversed assembly prepared with Vinnacoat/MEK (20 wt %), 120 μm/200 μm, 20 wt % SC(aq), 50 wt % CG8-H.

In an embodiment of the present invention, FIG. 14H shows water uptake of a reversed assembly prepared with Vinnacoat/MEK (20 wt %), 120 μm/200 μm, 40 wt % SC(aq), 50 wt % CG8-H.

In an embodiment of the present invention, FIG. 14I shows water uptake of a reversed assembly prepared with Vinnacoat/MEK (20 wt %), 120 μm/200 μm, 20 wt % SC(aq)+30 wt % KaMin 70C, 50 wt % CG8-H.

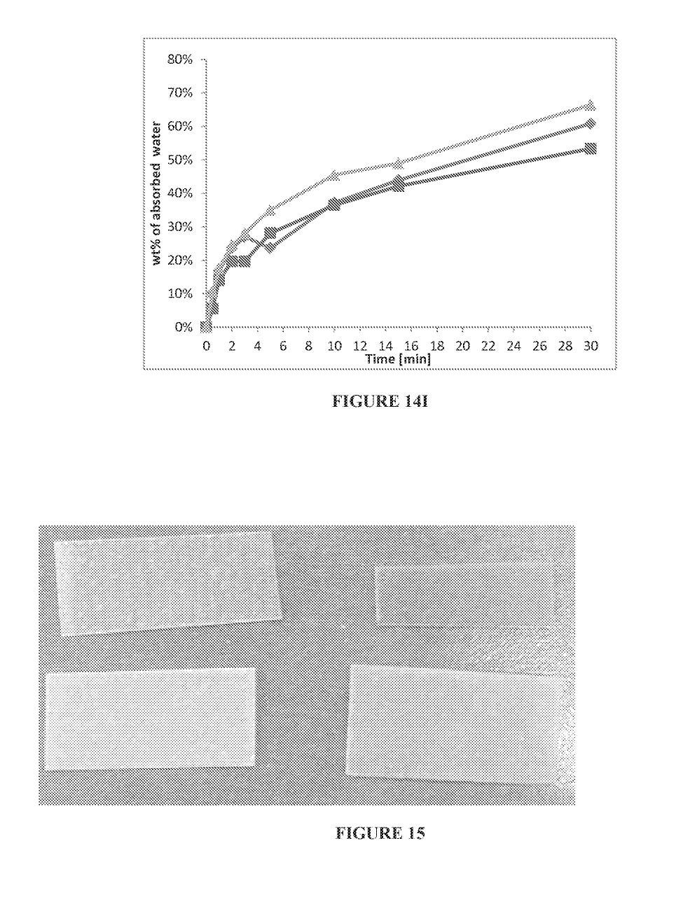

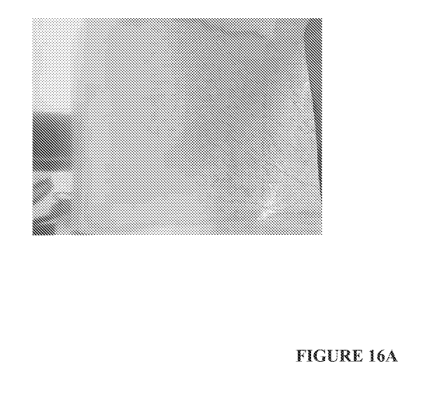

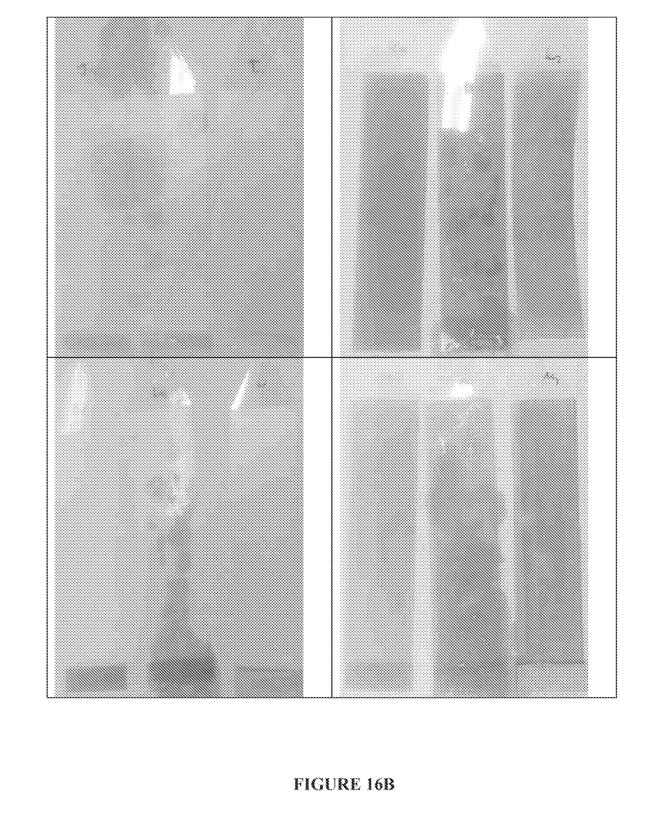

In an embodiment of the present invention, FIG. 15 shows regular (left) and reversed (right) assemblies with integrated indicator in the IX layer before (top) and after (bottom) AMA experiment. In some embodiments, the indicator reagents are tartrazine and phtalocyanine blue. The former is a yellow pigment, susceptible to oxidation and annihilation by CDO, while the latter is insusceptible blue pigment. Following hydration and CDO burst release the tartrazine is consumed. The yellow color disappears and the initially green assembly turns blue. The rate and kinetics of the indicator color change are investigated to determine whether this solution is fit to provide both indication demands.

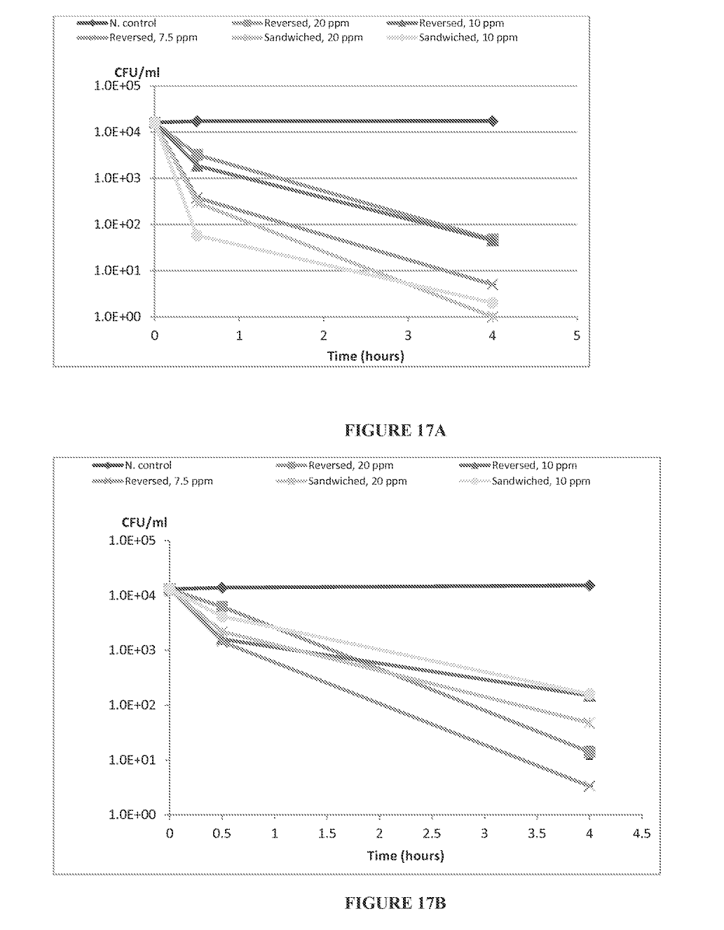

In an embodiment of the present invention, FIG. 16A shows a sandwiched assembly after 4 weeks in HALT of 40° C. and 80% RH in and evacuated Al bag. In another embodiment, FIG. 16B shows indicator assemblies before (leftmost in each picture), straight after use (middle) and after use and dry (rightmost). (Top) regular assemblies, (bottom) reversed assemblies.

In an embodiment of the present invention, FIG. 17A shows Clostridium perfringens viable counts. In an embodiment, the sandwiched (10 ppm and 20 ppm) assemblies have the lowest CFU/mL (CFU is a colony forming unit). The Clostridium genus also includes many known pathogenic strains such as the C. Botulinum (produces botulinum toxin, aka Botox, one of the strongest natural toxins), C. Tetani (causative of tetanus) and others. Most clostridium species flourish in the GI system when its natural flora is killed by antibiotic treatment. FIG. 17B also shows Clostridium perfringens viable counts. Most clostridium species flourish in the GI system when its natural flora is killed by antibiotic treatment.

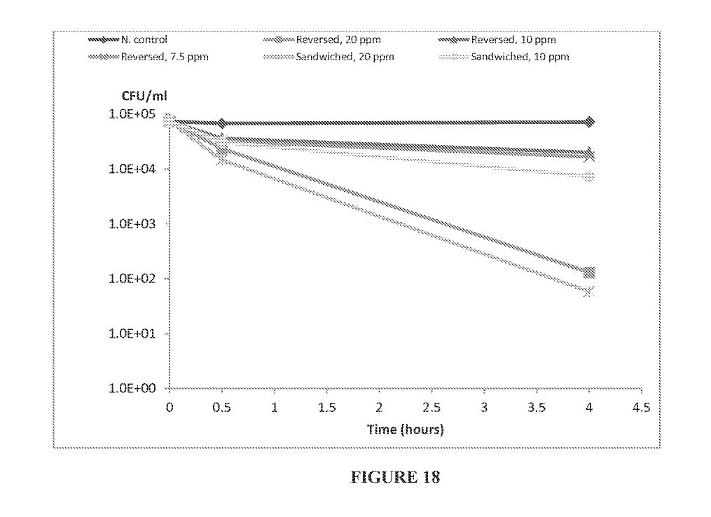

In an embodiment of the present invention, FIG. 18 shows Legionella viable counts (CFU/ml). In some embodiments, the sandwiched (20 ppm) apparatus had the highest kill rate/highest efficacy.

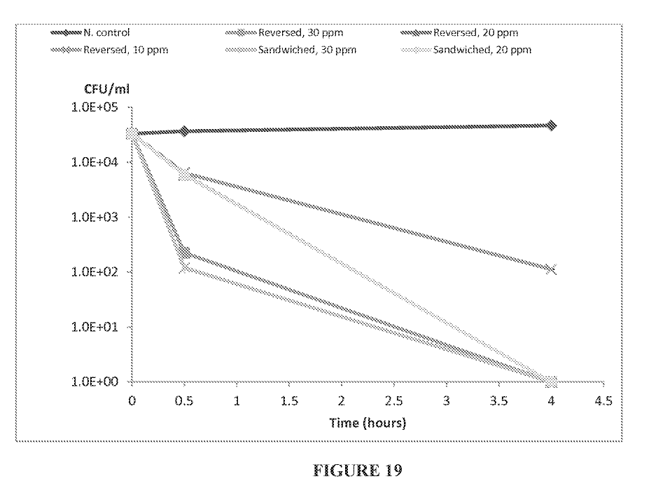

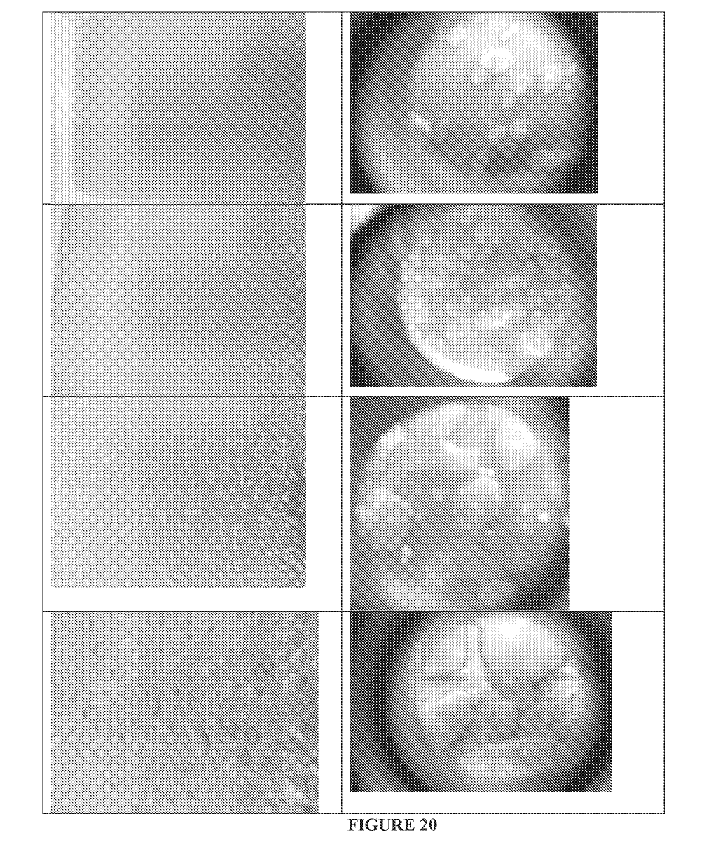

In an embodiment of the present invention, FIG. 19 shows Legionella viable counts (CFU/ml). In this experiment (contrary to AWT043), total kill rate was observed after 4 hours in 20 ppm.

In an embodiment of the present invention, FIG. 20 shows reversed assemblies prepared with hycar 26288 based formulation w/SC(aq). From top to bottom: 10 wt % SC, 20 wt % SC, 50 wt % SC, and 85 wt % SC.

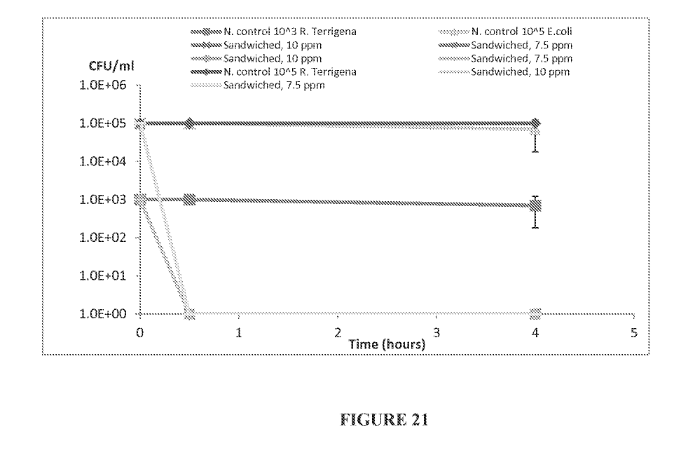

In an embodiment of the present invention, FIG. 21 shows viability counts of microorganisms after subjected to a variety of experiments.

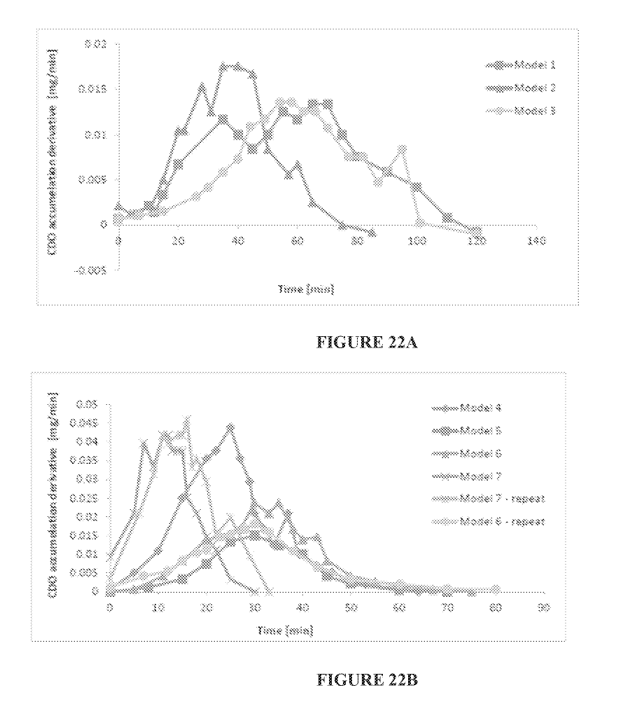

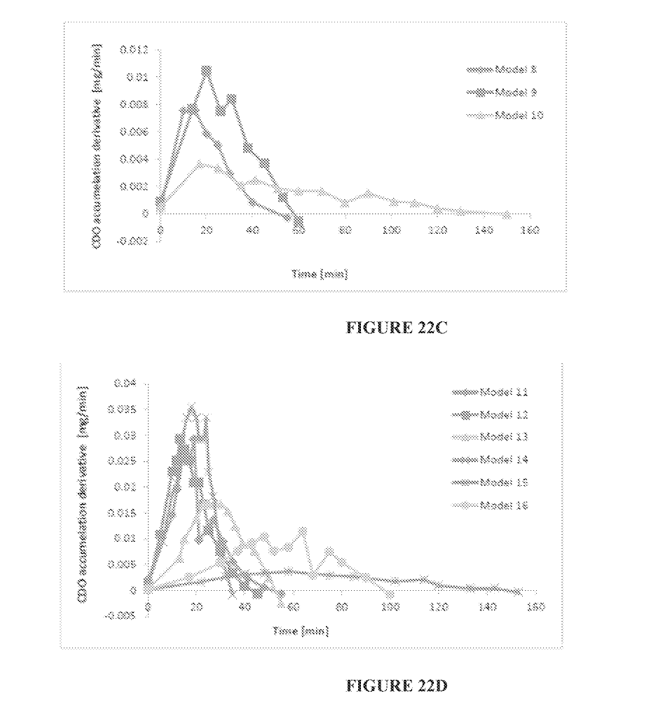

In some embodiments of the present invention, FIGS. 22A-D show CDO accumulation time derivative results. FIG. 22A shows vinnol based inserts (20 wt % vinnol in ethyl acetate), specifically illustrating CDO acculuation time derivative of vinnol based inserts. FIG. 22B illustrates elvacite based inserts (30 wt % elvacite in ethyl acetate), specifically illustrating CDO accumulation time derivative of elvacite based inserts. FIG. 22C shows vinnacoat based inserts (20 wt % vinnacoat in methyl ethyl ketone), specifically illustrating CDO accumulation time derivative of vinnacoat inserts. FIG. 22D shows blends of elvacite/vinnacoat based inserts, specifically illustrating CDO accumulation time derivative of elvacite/vinnacoat based inserts.

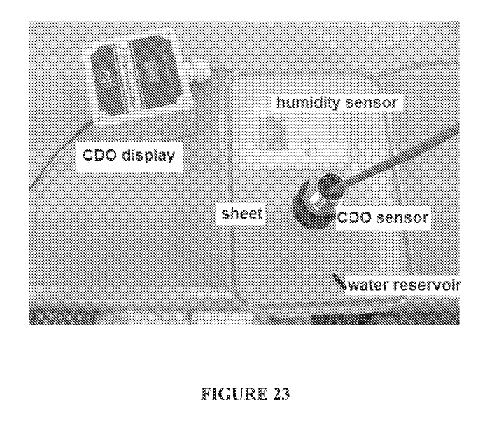

In an embodiment of the present invention, FIG. 23 illustrates a CDO measurement array, including a CDO display, a sheet, a humidity sensor, a CDO sensor, and/or a water reservoir.

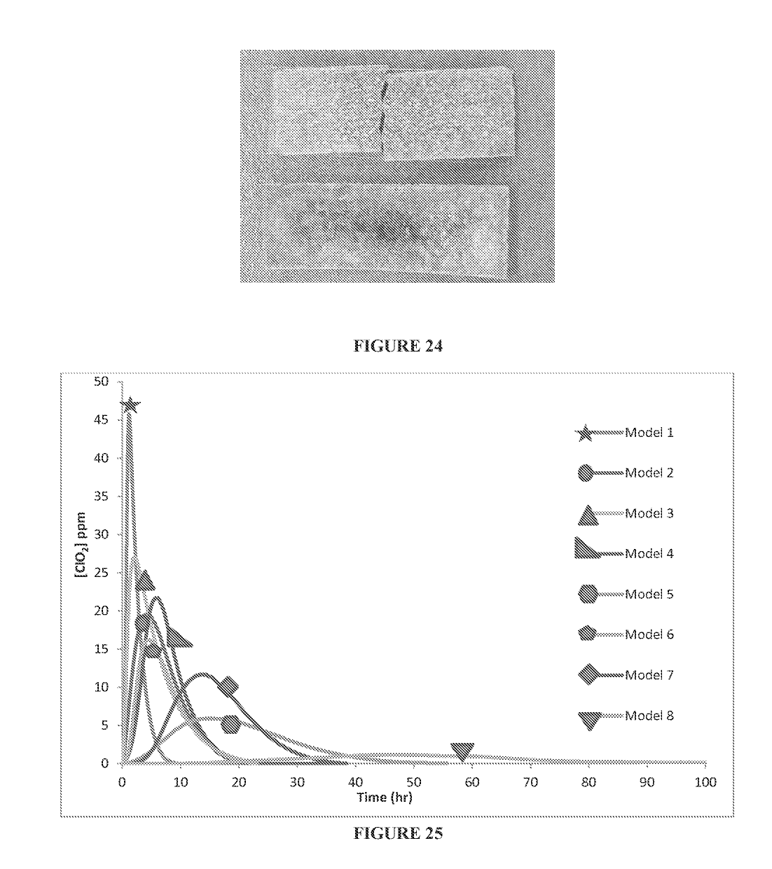

In an embodiment of the present invention, FIG. 24 illustrates sandwiched assemblies after 4 hours of immersion, bare-faced (top) and Vaseline-coated (bottom).

In an embodiment, FIG. 25 shows 8 models of the present invention tested for CDO release over time.

In an embodiment, FIGS. 27A and 27B show the measurement apparatus without fruit (FIG. 27A) and with fruit (FIG. 27B). FIGS. 27C-E show release kinetics, measuring CDO (ppm) over time (hours).

Water Purification Inserts

Assembly Geometry

In some embodiments of the present invention, an assembly geometry is selected from the group consisting of: i) regular, where an active material precursor (AMP) is loaded layer on top of activation agent (AA) loaded layer; ii) reversed, where an AA loaded layer is placed on top of AMP loaded layer; and iii) sandwiched, where an AMP layer is sandwiched between two AA loaded layers.

In some embodiments of the present invention, any rearrangement or multi-stacking of the layers is under the same scope.

In some embodiments of the present invention, additional material layers may also be added, the material layers are selected from the group consisting of: i) a protection layer, where the protection layer is composed of a material protecting the assembly form premature activation by humidity, light, air, heat, etc. (e.g., PVP (Kollidon 30 or VA64), PVAc/PEG (Kollicoat Protect, IR) or PVAc (Kollicoat SR), clear Vinnol layer), and protection layers are either applied on top of the assembly active area or between active layers; ii) a substrate layer, where the substrate layer is applied for purposes of improved activation, adhesion, visibility, or any commercial use; and iii) any combination thereof.

In some embodiments of the present invention, a layer comprises calculated thicknesses. In some embodiments of the present invention, a variety of layer thicknesses were applied and tested. In some embodiments of the present invention, each layer wet thickness is typically varied between few micrometers to several hundreds. In some embodiments of the present invention, examples of assemblies of varying layers' thicknesses are: i) regular (AA-layer [μm]/AMP-layer [μm]): 200/120, 200/12, 200/24, 200/40, 200/100; ii) reversed (AMP-layer [μm]/AA-layer [μm]): 120/200, 12/200, 24/200, 40/200, 100/200, 120/12, 120/24, 120/40, 120/100, 3/3, 120/400, 120/600; and iii) sandwiched (AA-layer [μm]/AMP-layer [μm]/AA-layer hump: 200/120/200, 200/12/200, 200/24/200, 200/40/200, 200/120/400, 200/120/600, 400/120/200, 400/120/400.

Substrate

In some embodiments of the present invention, the substrate may be any material complies with the target application or fabrication method, either of polymeric nature or other. In some embodiments of the present invention, a substrate used is polyethylene terephthalate (PET). In some embodiments of the present invention, any other polyester or other commonly used polymers may be used as the substrate, e.g., HDPE, LDPE, PP, PS, polyamides, etc. In some embodiments of the present invention, the substrate is selected from the group consisting of: paper, non-woven tissue paper, waxed paper, cardboard paper, PE-coated cardboard paper, Al-foil, etc. In some embodiments of the present invention, the substrate is typically corona treated prior to the coating application in order to modify the substrate surface energy to obtain better adhesion.

Methods of Application/Fabrication

In some embodiments of the present invention, a fabrication method complies with the handling constraints of the assembly materials, yield effective assemblies, and possess high efficiency and cost-effectiveness. In some embodiments of the present invention, possible fabrication techniques, lab or larger scale comprise: i) coating, where coating comprises draw-down and draw-down variants, dip-coating, manual coating, and nozzle-applied coating; ii) printing, where printing comprises Flexo and Flexo variants, Gravure and Gravure variants, Offset and Offset variants, screen printing and screen printing variants, and Ink-Jet and Ink-Jet variants; iii) wet and dry spraying and spraying variants; dripping and dripping variants; iv) sputtering; and chemical vapor deposition (at low enough T) techniques, e.g., aerosol-assisted.

### Fabrication Example

The following embodiment is a non-limiting lab-scale fabrication example. Lab scale fabrication was carried out using a draw-down coating. RK K101 or K202 control coater or K303 multicoater (RK printcoat instruments, UK) equipped with a vacuum bas was used. Coating rods that used were: Bird, 4-sided, Micrometer adjusted, close-wound meter bar, spirally-wound meter bar. Regular assembly fabrication comprised: 175 μm thick PET (190 mm×297 mm) sheets were double-corona treated; 1st layer of WE003 or WE018 was applied using a 200 μM rod and the formulation was transferred using a 10 ml sterile plastic syringe, where the syringe was cleaned prior to its use by 2-propanol and ethyl acetate and dried (to eliminate silicon oils residues); the sheet was inserted to a dry oven working at 60° C. for 30 min; the sheet was removed from the oven and left to cool in a sealed PE bag; a 2nd layer of WE004 was applied using a 120 μM close-wound meter bar (#9); the sheet was inserted to a dry oven working at 60° C. for 30 min; the sheet was removed from the oven and left to cool in a sealed PE bag; the sheet was sliced into assemblies of the required active area. Reversed assembly fabrication comprised: 175 μm thick PET (190 mm×297 mm) sheets were double-corona treated. 1st layer of WE004 was applied using a 120 μM rod. Formulation was transferred using a 10 ml sterile plastic syringe. The syringe was cleaned prior to its use by 2-propanol and ethyl acetate and dried (to eliminate silicon oils residues). The sheet was instantly inserted to a dry oven working at 60° C. for 30 min. The sheet was removed from the oven and left to cool in a sealed PE bag. A 2nd layer of WE003 or WE018 was applied using a 200 μM spirally-wound meter bar (#200). The sheet was instantly inserted to a dry oven working at 60° C. for 30 min. The sheet was removed from the oven and left to cool in a sealed PE bag. The sheet was sliced into assemblies of the required active area. Sandwiched assembly fabrication comprised: 175 μm thick PET (190 mm×297 mm) sheets were double-corona treated; 1st layer of WE003 or WE018 was applied using a 200 μM rod, and formulation was transferred using a 10 ml sterile plastic syringe, where the syringe was cleaned prior to its use by 2-propanol and ethyl acetate and dried (to eliminate silicon oils residues); the sheet was inserted to a dry oven working at 60° C. for 30 min; the sheet was removed from the oven and left to cool in a sealed PE bag; a 2nd layer of WE004 was applied using a 120 μVI close-wound meter bar (#9); the sheet was inserted to a dry oven working at 60° C. for 30 min; the sheet was removed from the oven and left to cool in a sealed PE bag; the 3rd layer of WE003 or WE018 was applied using a 200 μM spirally-wound meter bar (#200) the sheet was instantly inserted to a dry oven working at 60° C. for 30 min; the sheet was removed from the oven and left to cool in a sealed PE bag; the sheet was sliced into assemblies of the required active area.

In some embodiments, a protection layer may be applied, where the protection layer comprises a solution, the solution selected from a group consisting of: Kollidon 30 in 2-Propanol (16.67 wt %), Kollidon VA64 in 2-Propanol (16.67 wt %), Kollicoat Protect in water (10%). In some embodiments, a 12 μm to 120 μm layer of the protection layer formulation is applied on to or between active layers. In some embodiments, a sheet is inserted into a dry oven working at 60° C. for 30 min.

Quality Control for the Generation of CDO in the Polymer Matrix:

In some embodiments, ClOₓ-species analytic measurement, for a medium: double deionized water, comprise methods including: amperometric titration using Hach Autocat 9000—method 4500-ClO₂ D; iodometric titration—method 4500-ClO₂ B; spectrophotometry—EPA method 327.0; quick ClOₓ determination method, e.g., DPD; voltammetric, coulometric, potentiometric, or amperometric liquid or gas phase on-line sensor. In some embodiments, an efficacy trial is performed against a target microorganism.

Drying Schemes

The freshly-applied layers are typically inserted into a working oven immediately after application to encourage rapid volatilization of the solvent. The drying time and temperature is a derivative of the solvent or solvent mixture boiling point (i.e., volatility) and glass transition temperature of the binder. The drying scheme influences the acquired microstructure of the active layers and subsequently on the assembly efficacy, shelf-life and organoleptic attributes. However, several other schemes are possible: i) immediate introduction after application, 10-60 min, where the oven temperature is between 30° C. to 120° C.; iii) immediate introduction after application for 0.5 to 5 min at 80-120° C. followed by 10-60 min at 40-80° C.; iv) 1 min, 5 min, 10 min, 1 h, 24, etc. at room temperature (RT) and then 10-60 min at 30-120° C.; v) 1 min, 5 min, 10 min, 1 h, 24, etc. at room temperature (RT) and then 0.5 to 5 min at 80-120° C. followed by 10-60 min at 40-80° C.; vi) 1 min, 5 min, 10 min, 1 h, 24, etc. at room temperature (RT) without further dying steps.

In one embodiment, the influence of the drying scheme on the solvent evaporation rate was measured: Formulation: WE003.

In an embodiment, the geometry layer I is regular (i.e., AA formulation over PET substrate), 200 μm thick wet formulation layer.

In an embodiment, a drying scheme is selected from the group consisting of: i) application, followed by 30 min at 60° C.; ii) application, followed by 5 min at RT, then followed by 30 min at 60° C.; iii) application, followed by 1 hour at RT, then 30 min at 60° C.; and iv) application, then 24 hours at RT, and then 30 min at 60° C.

In some embodiments, the assemblies will be weighed after each step and the non-volatile substances and the solvent content will be calculated.

The present non-limiting example produced the following results:

i) 30 min in an oven working at 60° C. (or even shorter duration) completely eliminates all solvent residues, or reaches a steady state. Placing these sheets in an oven working at temperature higher than the solvent boiling temperature does not result in further loss of weight. Hence, the solvent evaporates after several minutes at 60° C.

ii) Approximately 50% of the solvent is evaporated in the first seconds after application, another 40% after 5 min and another 2% after an hour. After 24 h at RT, approx. additional 3% of the solvent is evaporated (total of 95%).

iii) Hence, in order to obtain the desired structure, the solvent will be volatized rapidly. After a few minutes, most of the solvent evaporates also at room temperature, but evaporates at a slower rate as compared to a dry oven, resulting in a different microstructure.

In a non-limiting example, binders are characterized as follows:

1) Demands: The binder for either the active material precursor layer or the activation agent layer provides the following: i) compatibility with the system ingredients; ii) resistance to water penetration through the gas phase during storage (e.g., AWT081 and AWT082); iii) release of active material precursor and activation agent (within the film matrix) upon introduction of the target medium, typically liquid water or beverage; iv) mechanical stability, specifically upon water penetration (to avoid decomposition into the target medium); and v) FDA direct food contact compliance of the binder itself, its solvents and any other additive (e.g., plasticizer, defoamer, dispersing and stabilizing agents, etc.) vi) adhesion to substrate surface.

2) Binder may be provided as dry material or as water- or solvent-borne emulsion, suspension or dispersion.

3) Binders:

i) Acrylic emulsions and resins, e.g., Hycar 26288, 26083, 26084, etc. (Lubrizol), Vinamul 3171 (Celanese), Elvacite (Lucite);

ii) Styrene acrylate emulsions, e.g., Neocryl A-2091, A-2092, A-1095 (DSM), Joncryl DFC 3030, DFC 3040 (BASF).

iii) Water or solvent borne polyurethanes, e.g., NeoRez (DSM).

iv) Celluloses, e.g. ethyl cellulose (e.g. DOW Ethocel), methyl cellulose (e.g. Dow methocel), hydroxypropyl methyl cellulose.

v) Poly vinyl chloride, poly vinyl acetate (PVAc), poly vinyl alcohol (PVA), poly vinyl pyrrolidone (PVP), poly vinyl botyral (PVB) and their co-polymers, grafts, and mixtures, either in solid, emulsion, solution or dispersion form. E.g., Vinnol (Poly vinyl chloride, PVAc and dicarboxylic acid, Wacker), VAGH (Poly vinyl chloride and PVAc, DOW), Kollidon (PVP, BASF), Kollicoat (PVA, PEG, PVAc, BASF), Mowital, Exceval and Mowiol (PVB and PVA, Kuraray), Butvar (PVB, Eastman), Vinnacoat LL 8100 (Styrene-olefin, Wacker), Vinnapas EP 8010 and EVA 202 (evinyl acetate ethylene, Wacker and Vinavil, respectively)

vi) sulfonated polystyrene polymers e.g. Kraton® NEXAR™ MD9200

In a non-limiting example, solvents/dispersants are characterized and/or generated as follows:

1) The solvent/dispersant provides dissolution and/or dispersion of the binder, active ingredients materials and other additives, forming a film upon drying.

2) Class III solvent. Class II solvents may be considered for limited and specific tasks.

3) Boiling temperature between 30° C. (to allow application without instant volatilization) to 120° C. (to allow drying and solvent residues removal at temperatures low enough to avoid active materials decomposition)

4) Solvents:

i) Water and other aqueous solutions—for water soluble/dispersible binders and materials.

ii) Alcohols, e.g. ethanol, 2-propanol, n-butanol are used to dissolve ethyl cellulose, PVA, PVB, etc.

iii) Esters, e.g. ethyl acetate, butyl acetate, are used to dissolve PVAc, ethyl cellulose, PVB, etc.

iv) Ketones, e.g., acetone, methyl ethyl ketone, methyl isobutyl ketone are used to dissolve PVB, PVAc/Poly vinyl chloride, etc.

v) Aliphatic and aromatic hydrocarbons, e.g., n-hexane, n-heptane, toluene, xylene.

vi) Ethers and glycol ethers, e.g., diethyl ether, dioxane, tetrahydrofuran.

vii) Polar aprotic solvents, e.g., dimethyl sulfoxide, dimethyl formamide, dimethyl acetamide.

In a non-limiting example, active material precursor is characterized and/or generated as follows:

1) The active material precursor is the raw material of the system active ingredient, chlorine dioxide, ClO₂ (CDO).

2) The precursor should be stable and safe to handle throughout formulation and assembly fabrication.

3) Upon activation by supply of protons and water the precursor reacts to form CDO.

4) CDO precursors:

i) Chlorite salts, e.g., sodium chlorite (NaClO₂), potassium chlorite, magnesium chlorite, etc.

ii) Chlorite bearing polymers and cation exchange resins.

iii) Hypochlorite salts, e.g. sodium hypochlorite.

iv) Chlorate (ClO₃⁻) and perchlorate (ClO₄⁻) salts.

In a non-limiting example, activation agent material is characterized or generated as follows:

1) The activation agent is a strong acid, e.g., with a pKa less than 2 (to enable chlorous acid formation), bounded or impregnated into a matrix, typically polymeric, glassy, or ceramic.

2) List of candidate active agents: A) strong acid cation exchange resins (with sulfonic or phosphonic groups), e.g., in their H-form. E.g.: i) ResinTech CG8-H. ii) DOW Rohm&Haas Amberlyst 15Dry or 15Wet; iii) DOW Rohm&Haas FPC-23 H; iv) Purolite NRW 1160; and v) Purolite C-100. B) Acidic zeolites and other minerals. E.g., i) Zeolyst Zeolite Beta CP811C-300 or equivalent; ii) Zeolyst Zeolite Y CBV 720 or equivalent; and iii) Zeolyst mordenite CBV 10A (Na-form, converted here to H-form) or equivalent. C) Bound or dry form of strong acids e.g. phosphoric acid, iodic acid, oxalic acid.

3) Weaker acids may also be used for yield enhancement and active fillers. Candidates: weak acid cation exchange resins (with carboxylic groups), e.g., in their H-form. E.g., i) DOW Rohm&Haas Amberlite IRP-64; ii) ResinTech WACG-H; iii) Purolite C115 or C115E; iv) Purolite C104Plus or C104EPlus. Acidic zeolites and other minerals and clays, as well as bound or dry form of weak acids, e.g., citric acid, may be used.

In a non-limiting example, indicator reagents are characterized or generated as follows:

1) The indicator reagent forms an observable color change after the polymer matrix system assembly has been exposed to liquid water.

2) The color change is observed and prevents the user from re-using a depleted assembly.

3) The indicator may also indicate the termination of the assembly action, i.e., the target medium is safe for consumption.

4) The indicator is typically composed of two colorants or pigments. The first pigment is sensitive for oxidation by the active material while the second one is oxidation resistant. Therefore, the combination of the two colors creates one color at the dry and unused state and different color (of the resistant pigment only) after the assembly has been used and the oxidation-susceptible pigment has been consumed.

5) Example for indicator reagents combination: tartrazine and phtalocyanine blue. In the unused state the assembly color is green. After the assembly is soaked in water and the active material is released, the yellow tartrazine is consumed and the assembly is left only with the phtalocyanine blue, yielding a faint blue color.

6) Both indicator reagents should be safe for use and authorized for food contact.

7) Reagents/active agents are not consumed prior to the assembly intended use. Furthermore, the oxidation of the pigment to-be-consumed does not affect the active material balance.

In a non-limiting example, alkali-stabilizing components are characterized or generated as follows:

1) The formulae are added with a stabilizing agent based on alkalizing species. The alkalization prevents premature activation of the active material precursor during formulation, fabrication or storage.

2) The alkalization agents are volatile or inferior to the activation agent strength to avoid deactivating the system.

3) Candidates: i) diluted ammonia solution (e.g., 25%); ii) organic base (e.g., methylamine); iii) strong or weak bases, e.g. hydroxides, glycine; iv) strong and weak anion exchange resins, e.g. Purolite A200-MBOH, Amberlite FPA-55; and v) basic zeolites, e.g., 4A, 13X.

In a non-limiting example, binder stock solution preparation is characterized or generated as follows:

1) The binder stock solution is the master solution for the preparation of the various formulae of the active assemblies.

2) The binder and solvents may be those (but not restricted to) which appear in the relevant sections.

3) The binder-solvent system yields the best results in terms of efficacy and stability. (See, e.g., AWT048 compared to AWT077, AWT078, AWT079, and AWT080)

4) Non-limiting Examples: Vinnol in ethyl acetate stock solution.

i) The Vinnol/EtOAc solution is composed of Vinnol powder (Wacker Chemie AG, Germany) dissolved in ethyl acetate.

ii) Vinnol grades vary in their composition (PVAc to Poly vinyl chloride and additives) and their subsequent physical properties (e.g., viscosity, molecular weight).

iii) The grade of choice is H30/48M, a terpolymer of 70% PVAc, 29% Poly vinyl chloride and 1% dicarboxylic acid.

iv) The solvent of choice is ethyl acetate (>99.9%) but an alternative solvent which dissolves Vinnol can be used, e.g., butyl acetate, acetone, methyl ethyl ketone, etc.

v) The Vinnol content of the solution should be such that enables film formation but not forming an impermeable film or exceeding the solubility limit. Typical Vinnol content are 5 wt % to 40 wt %. Non-limiting example: Vinnol/EtOAc 20/80 stock solution: A) the materials comprise a binder (Vinnol H30/48M (Wacker Chemie AG, Germany)) and a solvent/dispersant (Ethyl Acetate (EtOAc)>99.9% (Carlo Erba, France)); B) preparation (1 L) comprises pouring 800 g of EtOAc into a large beaker, stirring slowly using a paddle stirrer, adding slowly 200 g of Vinnol H30/48M powder (to avoid lumping), and continued stirring (500-1000 rpm) until a clear solution is obtained; C) QC is performed by requiring a clear translucent solution with a viscosity measuring: 200±50 cP (10-100 rpm, Brookfield spindle RV2).

In a non-limiting example, WE003—standard activation layer formula is characterized or generated as follows:

i) Materials include: a binder/solvent and an activation agent. Binder/Solvent: Vinnol/EtOAc 20/80 stock solution, from 97.5 wt % down to 55 wt % in the wet formulation. Activation agent: CG8-H Industrial grade cation exchange resin (ResinTech, NJ, USA), from 2 wt % up to 45 wt % in the wet formulation.

ii) Preparation of 50 wt % CG8-H in the dry solution (16.7 wt % in the wet formulation):

A) Elimination by wash of unreacted residual monomers from the CG8-H IX: 700 gr of CG8-H was inserted into a Poly vinyl chloride column (D=5.2 cm, H=50 cm) with screened bottom and the exit flow was controlled with a valve; B) resin was washed through the column with approximately 5 L of DDW (IONEX) for 1 hour; C) conductivity of the wash water was measured at the end of the process and found to be in the range of 1-3[μS/cm2]; and D) resin was transferred to a Pyrex dish and kept in the drying oven overnight at 80° C., where water content of up to 6% is applicable.

B) CG8-H milling and sieving: dry CG8-H beads were vortex-milled (at SuperFine, Israel) and sealed in evacuated water resistant aluminum bags; milled CG8-H was sieved and sieving fractions were sealed in evacuated water resistant aluminum bags; sieving fraction of D₅₀˜12 μm (D₉₀<50 μm).

C) Formula preparation (600 g): 500 g of Vinnol/EtOAc 20/80 stock solution was poured into a sufficient receptacle; the solution was stirred at 1500 rpm using 22 mm dispersion blade; 100 g of CG8-H powder (D50=12 μm, water content<6%) was added slowly; the solution was stirred at 1500 rpm for 10 min; the solution was evaluated for QC by viscosity, measuring 200-300 cP (10-100 rpm, Brookfield spindle RV3), % NVS=32.5%, and appearance, being a tan-colored homogeneous dispersion; after preparation or application excess formula may be stored indefinitely in a case receptacle that is kept air tight (to avoid evaporation). In the event of solid sedimentation, the formulation should be re-homogenized by stirring before application.

In a non-limiting example, WE018—indicator-integrated activation layer formulations are characterized or generated as follows:

i) Materials include: a binder/solvent and an activation agent. Binder/Solvent: Vinnol/EtOAc 20/80 stock solution, from 97.5 wt % down to 55 wt % in the wet formulation. Activation agent: CG8-H Industrial grade cation exchange resin (ResinTech, NJ, USA), from 2 wt % up to 45 wt % in the wet formulation. Tartrazine pigment, from 0.03 up to 1 wt % in the wet formulation. Phtalocyanine blue—Meghafast Blue BD 909 KNP (Florma, Israel), from 0.03 up to 1 wt % in the wet formulation.

ii) Preparation: A) Elimination by wash of unreacted residual monomers from the CG8-H IX: 700 gr of CG8-H was inserted into a Poly vinyl chloride column (D=5.2 cm, H=50 cm) with screened bottom and the exit flow was controlled with a valve; B) resin was washed through the column with approximately 5 L of DDW (IONEX) for 1 hour; C) conductivity of the wash water was measured at the end of the process and found to be in the range of 1-3[μS/cm2]; and D) resin was transferred to a Pyrex dish and kept in the drying oven overnight at 80° C., where water content of up to 6% is applicable.

B) CG8-H milling and sieving: dry CG8-H beads were vortex-milled (at SuperFine, Israel) and sealed in evacuated water resistant aluminum bags; milled CG8-H was sieved and sieving fractions were sealed in evacuated water resistant aluminum bags; sieving fraction of D₅₀-12 μm (D₉₀<50 μm). Additionally, formula preparation (for 600 g of formulation) was conducted by: pouring 400 g of Vinnol/EtOAc 20/80 stock solution into a sufficient receptacle; stirring at 1500 rpm using 22 mm dispersion blade; adding 100 g of CG8-H powder (D50=12 μm, water content<6%) slowly; adding 1 g of tartrazine while stirring; adding 1 g of phtalocyanine blue while stirring; stirring at 1500 rpm for 10 min; and QC was conducted by: viscosity: 200-300 cP (10-100 rpm, Brookfield spindle RV3), % NVS=32.7%, and appearance: dark green colored homogeneous dispersion.

In some embodiments of the present invention, after preparation or application formula reminders may be stored indefinitely in case receptacle was properly sealed (to avoid evaporation). In case of solid sedimentation the formula is re-homogenized by stirring before application.

In a non-limiting example, WE004—active ingredient precursor layer formula is characterized as follows:

i) Materials comprise a binder/solvent and an active material precursor. Binder/Solvent: Vinnol/EtOAc 20/80 stock solution, from 98 wt % down to 62.5 wt % in the wet formulation. Active material precursor: Sodium chlorite (NaClO₂) 80% (Sigma), from 2 wt % up to 37.5 wt % in the wet formulation.

ii) Preparation: Formula preparation (for 600 g of formulation): 400 g of Vinnol/EtOAc 20/80 stock solution was poured into a sufficient receptacle; the solution was stirred at 1500 rpm using 22 mm dispersion blade; 100 g of NaClO₂ was added; the solution was stirred at 1500 rpm for 10 min; for QC conditions, the following measurements were evaluated: Viscosity: 200-300 cP (10-100 rpm, Brookfield spindle RV3); % NVS=33.3%; and Appearance: white to pale yellow, opaque homogeneous dispersion. In some embodiments of the present invention, after preparation or application formula reminders may be stored indefinitely in case receptacle is properly sealed (mainly to avoid solvent evaporation). In case of solid sedimentation the formula should be re-homogenized by stirring before application.

In a non-limiting example, WE007—Alkali-stabilized active ingredient precursor layer formula is characterized or generated as follows:

i) Materials comprise a binder/solvent, an active material precursor, and ammonia. Binder/Solvent: Vinnol/EtOAc 20/80 stock solution, from 98 wt % down to 62.5 wt % in the wet formulation. Active material precursor: Sodium chlorite (NaClO₂) 80% (Sigma), from 2 wt % up to 37.5 wt % in the wet formulation. Ammonia (NH₃) 25% solution in water, from 0.1 wt % to 2 wt % in the wet formulation.

ii) Preparation: Formula preparation (for 600 g of formulation): A) 400 g of Vinnol/EtOAc 20/80 stock solution was poured into a sufficient receptacle; the solution was stirred at 1500 rpm using 22 mm dispersion blade; 5 ml of NH3 25% was added; 100 g of NaClO₂ was added; the solution was stirred at 1500 rpm for 10 min; and QC was performed by measuring: Viscosity: 200-300 cP (10-100 rpm, Brookfield spindle RV3), % NVS=33.3%, and Appearance: white opaque homogeneous dispersion. In some embodiments of the present invention, after preparation or application formula reminders may be stored indefinitely in case receptacle was properly sealed (to avoid evaporation). In some embodiments of the present invention, in the event of solid sedimentation, the formula should be re-homogenized by stirring before application.

Non-Limiting Examples

The following water purification inserts (“WE0_”, where _ indicates an identifying number) are used in the non-limiting examples illustrated below.

WE003-CG8-H(50 wt %): Vinnol/EtOAc formula

| | | | Material | | |
|---|---|---|---|---|---|
| Material | Supplier | CAS# | NVS | % wt | Wt (gr) |
| Vinnol H 30/48 M | wacker | N/A | 100% | 16.7% | 100.0 |
| Ethyl acetate | Carlo Erba | 141-78-6 | 0% | 66.7% | 400.0 |
| CG8-H | Resintech | 69011-20-7 | 95% | 16.7% | 100.0 |

| | | |
|---|---|---|
| Total | 100.0% | 600.0 |

WE004-SC(s, 40 wt %): Vinnol/EtOAc formula

| | | | Material | | |
|---|---|---|---|---|---|
| Material | Supplier | CAS# | NVS | % wt | Wt (gr) |
| Vinnol H 30/48 M | wacker | N/A | 100% | 16.7% | 16.0 |
| Ethyl acetate | Carlo Erba | 141-78-6 | 0% | 66.7% | 64.0 |
| Sodium chlorite 80% | sigma | 7758-19-2 | 100% | 16.7% | 16.0 |

| | |
|---|---|
| Total | 96.0 |

WE007-Alkalized SC(s, 40 wt %): Vinnol/EtOAc formula

| | | | Material | | |
|---|---|---|---|---|---|
| Material | Supplier | CAS# | NVS | % wt | Wt (gr) |
| Vinnol H 30/48 M | wacker | N/A | 100% | 16.6% | 32.0 |
| Ethyl acetate | Carlo Erba | 141-78-6 | 0% | 66.5% | 128.0 |
| Sodium chlorite | sigma | 7758-19-2 | 100% | 16.6% | 32.0 |
| Ammonia 25% | Fischer | 1336-21-6 | 0% | 0.3% | 0.5 |

| | |
|---|---|
| Total | 192.5 |

WE018-CG8-H(50 wt %): Vinnol/EtOAc formula w/indicator

| | | | Material | | |
|---|---|---|---|---|---|
| Material | Supplier | CAS# | NVS | % wt | Wt ( gr) |
| Vinnol H 30/48 M | Wacker | N/A | 100% | 16.6% | 30.0 |
| Ethyl acetate | Carlo erba | 171-78-6 | 0% | 66.5% | 120.0 |
| CG8-H | ResinTech | 69011-20-7 | 95% | 16.6% | 30.0 |
| Phtalocyanine blue | Florma | 147-14-8 | 100% | 0.2% | 0.29 |
| Tartrazine | Sigma | 1934-21-0 | 100% | 0.2% | 0.29 |

| | |
|---|---|
| Total | 180.6 |

WE020-SC(aq, 20 wt %): Vinnol/EtOAc formula

| Material | Supplier | CAS | Material NVS | % wt | Wt (gr) |
|---|---|---|---|---|---|
| Vinnol H 30/48 M | wacker | N/A | 100% | 17.1% | 30.0 |
| Ethyl acetate | Carlo Erba | 144-78-6 | 0% | 68.5% | 120.0 |
| NaClO2 (aq) 31% | Treitel | 7758-19-2 | 31% | 13.8% | 24.2 |
| NH3 25% | sigma | 1336-21-6 | 0% | 0.6% | 1.0 |

| | |
|---|---|
| Total | 175.2 |

WE025-xx-SC(aq): Hycar 26288 formulae in various SC contents

| | | | | |
|---|---|---|---|---|
| Material | Supplier | CAS# | Material NVS | Wt (gr) |

WE025-10

| | | | | |
|---|---|---|---|---|
| Hycar 26288 | Lubrizol | N/A | 49.00% | 80.0 |
| Ammonia (25%) | Sigma | 1336-21-6 | 0% | 2.0 |
| Water | | 7732-18-5 | 0% | 0.0 |
| Textone XL | Oxychem | 7758-19-2 | 31% | 14.0 |

| | |
|---|---|
| Total | 96.0 |

WE025-20

| | | | | |
|---|---|---|---|---|
| Hycar 26288 | Lubrizol | N/A | 49.00% | 80.0 |
| Ammonia (25%) | Sigma | 1336-21-6 | 0% | 2.0 |
| Water | | 7732-18-5 | 0% | 0.0 |
| Textone XL | Oxychem | 7758-19-2 | 31% | 31.6 |

| | |
|---|---|
| Total | 113.6 |

WE025-50

| | | | | |
|---|---|---|---|---|
| Hycar 26288 | Lubrizol | N/A | 49.00% | 40.0 |
| Ammonia (25%) | Sigma | 1336-21-6 | 0% | 2.0 |
| Water | | 7732-18-5 | 0% | 0.0 |
| Textone XL | Oxychem | 7758-19-2 | 31% | 63.2 |

| | |
|---|---|
| Total | 105.2 |

WE025-85

| | | | | |
|---|---|---|---|---|
| Hycar 26288 | Lubrizol | N/A | 49.00% | 8.9 |
| Ammonia (25%) | Sigma | 1336-21-6 | 0% | 2.0 |
| Water | | 7732-18-5 | 0% | 0.0 |
| Textone XL | Oxychem | 7758-19-2 | 31% | 80.0 |

| | |
|---|---|
| Total | 90.9 |

WE037-CG8-H(50 wt %): Elvacite/EtOAc formula

| | | | Material | | |
|---|---|---|---|---|---|
| Material | Supplier | Lot No. | NVS | % wt | Wt (gr) |
| Elvacite 4044 | Lucite | 025608-33-7 | 100% | 23.1% | 15.0 |
| Ethyl acetate | Carlo Erba | 141-78-6 | 0% | 53.8% | 35.0 |
| CG8-H | Resintech | 69011-20-7 | 95% | 23.1% | 15.0 |

| | | |
|---|---|---|
| Total | 100.0% | 65.0 |

WE039 - SC(s, 20 wt %):Elvacite/EtOAc formula

| | | | Material | | Wt |
|---|---|---|---|---|---|
| Material | Suppler | CAS# | NVS | % wt | (gr) |
| Elvacite 4044 | Lucite | 025608-33-7 | 100% | 27.3% | 30.0 |
| Ethyl acetate | Carlo Erba | 141-78-6 | 0% | 63.6% | 70.0 |
| NaClO2 | Sigma | 7758-19-2 | 100% | 9.1% | 10.0 |

| | | |
|---|---|---|
| Total | 100.0% | 110.0 |

WE047 - SC(s, 40 wt %):Elvacite/EtOAc formula

| | | | Material | | Wt |
|---|---|---|---|---|---|
| Material | Suppler | CAS# | NVS | % wt | (gr) |
| Elvacite 4044 | Lucite | 025608-33-7 | 100% | 23.1% | 22.5 |
| Ethyl acetate | Carlo Erba | 141-78-6 | 0% | 53.8% | 52.5 |
| NaClO2 | Sigma | 7758-19-2 | 100% | 23.1% | 22.5 |

| | | |
|---|---|---|
| Total | 100.0% | 97.5 |

WE048 - SC(aq, 20 wt %):Elvacite/EtOAc formula

| | | | Material | | Wt |
|---|---|---|---|---|---|
| Material | Suppler | CAS# | NVS | % wt | (gr) |
| Elvacite 4044 | Lucite | 025608-33-7 | 100% | 24.2% | 22.5 |
| Ethyl acetate | Carlo Erba | 141-78-6 | 0% | 56.4% | 52.5 |
| Textone XL | Oxy | 7758-19-2 | 31% | 19.4% | 18.1 |

| | | |
|---|---|---|
| Total | 100.0% | 93.1 |

WE049 - SC(aq, 40 wt %):Elvacite/EtOAc formula

| | | | Material | | Wt |
|---|---|---|---|---|---|
| Material | Supplier | CAS# | NVS | % wt | (gr) |
| Elvacite 4044 | Lucite | 025608-33-7 | 100% | 18.2% | 30.0 |
| Ethyl acetate | Carlo Erba | 141-78-6 | 0% | 42.6% | 70.0 |
| Textone XL | Oxy | 7758-19-2 | 31% | 39.2% | 64.4 |

| | | |
|---|---|---|
| Total | 100.0% | 164.4 |

WE050 - SC(aq, 20 wt %):Elvacite/EtOAc
formula w/KaMin 70 C. (30 wt %)

| | | | Material | | Wt |
|---|---|---|---|---|---|
| Material | Suppler | CAS# | NVS | % wt | (gr) |
| Elvacite 4044 | Lucite | 025608-33-7 | 100% | 19.1% | 30.0 |
| Ethyl acetate | Carlo Erba | 141-78-6 | 0% | 44.5% | 70.0 |
| KaMin 70 C. | KaMin LLC | 92704-41-1 | 99% | 11.6% | 18.2 |
| Ammonia sol. | Sigma | 1336-21-6 | 0% | 0.3% | 0.5 |
| 25% | | | | | |
| Treitel SC(aq) | Oxy | 7758-19-2 | 31% | 24.6% | 38.7 |
| 31% | | | | | |

| | | |
|---|---|---|
| Total | 100.0% | 157.4 |

WE051 - SC(s, 20 wt %):Vinnacoat/MEK formula

| | | | Material | | Wt |
|---|---|---|---|---|---|
| Material | Supplier | CAS# | NVS | % wt | (gr) |
| Vinnacoat LL8100 | Wacker | N/A | 100% | 18.9% | 17.8 |
| Methyl ethyl ketone | Biolab | 78-93-3 | 0% | 75.7% | 71.2 |
| NaClO2 | Sigma | 7758-19-2 | 100% | 5.3% | 5.0 |

| | | |
|---|---|---|
| Total | 100.0% | 94.0 |

WE052 - CG8-H(50 wt %):Vinnacoat/MEK formula

| | | | Material | | Wt |
|---|---|---|---|---|---|
| Material | Suppler | CAS# | NVS | % wt | (gr) |
| Vinnacoat LL8100 | Wacker | N/A | 100% | 16.7% | 30.0 |
| Methyl ethyl ketone | Biolab | 78-93-3 | 0% | 66.7% | 120.0 |
| CG8-H | Resintech | 69011-20-7 | 95% | 16.7% | 30.0 |

| | | |
|---|---|---|
| Total | 100.0% | 180.0 |

WE053 - SC(aq, 20 wt %):Vinnacoat/MEK formula

| | | | Material | | Wt |
|---|---|---|---|---|---|
| Material | Suppler | CAS# | NVS | % wt | (gr) |
| Vinnacoat LL8100 | Wacker | N/A | 100% | 17.6% | 17.8 |
| Methyl ethyl ketone | Biolab | 78-93-3 | 0% | 70.4% | 71.2 |
| Textone XL | OxyChem | 7758-19-2 | 100% | 12.0% | 12.1 |

| | | |
|---|---|---|
| Total | 100.0% | 101.1 |

WE054 - SC(aq, 40 wt %):Vinnacoat/MEK formula

| | | | Material | | Wt |
|---|---|---|---|---|---|
| Material | Supplier | CAS# | NVS | % wt | (gr) |
| Vinnacoat LL8100 | Wacker | N/A | 100% | 14.0% | 20.0 |
| Methyl ethyl ketone | BioLab | 78-93-3 | 0% | 55.9% | 80.0 |
| Textone XL | Oxy | 7758-19-2 | 31% | 30.1% | 43.0 |

| | | |
|---|---|---|
| Total | 100.0% | 143.0 |

WE055 - SC(aq, 20 wt %):Vinnacoat/MEK
formula w/KaMin 70 C. (30 wt %)

| | | | Material | | Wt |
|---|---|---|---|---|---|
| Material | Supplier | CAS# | NVS | % wt | (gr) |
| Vinnacoat LL8100 | Wacker | N/A | 100% | 15.1% | 17.8 |
| Methyl ethyl | Biolab | 78-93-3 | 0% | 60.3% | 71.2 |
| ketone | | | | | |
| KaMin 70 C. | KaMin | 92704-41-1 | 99% | 7.7% | 9.1 |
| | LLC | | | | |
| Ammonia sol. | Sigma | 1336-21-6 | 0% | 0.4% | 0.5 |
| 25% | | | | | |
| Textone XL | Oxy | 7758-19-2 | 31% | 16.4% | 19.4 |

| | | |
|---|---|---|
| Total | 100.0% | 118.0 |

WE056 - SC(aq, 40 wt %):Vinnol/EtOAc formula

| | | | Material | | Wt |
|---|---|---|---|---|---|
| Material | Supplier | CAS# | NVS | % wt | (gr) |
| Vinnol H 30/48 M | wacker | N/A | 100% | 13.9% | 30.0 |
| Ethyl acetate | Carlo Erba | 144-78-6 | 0% | 55.7% | 120.0 |
| NaClO2 (aq) 31% | Treitel | 7758-19-2 | 31% | 29.9% | 64.5 |
| NH3 25% | Sigma | 1336-21-6 | 0% | 0.5% | 1.0 |

| | |
|---|---|
| Total | 215.5 |

WE057 - SC(aq, 20 wt %):Vinnol/EtOAc
formula w/KaMin 70 C. (30 wt %)

| | | | Material | | Wt |
|---|---|---|---|---|---|
| Material | Supplier | CAS# | NVS | % wt | (gr) |
| Vinnol H30/48M | Wacker | N/A | 100% | 14.5% | 18.0 |
| Ethyl acetate | Carlo Erba | 144-78-6 | 0% | 58.0% | 72.0 |
| KaMin 70 C. | KaMin LLC | 7758-19-2 | 99% | 8.8% | 10.9 |
| Textone XL | Oxy | 1336-21-6 | 31% | 18.7% | 23.2 |

| | | |
|---|---|---|
| Total | 100.0% | 124.1 |

WE058 - CG8-H(50 wt %):Elvacite/EtOAc formula w/indicator

| | | | Material | | Wt |
|---|---|---|---|---|---|
| Material | Supplier | CAS# | NVS | % wt | (gr) |
| Elvacite 4044 | Lucite | 025608-33-7 | 100% | 23.0% | 150.0 |
| Ethyl acetate | Carlo Erba | 171-78-6 | 0% | 53.6% | 350.0 |
| CG8-H | ResinTech | 69011-20-7 | 95% | 23.0% | 150.0 |
| Phtalocyanine | Florma | 147-14-8 | 100% | 0.2% | 1.46 |
| blue | | | | | |
| Tartrazine | Sigma | 1934-21-0 | 100% | 0.2% | 1.46 |

| | |
|---|---|
| Total | 652.9 |

WE075 - CG8-H(50 wt %):[Elvacite/Vinnacoat =
4/1]/EtOAc formula w/indicator

| | | | Material | | Wt |
|---|---|---|---|---|---|
| Material | Supplier | CAS# | NVS | % wt | (gr) |
| Elvacite 4044/ | Lucite | 025608-33-7 | 30% | 56.9% | 80.0 |
| EtOAc | | | | | |
| Vinnacoat LL8100/ | Wacker | N/A | 20% | 21.3% | 30.0 |
| EtOAc | | | | | |
| Ethyl acetate | Carlo Erba | 141-78-6 | 0% | 0.0% | 0.0 |
| Tartrazine | Sigma | 1934-21-0 | 100% | 0.2% | 0.3 |
| Phatalocyanine | Florma | 147-14-8 | 100% | 0.2% | 0.3 |
| blue | | | | | |
| CG8-H | Resintech | 69011-20-7 | 95% | 21.3% | 30.0 |

| | | |
|---|---|---|
| Total | 100.0% | 140.6 |

WE076 - CG8-H(50 wt %):[Elvacite/Vinnacoat =
1/1]/EtOAc formula w/indicator

| | | | Material | | Wt |
|---|---|---|---|---|---|
| Material | Supplier | CAS# | NVS | % wt | (gr) |
| Elvacite 4044/ | Lucite | 025608-33-7 | 30% | 32.3% | 100.0 |
| EtOAc | | | | | |
| Vinnacoat LL8100/ | Wacker | N/A | 20% | 48.4% | 150.0 |
| EtOAc | | | | | |
| Ethyl acetate | Carlo Erba | 141-78-6 | 0% | 0.0% | 0.0 |
| Tartrazine | Sigma | 1934-21-0 | 100% | 0.0% | 0.0 |
| Phatalocyanine | Florma | 147-14-8 | 100% | 0.0% | 0.0 |
| blue | | | | | |
| CG8-H | Resintech | 69011-20-7 | 95% | 19.4% | 60.0 |

| | | |
|---|---|---|
| Total | 100.0% | 310.0 |

WE077-CG8-H(50 wt %): [Elvacite/Vinnacoat = 1/4]/
EtOAc formula w/indicator

| | | | Material | | Wt |
|---|---|---|---|---|---|
| Material | Supplier | CAS# | NVS | % wt | (gr) |
| Elvacite 4044/EtOAc | Lucite | 025608-33-7 | 30% | 11.7% | 20.0 |
| Vinnacoat | Wacker | N/A | 20% | 70.3% | 120.0 |
| LL8100/EtOAc | | | | | |
| Ethyl acetate | Carlo Erba | 141-78-6 | 0% | 0.0% | 0.0 |
| Tartrazine | Sigma | 1934-21-0 | 100% | 0.2% | 0.3 |
| Phatalocyanine | Florma | 147-14-8 | 100% | 0.2% | 0.3 |
| blue | | | | | |
| CG8-H | Resintech | 69011-20-7 | 95% | 17.6% | 30.0 |

| | | |
|---|---|---|
| Total | 100.0% | 170.6 |

WE078-SC(aq, 40 wt %): [Elvacite/Vinnacoat = 1/4]/
EtOAc formula

| | | | Material | | |
|---|---|---|---|---|---|
| Material | Supplier | CAS# | NVS | % wt | Wt (gr) |
| Elvacite | Lucite | 025608-33-7 | 30% | 45.7% | 100.0 |
| 4044/EtOAc | | | | | |
| Vinnacoat | Wacker | N/A | 20% | 17.2% | 37.5 |
| LL8100/EtOAc | | | | | |
| Ethyl acetate | Carlo Erba | 141-78-6 | 0% | 0.0% | 0.0 |
| SC(aq) 31% | Treitel | 7758-19-2 | 31% | 36.9% | 80.6 |
| Ammonia 25% | Sigma | 1336-21-6 | 0% | 0.2% | 0.5 |

| | | |
|---|---|---|
| Total | 100.0% | 218.6 |

WE079-SC(aq, 20 wt %): [Elvacite/Vinnacoat = 4/1]/
EtOAc formula w/KaMin 70 C. (30 wt %)

| | | | Material | | |
|---|---|---|---|---|---|
| Material | Supplier | CAS# | NVS | % wt | Wt (gr) |
| Elvacite | Lucite | 025608-33-7 | 30% | 47.9% | 100.0 |
| 4044/EtOAc | | | | | |
| Vinnacoat | Wacker | N/A | 20% | 18.0% | 37.5 |
| LL8100/EtOAc | | | | | |
| Ethyl acetate | Carlo Erba | 141-78-6 | 0% | 0.0% | 0.0 |
| KaMin 70 C. | KaMin LLC | 92704-41-1 | 99% | 10.8% | 22.5 |
| SC(aq) 31% | Treitel | 7758-19-2 | 31% | 23.2% | 48.4 |
| Ammonia 25% | Sigma | 1336-21-6 | 0% | 0.2% | 0.5 |

| | | |
|---|---|---|
| Total | 100.0% | 208.9 |

WE080-SC(aq, 40 wt %): [Elvacite/Vinnacoat = 1/1]/
EtOAc formula

| | | | Material | % | Wt |
|---|---|---|---|---|---|
| Material | Supplier | CAS# | NVS | wt | (gr) |
| Elvacite | Lucite | 025608-33-7 | 30% | 26.3% | 40.0 |
| 4044/EtOAc | | | | | |
| Vinnacoat | Wacker | N/A | 20% | 39.4% | 60.0 |
| LL8100/EtOAc | | | | | |
| Ethyl acetate | Carlo Erba | 141-78-6 | 0% | 0.0% | 0.0 |
| SC(aq) 31% | Treitel | 7758-19-2 | 31% | 33.9% | 51.6 |
| Ammonia 25% | Sigma | 1336-21-6 | 0% | 0.3% | 0.5 |

| | | |
|---|---|---|
| Total | 100.0% | 152.1 |

WE081-SC(aq, 20 wt %): [Elvacite/Vinnacoat = 1/1]/
EtOAc formula w/KaMin 70 C. (30 wt %)

| | | | Material | | |
|---|---|---|---|---|---|
| Material | Supplier | CAS# | NVS | % wt | Wt (gr) |
| Elvacite | Lucite | 025608-33-7 | 30% | 27.4% | 40.0 |
| 4044/EtOAc | | | | | |
| Vinnacoat | Wacker | N/A | 20% | 41.1% | 60.0 |
| LL8100/EtOAc | | | | | |
| Ethyl acetate | Carlo Erba | 141-78-6 | 0% | 0.0% | 0.0 |
| KaMin 70 C. | KaMin LLC | 92704-41-1 | 99% | 9.9% | 14.4 |
| SC(aq) 31% | Treitel | 7758-19-2 | 31% | 21.2% | 31.0 |
| Ammonia 25% | Sigma | 1336-21-6 | 0% | 0.3% | 0.5 |

| | | |
|---|---|---|
| Total | 100.0% | 145.9 |

WE082-SC(aq, 40 wt %): [Elvacite/Vinnacoat = 1/4]/
EtOAc formula

| | | | Material | | |
|---|---|---|---|---|---|
| Material | Supplier | CAS# | NVS | % wt | Wt (gr) |
| Elvacite 4044/EtOAc | Lucite | 025608-33-7 | 30% | 9.7% | 10.0 |
| Vinnacoat | Wacker | N/A | 20% | 58.4% | 60.0 |
| LL8100/EtOAc | | | | | |
| Ethyl acetate | Carlo Erba | 141-78-6 | 0% | 0.0% | 0.0 |
| SC(aq) 31% | Treitel | 7758-19-2 | 31% | 31.4% | 32.3 |
| Ammonia 25% | Sigma | 1336-21-6 | 0% | 0.5% | 0.5 |

| | | |
|---|---|---|
| Total | 100.0% | 102.8 |

WE083-SC(aq, 20 wt %): [Elvacite/Vinnacoat = 1/4]/
EtOAc formula w/KaMin 70 C. (30 wt %)

| | | | Material | | |
|---|---|---|---|---|---|
| Material | Supplier | CAS# | NVS | % wt | Wt (gr) |
| Elvacite | Lucite | 025608-33-7 | 30% | 10.1% | 10.0 |
| 4044/EtOAc | | | | | |
| Vinnacoat | Wacker | N/A | 20% | 60.7% | 60.0 |
| LL8100/EtOAc | | | | | |
| Ethyl acetate | Carlo Erba | 141-78-6 | 0% | 0.0% | 0.0 |
| KaMin 70 C. | KaMin LLC | 92704-41-1 | 99% | 9.1% | 9.0 |
| SC(aq) 31% | Treitel | 7758-19-2 | 31% | 19.6% | 19.4 |
| Ammonia 25% | Sigma | 1336-21-6 | 0% | 0.5% | 0.5 |

| | | |
|---|---|---|
| Total | 100.0% | 98.9 |

PE032-SC(s, 7.4 wt %): Hycar 26288
formula w/KaMin 70 C. (2.2 wt %)

| | | | Material | Wt |
|---|---|---|---|---|
| Material | Supplier | CAS# | NVS | (gr) |
| Hycar 26288 | Lubrizol | N/A | 49.00% | 800.0 |
| Kamin 70 C. | Kamin LLC | 92704-41-1 | 99% | 10.0 |
| Ammonia (25%) | Sigma | 1336-21-6 | 0% | 20.0 |
| Water | | 7732-18-5 | 0% | 0.0 |
| Sodium chlorite | Sigma | 7758-19-2 | 100% | 41.0 |

| | |
|---|---|
| Total | 871.0 |

MP-SC015-SC(s, 12 wt %): Hycar 26288
formula w/KamMin 70 C. (23.4 wt %)

| | | | Material | | |
|---|---|---|---|---|---|
| Material | Supplier | CAS# | NVS | % wt | Wt (gr) |
| Hycar 26288 | Lubrizol | N/A | 49.00% | 65.6% | 160.0 |
| KaMin 70 C. | Kamin LLC | 92704-41-1 | 97% | 12.6% | 30.8 |
| Ammonia (25%) | | 1336-21-6 | 0% | 1.6% | 4.0 |
| Water | | 7732-18-5 | 0% | 12.3% | 30.0 |
| Sodium chlorite | Sigma | 7758-19-2 | 100% | 7.8% | 19.0 |
| 80% | | | | | |

| | | |
|---|---|---|
| Total | 100.0% | 244 |

WE032-CG8-H(50 wt %): Vinnol/EtOAc
formula w/KaMin 70 C. (10 wt %)

| | | | Material | | |
|---|---|---|---|---|---|
| Material | Supplier | CAS# | NVS | % wt | Wt (gr) |
| Vinnol H | wacker | N/A | 100% | 15.3% | 15.0 |
| 30/48 M | | | | | |
| Ethyl acetate | Carlo Erba | 141-78-6 | 0% | 61.2% | 60.0 |
| KaMin 70 C. | KaMin LLC | 92704-41-1 | 99% | 4.1% | 4.0 |
| CG8-H | Resintech | 69011-20-7 | 95% | 19.4% | 19.0 |

| | | |
|---|---|---|
| Total | 100.0% | 98.0 |

WE033-CG8-H(50 wt %):
Vinnol/EtOAc formula w/KaMin 70 C. (20 wt %)

| | | | Material | | |
|---|---|---|---|---|---|
| Material | Supplier | CAS# | NVS | % wt | Wt (gr) |
| Vinol H 30/48 M | wacker | N/A | 100% | 13.7% | 15.0 |
| Ethyl acetate | Carlo Erba | 141-78-6 | 0% | 54.9% | 60.0 |
| KaMin 70 C. | KaMin LLC | 92704-41-1 | 99% | 8.9% | 9.7 |
| CG8-H | Resintech | 69011-20-7 | 95% | 22.5% | 24.6 |

| | | |
|---|---|---|
| Total | 100.0% | 109.3 |

WE009-CG8-H(58 wt %) and SC(s, 30 wt %)
aqueous formula in Hycar 26288

| | | | Material | | |
|---|---|---|---|---|---|
| Material | Supplier | CAS# | NVS | % wt | Wt (gr) |
| DDW | | 7732-18-5 | 0% | 5.2% | 2.8 |
| TEGO 740W | Evonik | N/A | 99% | 1.0% | 0.5 |
| Surfynol SE-F | Air Products | 9014-85-1 | 50% | 0.1% | 0.1 |
| Tego Foamex | Evonik | 9005-00-9; | 25% | 0.2% | 0.1 |
| 825 | | 141-43-5; | | | |
| | | 128-37-0; | | | |
| | | 110-82-7; | | | |
| | | 64-17-5 | | | |
| CG8-H | ResinTech | 69011-20-7 | 17% | 72.5% | 39.6 |
| Netzsch grind | | | | | |
| Ammonia 25% | Fischer | 1336-21-6 | 0% | 6.4% | 3.5 |
| Sodium chlorite | Sigma | 7758-19-2 | 100% | 1.8% | 1.0 |
| Hycar 26288 | Lubrizol | N/A | 49% | 12.8% | 7.0 |

| | | |
|---|---|---|
| Total | 100.0% | 54.6 |

WE012-SC(s, 40 wt %): Vinnol/EtOAc
formula w/ wheat fibers

| | | | Material | | |
|---|---|---|---|---|---|
| Material | Supplier | CAS# | NVS | % wt | Wt (gr) |
| Vinnol H | Wacker | N/A | 100% | 14.2% | 10.0 |
| 30/48 M | | | | | |
| Ethyl acetate | Carlo Erba | 141-78-6 | 0% | 56.8% | 40.0 |
| Sodium chlorite | sigma | 7758-19-2 | 100% | 21.3% | 15.0 |
| Wheat fibers | Hashlosha | N/A | 93% | 7.7% | 5.4 |
| Ammonia 25% | Fischer | 1336-21-6 | 0% | 0.0% | 0.0 |

| | |
|---|---|
| Total | 70.4 |

WE013-CG8-H(44 wt %): Vinnol/EtOAc
formula w/KaMin 70 C. (10 wt %)

| | | | Material | | |
|---|---|---|---|---|---|
| Material | Supplier | CAS# | NVS | % wt | Wt (gr) |
| Vinol H | Wacker | N/A | 100% | 16.1% | 10.0 |
| 30/48 M | | | | | |
| Ethyl acetate | Carlo Erba | 141-78-6 | 0% | 64.3% | 40.0 |
| KaMin 70 C. | KaMin LLC | 92704-41-1 | 99% | 3.5% | 2.2 |
| CG8-H | Resintech | 69011-20-7 | 95% | 16.1% | 10.0 |

| | | |
|---|---|---|
| Total | 100.0% | 62.2 |

WE016-CG8-H(44 wt %): Vinnol/EtOAc
formula w/WACG-H (10 wt %)

| | | | Material | | |
|---|---|---|---|---|---|
| Material | Supplier | CAS# | NVS | % wt | Wt (gr) |
| Vinol H | Wacker | N/A | 100% | 16.1% | 10.0 |
| 30/48 M | | | | | |
| Ethyl acetate | Carlo Erba | 141-78-6 | 0% | 64.3% | 40.0 |
| WACG-H-HP | Resintech | 9052-45-3 | 96% | 3.6% | 2.25 |
| CG8-H | Resintech | 69011-20-7 | 95% | 16.1% | 10.0 |

| | | |
|---|---|---|
| Total | 100.0% | 62.3 |

WE019 - SC(s, 10%, Shengya chem.):Vinnol/EtOAc formula

| | | | Material | | |
|---|---|---|---|---|---|
| Material | Suplier | CAS# | NVS | % wt | Wt (gr) |
| Vinnol H 30/48 M | wacker | N/A | 100% | 19.6% | 30.0 |
| Ethyl acetate | Daejung | 141-78-6 | 0% | 78.3% | 120.0 |
| Sodium chlorite | Shengya | 7758-19-2 | 100% | 2.2% | 3.3 |

| | | |
|---|---|---|
| Total | 100.0% | 153.3 |

WE022 - SC(aq, 20 wt %):Vinnol/BuOAc formula

| | | | Material | | |
|---|---|---|---|---|---|
| Material | Supplier | CAS# | NVS | % wt | Wt (gr) |
| Vinnol H 30/48 M | Wacker | N/A | 100% | 16.6% | 30.0 |
| Butyl acetate | BioLab | 123-86-4 | 0% | 66.3% | 120.0 |
| Textone L | Oxychem | 7758-19-2 | 25% | 16.6% | 30.0 |
| (SC 25%) | | | | | |
| NH3 25% | Sigma | 1336-21-6 | 0% | 0.6% | 1.0 |

| | | |
|---|---|---|
| Total | 100.0% | 181.0 |

WE024 - SC(aq, 5 wt %):Kollicoat Protect/water fromula

| | | | Material | | |
|---|---|---|---|---|---|
| Material | Supplier | CAS# | NVS | % wt | Wt (gr) |
| Kollicoat Protect | Sigma/ | 96734-3-3; | 100% | 9.8% | 8.0 |
| | BASF | 9002-89-5 | | | |
| DDW | | 7732-18-5 | 0% | 88.0% | 72.0 |
| Textone L | Oxychem | 7758-19-2 | 25% | 2.1% | 1.69 |
| (SC 25%) | | | | | |
| NH3 25% | Sigma | 1336-21-6 | 0% | 0.1% | 0.1 |

| | | |
|---|---|---|
| Total | 100.0% | 81.8 |

Non-Limiting Examples for Experiments Examining AMA v. Specific Microorganisms:

Escherichia coli (E. coli, ATCC:11229, 25922): In a non-limiting example, sample 69 [AWT069] was examined regarding the efficacies of reversed and sandwiched assemblies in varying SC contents from 2.5 to 10 ppm by utilizing the world health organization (WHO) protocol. Efficacy was examined against E. coli (ATCC 25922) in two types of media, General test water (GTW: pH˜7, TOC˜1 mg/L, T˜20° C., TDS˜50-500 mg/L, alkalinity˜40 mg/L, typically replaced by TSB 1:500) and challenge test water (CTW: TOC˜30 mg/L, turbidity˜40 mg/L, T˜4° C., TDS˜1500 mg/L, alkalinity˜200 mg/L). In both GTW and CTW, reversed assemblies were fully effective (total eradication of 10³ cfu/ml) in 0.5 h down to 7.5 ppm while sandwiched assemblies brought total eradication after 30 min down to 5 ppm.

In a non-limiting example, Sample 20 [AWT021] demonstrated the efficacy of regular, reversed and sandwiched assemblies (10 ppm of SC) vs. Escherichia coli (ATCC 25922) and P. aeruginosa (10⁵ cfu/ml in TSB 1/500, 1 h).

In a non-limiting example, AWT086 demonstrated the efficacy of reversed and sandwiched assemblies vs. E. coli (ATCC 11229, 10³ cfu/ml). Reversed assemblies were effective down to 7.5 ppm, sandwiched assemblies were effective down to 5 ppm.

Raoultella (Klebsiella) terrigena (R. terrigena, ATCC 33257): In a non-limiting example, Sample 52 [AWT048] was tested regarding efficacies of 2 m old assemblies stored in RT. All examined assemblies (regular, reversed, and sandwiched, alkalized and not, w/or w/o PVP layer). All assemblies were effective under EPA #1 conditions. Reversed and sandwiched assemblies were effective after 0.5 h under EPA #2 conditions also. Regular assemblies were not effective under EPA #2 at all. Reversed and sandwiched assemblies with PVP layer (reversed only) or alkalization were effective only after the 4 h sampling. Analytic ClOₓ-species determination yielded the same conclusions.

Pseudomonas aeruginosa (P. aeruginosa, ATCC 9027): In a non-limiting example, Sample 20 [AWT021] demonstrated the efficacy of regular, reversed and sandwiched assemblies (10 ppm of SC) vs. Escherichia coli and P. aeruginosa (10⁵ cfu/ml in TSB 1/500, 1 h).

Legionella spp. (ATCC 33152): In a non-limiting example, Samples 29-31 [AWT031, AWT043, and AWT055] examined the efficacy of reversed and sandwiched assemblies against Legionella (10⁴ cfu/ml) in TSB 1/500. Reversed and sandwiched assemblies were effective with 20 ppm of SC and above after 4 h. 1- to 2-log reduction was obtained after 0.5 h.

Clostridium perfringens (C. Perfringens, ATCC 13124): In a non-limiting example, Sample 43 [AWT041] was tested regarding the efficacy of reversed and sandwiched assemblies vs. Clostridium Perfringens spores. 3-log reduction was obtained for 10 ppm assemblies after 4 h.

In a non-limiting example, sample 70 [AWT070] was examined regarding the efficacies of reversed and sandwiched assemblies vs. Clostridium Perfringens spores. 3-log reduction was obtained for 10 ppm assemblies after 4 h, as in sample 43.

MS2-coliphage (ATCC 15597B1). In a non-limiting example, AWTvir001 demonstrated the efficacies of reversed and sandwiched Vinnol-based assemblies vs. MS2-coliphage (10⁵ pfu/ml). Sandwiched assemblies (10 ppm) was totally effective after 30 min while reversed assemblies were only effective after 4 h (˜2-log reduction after 30 min, 10 and 7.5 ppm).

In a non-limiting example, AWTvir002 demonstrated the efficacies of reversed and sandwiched Vinnol-based assemblies vs. MS2-coliphage (10⁵ pfu/ml). Sandwiched assemblies (7.5 and 10 ppm) was, once again, totally effective after 30 min while reversed assemblies were only effective after 4 h (˜2-log reduction after 30 min, 10 ppm).

Poliovirus (ATCC: VR-59) and Rotavirus (ATCC VR-899): In a non-limiting example, AWTvir003 examined the efficacy of reversed assemblies vs. Poliovirus and Rotavirus (˜10⁵ pfu/ml). in GTW, only 25 ppm of SC brought total eradication after 30 min. 10 ppm yielded only approx. 2.5-log reduction. Both SC contents were ineffective in CTW.

Cryptosporidium parvum (C. parvum): In a non-limiting example, AWTprot001 examined the efficacy of reversed assemblies vs. C. parvum (˜10⁶-10⁷ oocysts/L). In GTW, only 2- and 3-log reductions were exhibited after 4 h woth 10 and 25 ppm inserts, respectively. Efficacy of less than 2-log reduction was obtained after 30 min. both SC content were ineffective in CTW (less than 2-log reduction).

Microorganisms for Further Examination:

Bacteria (e.g., but not limited to, Enterococcus faecalis, Vibrio cholera, Salmonella typhi, Shigella spp., and Campylobacter jejuni), viruses (e.g., but not limited to, PhiX-174 bacteriophage), and Protozoa (e.g., but not limited to, Giardia lamblia)

AWT001

In a non-limiting example, Sample 1 [AWT001-003] was prepared with Hycar 26288 based sodium chlorite (“SC”) formulation in contact with CG8-H beads and the efficacy was examined. Sample 1 was ineffective after 4 h vs. Pseudomonas Aeruginosa.

Experimental Goals: Testing the current formulation for efficacy, organoleptic and kill kinetics in water like medium (TSB 1:500) in 2 concentrations of ClO₂: (1) Last active (100 ppm) and (2) comparison to competitors products (4 ppm). Testing Aseptrol based products and similar water disinfecting agents for efficacy against these formulations. Testing advantage over competitors' products regarding organoleptic. Growth curves and efficacy of Aseptrol on Klebsiela.

Experiment Content

| | | | | | SC Active | | | | | | |
|---|---|---|---|---|---|---|---|---|---|---|---|
| Model | Active | Additional | Model | | conc. | Vol. | | Inoculation | Sampling | incubation | Temp, |
| No. | material | materials | description | Medium | [ppm] | [ml] | Microorganism | [Log cfu/ml] | times | position | C. |
| I | SC + CG8- | Hycar | (SC + CG8- | TSB | 100 | 500 | P. Aeruginosa | 1 | 4, 24, | Static - | RT |
| | H | 26288, | H) 100 ppm | 1:500 | | | | | 168 | upside | |
| | | NH3 | | | | | | | hours | down | |
| K | SC + | Hycar | (SC + | TSB | 100 | 500 | P. Aeruginosa | 1 | 4, 24, | Static - | RT |
| | Kamin 70 | 26288, | Kamin 70) | 1:500 | | | | | 168 | upside | |
| | | NH3 | 100 ppm | | | | | | hours | down | |
| J | SC + CG8- | Hycar | (SC + CG8- | TSB | 4 | 500 | P. Aeruginosa | 1 | 4, 24, | Static - | RT |
| | H | 26288, | H) 4 ppm | 1:500 | | | | | 168 | upside | |
| | | NH3 | | | | | | | hours | down | |
| L | SC + | Hycar | (SC + | TSB | 4 | 500 | P. Aeruginosa | 1 | 4, 24, | Static - | RT |
| | Kamin 70 | 26288, | Kamin 70) | 1:500 | | | | | 168 | upside | |
| | | NH3 | 4 ppm | | | | | | hours | down | |
| A | Aseptrol | | Aseptrol | TSB | | 500 | Klebsiella | 1 | 4, 24, | Static - | RT |
| | (Potable | | (Potable | 1:500 | | | | | 168 | upside | |
| | Aqua) | | Aqua) | | | | | | hours | down | |
| | | | Klebsiela | | | | | | | | |
| B | Aseptrol | | Aseptrol | TSB | 4 | 500 | P. Aeruginosa | 1 | 4, 24, | Static - | RT |
| | (Potable | | (Potable | 1:500 | | | | | 168 | upside | |
| | Aqua) | | Aqua) | | | | | | hours | down | |
| C | MSR | | MSR | TSB | 6.5 | 500 | P. Aeruginosa | 1 | 4, 24, | Static - | RT |
| | Aquatabs | | Aquatabs | 1:500 | | | | | 168 | upside | |
| | | | | | | | | | hours | down | |
| D | Aquamira | | Aquamira | TSB | ^{~}10 | 500 | P. Aeruginosa | 1 | 4, 24, | Static - | RT |
| | | | | 1:500 | | | | | 168 | upside | |
| | | | | | | | | | hours | down | |
| NP | | | Control | TSB | | 50 | P. Aeruginosa | 1 | 4, 24, | Static - | RT |
| | | | P. Aeruginosa | 1:500 | | | | | 168 | upside | |
| | | | | | | | | | hours | down | |
| NK | | | Control | TSB | | 500 | Klebsiella | 1 | 4, 24, | Static - | RT |
| | | | Kelbsiela | 1:500 | | | | | 168 | upside | |
| | | | | | | | | | hours | down | |

Measurements of pH were taken once after 4 hours. One bottle from groups A, B, and C were used for taste testing (not inoculated).

AWT004

In a non-limiting example, Sample 2 [AWT004] was prepared with Hycar 26288 based sodium chlorite formulation in contact with CG8-H beads and utilized against 10⁵ cfu/ml of Raoultella (Klebsiella) Terrigena. 100 parts per million (ppm) assemblies were not effective.

AWT005

In a non-limiting example, Sample 3 [AWT005] was prepared with 25 and 100 ppm assemblies (with Hycar 26288 based SC formulation with KaMin at 70° C. calcined clay as an activating agent). Sample 3 was ineffective against 10⁵ cfu/ml of R. Terrigena.

AWT005 and AWT006

In a non-limiting example, Samples 4 and 5 [AWT006 and AWT005] performed with assemblies based on Hycar 26288, SC and KaMin at 70. Acidification of the medium to a pH of 4.0 (by direct addition of H₃PO₄) mitigated efficacy.

AWT007 and AWT008

In a non-limiting example, Samples 6 and 7 [AWT007 and AWT008] tested assemblies based on two separate formulations of SC and CG8 in Vinnol H30/48M in ethyl acetate were found effective (5-log reduction of R. Terrigena). A single layer of the combined formulations was ineffective. Hycar 26288 based SC formulation together with Hycar 26288 based CG8-h formulation (as activator) was not effective.

AWT009

In a non-limiting example, Sample 8 [AWT009] repeated the examination of 2-layers hand-applied Vinnol assemblies. Different drying schemes and formulation's solid contents were examined. Water-borne combined formulation of CG8-H and SC (alkalized by NH₃) in Hycar 26288 assemblies were found ineffective.

AWT010

In a non-limiting example, Sample 9 [AWT010] repeated the examination of 2-layers hand-applied Vinnol assemblies with different drying schemes. The assemblies were ineffective.

AWT011

In a non-limiting example, Sample 10 [AWT011] demonstrated efficacy of 10 ppm coated assemblies. Coated assemblies were prepared using RK K101 coater using 200 μm bar for the CG8-H:Vinnol/EtOAc formulation and 120 μm bar for the SC:Vinnol/EtOAc formulation. Regular assemblies (i.e. WE004 on top of WE003) were found effective.

AWT012

In a non-limiting example, Sample 11 [AWT012] proved the importance of correct drying scheme for the efficacy of coater-applied Vinnol assemblies. Hand applied assemblies demonstrated the stability of the wet formulations over time.

AWT013

In a non-limiting example, Sample 12 [AWT013] examined the effect of formulation foaming prior to the application and the influence of the durations the sheet is left at RT before it is introduced to the oven (working at 60° C.). Immediate introduction of the sheet to the oven was found to be crucial for efficacy. In-situ aeration of the formulation by vigorous mixing was found to be less important.

AWT014

In a non-limiting example, Sample 13 [AWT014] yielded the same conclusions as AWT013. Addition of wheat fibers yielded ineffective assemblies. Higher solid content (75%) of either CG8-H or SC did not affect the obtained efficacy significantly.

AWT015

In a non-limiting example, Sample 14 [AWT015] was tested at different drying temperature(s). Drying at 80° C. was found to be as efficient as drying at 60° C. Drying at 100° C. for 1 min and additional 30 min at 60° C. was found to yield less effective assemblies. This is the first trial to demonstrate the reversed assembly where the WE003 (CG8-H Vinnol/EtOAc formulation) layer is on top of the WE004 (SC:Vinnol/EtOAc formulation) layer which was found effective. 2 d old sheets were also effective.

AWT016

In a non-limiting example, Sample 15 [AWT016] demonstrated the efficacy of 10 ppm reversed and sandwiched assemblies. The importance of the rapid introduction of the sheet into the oven was once again exhibited. Reversed assemblies are coater-applied assemblies where the SC:Vinnol/EtOAc formulation layer (WE004, 120 μm) is deposited first and the CG8-H:Vinnol/EtOAc formulation layer (WE003, 200 μm) is deposited second. Sandwiched assemblies are constructed out of WE004 layer between two WE003 layers (200 μm). Aged regular assemblies demonstrated 1 week (w) shelf-life.

AWT017

In a non-limiting example, Sample 16 [AWT017] examined efficacy of regular assemblies in concentrated medium (i.e., lower dilution of the nutrient TSB 1/100 and 1/10 instead of the standard dilution of 1/500) and in lower temperature (4° C.). Temperature was found to be unimportant. Assemblies were not effective at TSB 1:100 and 1:10 media.

AWT018 and Additional Conclusions Comparing to AWT018 to AWT013-017

In a non-limiting example, Sample 17 [AWT018] demonstrated that all geometries, regular, reversed, and sandwiched, possess at least 1 w of shelf life at RT(˜25° C. and ˜30-50 % humidity). Solvent (EtOAc). binder (Vinnol H30/48M) and additives (CG8-H) were tested and found to possess no antimicrobial potential.

Experiments Goals: Focus on formulation WK003+WE004, LbL, coated that showed efficacy. Different parameters that will be tested for impact on efficacy are: (1) Reproducibility of benchmark formulation/fabrication parameters: Regular, reversed, and sandwiched geometries; (2) Shelf-life of benchmark assemblies—different geometries and utilization of protective tape; (3) Inefficacy of additives: WE003 (CGS-H+Vinnol/EtOAc), Vinnol/EtOAc. EtOAc, EtOAc.

| | | | | | | SC Active | | | | | |
|---|---|---|---|---|---|---|---|---|---|---|---|
| Model | Active | Additional | Formulation | Model | | conc. | Vol. | | Inoculation | Sampling | Temp, |
| No. | material | materials | number | description | Medium | [ppm] | [ml] | Microorganism | [Log cfu/ml] | times | C. |
| A | SC + | VInnol/ | WE003/8 + | Regular | TSB | 10 | 500 | Raoultella | 5 | 1, 4 | RT |
| B | CG8-H | EtOAc | WE004/12 | Reversed | 1:500 | | | terrigena | | hours | |
| C | | | | Sandwiched | | | | | | | |
| D | | | WE003/7 + | Regular, 6 | | | | | | | |
| | | | WE004/10 | d old | | | | | | | |
| E | | | | Regular, 6 | | | | | | | |
| | | | | d old, | | | | | | | |
| | | | | w/tape | | | | | | | |
| F | | | | Reversed, | | | | | | | |
| | | | | 6 d old | | | | | | | |
| G | | | | Sandwiched, | | | | | | | |
| | | | | 6 d old | | | | | | | |
| H | CG8-H | Vinnol/ | WE003/8 | WE003 | | N/A | | | | | |
| | | EtOAc | | only | | | | | | | |
| I | N/A | Vinnol/ | Vinnol/ | Vinnol/ | | | | | | | |
| | | EtOAc | EtOAc 20% | EtOAc | | | | | | | |
| | | | stock | | | | | | | | |
| J | N/A | EtOAc | N/A | 50 ml | | | | | | | |
| | | | | EtOAc | | | | | | | |
| NC | N/A | N/A | N/A | Negative | | N/A | 50 | Raoultella | 5 | 1, 4 | RT |
| | | | | Control | | | | terrigena | | hours | |

This is a yes/no experiment, thus counting is needed only for 0, 1 dilutions. Measure CIOₓ concentrations and swelling at 1, 4 hours. If any of formulations arc active will be tested for organoleptic attributes. Measure pH once after 4 hours.

Results: Microbiology Results

uptake, and most of all, higher CDO content. CDO is the only detected ClOx species. This hints that the local protonation is more significant. Formulation additives, i.e., CG8-H, Vinnol H30/48M, and Ethyl acetate, on the appropriate amounts, do not possess any AMA on their own or without the addition of SC.

Repeat benchmark formulation/fabrication parameters (regular, reversed, and sandwiched) as positive control/reproducibility study. Explore shelf life of sheets in various storage conditions; examine the efficiency of a protective tape. Explore formulation aging characteristics. Explore if and which layer porosity is of significance. Explore the influence of pH on TSB 1:100. Perform challenge test according to the EPA protocol (vary total of carbon, turbidity, temperature and total dissolved solids).

AWT019

In a non-limiting example, Sample 18 [AWT019] tested regular, reversed, and sandwiched assemblies prepared with alkalized (by addition of 25% NH₃ solution) SC formulation (WE007) that were found to be effective. Regular assemblies were found to be more susceptible for humidity-driven degradation. Assemblies were left exposed overnight under 40° C. and 80% humidity and the regular assembly efficacy degraded the most.

Experiments Goals: Focus on formulation WE003+WE004, LbL, coated that showed efficacy. Different parameters that will be tested for impact on efficacy are: (1) Reproducibility of benchmark formulation/fabrication parameters: Regular, reversed, and sandwiched geometries, (2) pH-dependence of efficacy in TSB 1:100, (3) Shelf-life of dry sheets—efficiency of protective tape—overnight @ 40° C./80% RH, and (4) NH₃-alkalized SC formulation—coater LbL geometries.

| | | | | | | SC Active | | | | | |
|---|---|---|---|---|---|---|---|---|---|---|---|
| Model | Active | Additional | Formulation | Model | | conc. | Vol. | | Inoculation | Sampling | Temp, |
| No. | material | materials | number | description | Medium | [ppm] | [ml] | Microorganism | [Log cfu/ml] | times | C. |
| A | SC + | VInnol/ | WE003/9 + | Regular | TSB | 10 | 500 | Raoultella | 5 | 1, 4 | RT |
| B | CG8-H | EtOAc | WE004/13 | Reversed | 1:500 | | | terrigena | | hours | |
| C | | | | Sandwiched | | | | | | | |
| D | SC | | WE004/13 | pH ~3.5 | TSB | | | | | | |
| E | | | | pH ~4.5 | 1:100 | | | | | | |
| F | | | | pH ~5.5 | | | | | | | |
| G | SC + | | WE003/8 + | Regular, overnight | TSB | | | | | | |
| | CG8-H | | WE004/12 | 40° C./80% RH | 1:500 | | | | | | |
| H | | | | Regular w/tape, | | | | | | | |
| | | | | overnight | | | | | | | |
| | | | | 40° C./80% RH | | | | | | | |
| I | | | | Reversed, | | | | | | | |
| | | | | overnight | | | | | | | |
| | | | | 40° C./80% RH | | | | | | | |
| J | | | | Reversed w/tape, | | | | | | | |
| | | | | overnight | | | | | | | |
| | | | | 40° C./80% RH | | | | | | | |
| K | | | | sandwiched, | | | | | | | |
| | | | | overnight | | | | | | | |
| | | | | 40° C./80% RH | | | | | | | |
| L | | | | Sandwiched | | | | | | | |
| | | | | w/tape, overnight | | | | | | | |
| | | | | 40° C./80% RH | | | | | | | |
| M | | Vinnol/ | WE003/9 + | Regular/007 | | | | | | | |
| N | | EtOAc/ | WE007/4 | Reversed/007 | | | | | | | |
| O | | NH₃ | | Sandwiched/007 | | | | | | | |
| NC(Q) | N/A | N/A | N/A | Negative | TSB | N/A | 50 | Raoultella | 5 | 1, 4 | RT |
| | | | | Control | 1:500 | | | terrigena | | hours | |
| NC(R) | N/A | N/A | N/A | Negative | TSB | N/A | 50 | Raoultella | 5 | 1, 4 | RT |
| | | | | Control | 1:100 | | | terrigena | | hours | |

This is a yes/no experiment, thus counting is needed for only 0,1 dilutions. Measure CIOₓ concentrations and swelling at 1,4 hours. If any of formulations are active will be tested for organoleptic attributes. Measurements of pH are recorded.

Results: Microbiology Results

solids/temperature. Explore if and which layer porosity is of significance. Spray applications are tested.

AWT020

In a non-limiting example, Sample 19 [AWT020] enabled efficacy in TSB 1:100 medium. A thinner WE004 (SC formulation) layer (12 μm) was applied to obtain assemblies with higher CG8-H loadings (since the area of the assembly is dictated by the SC layer density, applying a thinner SC layer will result in a larger assembly. A larger assembly will thereof hold a larger amount of CG8-H per bottle than a smaller assembly). The trial was unsuccessful, either because the CG8-H content was still insufficient or because the thin WE004 layers were of inconsistent thickness.

AWT021

In a non-limiting example, Sample 20 [AWT021] demonstrated the efficacy of regular, reversed and sandwiched assemblies (100 ppm of SC) vs. Escherichia coli and P. aeruginosa (10⁵ cfu/ml in TSB 1/500, 1 h). Efficacy was also demonstrated to be the same for 1 L receptacles (instead of 0.5 L).

Experimental Goals: Focus on formulation WE003+WE004, LbL, coated that showed efficacy. Different parameters that will be tested for impact on efficacy are: (1) reproducibility of benchmark formulation/fabrication parameters: Regular, reversed, and sandwiched geometries, (2) Challenge tests: (a) high volume—1 L and (b) different organisms: E. Coli and P. Aeruginosa, (3) elevating CG8-H actual concentration by applying thinner SC:Vinnol/EtOAc layer and thicker WE003 layer. Regular, reversed, and sandwiched geometries, TSB 1:500 and 1:100, and (4) efficacy of 20 ppm sheet in TSB 1:100.

Experiment Content

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | WE003/12 + | Regular, 0.5 L, | TSB | 10 | 500 | Raoultella | 5 | 1, 4 | RT |
| | CG8-HSC | EtOAc | WE004/15 | R. Terrigena | 1:500 | | | terrigena | | hours | |
| B | | | | Reversed, 0.5 L, | | | | | | | |
| | | | | R. Terrigena | | | | | | | |
| C | | | | Sandwiched, 0.5 L, | | | | | | | |
| | | | | R. Terrigena | | | | | | | |
| D | | | | Regular, 0.5 L, | | | | E. Coli | | | |
| | | | | E. Coli | | | | | | | |
| E | | | | Reversed, 0.5 L, | | | | | | | |
| | | | | E. Coli | | | | | | | |
| F | | | | Sandwiched, 0.5 L, | | | | | | | |
| | | | | E. Coli | | | | | | | |
| G | | | | Regular, 0.5 L, | | | | P. Aeruginosa | | | |
| | | | | P. Aeruginosa | | | | | | | |
| H | | | | Reversed, 0.5 L, | | | | | | | |
| | | | | P. Aeruginosa | | | | | | | |
| I | | | | Sandwiched, 0.5 L, | | | | | | | |
| | | | | P. Aeruginosa | | | | | | | |
| J | | | | Regular, 1 L, | | | 1000 | Raoultella | | | |
| | | | | R. Terrigena | | | | terrigena | | | |
| K | | | | Reversed, 1 L, | | | | | | | |
| | | | | R. Terrigena | | | | | | | |
| L | | | | Sandwiched, 1 L, | | | | | | | |
| | | | | R. Terrigena | | | | | | | |
| N | | | | Sandwiched, extra | | | 500 | | | | |
| | | | | CG8-H, 1:500 | | | | | | | |
| N | | | | Sandwiched, extra | TSB | | | | | | |
| | | | | CG8-H, 1:100 | 1:100 | | | | | | |
| O | | | | Sandwiched, 20 | | 20 | | | | | |
| | | | | ppm, 1:100 | | | | | | | |
| NC(P) | N/A | N/A | N/A | Negative Control | TSB | N/A | 50 | Raoultella | 5 | 1, 4 | RT |
| | | | | | 1:500 | | | terrigena | | hours | |
| NC(Q) | | | | | TSB | | | Raoultella | | | |
| | | | | | 1:100 | | | terrigena | | | |
| NC(R) | | | | | TSB | | | E. Coli | | | |
| | | | | | 1:500 | | | | | | |
| NC(S) | | | | | TSB | | | P. Aeruginosa | | | |
| | | | | | 1:500 | | | | | | |

This is a yes/no experiment, thus counting is needed only for 0,1 dilutions. Measure CIOₓ concentrations and swelling at 1, 4 hours. If any of formulations are active will be tested for organoleptic attributes Measure pH.

Results: Microbiology Results

CIOₓ and water uptake measurements

| | model | 1 h | | | | | 4 h | | | | | Water |
|---|---|---|---|---|---|---|---|---|---|---|---|---|
| Sample | description | Cl2 | CDO | ClO2— | total ClO | pH | Cl2 | CDO | ClO2— | total ClO | pH | uptake |
| A | Regular | 0 | 0 | 0 | 0 | 5.7 | 0 | 0 | 0 | 0 | 4.77 | 10.7% |
| B | Reversed | 0 | 0 | 0 | 0 | 5.1 | 0 | 4.8 | 0 | 4.8 | 4.52 | 23.1% |
| C | Sandwiched | 0 | 0 | 0 | 0 | 4.54 | 0 | 0 | 0 | 0 | 4.51 | 54.7% |
| M | Sandwiched, | 0 | 0 | 0 | 0 | 4.18 | 0 | 0 | 0 | 0 | 4.1 | 33.2% |
| | extra CG8-H | | | | | | | | | | | |

Summary and conclusions

the additional SC and CG8-H providing more CDO. Assemblies with higher loading of CG8-H and additional CG8-H only (WE003) slide succeeded in reducing 4 log in 4 h. the late efficacy is probably related to the slow release of the SC through two deposited layers of WE003.

Additional experiments: repeat benchmark formulation/fabrication parameters (regular, reversed, and sandwiched) as positive control/reproducibility study. Explore shelf life of sheets and wet formulation in various storage conditions; examine the efficiency of a protective tape. Perform challenge tests: (1) thicker nutrient: TSB 1:100 by increasing IX content in different geometries, (2) lower SC active concentration: 5 ppm, (3) sampling time: ½ h, (4) different microorganisms (viruses and protozoa), (5) varying turbidity/total of carbon/total of dissolved solids/temperature. Explore if and which layer porosity is of significance. Spray application utilization.

AWT022

In a non-limiting example, Sample 21 [AWT022] examined the efficacy of all assemblies at different initial pH of the medium (TSB 1/500 with initial pH-values of 4, 7, and 9). Reversed and sandwiched assemblies were found superior to the regular assembly at higher initial medium's pH (9). This is probably due to the higher CG8-H loading of the reversed and sandwiched assemblies and their geometry that forces the CDO precursor, SC, to flux through the top layer, loaded with CG8-H, enhancing its activation yield. Generally, reversed and sandwiched assemblies continuously exhibit significantly improved activation yield of SC to CDO in analytic measurements (Hach Autocat 9000). All assemblies demonstrated efficacy after 1 w of dry storage at RT.

AWT023

In a non-limiting example, Sample 22 [AWT023] assemblies were tested at 5 ppm (instead of 10 ppm) and were found to be partially effective, where the sandwiched assembly is superior. Furthermore, CG8-H content of reversed and sandwiched assemblies was significantly increased by applying additional layers of WE003. By this method it was possible to obtain efficacy in TSB 1:100 (5-log reduction of R. terrigena in 4 h).

AWT024

In a non-limiting example, Sample 23 [AWT024] demonstrated the feasibility of sprayed assemblies (using an airless automated spraying system, instead of the standard coater applied). 10 ppm reversed assemblies were prepared using airless spraying system and found to be effective. Coated assemblies of 5 ppm SC efficacy were slightly improved by increasing the CG8-H content (externally. i.e. addition of CG8-H only slide to the bottle). 7.5 ppm assemblies were found to be effective.

Experiments Goals: focus on formulation WE003+WE004, LbL, coated that showed efficacy. Different parameters that will be tested for impact on efficacy are (1) reproducibility of benchmark formulation/fabrication parameters: regular, reversed, and sandwiched geometries—concentration ladder trial and (2) preliminary inspection of sprayed sheets.

Experiment Content

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | WE003/12 + | Regular, 10 ppm | TSB | 10 | 500 | Raoultella | 5 | 1, | RT |
| B | CG8-HSC | EtOAc | WE004/16 | Reversed, 10 ppm | 1:500 | | | terrigena | | 4 hours | |
| C | | | | Sandwiched, 10 ppm | | | | | | | |
| D | | | | Regular, 7.5 ppm | | 7.5 | | | | | |
| E | | | | Reversed, 7.5 ppm | | | | | | | |
| F | | | | Sandwiched, 7.5 ppm | | | | | | | |
| G | | | | Regular, 5 ppm | | 5 | | | | | |
| H | | | | Reversed, 5 ppm | | | | | | | |
| I | | | | Sandwiched, 5 ppm | | | | | | | |
| J | | | | Regular, 5 ppm + CG8 | | | | | | | |
| | | | | (x2) | | | | | | | |
| K | | | | Reversed, 5 ppm + CG8 | | | | | | | |
| | | | | (x2) | | | | | | | |
| L | | | | Sandwiched, 5 ppm + | | | | | | | |
| | | | | CG8 (x2) | | | | | | | |
| N | | | | Reversed, Sprayed I, 10 | | 10 | | | | | |
| | | | | ppm | | | | | | | |
| N | | | | Reversed, Sprayed II, 10 | | | | | | | |
| | | | | ppm | | | | | | | |
| O | | | | Sprayed, SC only, 10 | | | | | | | |
| | | | | ppm + H3PO4 | | | | | | | |
| NC(P) | N/A | N/A | N/A | NC, TSB 1:500 | TSB | N/A | 50 | Raoultella | 5 | 1, | RT |
| | | | | | 1:500 | | | terrigena | | 4 hours | |

This is a yes/no experiment, thus counting is needed for only 0,1 dilutions. Measure CIOₓ concentrations and swelling at 1,4 hours. If any of formulations are active will be tested for organoleptic attributes. Measurements of pH are recorded.

Results: Microbiology Results

sample differ by the belt speed during the spraying of the WE004 (SC:Vinnol/EtOAc) layer. Faster belt velocity will dictate a lower surface concentration of SC.

Additional experiments: (1) repeat benchmark formulation/fabrication parameters (regular, reversed, and sandwiched) as positive control/reproducibility study, (2) explore shelf life of sheets and wet formulation in various storage conditions; examine the efficiency of a protective tape, (3)perform challenge tests: (a) Medium temperature and pH combined test, (b) different microorganisms (viruses and protozoa), (c) varying turbidity/total of carbon/total of dissolved solids/temperature, and (4) spray application utilization—retrial.

AWT025

In a non-limiting example, Sample 24 [AWT025] tested all 3 assemblies' efficacy in a combined challenge trial of low temperature (4° C.) and initial pH (4, 7, or 9). Full efficacy was obtained for all assemblies (regular, reversed, and sandwiched) at all conditions (10⁵ cfu/ml of R. terrigena, 1 h). In addition, application of BASF Kollidon 30 PVP (polyvinyl pyrrolidon, 16.67 wt % in 2-propanol, 30 min drying at 60° C.) “humidity protection” layer (12 μm or 120 μm) on top or between the layers of a reversed assembly was examined. The additional PVP layer was found to not impede efficacy or release kinetics (measured by Hach).

AWT026

Experiments Goals: focus on formulation WE003+WE004, LbL, coated that showed efficacy. Different parameters that are tested for impact on efficacy: (1) reproducibility of benchmark formulation/fabrication parameters: Regular, reversed, and sandwiched geometries—concentration ladder trial, and (2) shelf-life of two week old wet formulations and dry assemblies.

Experiment Content

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | PE038/2 + | Regular, fresh | TSB | 10 | 500 | Raoultella | 5 | 1, | RT |
| B | CG8-HSC | EtOAc | WE004/17 | Reversed, fresh | 1:500 | | | terrigena | | 4 hours | |
| C | | | | Sandwiched, fresh | | | | | | | |
| D | | | PE038/2 + | Reversed, 2 w old SC | | | | | | | |
| | | | WE004/13 | formulation (004) | | | | | | | |
| E | | | PE038/2 + | Reversed, 2 w old SC | | | | | | | |
| | | | WE007/4 | formulation (007) | | | | | | | |
| F | | | WE003/9 + | Regular, 2 w old | | | | | | | |
| G | | | WE004/11 | Reversed, 2 w old | | | | | | | |
| H | | | | Sandwiched, 2 w old | | | | | | | |
| I | | | | Regular, 2 w old | | | | | | | |
| | | | | w/protective tape | | | | | | | |
| J | | | | Reversed, 2 w old | | | | | | | |
| | | | | w/protective tape | | | | | | | |
| K | | | | Sandwiched, 2 w old | | | | | | | |
| | | | | w/protective tape | | | | | | | |
| L | | | WE003/9 + | Alk. Regular, 2 w old | | | | | | | |
| N | | | WE007/4 | Alk. Reversed, 2 w old | | | | | | | |
| N | | | | Alk. Sandwiched, | | | | | | | |
| | | | | 2 w old | | | | | | | |
| O | | | WE003/13 + | Sprayed (27/27), | | | | | | | |
| | | | WE004/14 | 1 w old | | | | | | | |
| P (NC) | N/A | N/A | N/A | NC | TSB | N/A | 50 | Raoultella | 5 | 1, | RT |
| | | | | | 1:500 | | | terrigena | | 4 hours | |
| Q (GC) | N/A | Humic acid | N/A | Growth curve, Humic | TSB | N/A | 50 | Raoultella | 5 | 1, | RT |
| | | | | acid 1:500 | 1:500 | | | terrigena | | 4 h | |

This is a yes/no experiment, thus counting is needed only for 0,1 dilutions. Measure CIOₓ concentrations and swelling at 1, 4 hours. If any of formulations are active will be tested for organoleptic attributes. Measure pH.

Results: Microbiology Results

the formation and consequent volatilization of CDO. The NH₃ residues are eliminated when the assembly is subjected to liquid water which penetrates and release large amount of protons form the IX.

Reverse-sprayed assembly is effective after 1 w of dry storage.

Additional experiments: (1) repeat benchmark formulation/fabrication parameters (regular, reversed, and sandwiched) as positive control/reproducibility study, (2) explore shelf life of sheets and wet formulation in various storage conditions; examine the efficiency of a protective tape; case studies regarding (a) protective tape, (b) evacuated Al and PE bags in 40° C./80% RH, (c) PVP (Kollidon 30) protection layer on reversed assemblies (top and intermediate), and (d) wet formulation alkalization (by NH₃), (3) perform challenge tests: (a) different microorganisms (viruses and protozoa), and (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(c) spray application utilization—optimization, and elaborated kinetics and residuals analysis (Hach).

AWT026

In a non-limiting example, Sample 25 [AWT026] examined the shelf life of all 3 assemblies after 2 w at RT conditions. While the reversed and sandwiched assembly preserved their efficacy, the aged regular sample was found ineffective. Application of top protection tape did not improved or damaged efficacy. Alkalization of the SC layer formulation (WE007, see, e.g., AWT019) managed to prolong the shelf life of the reversed assembly also to 2 w at RT. Sprayed assemblies (see, e.g., AWT024) exhibited shelf life of 1 w.

AWT027

In a non-limiting example, Sample 26 [AWT027] demonstrated sandwiched assemblies to possess efficacy (5-log reduction of R. terrigena, TSB 1:500, 4 h) also in large volume of 10 L. efficacy was also demonstrated at a medium with a total dissolved solids content of >1500 mg/L (using NaCl). Efficacy was not obtained at a medium with total organic carbon content (TOC) of more than 50 mg/L (by Humic acid).

AWT028

In a non-limiting example, Sample 27 [AWT028] made an effort in integrating weak acid cation exchange resin (ResinTech WACG-H) and calcined clay (KaMin 70C) as alternative activation agents (for CG8-H) which can be potentially integrated within the SC formulation layer itself). WACG-H and KaMin 70° C. where dispersed within alkalized SC:Vinnol/EtOAc formulation (WE007) and applied in reversed geometry. These weak acidifiers did not yield efficacy on their own without the presence of CG8-H layer. Nevertheless, their presence did not impede efficacy (together with CG8-H).

AWT029

In a non-limiting example, Sample 28 [AWT029] tested sandwiched vs 10⁵ cfu/ml of R. terrigena in media with varying TOC (by humic acid sodium salt). Efficacy was obtained uo to a TOC level of 50 mg/L.

AWT031, AWT043 and AWT055

In a non-limiting example, Samples 29-31 [AWT031, AWT043, and AWT055] examined the efficacy of reversed and sandwiched assemblies against Legionella (10⁴ cfu/ml) in TSB 1/500. Reversed and sandwiched assemblies were effective with 20 ppm of SC and above after 4 h. 1- to 2-log reduction was obtained after 0.5 h.

AWT031

Experiments Goals: focus on formulation WE003+WE004, LbL, coated that showed efficacy. Different parameters that will be tested for impact on efficacy are: (1) repeat examination of benchmark assemblies for demonstration of reproducibility and positive control and (2) explore efficacy vs. Legionella.

Experiment Content:

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | WE003/13 + | Regular, 10 ppm | TSB | 10 | 500 | Legionella | 4 | 1, | RT |
| B | CG8-H | EtOAc | WE004/17 | Reversed, 10 ppm | 1:500 | | | | | 4 hours | |
| C | | | | Sandwiched, 10 ppm | | | | | | | |
| D | | | | Reversed, 20 ppm | | | | | | | |
| E | | | | Reversed, 7.5 ppm | | | | | | | |
| F | | | | Sandwiched, 5 ppm | | | | | | | |
| NC-J | N/A | N/A | N/A | NC | | N/A | 50 | Legionella | 4 | 1, | RT |
| | | | | | | | | | | 4 hours | |

This is a yes/no experiment, thus counting is needed only for 0, 1 dilutions. Measure ClOₓ concentrations and swelling at 1, 4 hours. If any of formulations are active will be tested for organoleptic attributes. Measure pH.

AWT043

Experiments goals: focus on formulation WE003+WE004, LbL, coated that showed efficacy. Different parameters that will be tested for impact on efficacy are: (1) repeat examination of benchmark assemblies for demonstration of reproducibility and positive control and (2) explore efficacy vs. Legionella.

Experiments Content

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | MI038/14 + | Reversed, 20 ppm | TSB | See model | 500 | Legionella | 4 | ½, | RT |
| B | CG8-H | EtOAc | MI044/11 | Reversed, 10 ppm | 1:500 | description | | | | 4 hours | |
| C | | | | Reversed, 7.5 ppm | | | | | | | |
| D | | | | Sandwiched, 20 ppm | | | | | | | |
| E | | | | Sandwiched, 10 ppm | | | | | | | |
| F | | | | Sandwiched, 7.5 ppm | | | | | | | |
| NC-J | N/A | N/A | N/A | NC | | N/A | 50 | Legionella | 4 | ½, | RT |
| | | | | | | | | | | 4 hours | |

This is a yes/no experiment, thus counting is needed for only 0,1 dilutions. Measure CIOₓ concentrations and swelling at 1,4 hours. If any of formulations are active will be tested for organoleptic attributes. Measurements of pH are recorded.

Results: Microbiology Results

examination of benchmark assemblies for demonstration of reproducibility and positive control and (2) explore eficacy vs. Legionella.

Experiments Content

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | MI038/14 + | Reversed, 30 ppm | TSB | See model | 500 | Legionella | 4 | ½, | RT |
| B | CG8-H | EtOAc | MI044/11 | Reversed, 20 ppm | 1:500 | description | | | | 4 hours | |
| C | | | | Reversed, 10 ppm | | | | | | | |
| D | | | | Sandwiched, 30 ppm | | | | | | | |
| E | | | | Sandwiched, 20 ppm | | | | | | | |
| F | | | | Sandwiched, 10 ppm | | | | | | | |
| NC-J | N/A | N/A | N/A | NC | | N/A | 50 | Legionella | 4 | ½, | RT |
| | | | | | | | | | | 4 hours | |

This is a yes/no experiment, thus counting is needed only for 0, 1 dilutions. Measure CIOₓ concentrations and swelling at 1, 4 hours. If any of formulations are active will be tested for organoleptic attributes. Measure pH.

Results: Microbiology Results

See FIG. 19 for Legionella viable counts.

AWT032

In a non-limiting example, Sample 32 [AWT032] yielded results indicating all 3 assemblies' 30 min efficacy vs. Aspergillus Niger (mold. 10² cfu/ml) and Candida Albicans (Yeast 10² cfu/ml). Regular, reversed, and sandwiched assemblies were also first tested vs. R. Tenigena under EPA #2 conditions (4° C., TOC>10 mg/L, TDS>1500 mg/L, pH˜9) turbidity simulant was not available) and were found ineffective.

Experiments Goals: Focus on formulation WE003+WE004, LbL, coated that showed efficacy. Different parameters that will be tested for impact on efficacy arc: (1) repeat examination of benchmark assemblies for demonstration of reproducibility and positive control,(2) combined challenge test vs. R. Terrigena —EPA tests #1 and #2—experimental formulae and Aquamira (TOC w/Humic acid sodium salt. TDS w/NaCl), and (3) challenge test: new organisms: Aspergillus Niger and Candida Albicans.

Experiment Content

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | WE003/13 + | EPA #1 - Reversed | TSB | 10 | 500 | R. Terrigena | 5 | 0.5, | RT |
| B | CG8-H | EtOAc | WE004/19 | EPA #2 - Reversed | 1:500 | | | | | 4 hours | 4° C. |
| C | | | | EPA #1 - Sandwiched | | | | | | | RT |
| D | | | | EPA #2 - Sandwiched | | | | | | | 4° C. |
| E | | | | Regular, Niger | PDB | | | A. Niger | 2 | | RT |
| F | | | | Sandwiched, Niger | 1:500 | | | | | | RT |
| G | | | | Regular, Candida | | | | C. Albicans | 2 | | RT |
| H | | | | Sandwiched, Candida | | | | | | | RT |
| I | Aquamira | Aquamira | N/A | EPA #1, Aquamira | TSB | | | R. Terrigena | 5 | | RT |
| J | A | B | | EPA #2, Aquamira | 1:500 | | | | | | 4° C. |
| K | | | | Niger, Aquamira | PDB | | | A. Niger | 2 | | RT |
| L | | | | Candida, Aquamira | 1:500 | | | C. Albicans | | | RT |
| NC-M | N/A | N/A | N/A | EPA #1 - NC | TSB | N/A | 50 | R. Terrigena | 5 | 0.5, | RT |
| NC-N | | | | EPA #2 - NC | 1:500 | | | | | 4 hours | 4° C. |
| NC-O | | | | A. Niger - NC | PDB | | | A. Niger | 2 | | RT |
| NC-P | | | | C. Albicans- NC | 1:500 | | | C. Albicans | | | RT |

40° C./80% RH, (c) PVP (Kollidon 30) protection layer on reversed assemblies (top and intermediate), and (d) wet formulation alkalization (by NH₃), (3) challenge tests (all w.r.t reference product, Aquamira/Aseptrol efficacy): (a) different microorganisms (viruses and protozoa), (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

perform turbidity measurements, (4) elaborated kinetics and residuals analysis (Hach), (5) explore and develop know-how and demands for future large-scale production: method, production parameters, packaging, etc., and (6) develop a “one-shot” (indicating that the assembly was not used or exposed to destructive conditions prior to the intended usage) and “ready-to-use” (all CDO is leached into the medium and performed its AMA) indicators.

AWT033

In a non-limiting example, Sample 33 [AWT033] was tested for 4 weeks (at RT) old assemblies efficacy (predecessors: t₀:AWT018/19, t_{2w}:AWT026). Reversed and sandwiched assemblies retained their efficacy, regular assembly do not, except when the SC layer formulation is alkalized (i.e., WE007 is used instead if WE004). Application of protective tape or PVP protection top or intermediate layer did not improved or impact efficacy.

Experiments Goals: focus on formulation WE003+WE004, LbL, coated that showed efficacy. Different parameters that will be tested for impact on efficacy are: (1) repeat examination of benchmark assemblies for demonstration of reproducibility and positive control, (2) examine shelf-life of printed assemblies, 4 w old and explore parameters: (a) protective tape, (b) SC:Vinnol/EtOAc alkalization, (c) PVP barrier layer, and (3) shelf-life of wet Vinnol/EtOAc formulations—ppd.

| | | | | | | SC | | | | | |
|---|---|---|---|---|---|---|---|---|---|---|---|
| | | | | | | Active | | | | | |
| Model | Active | Additional | Formulation | | | conc. | Vol. | | Inoculation | Sampling | Temp, |
| No. | material | materials | number | Model description | Medium | [ppm] | [ml] | Microorganism | [Log cfu/ml] | times | C. |
| A | SC + | Vinnol/ | WE003/13 + | Regular, fresh/PC | TSB 1:500 | 10 | 500 | R. Terrigena | 5 | 0.5, 4 | RT |
| B | CG8-H | EtOAc | WE004/20 | Reversed, fresh/PC | | | | | | hours | |
| C | | | | Sandwiched, fresh/PC | | | | | | | |
| D | | | WE003/8 + | Regular, PE bag | | | | | | | |
| E | | | WE004/12 | Reversed, PE bag | | | | | | | |
| F | | | | Sandwiched, PE bag | | | | | | | |
| G | | | | Regular, PE bag | | | | | | | |
| | | | | w/protective tape | | | | | | | |
| H | | | | Reversed, PE bag | | | | | | | |
| | | | | w/protective tape | | | | | | | |
| I | | | | Sandwiched, PE bag | | | | | | | |
| | | | | w/protective tape | | | | | | | |
| J | | Vinnol/ | WE003/9 + | Regular, alkalized | | | | | | | |
| K | | EtOAc, | WE007/4 | Reversed, alkalized | | | | | | | |
| L | | NH3 | | Sandwiched, alkalized | | | | | | | |
| M | | Vinnol/ | PE038/2 + | Reversed, 12 mm PVP | | | | | | | |
| | | EtOAc + | WE004/16 | top-coat | | | | | | | |
| N | | Kollidon | | Reversed, 120 mm | | | | | | | |
| | | 30/EtOH | | PVP top-coat | | | | | | | |
| O | | | | Reversed, 12 mm PVP | | | | | | | |
| | | | | interim barrier | | | | | | | |
| NC-P | N/A | N/A | N/A | NC | | N/A | 50 | | | | |

This is a yes/no experiment, thus counting is needed only for 0, 1 dilutions. Measure CIOₓ concentrations and swelling at 1, 4 hours. If any of formulations are active will be tested for organoleptic attributes. Measure pH.

Results: Microbiology Results

WE003 layer application. The efficacy of the sandwiched assembly hints that sufficient amount of IX may overcome fabrication tweaks and imperfections.

Additional experiments: (1) repeat benchmark formulation/fabrication parameters (regular, reversed, and sandwiched) as positive control/reproducibility study. (2) Explore shelf life of sheets and wet formulation in various storage conditions; examine the efficiency of a protective tape and perform case studies: (a) protective tape, (b) evacuated Al and PE bags in 40° C./80% RH, (c) PVP (Kollidon 30) protection layer on reversed assemblies (top and intermediate), (d) wet formulation alkalization (by NH₃). Perform challenge tests (all w.r.t reference product, Aquamira/Aseptrol efficacy): (a) different microorganisms (viruses and protozoa), and (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) Elaborated kinetics and residuals analysis (Hach). (5) Explore and develop know-how and demands for future large-scale production: method, production parameters, packaging, etc. (6) Develop a “one-shot” (indicating that the assembly was not used or exposed to destructive conditions prior to the intended usage) and “ready-to-use” (all CDO is leached into the medium and performed its AMA) indicators.

AWT034 and AWT036

In a non-limiting example, Samples 34 and 35 [AWT034 and AWT036] were examined for the efficacy of reversed and sandwiched (against Aquamira drops reference) assemblies in 4 media/conditions cases: EPA#1 (TOC-0.1 mg/L, TDS-100 mg/L, pH-7, T-25° C.), EPA#1+TSB1:500, EPA#2 (4° C., TOC>10 mg/L, TDS>1500 mg/L, pH˜9), EPA#2+TSB 1:500. Both reversed and sandwiched assemblies were effective under all cases (Aquamira drops were not effective under EPA#2+TSB 1:500 and less effective under EPA#2).

AWT034

Experiments Goals: focus on formulation WE003+WE004, LbL, coated that showed efficacy. Different parameters that will be tested for impact on efficacy are: (1) repeat examination of benchmark assemblies for demonstration of reproducibility and positive control, (2) combined challenge test, EPA tests #1 and #2, w/ and w/o TSB 1:500, reversed and sandwiched assemblies vs. Aquamira, and (3) shelf-life of wet Vinnol/EtOAc formulations—ppd.

Experiments Content

| | | | | | | SC | | | | | |
|---|---|---|---|---|---|---|---|---|---|---|---|
| | | | | | | Active | | | | | |
| Model | Active | Additional | Formulation | | | conc. | | Micro- | Inoculation | Sampling | Temp, |
| No. | material | materials | number | Model description | Medium | [ppm] | Vol. [ml] | organism | [Log cfu/ml] | times | C. |
| A | SC + | Vinnol/ | WE003/13 + | Reversed, EPA #1 | TSB | 10 | 500 | R. Terrigena | 5 | 0.5, 4 | RT |
| | CG8-H | EtOAc | WE004/22 | w/TSB | 1:500 | | | | | hours | |
| B | | | | Reversed, EPA #1 | | | | | | | |
| | | | | w/o TSB | | | | | | | |
| C | | | | Reversed, EPA #2 | | | | | | | |
| | | | | w/TSB | | | | | | | |
| D | | | | Reversed, EPA #2 | | | | | | | |
| | | | | w/o TSB | | | | | | | |
| E | | | | Sandwiched, EPA | | | | | | | |
| | | | | #1 w/TSB | | | | | | | |
| F | | | | Sandwiched, EPA | | | | | | | |
| | | | | #1 w/o TSB | | | | | | | |
| G | | | | Sandwiched, EPA | | | | | | | |
| | | | | #2 w/TSB | | | | | | | |
| H | | | | Sandwiched, EPA | | | | | | | |
| | | | | #2 w/o TSB | | | | | | | |
| I | Aquamira | Aquamira | N/A | Aquamira, EPA #1 | | | | | | | |
| | A | B | | w/TSB | | | | | | | |
| J | | (H3PO4) | | Aquamira, EPA #1 | | | | | | | |
| | | | | w/o TSB | | | | | | | |
| K | | | | Aquamira, EPA #2 | | | | | | | |
| | | | | w/TSB | | | | | | | |
| L | | | | Aquamira, EPA #2 | | | | | | | |
| | | | | w/o TSB | | | | | | | |
| NC-M | N/A | N/A | N/A | NC, EPA #1 w/TSB | | | | | | | |
| NC-N | | | | NC, EPA #1 w/o | | | | | | | |
| | | | | TSB | | | | | | | |
| NC-O | | | | NC, EPA #2 w/TSB | | | | | | | |
| NC-P | | | | NC, EPA #2 w/o | | N/A | 50 | | | | |
| | | | | TSB | | | | | | | |

This is a yes/no experiment, thus counting is needed only for 0, 1 dilutions. Measure ClOₓ concentrations and swelling at 1, 4 hours. If any of formulations are active will be tested for organoleptic attributes. Measure pH. The conditions are: (1) EPA #1 w/TSB-TSB

Summary and conclusions

Reversed and sandwiched assemblies were found effective (after 4 h, shorter times were not sampled due to work overload of over priorities in the microbiology laboratory) in all test conditions. Both assemblies were effective even in the harder test condition (unlike in AWT032). EPA test #2 condilions with TSB 1:500 (TSB 1:500, 10 mg/L of humic acid Na-salt. 1500 mg/L of NaCl, pH˜9 by NH₃, 4° C.).

Aquamira was not effective under the EPA test #2 conditions with TSB 1:500 (as in AWT032).

Additional experiments: (1) repeat benchmark formulation/fabrication parameters (regular, reversed, and sandwiched) as positive control/reproducibility study. (2) explore shelf life of sheets and wet formulation in various storage conditions; examine the efficiency of a protective tape. Case studies: (a) protective tape, (b) evacuated Al and PE bags in 40° C./80%RH, (c) PVP (Kollidon 30) protection layer on reversed assemblies (top and intermediate), (d) wet formulation alkalization (by NH₃). (3) Challenge tests (all w.r.t reference product, Aquamira/Aseptrol efficacy): (a) different microorganisms (viruses and protozoa) and (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) explore and develop know-how and demands for future large-scale production: method, production parameters, packaging, etc., and (6) develop a “one-shot” (indicating that the assembly was not used or exposed to destructive conditions prior to the intended usage) and “ready-to-use” (all CDO is leached into the medium and performed its AMA) indicators.

AWT036

Experiments Goals: focus on formulation WE003+WE004, LbL, coated that showed efficacy. Different parameters that will be tested for impact on efficacy are: (1) Repeat examination of benchmark assemblies for demonstration of reproducibility and positive control and (2) continue testing using a combined challenge test, EPA tests #1 and #2, w/and w/o TSB 1:500, reversed and sandwiched assemblies vs. Aquamira.

Experiments Content

| | | | | | | SC | | | | | |
|---|---|---|---|---|---|---|---|---|---|---|---|
| | | | | | | Active | | | | | |
| Model | Active | Additional | Formulation | | | conc. | | Micro- | Inoculation | Sampling | Temp, |
| No. | material | materials | number | Model description | Medium | [ppm] | Vol. [ml] | organism | [Log cfu/ml] | times | C. |
| A | SC + | Vinnol/ | WE003/14 + | Reversed, | TSB | 10 | 500 | R. Terrigena | 5 | 0.5, 4 | RT |
| | CG8-H | EtOAc | WE004/24 | EPA #1 w/TSB | 1:500 | | | | | hours | |
| B | | | | Reversed, | | | | | | | |
| | | | | EPA #1 w/o TSB | | | | | | | |
| C | | | | Reversed, | | | | | | | |
| | | | | EPA #2 w/TSB | | | | | | | |
| D | | | | Reversed, | | | | | | | |
| | | | | EPA #2 w/o TSB | | | | | | | |
| E | | | | Sandwiched, | | | | | | | |
| | | | | EPA #1 w/TSB | | | | | | | |
| F | | | | Sandwiched, | | | | | | | |
| | | | | EPA #1 w/o TSB | | | | | | | |
| G | | | | Sandwiched, | | | | | | | |
| | | | | EPA #2 w/TSB | | | | | | | |
| H | | | | Sandwiched, | | | | | | | |
| | | | | EPA #2 w/o TSB | | | | | | | |
| I | Aquamira | Aquamira | N/A | Aquamira, | | | | | | | |
| | A | B | | EPA #1 w/TSB | | | | | | | |
| J | | (H3PO4) | | Aquamira, | | | | | | | |
| | | | | EPA #1 w/o TSB | | | | | | | |
| K | | | | Aquamira, | | | | | | | |
| | | | | EPA #2 w/TSB | | | | | | | |
| L | | | | Aquamira, | | | | | | | |
| | | | | EPA #2 w/o TSB | | | | | | | |
| NC-M | N/A | N/A | N/A | NC, EPA #1 w/TSB | | | | | | | |
| NC-N | | | | NC, EPA #1 w/o | | | | | | | |
| | | | | TSB | | | | | | | |
| NC-O | | | | NC, EPA #2 w/TSB | | | | | | | |
| NC-P | | | | NC, EPA #2 w/o | | N/A | 50 | | | | |
| | | | | TSB | | | | | | | |

This is a yes/no experiment, thus counting is needed only for 0, 1 dilutions. Measure ClOₓ concentrations and swelling at 1, 4 hours. If any of formulations are active will be tested for organoleptic attributes. Measure pH. Conditions: (1) EPA #1 w/TSB-TSB 1:500, (2) EPA #1 w/o TSB-1 mg/L Humic acid sodium salt and 100 mg/L NaCl, (3) EPA #2 w/TSB-TSB 1:500+10 mg/L Humic acid sodium salt, 1500 mg/L NaCl, 1 drop of NH₃ 25% in 4° C.,

Additional Experiments: (1) repeat benchmark formulation/fabrication parameters (regular, reversed, and sandwiched) as positive control/reproducibility study, (2) explore shelf life of sheets and wet formulation in various storage conditions; examine the efficiency of a protective tape. Case studies: (a) protective tape—see AWT033 and future trials, (b) evacuated Al and PE bags in 40° C./80% RH—see AWT037. (c) PVP (Kollidon 30) protection layer on reversed assemblies (top and intermediate)—see AWT033 and future trials, (d) wet formulation alkalization (by NH₃)—see AWT033 and future trials, (3) challenge tests (all w.r.t reference product, Aquamira/Aseptrol efficacy): (a) different microorganisms (viruses and protozoa)—see AWT031 (Legionella) and AWT041 (Clostridium), (b) EPA protocol: see AWT034, AWT036 (current) and future trials.

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), explore and develop know-how and demands for future large-scale production: method, production parameters, packaging, etc., and (6) develop a “one-shot” (indicating that the assembly was not used or exposed to destructive conditions prior to the intended usage) and “ready-to-use” (all CDO is leached into the medium and performed its AMA) indicators—see AWT035 and AWT037.

AWT035

In a non-limiting example, Sample 36 [AWT035] was tested regarding the efficacy of assemblies prepared with 1 m old SC formulations (with or without alkalization, WE004 and WE007, respectively). The assemblies' efficacy is not impacted by the age of the formulation. 1 month (m) old sprayed assembly was found to be ineffective (see AWT024). Indicator-integrated assemblies were also tested. Indicator reagents are the oxidation susceptible tartrazine (yellow pigment) and the oxidation-tolerant phtalocyanine blue (0.5 wt % in the dry film, each). Assemblies with indicator reagents integrated in the CG8-H layer (WE018 instead if WE003) yielded color change from green to blue several minutes after introduction of the assembly to the medium. This effect was observed both for regular and reversed assemblies. Integrating the indicator reagents into the SC layer formulation did not yield color change. Indicator integration did not impact efficacy (5-log reduction of R. terrigena in 30 m).

Experiments Goals: focus on formulation WE003+WE004, LbL, coated that showed efficacy. Different parameters that will be tested for impact on efficacy are: (1) repeat examination of benchmark assemblies for demonstration of reproducibility and positive control, (2) shelf-life of wet Vinnol/EtOAc formulations (>1 month) w/and w/o alkalization of the SC:Vinnol/EtOAc formulation (WE007 and WE004, respectively), (3) efficacy of indicator-integrated assemblies, and (4) shelf-life of sprayed reverse assemblies.

Experiments Content

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/13 + | Regular, PC | TSB | 10 | 500 | R. Terrigena | 5 | 0.5, 4 | RT |
| B | CG8-H | EtOAc | WE004/22 | Reversed, PC | 1:500 | | | | | hours | |
| C | | | | Sandwiched, PC | | | | | | | |
| D | | | WE003/14 + | Regular, aged WE004 | | | | | | | |
| E | | | WE004/14 | Reversed, aged | | | | | | | |
| | | | | WE004 | | | | | | | |
| F | | | | Sandwiched, aged | | | | | | | |
| | | | | WE004 | | | | | | | |
| G | | Vinnol/ | WE003/14 + | Regular, aged WE007 | | | | | | | |
| H | | EtOAc + | WE007/4 | Reversed, aged | | | | | | | |
| | | | | WE007 | | | | | | | |
| I | | NH₃ | | Sandwiched, aged | | | | | | | |
| | | | | WE007 | | | | | | | |
| J | | Vinnol/ | WE018/1 + | Regular, indicator on | | | | | | | |
| | | EtOAc + | WE004/22 | bottom (WE003) | | | | | | | |
| K | | Phtalocyanine | WE003/14 + | Regular, indicator on | | | | | | | |
| | | blue + | WE017/1 | top (WE004) | | | | | | | |
| L | | Tartrazine | WE003/14 + | Reversed, indicator on | | | | | | | |
| | | | WE017/1 | bottom (WE004) | | | | | | | |
| M | | | WE018/1 + | Reversed, indicator on | | | | | | | |
| | | | WE004/22 | top (WE003) | | | | | | | |
| N | | Vinnol/ | WE003/12 + | Sprayed, 1 month old | | | | | | | |
| | | EtOAc | WE004/16 | | | | | | | | |
| NC-O | N/A | N/A | N/A | NC | | N/A | 50 | | | | |

This is a yes/no experiment, thus counting is needed only for 0, 1 dilutions. Measure ClOₓ concentrations and swelling at 1, 4 hours. If any of formulations are active will be tested for organoleptic attributes. Measure pH.

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temprature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) explore and develop know-how and demands for future large-scale production: method, production parameters, packaging, etc., and develop a “one-shot” (indicating that the assembly was not used or exposed to destructive conditions prior to the intended usage) and “ready-to-use” (all CDO is leached into the medium and performed its AMA) indicators.

AWT037

In a non-limiting example, Sample 37 [AWT037] was examined regarding indicator-integrated assemblies and yielded the same results as AWT035. Efficacies of all 3 geometries were examined after HALT (Highly-accelerated life test) of 40° C. and 80% humidity. Reversed and sandwiched assemblies were found effective after 1 month, regular was not. Placing the assembly in humidity resistant aluminum bag retained the efficacy of the regular geometry also. This trial does not come in agreement with later trials conducted in HALT. This may be due to malfunction in the humidity chamber resulted in lower actual humidity that programmed).

Experiments Goals: focus on formulation WE003+WB004, LbL, coated that showed efficacy. Different parameters that will be tested for impact on efficacy are: (1) repeat examination of benchmark assemblies for demonstration of reproducibility and positive control, (2) shelf-life of wet VinnoL/EtOAc formulations (>1 month) in evacuated PE and Al bags, 40° C. /80%RH, (3) efficacy of indicator-integrated assemblies—whole assemblies.

Experiments Content

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/14 + | Regular, PC | TSB | 10 | 500 | R. Terrigena | 5 | 0.5, 4 | RT |
| B | CG8-H | EtOAc | WE004/24 | Reversed, PC | 1:500 | | | | | hours | |
| C | | | | Sandwiched, PC | | | | | | | |
| D | | | PE038/2 + | Regular, 4 w old, PE | | | | | | | |
| | | | WE004/17 | bag, 40/80 | | | | | | | |
| E | | | | Reversed, 4 w old, PE | | | | | | | |
| | | | | bag, 40/80 | | | | | | | |
| F | | | | Sandwiched, 4 w old, | | | | | | | |
| | | | | PE bag, 40/80 | | | | | | | |
| G | | | | Regular, 4 w old, Al | | | | | | | |
| | | | | evac. bag, 40/80 | | | | | | | |
| H | | | | Reversed, 4 w old, Al | | | | | | | |
| | | | | evac. bag, 40/80 | | | | | | | |
| I | | | | Sandwiched, 4 w old, | | | | | | | |
| | | | | Al evac. bag, 40/80 | | | | | | | |
| J | | Vinnol/ | WE018/1 + | Regular w/indicator, | | | | | | | |
| | | EtOAc + | WE004/22 | transparent BM | | | | | | | |
| K | | Phtalocyanine | | Regular w/indicator, | | | | | | | |
| | | blue + | | Blue BM | | | | | | | |
| L | | Tartrazine | | Reversed w/indicator, | | | | | | | |
| | | | | transparent BM | | | | | | | |
| M | | | | Reversed w/indicator, | | | | | | | |
| | | | | Blue BM | | | | | | | |
| N | | Vinnol/ | WE003/12 + | BM w/o SC | | | | | | | |
| | | EtOAc | WE004/16 | | | | | | | | |
| NC-O | N/A | N/A | N/A | NC | | N/A | 50 | | | | |

This is a yes/no experiment, thus counting is needed only for 0, 1 dilutions. Measure CIOₓ concentrations and swelling at 1, 4 hours. If any of formulations are active will be tested for organoleptic attributes. Measure pH.

Results: Microbiology Results

parameters: (a) assembly type—reversed, (b) alkalization of SC formulation—Yes/No, (c) Indicator presence—Yes/No, (d) PVP (Kollidon 30, Luvitec VA64) or different top-coat and its thickness, (e) packaging—protective tape (if and when available), plastic bag, Al, (3) challenge tests (all w.r.t reference product, Aquamira/Aseptrol efficacy): (a) different microorganisms (viruses and protozoa) and (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

and (4) elaborated kinetics and residuals analysis (Hach).

AWT038 (t₀), AWT059, and AWT060 (t₁ₘ)

In a non-limiting example, Samples 38-40 [AWT038 (t₀), AWT059, and AWT060 (t₁ₘ)] examined shelf life of reversed assemblies under RT and HALT. Assemblies were not effective after 1 m in HALT of 40° C. and 80% humidity but was effective when stored in RT. Addition of indicator reagents, top protection layer of Kollidon 30 (12 or 120 μm, 16.67 wt % in 2-propanol) or Luvitec VA64 (12 or 120 μm, 16.67 wt % in 2-propanol) did not influence resulting efficacy. Analytical measurements of ClOₓ-species supported the efficacy trial results.

Experiments Goals: focus on formulation WE003+WE004, LbL, coated that showed efficacy. Different parameters that will be tested for impact on efficacy are: examine shelf-life of reversed assemblies stored under RT and HALT (40° C./80% RH) and examine parameters: alkalization, indicator, protection layer type and width. This is a time zero trial.

Experiments Content

| | | | | | | | | | SC | | | | | |
|---|---|---|---|---|---|---|---|---|---|---|---|---|---|---|
| | | | | | | | | | Active | | | | | |
| Model | Active | Additional | Formulation | Model description | | | | | conc. | Vol. | Micro- | Inoculation | Sampling | Temp, |
| No. | material | materials | number | (alk/ind/coat/thk) | | | | Medium | [ppm] | [ml] | organism | [Log cfu/ml] | times | C. |
| A | SC + | Vinnol/ | WE003/15 + | N | N | N | — | TSB | 10 | 500 | R. Terrigena | 5 | 0.5, 4 | RT |
| B | CG8-H | EtOAc (see | WE004/24 | N | Y | N | — | 1:500 | | | | | hours | |
| C | | also model | | Y | N | N | — | | | | | | | |
| D | | description) | | Y | Y | N | — | | | | | | | |
| E | | | | N | N | Kol30 | 12 | | | | | | | |
| F | | | | N | Y | Kol30 | 12 | | | | | | | |
| G | | | | Y | N | Kol30 | 12 | | | | | | | |
| H | | | | Y | Y | Kol30 | 12 | | | | | | | |
| I | | | | N | N | Kol30 | 120 | | | | | | | |
| J | | | | N | Y | Kol30 | 120 | | | | | | | |
| K | | | | Y | N | Kol30 | 120 | | | | | | | |
| L | | | | Y | Y | Kol30 | 120 | | | | | | | |
| M | | | | N | N | VA64 | 12 | | | | | | | |
| N | | | | N | V | VA64 | 12 | | | | | | | |
| O | | | | Y | N | VA64 | 12 | | | | | | | |
| P | | | | Y | Y | VA64 | 12 | | | | | | | |
| Q | | | | N | N | VA64 | 120 | | | | | | | |
| R | | | | N | Y | VA64 | 120 | | | | | | | |
| S | | | | Y | N | VA64 | 120 | | | | | | | |
| T | | | | Y | V | VA64 | 120 | | | | | | | |

| | | | | | | |
|---|---|---|---|---|---|---|
| NC-O | N/A | N/A | N/A | NC | N/A | 50 |

This is a yes/no experiment, thus counting is needed for only 0,1 dilutions. Measure CIOₓ concentrations and swelling at 1,4 hours. If any of formulations are active will be tested for organoleptic attributes. Measure pH. Kol30—Kollidon 30, VA64—Luvitec VA64, 16.6% solutions in 2-propanol, 12 or 120 μm top-coats were utilized.

Results: Microbiology Results

Summary and Conclusions

All printed reversed assemblies are effective at time zero (after production) vs. R. Terrigena after 4 h (after inoculation, shorter times were not sampled).

The indicator-integrated IX layer did change its color as expected from green to light blue in all examined configurations (i.e., alkalization of the SC layer and application of PVP top coat do not influence indication)

Additional Experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions, using the following variable parameters: assembly type—reversed, alkalization of SC formulation (yes/no), indicator presence—(yes/no), PVP (Kollidon 30, Luvitec VA64) or different top-coat and its thickness, and packaging—protective tape (if and when available), plastic bag, Al, (3) challenge tests (all w.r.t reference product, Aquamira/Aseptrol efficacy): (1) different microorganisms (viruses and protozoa) and (2) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

and (4) elaborated kinetics and residuals analysis (Hach).

AWT059 and AWT060

AWT059-60 explored the shelf-life of reversed assemblies after storage of 1 m under HALT of 40 C and 80% RH. None of the assemblies preserved its efficacy (as in AWT057-60). Alkalization of the SC layer or application of additional PVP top coat (Kollidon 30 or Luvitec VA64) did not assist in preventing degradation. The indicator-integrated assemblies were observed to be pale blue indicating in the consumption of tartrazine by CDO action. RT-stored assemblies apparently do not appear to be degraded. Additional trials are examining the influence of temperature and humidity on the assemblies' shelf-life.

AWT059

Experiments Goals: Focus on formulation WE003+WE004, LbL, coated that showed efficacy. Different parameters that will be tested for impact on efficacy are: Examine shelf-life of reversed assemblies stored under RT and HALT (40° C./80% RH). Examine parameters: alkalization, indicator, protection layer type and width. This is 1 month trial. The trial will be divided into three experiments, AWT059 (9 samples), AWT060 (9 samples), and AWT061 (2 samples).

Experiments Content

| | | | | | | | | | SC | | | Inocu- | | |
|---|---|---|---|---|---|---|---|---|---|---|---|---|---|---|
| | | | | | | | | | Active | | | lation | | |
| Model | Active | Additional | Formulation | Model description | | | | | conc. | Vol. | Micro- | [Log | Sampling | Temp, |
| No. | material | materials | number | (alk/ind/coat/thk) | | | | Medium | [ppm] | [ml] | organism | cfu/ml] | times | C. |
| A | SC + | Vinnol/ | WE003/15 + | N | N | N | — | TSB 1:500 | 10 | 500 | R. Terrigena | 5 | 0.5, 4 | RT |
| B | CG8-H | EtOAc (see | WE004/24 | N | Y | N | — | | | | | | hours | |
| C | | also model | | Y | N | N | — | | | | | | | |
| D | | description) | | Y | Y | N | — | | | | | | | |
| E | | | | N | N | Kol30 | 12 | | | | | | | |
| F | | | | N | Y | Kol30 | 12 | | | | | | | |
| G | | | | Y | N | Kol30 | 12 | | | | | | | |
| H | | | | Y | Y | Kol30 | 12 | | | | | | | |
| I | | | | N | N | Kol30 | 120 | | | | | | | |
| J | | | | N | N | N | — | EPA #2 | | | | | | |
| K | | | | N | Y | N | — | medium | | | | | | |
| L | | | | Y | N | N | — | | | | | | | |
| M | | | | Y | Y | N | — | | | | | | | |
| N | | | | N | N | Kol30 | 12 | | | | | | | |
| O | | | | N | Y | Kol30 | 12 | | | | | | | |
| P | | | | Y | N | Kol30 | 12 | | | | | | | |
| Q | | | | Y | Y | Kol30 | 12 | | | | | | | |
| R | | | | N | N | Kol30 | 120 | | | | | | | |

| | | | | | | | |
|---|---|---|---|---|---|---|---|
| NCEPA#1 | N/A | N/A | N/A | NC EPA #1 | TSB 1:500 | N/A | 50 |
| NCEPA#2 | N/A | N/A | N/A | NC EPA #2 | EPA #2 | N/A | 50 |

This is a yes/no experiment, thus counting is needed only for 0, 1 dilutions. Measure ClOₓ concentrations and swelling at 1, 4 hours. If any of formulations are active will be tested for organoleptic attributes. Measure pH.

AWT060 (t₁ₘ)

Experiments Goals: Focus on formulation WE003+WE004, LbL, coated that showed efficacy. Different parameters that will be tested for impact on efficacy are: examine shelf-life of reversed assemblies stored under RT and HALT (40° C./80% RH). Examine parameters: alkalization, indicator, protection layer type and width. This is a 1 month trial. The trial will be divided into three experiments, AWT059 (8 samples), AWT060 (8 samples), and AWT061 (4 samples).

Experiments Content

| | | | | | | | | | SC | | | | | |
|---|---|---|---|---|---|---|---|---|---|---|---|---|---|---|
| | | | | | | | | | Active | | | | | |
| Model | Active | Additional | Formulation | Model description | | | | | conc. | Vol. | Micro- | Inoculation | Sampling | Temp, |
| No. | material | materials | number | (alk/ind/coat/thk) | | | | Medium | [ppm] | [ml] | organism | [Log cfu/ml] | times | C. |
| A | SC + | Vinnol/ | WE003/15 + | N | N | N | — | TSB | 10 | 500 | R. Terrigena | 5 | 0.5, 4 | RT |
| B | CG8-H | EtOAc (see | WE004/24 | N | Y | N | — | 1:500 | | | | | hours | |
| C | | also model | | Y | N | N | — | | | | | | | |
| D | | description) | | Y | Y | N | — | | | | | | | |
| E | | | | N | N | Kol30 | 12 | | | | | | | |
| F | | | | N | Y | Kol30 | 12 | | | | | | | |
| G | | | | Y | N | Kol30 | 12 | | | | | | | |
| H | | | | Y | Y | Kol30 | 12 | | | | | | | |

| I | Reverse, fresh | | | | |
|---|---|---|---|---|---|
| J | N | N | N | — | EPA #2 |
| K | N | Y | N | — | medium |
| L | Y | N | N | — | |
| M | Y | Y | N | — | |
| N | N | N | Kol30 | 12 | |
| O | N | Y | Kol30 | 12 | |
| P | Y | N | Kol30 | 12 | |
| Q | Y | Y | Kol30 | 12 | |

| | | | | | | | |
|---|---|---|---|---|---|---|---|
| R | | | | Reverse, fresh | | | |
| NCEPA#1 | N/A | N/A | N/A | NC EPA #1 | TSB | N/A | 50 |
| | | | | | 1:500 | | |
| NCEPA#2 | N/A | N/A | N/A | NC EPA #2 | EPA #2 | N/A | 50 |

This is a yes/no experiment, thus counting is needed only for 0, 1 dilutions. Measure ClOₓ concentrations and swelling at 1, 4 hours. If any of formulations are active will be tested for organoleptic attributes. Measure pH.

AWT039

In a non-limiting example, Sample 41 [AWT039] was examined regarding the efficacies of sandwiched and reversed assemblies. 15 min after insertion. The assemblies were found to be only partly effective (in EPA#1 medium) or not at all (in EPA#2 medium).

Experiments Goals: Focus on formulation WE003+WE004, LbL, coated that showed efficacy. Different parameters that will be tested for impact on efficacy are: (1) examine efficacy of the present assemblies vs. those of Aquatabs (chlorine tablets), and (2) examine importance of geometry against IX content.

Experiments Content:

| | | | | | | SC Active | | | | | |
|---|---|---|---|---|---|---|---|---|---|---|---|
| Model | Active | Additional | Formulation | Model description | | conc. | Vol. | | Inoculation | Sampling | Temp, |
| No. | material | materials | number | (alk/ind/coat/thk) | Medium | [ppm] | [ml] | Microorganism | [Log cfu/ml] | times | C. |
| A | SC + | Vinnol/ | WE003/15 + | Regular PC | TSB 1:500 | 10 | 500 | R. Terrigena | 5 | 1, 4 hours | RT |
| B | CG8-H | EtOAc | WE004/26 | Reversed PC | | | | | | | |
| C | | (see also | | Sandwiched PC | | | | | | | |
| D | | model | | Regular 12 mic | | | | | | | |
| | | description) | | WE004 | | | | | | | |
| E | | | | Regular 24 mic | | | | | | | |
| | | | | WE004 | | | | | | | |
| F | | | | Regular 40 mic | | | | | | | |
| | | | | WE004 | | | | | | | |
| G | | | | Regular 100 mic | | | | | | | |
| | | | | WE004 | | | | | | | |
| H | | | | Reversed 12 mic | | | | | | | |
| | | | | WE003 | | | | | | | |
| I | | | | Reversed 24 mic | | | | | | | |
| | | | | WE003 | | | | | | | |
| J | | | | Reversed 40 mic | | | | | | | |
| | | | | WE003 | | | | | | | |
| K | | | | Reversed 120 mic | | | | | | | |
| | | | | WE003 | | | | | | | |
| L | | | | Reversed EPA #2 | | | | | | | |
| M | NaDCC | N/A | N/A | Aquatabs EPA #1 | | | | | | | |
| N | | | | Aquatabs EPA #2 | | | | | | | |
| NC | N/A | N/A | N/A | NC | | N/A | 50 | | | | |

[Specimens B, L, and M are to be sampled at the 15 minutes time point.]

This is a yes/no experiment, thus counting is needed only for 0, 1 dilutions. Measure CIOₓ concentrations and swelling at 1, 4 hours. If any of formulations arc active will be tested for organoleptic attributes. Measure pH.

Results: Microbiology Results

penetration into the vinnol layer, releasing the CDO. This time is unnecessary when the biocide is directly administrated into the medium. One may solve this issue by applying thinner layers of vinnol or tweaking with the fabrication parameters. However, this may also influence the shelf life and degradation rate of the dry assembly.

Additional experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) perform challenge tests (all w.r.t reference product, Aquamira/Aseptrol efficacy): (a) different microorganisms (viruses and protozoa), (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial explores routes for improvement of the sensory effect of the assemblies. Possible solutions: (a) SC content reduction, (b) SC/IX ratio optimization, (c) utilization of ClOₓ-species' scavengers/neutralizers such as Na₂S₂O₃, phenols, alkaline zeolites, weak (and strong) base anion exchange resins, active carbon, etc., and (6) neutralize ClOₓ's traces in incubation stage.

AWT040

In a non-limiting example, Sample 42 [AWT040] was tested regarding the efficacies of regular, reversed and sandwiched assemblies when CDO-neutralizing solution (0.03% Na₂S₂O₃ in 0.85% Saline) is applied during sampling. During sampling, neutralizing solution was added to the seeded sample in the petri dish in similar volume (for each dilution). Efficacy was not impacted. Hence, the antimicrobial activity is concluded within the bottle and not within the seeded growth culture. Neutralizer solution did not possess antimicrobial feature.

Experiments Goals: focus on formulation WE003+WE004, LbL, coated that showed efficacy. Different parameters that will be tested for impact on efficacy are: (1) examine CDO/ClO₂⁻ neutralizing by sodium thiosulfate (Na₂S₂O₃): (a) negative control: does Na₂S₂O₃ possess AMA, (b) does neutralizer is practically needed, active assemblies efficacy sampling w/and w/o neutralization, (c) neutralizer efficiency—Hach trial, and (2) residual efficacy of assemblies.

Experiments Content

| | | | | | | SC | | | | | |
|---|---|---|---|---|---|---|---|---|---|---|---|
| | | | | | | Active | | | | | |
| Model | Active | Additional | Formulation | | | conc. | | Micro- | Inoculation | Sampling | Temp, |
| No. | material | materials | number | Model description | Medium | [ppm] | Vol. [ml] | organism | [Log cfu/ml] | times | C. |
| A | SC + | Vinnol/ | WE003/15 + | Regular | TSB | 10 | 500 | R. Terrigena | 5 | 1, 4 | RT |
| B | CG8-H | EtOAc (see | WE004/26 | Reversed | 1:500 | | | | | hours | |
| C | | also model | | Sandwiched | | | | | | | |
| D | | description) | | Aquamira | | | | | | | |
| E | | | | Regular | | | | | | | |
| | | | | w/Neutralizer | | | | | | | |
| F | | | | Reversed | | | | | | | |
| | | | | w/Neutralizer | | | | | | | |
| G | | | | Sandwiched | | | | | | | |
| | | | | w/Neutralizer | | | | | | | |
| H | | | | Aquamira | | | | | | | |
| | | | | w/Neutralizer | | | | | | | |
| I | | | | Neutralizer only | | | | | | | |
| | | | | @ t0 | | | | | | | |
| NC-J | N/A | N/A | N/A | NC | | N/A | 50 | | | | |
| NC-K | N/A | N/A | N/A | NC w/neutralizer @ | | N/A | 50 | | | | |
| | | | | t_sampling | | | | | | | |

Residual Efficacy Trial

| | | | | | | SC Active | | | | | |
|---|---|---|---|---|---|---|---|---|---|---|---|
| Model | Active | Additional | Formulation | | | conc. | Vol. | | Inoculation | Sampling | Temp, |
| No. | material | materials | number | Model description | Medium | [ppm] | [ml] | Microorganism | [Log cfu/ml] | times | C. |
| L | SC + | Vinnol/ | WE003/15 + | Regular RE | TSB 1:500 | 10 | 500 | R. Terrigena | 5 | 1, 4 hours | RT |
| M | CG8-H | EtOAc (see | WE004/26 | Reversed RE | | | | | | | |
| N | | also model | | Sandwiched RE | | | | | | | |
| | | description) | | | | | | | | | |
| NC-RE | N/A | N/A | N/A | NC RE | | N/A | 50 | | | | |

Hach: Measure samples A, B, C, and D with and without neutralizer, where neutralizing volumes are: equal volume (100 ml sample+100 ml neutralizer) and equal mass⁺(198 ml sample +2 ml neutralizer).

This is a yes/no experiment, Ihas counting is needed only for 0, 1 dilutions. Neutralizer composition: Saline 0.85% +Na₂S₂O₃ 0.3%, autoclaved. Method of application: add similar volume to the sample, mix thoroughly and seed. RE: fill assemblies' bottles with medium, incubate for 4 h, remove assembly, inoculate, incubate for additional 4 h and sample. Measure pH.

Results: Microbiology Results

Analytic Measurements

| | | | I | | | | II | | | | III | | | |
|---|---|---|---|---|---|---|---|---|---|---|---|---|---|---|
| | | | equivalent volume | | | | equivalent mass | | | | w/o Neutralizer | | | |
| | | | | | | total | | | | total | | | | total |
| # | Sample | pH | Cl2 | ClO2 | ClO2− | ox′ | Cl2 | ClO2 | ClO2− | ox′ | Cl2 | ClO2 | ClO2− | ox′ |
| A | Regular | 4.58 | 0 | 0 | 0 | 0 | 0 | 0 | 0 | 0 | 0.2 | 0 | 4.364 | 4.364 |
| B | Reversed | 4.39 | 0 | 0 | 0 | 0 | 0 | 0 | 0 | 0 | 0 | 1.15 | 1.932 | 3.08 |
| C | Sandwiched | 4.2 | 0 | 0 | 0 | 0 | 0 | 0 | 0 | 0 | 0 | 2.62 | 0 | 2.62 |
| D | Aquamira | 3.51 | 0 | 0 | 0 | 0 | 0.5 | 0 | 0.928 | 0.928 | 0 | 0.3 | 7.316 | 7.62 |

Summary and conclusions

All assemblies are effective vs. R. Terrigena. Addition of CIOₓ neutralizing solution did not affect efficacy (in active sample) or viability (in NC) of the assemblies and microorganisms, respectively.

The sampling protocol utilized dictated mixing of the sample with an equivalent volume of a neutralizes. Since the neutralizer solution is significantly more concentrated than the medium (ca. by 300), the neutralizer is far in excess. This can be also observed in the Hach results. Smaller excess of neutralizer (˜x2) was also examined. This trial was also successful in eliminating CIOₓ's, excluding the Aquamira (which may offer a larger CIOₓ concentration).

The sampling protocol utilized is efficient, but it is not as robust. It is actually adopted form Aseptrol® regulation protocol for viruses and protozoa.

All assemblies do possess residual efficacy. This is since the assemblies do release an active agent, CDO/ClO₂ into the medium which can be theoretically and practically retained for long time periods at some extent. The somewhat inconsistent results can originate from small extent CDO annihilation prior to the introduction of inoculation.

Additional Experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (b) assembly type—reversed, (b) alkalization of SC formulation (yes/no), (c) indicator presence—(yes/no), (d) PVP (Kollidon 30, Luvitec VA64) or different top-coat and its thickness, (e) packaging—protective tape (if and when available), plastic bag, Al, (3) challenge tests (all w.r.t reference product, Aquamira/Aseptrol efficacy): (a) different microorganisms (viruses and protozoa), (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), and (5) perform a combined AMA-analytic-sensory trial. This trial explores the sensory effect of the assemblies including: SC content reduction, SC/IX ratio optimization, utilization of ClOₓ-species' scavengers/neutralizers such as Na₂SₛO₃, phenols, alkaline zeolites, weak (and strong) base anion exchange resins, active carbon, etc., and (6) neutralizing ClOₓ's traces in incubation stage.

AWT041

In a non-limiting example, Sample 43 [AWT041] was tested regarding the efficacy of reversed and sandwiched assemblies vs. Clostridium Perfringens spores. 3-log reduction was obtained for 10 ppm assemblies after 4 h.

Experiments Goals: focus on formulation WE003+WE004, LbL, coated that showed efficacy. Different parameters that will be tested for impact on efficacy are: (1) repeat examination of benchmark assemblies for demonstration of reproducibility and positive control and (2) explore efficacy vs. Clostridium.

Experiments Content

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | MI038/14 + | Reversed, 20 ppm | TSB | See | 500 | Clostridium | 4 | ½, 4 | RT |
| B | CG8-H | EtOAc | MI044/11 | Reversed, 10 ppm | 1:500 | model | | | | hours | |
| C | | | | Reversed, 7.5 ppm | | description | | | | | |
| D | | | | Sandwiched, 20 ppm | | | | | | | |
| E | | | | Sandwiched, 10 ppm | | | | | | | |
| F | | | | Sandwiched, 7.5 ppm | | | | | | | |
| NC-J | N/A | N/A | N/A | NC | | N/A | 50 | Clostridium | 4 | 1, 4 hours | RT |

This is a yes/no experiment, thus counting is needed only for 0, 1 dilutions. Measure CIOₓ concentrations and swelling at 1, 4 hours. If any of formulations are active will be tested for organoleptic attributes. Measure pH.

Results: Microbiology Results

AWT042

In a non-limiting example, Sample 44 [AWT042] was examined regarding the efficacy of reversed and sandwiched (with 120 or 12 μm SC layer) assemblies at reduced SC contents (2.5, 5, 7.5, and 10 ppm). Efficacy was obtained after 0.5 h down to 7.5 ppm in reversed assemblies and down to 5 ppm in sandwiched assemblies. 5 ppm yielded efficacy after 5 h in both assemblies. 2.5 ppm was partially effective in the standard sandwiched assembly (2-log reduction after 0.5 h, 4-log reduction after 4 h). Sandwiched assemblies with 12 μm SC layer were not as effective as the standard sandwiched assemblies, probably due to incoherent deposition of the thin layer.

Experiments Goals: focus on formulation WE003+WE004, LbL, coated that showed efficacy. Different parameters that will be tested for impact on efficacy are: (1) examine efficacy, kinetics and organoleptics of the present assemblies with varying concentrations, (2) utilization of ClOₓ's-neutralizer, and (3) efficacy of potable aqua I₂ tablets.

Experiments Content (AMA Only)

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/15 + | Reversed 10 ppm | TSB 1:500 | 10 | 500 | R. Terrigena | 5 | 0.5, 4 | RT |
| B | CG8-H | EtOAc (see | WE004/26 | Reversed 7.5 ppm | | | | | | hours | |
| C | | also model | | Reversed 5 ppm | | | | | | | |
| D | | description) | | Reversed 2.5 ppm | | | | | | | |
| E | | | | Sandwiched 10 ppm | | | | | | | |
| F | | | | Sandwiched 7.5 ppm | | | | | | | |
| G | | | | Sandwiched 5 ppm | | | | | | | |
| H | | | | Sandwiched 2.5 ppm | | | | | | | |
| I | | | | Sandwiched (12 μm) | | | | | | | |
| | | | | 10 ppm | | | | | | | |
| J | | | | Sandwiched (12 μm) | | | | | | | |
| | | | | 7.5 ppm | | | | | | | |
| K | | | | Sandwiched (12 μm) | | | | | | | |
| | | | | 5 ppm | | | | | | | |
| L | | | | Sandwiched (12 μm) | | | | | | | |
| | | | | 2.5 ppm | | | | | | | |
| M | | | | Reversed, 10 ppm, | | | | | | | |
| | | | | w/Neutralizer | | | | | | | |
| N | Iodine | | | Potable aqua I₂ | | | | | | | |
| | | | | tablets | | | | | | | |
| NC | N/A | N/A | N/A | NC | | N/A | 50 | | | | |

This is a yes/no experiment, thus counting is needed only for 0, 1 dilutions. Measure ClOₓ concentrations and swelling at 1, 4 hours. If any of formulations are active will be tested for organoleptic attributes. Measure pH.

Results: Microbiology Results

Analytic Measurements

| | | 0.5 hr | | | | | 4 hr | | | | |
|---|---|---|---|---|---|---|---|---|---|---|---|
| Material | | Cl2 | ClO2 | ClO2− | total ox′ | pH | Cl2 | ClO2 | ClO2− | total ox′ | pH |
| A - regular | A [I] | 0 | 0.388 | 2.696 | 3.08 | 5.47 | 0 | 1.072 | 4.396 | 5.47 | 4.72 |
| | A [II] | 0 | 0.22 | 2.732 | 2.95 | 5.4 | 0 | 0.844 | 4.932 | 5.78 | 4.72 |
| | A [III] | 0 | 0 | 2.916 | 2.92 | 5.39 | 0 | 0.924 | 5.212 | 6.14 | 4.64 |
| B - reversed | B [I] | 0 | 0 | 0 | 0 | 4.97 | 0 | 2.808 | 0 | 2.81 | 4.53 |
| | B [II] | 0 | 0 | 0 | 0 | 4.91 | 0 | 3.12 | 0 | 3.12 | 4.63 |
| | B [III] | 0 | 0 | 0 | 0 | 4.87 | 0 | 2.832 | 0 | 2.83 | 4.49 |
| C - sandwiched | C [I] | 0 | 0 | 0 | 0 | 5.03 | 0 | 1.2 | 2.712 | 3.91 | 4.32 |
| (200/120/200) | C [II] | 0 | 0 | 0 | 0 | 5.07 | 0 | 1.46 | 1.68 | 3.14 | 4.35 |
| | C [III] | 0 | 0 | 0 | 0 | 5.14 | 0.7 | 0 | 4.5 | 4.5 | 4.22 |
| D - sandwiched | D [I] | 0 | 2.592 | 0.612 | 3.2 | 4.97 | 1.044 | 0 | 3.612 | 3.61 | 4.28 |
| (200/12/200) | D [II] | 0 | 4.492 | 0 | 4.49 | 4.99 | 0 | 1.332 | 3.896 | 5.23 | 4.44 |
| | D [III] | 0.768 | 0 | 4.696 | 4.7 | 4.61 | 0 | 4.716 | 0 | 4.72 | 4.36 |

Summary and Conclusions

Reversed and sandwiched assemblies are fully effective down to 7.5 ppm of SC. The sandwiched assembly is also effective down to 5 ppm where the reversed is only effective after 4 h. the sandwiched assembly is even partially effective with only 2.5 ppm where the reversed assembly is ineffective.

The difference in efficacies probably originates form the higher IX content of the sandwiched assembly (ca. 20% more CG8-H per assembly) and better activation geometry, as indicated by the measured pH values. However, when one looks at the analytical results, only the reversed assembly exhibits total conversion of SC to CDO after 4 h. Additionally, the reversed and sandwiched assemblies were effective.

Another unexpected observation is the relatively low efficacy of the thin-SC-layer sandwiched assembly. We would have expected it to be higher due to the higher IX content.

Potable Aqua Iodine tablets were effective.

Addition of ClOₓ-neutralizer did not affect efficacy of the reversed assembly, also after only 30 min

Additional experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters are: assembly type—reversed, alkalization of SC formulation (yes/no), indicator presence (yes/no), PVP (Kollidon 30, Luvitec VA64) or different top-coat and its thickness, packaging—protective tape (if and when available), plastic bag, Al, (3) perform challenge tests (all w.r.t reference product, Aquamira/Aseptrol efficacy): (a) different microorganisms (viruses and protozoa), (b)EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) perform a combined AMA-analytic-sensory trial including testing: SC content reduction, SC/IX ratio optimization, and utilization of ClOₓ-species' scavengers/neutralizers such as Na₂SₛO₃, phenols, alkaline zeolites, weak (and strong) base anion exchange resins, active carbon, etc., and (6) neutralize ClOₓ's traces in incubation stage.

AWT044 and AWT052

In a non-limiting example, Samples 45 and 46 [AWT044 and AWT052] examined the influence of the treated bottle agitation and the location of the assembly within the bottle (either on the bottle's bottom or just underneath the water level). Agitation was found to be crucial for achieving efficacy, specifically in short times (i.e., 30 min). If and when the bottle is not agitated, placing the assembly at top of the bottle (immersed) is better than placing it in the bottle bottom. This is probably due to CDO higher density which caused it to sink in the bottle bottom, unless it is properly agitated. Analytic CIOₓ species determination yielded the same conclusions.

AWT044

Experiments Goals: Focus on formulation WE003+WE004, LbL, coated that showed efficacy. Efficacy and kinetics of the assemblies with varying locations in the bottle is examined.

Experiments Content

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003 + | EPA #2, | TSB 1:500 | 10 | 500 | R. Terrigena | 5 | 0.5, 4 hours | RT |
| | CG8-H | EtOAc (see | WE004 | shake, cap | | | | | | | |
| B | | also model | | EPA #2, | | | | | | | |
| | | description) | | shake, cone | | | | | | | |
| C | | | | EPA #2, | | | | | | | |
| | | | | shake, bottom | | | | | | | |
| D | | | | EPA #2, | | | | | | | |
| | | | | no shake, cap | | | | | | | |
| E | | | | EPA #2, | | | | | | | |
| | | | | no shake, cone | | | | | | | |
| F | | | | EPA #2, | | | | | | | |
| | | | | no shake, bottom | | | | | | | |
| G | | | | EPA #1, | | | | | | | |
| | | | | shake, cap | | | | | | | |
| H | | | | EPA #1, | | | | | | | |
| | | | | shake, cone | | | | | | | |
| I | | | | EPA #1, | | | | | | | |
| | | | | shake, bottom | | | | | | | |
| J | | | | EPA #1, | | | | | | | |
| | | | | no shake, cap | | | | | | | |
| K | | | | EPA #1, | | | | | | | |
| | | | | no shake, cone | | | | | | | |
| L | | | | EPA #1, | | | | | | | |
| | | | | no shake, bottom | | | | | | | |
| NC-1 | N/A | N/A | N/A | NC, EPA #1 | | N/A | 50 | | | | |
| NC-2 | N/A | N/A | N/A | NC, EPA #2 | | N/A | 50 | | | | |

This is a yes/no experiment, thus counting is needed only for 0, 1 dilutions. Measure CIOₓ concentrations and swelling at 1, 4 hours. If any of formulations are active will be tested for organoleptic attributes. Measure pH.

Results: Microbiology Results

AWT052

Experiments Goals: (1) Explore efficacy of blow molded bottles (GMPack) vs. R Tertigena in water under EPA #1 and #2 and (2) examine the influence of the location of the assembly in the bottle and the effect of agitation.

Experiments Content (AMA only)

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Blow-molded bottle, | See notes | 10 | 500 | R. Terrigena | 5 | 0.5, 4 hours | EPA#1: |
| | CG8-H | EtOAc (see | WE004 | 0.05 mg_IX/ml, | and | | | | | | RT |
| | | also model | | EPA#1 | model | | | | | | EPA#2: |
| B | | description) | | Blow-molded bottle, | descrip- | | | | | | 4° C. |
| | | | | 0.05 mg_IX/ml, | tion | | | | | | |
| | | | | EPA#2 | | | | | | | |
| C | | | | Blow-molded bottle, | | | | | | | |
| | | | | 0.08 mg_IX/ml, | | | | | | | |
| | | | | EPA#1 | | | | | | | |
| D | | | | Blow-molded bottle, | | | | | | | |
| | | | | 0.08 mg_IX/ml, | | | | | | | |
| | | | | EPA#2 | | | | | | | |
| E | | | | Rev, on bot., | | | | | | | |
| | | | | EPA#2 | | | | | | | |
| F | | | | Sandw, on bot., | | | | | | | |
| | | | | EPA#2 | | | | | | | |
| G | | | | Rev, on top, | | | | | | | |
| | | | | EPA#2 | | | | | | | |
| H | | | | Sandw, on top, | | | | | | | |
| | | | | EPA#2 | | | | | | | |
| I | | | | Rev, agitated, | | | | | | | |
| | | | | EPA#2 | | | | | | | |
| J | | | | Sandw, agitated, | | | | | | | |
| | | | | EPA#2 | | | | | | | |
| K | | | | Rev, on bot., | | | | | | | |
| | | | | EPA#1 | | | | | | | |
| L | | | | Sandw, on bot., | | | | | | | |
| | | | | EPA#1 | | | | | | | |
| M | | | | Rev, on top, | | | | | | | |
| | | | | EPA#1 | | | | | | | |
| N | | | | Sandw, on top, | | | | | | | |
| | | | | EPA#1 | | | | | | | |
| O | | | | Rev, agitated, | | | | | | | |
| | | | | EPA#1 | | | | | | | |
| P | | | | Sandw, agitated, | | | | | | | |
| | | | | EPA#1 | | | | | | | |
| NCQ | N/A | N/A | N/A | NC EPA #1 | | N/A | 50 | | | | |
| NCR | N/A | N/A | N/A | NC EPA #2 | | N/A | 50 | | | | |

This is a yes/no experiment, thus counting is needed only for 0, 1 dilutions. Refrain from agitating bottles (except from during sampling). _Hach: reversed and sandwiched,

It is also demonstrated that reversed assemblies tend to possess faster release kinetics than sandwiched assemblies, at least for protons. This may derive form the relatively larger area of the reversed slide dictating more available protons adjacent to the fluid (in the sandwiched assembly some of the protons are in the bottom layer, therefore expected to be liberated to the medium later than the top layer's protons)

When the assemblies were placed on the top of the bottle (just below the water level) efficacy after 4 h under EPA #2 condition was better than that of the bottles with bottom-located assemblies. This may hint us that CDO tends to sink down with time and that it is heavier than water. And indeed, density of CDO is 1.64 g/cm³. This may also suggest that gravitational convection of CDO is present as was also suggested above.

The trial also explored efficacies of blow-molded bottles (form GilPack) with 10 ppm of SC and 0.05 or 0.08 mg/ml of CG8-H in reversed geometry. The bottles with the higher content of IX were clearly superior in terms of efficacy. Moreover, the IX content of the blow-molded bottles is significantly higher than this of the printed assemblies (˜0.03 mg/ml) and therefore the pH drop of the medium is also more significant. It suggests that the required IX content of reversed and sandwiched assemblies is smaller than that of blown bottles.

Additional Steps: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) challenge tests (all w.r.t reference product, Aquamira/Aseptrol efficacy): (a) different microorganisms (viruses and protozoa), and (b) EPA protocol:

| | EPA test | EPA test |
|---|---|---|
| | water #1 | water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial explores routes for improvement of the sensory effect of the assemblies. Possible solutions: (a) SC content reduction, (b) SC/IX ratio optimization, (c) utilization of ClOₓ-species' scavengers/neutralizers such as Na₂SₛO₃, ferrous (Fe⁺²) salts, phenols, alkaline zeolites, Weak (and strong) base anion exchange resins, active carbon, etc., and (6) neutralize ClOₓ's traces in incubation stage.

AWT045, AWT046, AWT049, and AWT051

In a non-limiting example, Samples 47-50 [AWT045, AWT046, AWT049, and AWT051] were tested regarding reversed efficacies (10 and 7.5 ppm) and sandwiched (10 and 5 ppm) assemblies with addition of neutralizer solution during sampling (see, e.g., sample 42) and directly to the bottle. Addition of neutralizer during sampling did not impact efficacy. Addition of the neutralizing solution directly into the bottle seemed to impede or even stop efficacy. The antimicrobial action was found to be completed after 15 to 45 min when operating vs. 10⁵ R. Terrigena.

AWT045

Experiments Goals: Focus on formulation WE003+WE004, LbL, coated that showed efficacy. Different parameters that will be tested for impact on efficacy are: (1) efficacy of assemblies with reduced concentrations of SC under EPA #1 and #2 conditions and (2) efficacy of assemblies after ClOₓ-neutralization in different times.

Experiments Content (AMA only)

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/15 + | Reversed, 10 ppm, | TSB 1:500 | 10 | 500 | R. Terrigena | 5 | 0.5, 4 | RT |
| | CG8-H | EtOAc (see | WE004/26 | EPA #1 | | | | | | hours | |
| B | | also model | | Reversed, 7.5 ppm | | | | | | | |
| | | description) | | EPA #1 | | | | | | | |
| C | | | | Sandwiched, 10 ppm | | | | | | | |
| | | | | EPA #1 | | | | | | | |
| D | | | | Sandwiched, 7.5 ppm | | | | | | | |
| | | | | EPA #1 | | | | | | | |
| E | | | | Sandwiched, 5 ppm | | | | | | | |
| | | | | EPA #1 | | | | | | | |
| F | | | | Reversed, 10 ppm, | | | | | | | |
| | | | | EPA #2 | | | | | | | |
| G | | | | Reversed, 7.5 ppm | | | | | | | |
| | | | | EPA #2 | | | | | | | |
| H | | | | Sandwiched, 10 ppm | | | | | | | |
| | | | | EPA #2 | | | | | | | |
| I | | | | Sandwiched, 7.5 ppm | | | | | | | |
| | | | | EPA #2 | | | | | | | |
| J | | | | Sandwiched, 5 ppm | | | | | | | |
| | | | | EPA #2 | | | | | | | |
| K | | | | Reversed, | | | | | | | |
| | | | | neutralization | | | | | | | |
| | | | | after 15 min | | | | | | | |
| L | | | | Reversed, | | | | | | | |
| | | | | neutralization | | | | | | | |
| | | | | after 30 min | | | | | | | |
| M | | | | Reversed, | | | | | | | |
| | | | | neutralization | | | | | | | |
| | | | | after 60 min | | | | | | | |
| NCN | N/A | N/A | N/A | NC EPA #1 | | N/A | 50 | | | | |
| NCO | N/A | N/A | N/A | NC EPA #2 | | N/A | 50 | | | | |

This is a yes/no experiment, thus counting is needed only for 0, 1 dilutions. Measure CIOₓ concentrations and swelling at 1, 4 hours. If any of formulations are active will be tested for organoleptic attributes. Measure pH.

Results: Microbiology Results

Analytic measurements

| | 0.5 hr | | | | | 4 hr | | | | |
|---|---|---|---|---|---|---|---|---|---|---|
| Sample | Cl2 | ClO2 | ClO2− | total ox′ | pH | Cl2 | ClO2 | ClO2− | total ox′ | pH |
| Regular 1 | 0 | 0.12 | 4.056 | 4.176 | 4.8 | 0 | 1.108 | 6.276 | 7.384 | 3.6 |
| Regular 2 | 0 | 0 | 2.98 | 2.98 | 5.3 | 0 | 1.808 | 5.456 | 7.264 | 4.5 |
| Regular 3 | 0 | 0.188 | 4.132 | 4.32 | 5 | 0 | 1.388 | 5.128 | 6.516 | 4.6 |
| Reversed 1 | 0 | 0 | 0 | 0 | 5 | 0 | 4.192 | 0 | 4.192 | 4.4 |
| Reversed 2 | 0 | 0 | 0 | 0 | 4.8 | 0 | 3.104 | 0 | 3.104 | 4.4 |
| Reversed 3 | 0 | 0 | 0 | 0 | 5 | 0 | 2.36 | 0 | 2.36 | 4.5 |
| Sandwiched 1 | 0 | 0 | 0 | 0 | 4.9 | 0 | 12.716 | 0 | 12.716 | 4.2 |
| Sandwiched 2 | 0 | 0 | 0 | 0 | 4.8 | 0 | 0 | 0 | 0 | 4.2 |
| Sandwiched 3 | 2.08 | 0 | 3.96 | 3.96 | 4.8 | | 12.46 | 0 | 12.46 | 4.2 |
| Aquamira 1 | 0 | 2.328 | 15.024 | 17.352 | 3.7 | 0 | 14.936 | 15 | 29.932 | 3.7 |
| Aquamira 2 | 0 | 1.264 | 16.916 | 18.18 | 3.6 | 0 | 1.236 | 4.836 | 6.072 | 3.6 |
| Aquamira 3 | 0 | 0.684 | 5.776 | 6.46 | 3.6 | 0 | 0.376 | 6.052 | 6.428 | 3.6 |

Summary and Conclusions

Reversed and sandwiched assemblies were effective vs. R. Terrigena down to 7.5 ppm (after 1 h) under EPA #1 conditions. Assemblies were not effective after 1 h under EPA #2 conditions (+TSB 1:500), unlike precedent trials. This may be related to fabrication parameters (large RK applicator was used). This will be examined again this week.

In-bottle neutralization did possess negative influence in the efficacy. However, one would expect that the sample without neutralizer addition and the sample with addition at 60 min will exhibit similar results.

Analytic measurements performed exhibited once again that the reversed assemblies (and partially also the sandwiched assembly) form only CDO in the medium while the regular assembly as well as Aquamira forms also chlorite.

Additional Experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) challenge tests (all w.r.t reference product, Aquamira/Aseptrol efficacy): (a) different microorganisms (viruses and protozoa) and (b) EPA protocol:

| | | EPA |
|---|---|---|
| | EPA test water #1 | test water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial explores routes for improvement of the sensory effect of the assemblies. Possible solutions: (a) SC content reduction, (b) SC/IX ratio optimization, (c) utilization of ClOₓ-species' scavengers/neutralizers such as Na₂SₛO₃, phenols, alkaline zeolites, weak (and strong) base anion exchange resins, active carbon, etc., (6) neutralize ClOₓ's traces in incubation stage.

AWT046

Experiments Goals:

Focus on formulation WE003+WE004, LbL, coaled that showed efficacy. Different parameters that are tested for impact on efficacy are: explore various methods of ClOₓ- neutralization by SS+(0.3% Na₂S₂O₃ in saline) in-situ (directly within the treated bottle) and ex-situ (only during sampling).

Experiments Content (AMA only)

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Reversed, | TSB 1:500 | 10 | 500 | R. Terrigena | 5 | 0.5, 4 hours | RT |
| | CG8-H | EtOAc (see | WE004 | w/o neut. | | | | | | | |
| B | | also model | | Sandwiched, | | | | | | | |
| | | description) | | w/o neut. | | | | | | | |
| C | | | | Reversed, | | | | | | | |
| | | | | neut. @ sampling | | | | | | | |
| D | | | | Sandwiched, | | | | | | | |
| | | | | neut. @ sampling | | | | | | | |
| E | | | | Reversed, | | | | | | | |
| | | | | neut. After 15′ | | | | | | | |
| F | | | | Sandwiched, | | | | | | | |
| | | | | neut. After 15′ | | | | | | | |
| G | | | | Reversed, | | | | | | | |
| | | | | neut. After 30′ | | | | | | | |
| H | | | | Sandwiched, | | | | | | | |
| | | | | neut. After 30′ | | | | | | | |
| I | | | | Reversed, | | | | | | | |
| | | | | neut. After 60′ | | | | | | | |
| J | | | | Sandwiched, | | | | | | | |
| | | | | neut. After 60′ | | | | | | | |
| K | | | | Reversed, | | | | | | | |
| | | | | neut. After 240′ | | | | | | | |
| L | | | | Sandwiched, | | | | | | | |
| | | | | neut. After 2400′ | | | | | | | |
| NCM | N/A | N/A | N/A | NC | | N/A | 50 | | | | |
| NCN | N/A | N/A | N/A | NC w/neut. | | N/A | 50 | | | | |
| | | | | @ sampling | | | | | | | |
| NCO | N/A | N/A | N/A | Neut. Only | | N/A | 50 | Natural flora | N/A | | |
| | | | | w/TSB, natural | | | | | | | |
| | | | | flora | | | | | | | |

This is a yes/no experiment, thus counting is needed only for 0, 1 dilutions. Measure ClOₓ concentrations and swelling at 1, 4 hours. If any of formulations are active will be tested for organoleptic attributes. Measure pH.

Results: Microbiology Results

addition of SS+ after 4 h did not affect efficacy. This trial will be repeated soon together with kinetics and residuals' analysis.

SS+ solution was heat-sterilized (by autoclave) prior to the experiment. It was also tested and found to be negative for possible microbial contamination.

Additional Experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) challenge tests (all w.r.t reference product, Aquamira/Aseptrol efficacy): (a) different microorganisms (viruses and protozoa) and (b) EPA protocol:

| | | EPA |
|---|---|---|
| | EPA test water #1 | test water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial explores routes for improvement of the sensory effect of the assemblies. Possible solutions: (a) SC content reduction, (b) SC/IX ratio optimization, (c) utilization of ClOₓ-species' scavengers/neutralizers such as Na₂SₛO₃, phenols, alkaline zeolites, Weak (and maybe also strong) base anion exchange resins, active carbon, etc., and (6) neutralize ClOₓ's traces in incubation stage.

AWT049

Experiments Goals: explore various methods of CIOₓ-neutralization by SS+(0.3% Na₂S₂O₃ in 0.85% saline) in-situ (directly within the treated bottle) and ex-situ (only during sampling).

Experiments Content (AMA only)

| | | | | | | SC Active | | | | | |
|---|---|---|---|---|---|---|---|---|---|---|---|
| Model | Active | Additional | Formulation | | | conc. | vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Reversed, | TSB 1:500 | 10 | 500 | R. Terrigena | 5 | 0.5, 4 hours | RT |
| | CG8-H | EtOAc (see | WE004 | w/o neut. | | | | | | | |
| B | | also model | | Sandwiched, | | | | | | | |
| | | description) | | w/o neut. | | | | | | | |
| C | | | | Reversed, | | | | | | | |
| | | | | neut. @ | | | | | | | |
| | | | | sampling | | | | | | | |
| D | | | | Sandwiched, | | | | | | | |
| | | | | neut. @ | | | | | | | |
| | | | | sampling | | | | | | | |
| E | | | | Reversed, | | | | | | | |
| | | | | neut. After 15′ | | | | | | | |
| F | | | | Sandwiched, | | | | | | | |
| | | | | neut. After 15′ | | | | | | | |
| G | | | | Reversed, | | | | | | | |
| | | | | neut. After 30′ | | | | | | | |
| H | | | | Sandwiched, | | | | | | | |
| | | | | neut. After 30′ | | | | | | | |
| I | | | | Reversed, | | | | | | | |
| | | | | neut. After 45′ | | | | | | | |
| J | | | | Sandwiched, | | | | | | | |
| | | | | neut. After 45′ | | | | | | | |
| K | | | | Reversed, | | | | | | | |
| | | | | neut. After 60′ | | | | | | | |
| L | | | | Sandwiched, | | | | | | | |
| | | | | neut. After 60′ | | | | | | | |
| M | | | | Reversed, | | | | | | | |
| | | | | neut. After 240′ | | | | | | | |
| N | | | | Sandwiched, | | | | | | | |
| | | | | neut. After 240′ | | | | | | | |
| NCO | N/A | N/A | N/A | NC | | N/A | 50 | | | | |
| NCP | N/A | N/A | N/A | NC w/neut. | | N/A | 50 | | | | |
| | | | | @ sampling | | | | | | | |

This is a yes/no experiment, thus counting is needed only for 0, 1 dilutions. Hach. Kinetics: Reversed and sandwiched w/DDW alter 0.5 and 4 h, before and after SS+ addition (2 ml per 200 ml). Hach, residuals: measure after termination of AWA trial, 12 samples×1 sampling. SS+—conlamination control—sterilize before use and sample before and after use.

Results: Microbiology Results

Summary and Conclusions

The assemblies are effective vs. R. Terrigena whether or not SS+ ClOₓ-neutralizer is applied during sampling and post-incubation or not. It can be safely concluded that the AMA takes place solely within the bottle and not during the post-incubation.

SS+ neutralizer was added to the bottles to explore its influence on the efficacy in-situ. When the SS+ was added 15′ and 30′ after initiation the reversed assemblies were not effective (or at least not coherently effective). No significant difference was detected between reversed and sandwiched assemblies.

When SS+ was added after 45′ the efficacy was not impeded, hinting the killing process is done by then. As expected, addition of SS+ after 1 and 4 h did not affect efficacy.

Additional Experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) challenge tests (all w.r.t reference product, Aquamira/Aseptrol efficacy): (a) different microorganisms (viruses and protozoa) and (b) EPA protocol:

| | | EPA |
|---|---|---|
| | EPA test water #1 | test water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temprature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial explores routes for improvement of the sensory effect of the assemblies. Possible solutions: (a) SC content reduction, (b) SC/IX ratio optimization, (c) utilization of ClOₓ-species' scavengers/neutralizers such as Na₂S₃O₃, phenols, alkaline zeolites, weak (and strong) base anion exchange resins, active carbon, etc., and (6) neutralize ClOₓ's traces in incubation stage.

AWT051

Experiments Goals: Explore various methods of ClOₓ-neutralization by SS+ (0.3% Na₂S₂O₃ in 0.85% saline) in-siiu (directly within the treated bottle) and ex-situ (only during sampling).

Experiments Content (AMA only)

| | | | | | | SC Active | | | | | |
|---|---|---|---|---|---|---|---|---|---|---|---|
| Model | Active | Additional | Formulation | | | conc. | vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Reversed, | TSB 1:500 | 10 | 500 | R. Terrigena | 5 | 0.5, 1, 4 | RT |
| | CG8-H | EtOAc | WE004 | w/o neut. | | | | | | hours, | |
| B | | (see also | | Sandwiched, | | | | | | samples A-F | |
| | | model | | w/o neut. | | | | | | also 15′ | |
| | | description) | | | | | | | | | |
| C | | | | Reversed, | | | | | | | |
| | | | | neut. @ | | | | | | | |
| | | | | sampling | | | | | | | |
| D | | | | Sandwiched, | | | | | | | |
| | | | | neut. @ | | | | | | | |
| | | | | sampling | | | | | | | |
| E | | | | Reversed, | | | | | | | |
| | | | | neut. After 15′ | | | | | | | |
| F | | | | Sandwiched, | | | | | | | |
| | | | | neut. After 15′ | | | | | | | |
| G | | | | Reversed, | | | | | | | |
| | | | | neut. After 30′ | | | | | | | |
| H | | | | Sandwiched, | | | | | | | |
| | | | | neut. After 30′ | | | | | | | |
| I | | | | Reversed, | | | | | | | |
| | | | | neut. After 45′ | | | | | | | |
| J | | | | Sandwiched, | | | | | | | |
| | | | | neut. After 45′ | | | | | | | |
| K | | | | Reversed, | | | | | | | |
| | | | | neut. After 60′ | | | | | | | |
| L | | | | Sandwiched, | | | | | | | |
| | | | | neut. After 60′ | | | | | | | |
| M | | | | Reversed, | | | | | | | |
| | | | | neut. After 240′ | | | | | | | |
| N | | | | Sandwiched, | | | | | | | |
| | | | | neut. After 240′ | | | | | | | |
| NCO | N/A | N/A | N/A | NC | | N/A | 50 | | | | |
| NCP | N/A | N/A | N/A | NC | | N/A | 50 | | | | |
| | | | | w/neut. @ | | | | | | | |
| | | | | sampling | | | | | | | |

This is a yes/no experiment, thus counting is needed only for 0, 1 dilutions. Hach, Kinetics: Reversed and sandwiched w/DDW after 0.5 and 4 h, before and after SS+ addition (2 ml per 200 ml). Hach, residuals: measure after termination of AWA trial, 12 samples ×1 sampling, SS+—contamination control—sterilize before use and sample before and after use.

Results: Microbiology Results—R. Terrigena viable counts

However, “in-situ” addition of SS+ yields incoherent results (as in the previous trial). Theoretically after 30 min samples that were added with SS+ during sampling and samples that were added with SS+ after 30 should provide similar results but that is not the case (samples C and D vs. samples G and H). The same goes for samples that were not even added with SS+ (A, B, and K-N vs. I and J). The inconsistencies may derive form inconsistent timing of the specimens filling, samplings and SS+ direct additions. Since it was already demonstrated the 30 min is the threshold for efficacy, minor deviations in sampling times may result in large variations in efficacy.

Additional experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) challenge tests (all w.r.t reference product, Aquamira/Aseptrol efficacy): (a) different microorganisms (viruses and protozoa) and (b) EPA protocol:

| | | EPA |
|---|---|---|
| | EPA test water #1 | test water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial is to be set to explore routes for improvement of the sensory effect of the assemblies. Possible solutions: (a) SC content reduction, (b) SC/IX ratio optimization, (c) utilization of ClOₓ-species' scavengers/neutralizers such as Na₂SₛO₃, ferrous (Fe⁺²) salts, phenols, alkaline zeolites, Weak (and maybe also strong) base anion exchange resins, active carbon, etc., and (6) neutralize ClO₃'s traces in incubation stage.

AWT047

In a non-limiting example, Sample 51 [AWT047] was tested regarding the efficacy of reversed and sandwiched assemblies in SC contents of 10 and 7.5 ppm in 3 media, EPA#1, EPA#2, and EPA#2+TSB 1:500 (see above details). 10 ppm assemblies were effective under al conditions in both assemblies after 0.5 h. 7.5 ppm assemblies were less effective after 05 h than 10 ppm assemblies, but brought total eradication after 4 h.

Experiments Goals: Examine efficacy of reversed and sandwiched assemblies at different SC contents and under EPA #1 and #2 conditions.

| | | | | | | SC Active | | | | | |
|---|---|---|---|---|---|---|---|---|---|---|---|
| Model | Active | Additional | Formulation | | | conc. | vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Reversed, 10 ppm, | See | 10 | 500 | R. Terrigena | 5 | 0.5, 4 | RT |
| | CG8-H | EtOAc (see | WE004 | EPA#1 | model | | | | | hours | |
| B | | also model | | Reversed, 7.5 ppm, | descrip- | | | | | | |
| | | description) | | EPA#1 | tion | | | | | | |
| C | | | | Sandwiched, 10 ppm, | | | | | | | |
| | | | | EPA#1 | | | | | | | |
| D | | | | Sandwiched, 7.5 ppm, | | | | | | | |
| | | | | EPA#1 | | | | | | | |
| E | | | | Reversed, 10 ppm, | | | | | | | |
| | | | | EPA#2 w/TSB | | | | | | | |
| F | | | | Reversed, 7.5 ppm, | | | | | | | |
| | | | | EPA#2 w/TSB | | | | | | | |
| G | | | | Sandwiched, 10 ppm, | | | | | | | |
| | | | | EPA#2 w/TSB | | | | | | | |
| H | | | | Sandwiched, 7.5 ppm, | | | | | | | |
| | | | | EPA#2 w/TSB | | | | | | | |
| I | | | | Reversed, 10 ppm, | | | | | | | |
| | | | | EPA#2 w/o TSB | | | | | | | |
| J | | | | Reversed, 7.5 ppm, | | | | | | | |
| | | | | EPA#2 w/o TSB | | | | | | | |
| K | | | | Sandwiched, 10 ppm, | | | | | | | |
| | | | | EPA#2 w/o TSB | | | | | | | |
| L | | | | Sandwiched, 7.5 ppm, | | | | | | | |
| | | | | EPA#2 w/o TSB | | | | | | | |
| NCM | N/A | N/A | N/A | NC EPA #1 | | N/A | 50 | | | | |
| NCN | N/A | N/A | N/A | NC EPA #2 w/TSB | | N/A | 50 | | | | |
| NCO | N/A | N/A | N/A | NC EPA #2 w/o TSB | | N/A | 50 | | | | |

This is a yes/no experiment, thus counting is needed only for 0, 1 dilutions. Measure ClOₓ concentrations and swelling at 1, 4 hours. If any of formulations are active will be tested for organoleptic attributes. Measure pH.

Results: Microbiology Results

such circumstances to avoid consumption of high dosages of ClOx. Common neutralizers (see EPA CDO guide p. 4.25-4.26) such as sodium thiosulfate or ferrous salts may be considered (via sustained/controlled release mechanism). Alternatively, weak (or even strong) base anion exchange resins can also be used.

It is also suggested that the EPA water purifier protocol will be followed also in the case of media preparation. I.e., the media will be prepared to meet EPA #1 and EPA #2 demands without further addition of nutrient such as TSB. It was already demonstrated that the nutrient addition is not required for microbial growth where the relevant preparation demands are met. A follow up experiment will be designed to further investigate and validate this issue.

Additional experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) challenge tests (all w.r.t reference product, Aquamira/Aseptrol efficacy): (a) different microorganisms (viruses and protozoa) and (b) EPA protocol:

| | | EPA |
|---|---|---|
| | EPA test water #1 | test water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial is to be set to explore routes for improvement of the sensory effect of the assemblies. Possible solutions: (a) SC content reduction, (b) SC/IX ratio optimization, (c) utilization of ClOₓ-species' scavengers/neutralizers such as Na₂SₛO₃, ferrous salts, phenols, alkaline zeolites, weak (and strong) base anion exchange resins, active carbon, etc., and (6) neutralize ClOₓ's traces in incubation stage.

AWT048

In a non-limiting example, Sample 52 [AWT048] was tested regarding efficacies of 2 m old assemblies stored in RT. All examined assemblies (regular, reversed, and sandwiched, alkalized and not, w/or w/o PVP layer). All assemblies were effective under EPA #1 conditions. Reversed and sandwiched assemblies were effective after 0.5 h under EPA #2 conditions also. Regular assemblies were not effective under EPA #2 at all. Reversed and sandwiched assemblies with PVP layer (reversed only) or alkalization were effective only after the 4 h sampling. Analytic ClOₓ-species determination yielded the same conclusions.

Experiments Goals: Examine the shelf-life of various assemblies under room conditions. Test: influence of geometry, influence of SC:Vinnol/EtOAc formulation alkalization, and influence of application of PVP (kollidon 30) top and intermediate barrier layers. Efficacy is examined under EPA #1 and #2 conditions.

Experiments Content (AMA Only)

| | | | | | | SC | | | | | |
|---|---|---|---|---|---|---|---|---|---|---|---|
| | | | | | | Active | | | | | |
| Model | Active | Additional | Formulation | | | conc. | vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | Fresh: see | Regular, fresh, | Dee | 10 | 500 | R. Terrigena | 5 | 0.5, 4 hours | RT |
| | CG8-H | EtOAc (see | AWT046 | EPA #1 | model | | | | | | |
| B | | also model | 2 m old std.: | Reversed, fresh, | descrip- | | | | | | |
| | | description) | see AWT018 | EPA #1 | tion | | | | | | |
| C | | | 2 m old alk.: | Sandwiched, fresh, | | | | | | | |
| | | | see AWT019 | EPA #1 | | | | | | | |
| D | | | 6 2 old | Reversed, 2 m old, | | | | | | | |
| | | | w/PVP: see | EPA #1 | | | | | | | |
| E | | | AWT024 | Sandwiched, 2 m old, | | | | | | | |
| | | | | EPA #1 | | | | | | | |
| F | | | | Regular, 2 m old, | | | | | | | |
| | | | | alk., EPA #1 | | | | | | | |
| G | | | | Reversed, 2 m old, | | | | | | | |
| | | | | alk., EPA #1 | | | | | | | |
| H | | | | Sandwiched, 2 m old, | | | | | | | |
| | | | | alk., EPA #1 | | | | | | | |
| I | | | | Reversed, 6 w old, | | | | | | | |
| | | | | PVP 12 mic | | | | | | | |
| | | | | top, EPA #1 | | | | | | | |
| J | | | | Reversed, 6 w old, | | | | | | | |
| | | | | PVP 120 mic | | | | | | | |
| | | | | top, EPA #1 | | | | | | | |
| K | | | | Reversed, 6 w old, | | | | | | | |
| | | | | PVP 12 mic interim, | | | | | | | |
| | | | | EPA #1 | | | | | | | |
| L | | | | Regular, fresh, | | | | | | | |
| | | | | EPA #2 | | | | | | | |
| M | | | | Reversed, fresh, | | | | | | | |
| | | | | EPA #2 | | | | | | | |
| N | | | | Sandwiched, fresh, | | | | | | | |
| | | | | EPA #2 | | | | | | | |
| O | | | | Reversed, 2 m old, | | | | | | | |
| | | | | EPA #2 | | | | | | | |
| P | | | | Sandwiched, 2 m old, | | | | | | | |
| | | | | EPA #2 | | | | | | | |
| Q | | | | Regular, 2 m old, | | | | | | | |
| | | | | alk., EPA #2 | | | | | | | |
| R | | | | Reversed, 2 m old, | | | | | | | |
| | | | | alk., EPA #2 | | | | | | | |
| S | | | | Sandwiched, 2 m old, | | | | | | | |
| | | | | alk., EPA #2 | | | | | | | |
| T | | | | Reversed, 6 w old, | | | | | | | |
| | | | | PVP 12 mic | | | | | | | |
| | | | | top, EPA #2 | | | | | | | |
| U | | | | Reversed, 6 w old, | | | | | | | |
| | | | | PVP 120 mic | | | | | | | |
| | | | | top, EPA #2 | | | | | | | |
| V | | | | Reversed, 6 w old, | | | | | | | |
| | | | | PVP 12 mic | | | | | | | |
| | | | | interim, | | | | | | | |
| | | | | EPA #2 | | | | | | | |
| NC-W | N/A | N/A | N/A | NC EPA #1 | | N/A | 50 | | | | |
| NC-X | N/A | N/A | N/A | NC EPA #2 | | N/A | 50 | | | | |

This is a yes/no experiment, thus counting is needed only for 0, 1 dilutions. Measure ClOₓ concentrations and swelling at 1, 4 hours. If any of formulations are active will be tested for organoleptic attributes. Measure pH.

Results: Microbiology Results

Analytic measurements

| | 0.5 h | | | | | 4 h | | | | |
|---|---|---|---|---|---|---|---|---|---|---|
| Material | Cl2 | ClO2 | ClO2− | total ox′ | pH | Cl2 | ClO2 | ClO2− | total ox′ | pH |
| A | 0 | 0.35 | 3.36 | 3.708 | 4.32 | 0 | 1.892 | 4.696 | 6.588 | 4.77 |
| B | 0 | 0.91 | 2.1 | 3.012 | 4.5 | 0 | 3.868 | 0 | 3.868 | 4.34 |
| C | 0 | 3.66 | 0 | 3.656 | 4.59 | 0 | 0 | 4.2 | 4.2 | 4.31 |
| D | 0 | 2.1 | 0 | 2.096 | 4.75 | 0 | 4.184 | 0 | 4.184 | 4.44 |
| E | 0 | 4.82 | 0 | 4.816 | 4.62 | 0 | 2.616 | 0 | 2.616 | 4.34 |
| F | 0.42 | 0 | 4.72 | 4.72 | 4.66 | 0 | 0.576 | 4.028 | 4.604 | 4.57 |
| G | 0 | 4.84 | 0 | 4.84 | 4.69 | 0 | 1.648 | 3.12 | 4.768 | 4.33 |
| H | 0 | 2.44 | 0 | 2.436 | 4.63 | 0 | 2.968 | 0 | 2.968 | 4.19 |
| I | 0 | 0.73 | 2.436 | 3.164 | 4.67 | 0.3 | 0 | 6.816 | 6.816 | 4.48 |
| I | 0 | 2.94 | 0 | 2.936 | 4.66 | 0 | 1.032 | 3.108 | 4.14 | 4.41 |
| K | 0 | 4.09 | 0 | 4.092 | 4.89 | 0 | 3.344 | 0 | 3.344 | 4.39 |

Summary and conclusions

All of the assemblies, fresh and aged, exhibited efficacy under EPA #1 conditions. EPA #2 trial conditions were more challenging. Only the fresh reversed and sandwiched assemblies exhibited full efficacy after 0.5 h. the rest of the assemblies, excluding the fresh and aged regular ones, presented full efficacy after 4 h.

Regular assemblies tend to possess weaker efficacies and shorter shelf-life. One can also observe that regular assemblies CDO activation yield (i.e., larger chlorite content) is significantly lower than in reversed and sandwiched assemblies. This might cause the regular assembly inefftcacy under harder conditions which require stronger activation/protonation. Stronger activation might also mitigate efficacy when CDO precursor is slightly depleted during storage.

Alternatively, aging of the dry sheet may simply impede the release of CDO into the medium, therefore slowing down the efficacy in tough conditions.

Additional experiments: (1) repeat benchmark formulation/fabrication parameters (regular, reversed, and sandwiched) as positive control/reproducibility study, (2) explore shelf life of sheets in RT and under HALT of 40° C./80% RH. Tests will be conducted under EPA #1 and #2 test conditions. Variable parameters: (a) assembly type—reversed, (b) alkalization of SC formulation (yes/no), (c) indicator presence (yes/no), (d) PVP (Kollidon 30, Luvitec VA64) or different top-coat and its thickness, (e) packaging—protective tape (if and when available), plastic bag, Al., (3) challenge tests (all w.r.t reference product, Aquamira/Aseptrol efficacy): (a) different microorganisms (viruses and protozoa) and (b) EPA protocol:

| | | EPA |
|---|---|---|
| | EPA test water #1 | test water #2 |
| pH | 6.5-8.5 | 9 ± 0.2 |
| Total organic carbon (TOC) [mg/L] | 0.1-5 | >10 |
| Turbidity [NTU] | 0.1-5 | >30 |
| Temperature [° C.] | 20 ± 5 | 4 ± 0.1 |
| Total dissolved solids (TDS) [mg/L] | 50-500 | 1500 ± 150 |

(4) elaborated kinetics and residuals analysis (Hach), (5) combined AMA-analytic-sensory trial. This trial is to be set to explore routes for improvement of the sensory effect of the assemblies. Possible solutions: (a) SC content reduction, (b) SC/IX ratio optimization, (c) utilization of ClOₓ-species' scavengers/neutralizers such as Na₂SₛO₃, ferrous salts, phenols, alkaline zeolites, weak (and strong) base anion exchange resins, active carbon, etc., and (6) neutralize ClOₓ's traces in incubation stage.

AWT053 and AWT057

In a non-limiting example, Samples 53 and 54 [AWT053 and AWT057] examined the efficacy of samples stored under HALT (40° C., 80% Humidity) and under RT for 1 m. All samples were ineffective when stored under HALT. RT stored assemblies were effective. Analytic ClOₓ-specics determination yielded the same conclusions.

AWT053

Experiments Goals; explore efficacy of assemblies after 1 month under HALT of 40° C. and 80%RH

Experiments Content (AMA only)

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Regular, 1 m old, | See notes | 10 | 500 | R. Terrigena | 5 | 0.5, 4 | EPA#1: |
| | CG8-H | EtOAc | WE004 | PE, EPA#1 | and | | | | | hours | RT |
| B | | (see | | Reversed, 1 m old, | model | | | | | | EPA#2: |
| | | also | | PE, EPA#1 | descrip- | | | | | | 4° C. |
| C | | model | | Sandwiched, 1 m old, | tion | | | | | | |
| | | description) | | PE, EPA#1 | | | | | | | |
| D | | | | Regular, 1 m old, | | | | | | | |
| | | | | PE + tape, EPA#1 | | | | | | | |
| E | | | | Reversed, 1 m old, | | | | | | | |
| | | | | PE + tape, EPA#1 | | | | | | | |
| F | | | | Sandwiched, 1 m old, | | | | | | | |
| | | | | PE + tape, EPA#1 | | | | | | | |
| G | | | | Regular, 1 m old, | | | | | | | |
| | | | | Al, EPA#1 | | | | | | | |
| H | | | | Reversed, 1 m old, | | | | | | | |
| | | | | Al, EPA#1 | | | | | | | |
| I | | | | Sandwiched, 1 m old, | | | | | | | |
| | | | | Al, EPA#1 | | | | | | | |
| J | | | | Regular, 1 m old, | | | | | | | |
| | | | | PE, EPA#2 | | | | | | | |
| K | | | | Reversed, 1 m old, | | | | | | | |
| | | | | PE, EPA#2 | | | | | | | |
| L | | | | Sandwiched, 1 m old, | | | | | | | |
| | | | | PE, EPA#2 | | | | | | | |
| M | | | | Regular, 1 m old, | | | | | | | |
| | | | | PE + tape, EPA#2 | | | | | | | |
| N | | | | Reversed, 1 m old, | | | | | | | |
| | | | | PE + tape, EPA#2 | | | | | | | |
| O | | | | Sandwiched, 1 m old, | | | | | | | |
| | | | | PE + tape, EPA#2 | | | | | | | |
| P | | | | Regular, 1 m old, | | | | | | | |
| | | | | Al, EPA#2 | | | | | | | |
| Q | | | | Reversed, 1 m old, | | | | | | | |
| | | | | Al, EPA#2 | | | | | | | |
| R | | | | Sandwiched, 1 m old, | | | | | | | |
| | | | | Al, EPA#2 | | | | | | | |
| NCQ | N/A | N/A | N/A | NC EPA #1 | | N/A | 50 | | | | |
| NCR | N/A | N/A | N/A | NC EPA #2 | | N/A | 50 | | | | |

This is a yes/no experiment, thus counting is needed only for 0, 1 dilutions. Refrain from agitating bottles (except from during sampling). Hach: reversed and sandwiched, Bottom, top and agitated (6 samples). Sample was taken from the middle of the bottle. Measure pH. Media preparation: EPA #1:1 mg/L Humic acid sodium salt, 50 mg/L NaCl in 2L of sterile DDW, EPA #2: 10 mg/L Humic acid sodium salt. 1500 mg/L NaCl and 1 drop of NH3 25% in 2L of sterile DDW.

Results: Microbiology Results

Analytic Results

| | 0.5 hr | | | | | 4 hr | | | | |
|---|---|---|---|---|---|---|---|---|---|---|
| Sample | cl2 | clo2 | clo2− | Total ox′ | pH | cl2 | clo2 | clo2− | Total ox′ | pH |
| A - Reg, | 1.2 | 2.92 | 0 | 2.916 | 5.06 | 0 | 0 | 0 | 0 | 5.43 |
| PE | | | | | | | | | | |
| B - Rev, | 0 | 0.64 | 4.036 | 4.676 | 5.1 | 0 | 0 | 0 | 0 | 4.24 |
| PE | | | | | | | | | | |
| C - Sandw, | 1.2 | 0 | 5.292 | 5.292 | 4.82 | 0 | 0 | 0 | 0 | 4.11 |
| PE | | | | | | | | | | |
| D - Reg, | 1 | 0 | 4.68 | 4.68 | 5.64 | 0.41 | 0 | 5.54 | 5.54 | 5.22 |
| Tape | | | | | | | | | | |
| E - Rev, | 0 | 0 | 0 | 0 | 4.9 | 2.26 | 0 | 4.288 | 4.288 | 4.32 |
| Tape | | | | | | | | | | |
| F - Sandw, | 0 | 0 | 0 | 0 | 4.64 | 0 | 0 | 0 | 0 | 4.34 |
| Tape | | | | | | | | | | |
| G - Reg, Al | 0 | 0.9 | 3.8 | 4.696 | 5.44 | 0 | 0 | 0 | 0 | 4.81 |
| H - Rev, Al | 0 | 4.46 | 0 | 4.456 | 4.52 | 0 | 0.644 | 4.508 | 5.152 | 4.36 |
| I - Sandw, | 1.2 | 0 | 4.24 | 4.24 | 4.43 | 0.05 | 0 | 4.928 | 4.928 | 4.29 |
| Al | | | | | | | | | | |

All assemblies were not effective except the sandwiched assembly stored in evacuated Al-bags. This comes in partial disagreement with precedent results (see AWT037) and with the Hach results (which are also somewhat inconsistent, e.g., positive values after 0.5 h and negative after 4 h).

AWT057

Experiments Goals: (1) Failure analysis of AWT053 and (2) explore the efficacy of assemblies after 5 weeks under RT and HALT of 40° C. and 80% RH.

Experiment Content (AMA Only)

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Regular, 1 m old, | TSB 1:500 | 10 | 500 | R. Terrigena | 5 | 0.5, 4 | EPA#1: |
| | CG8-H | EtOAc (see | WE004 | HALT | | | | | | hours | RT |
| B | | also model | | Reversed, 1 m old, | | | | | | | EPA#2: |
| | | description) | | PE, HALT | | | | | | | 4° C. |
| C | | | | Sandwiched, 1 m old, | | | | | | | |
| | | | | PE, HALT | | | | | | | |
| D | | | | Regular, 1 m old, | | | | | | | |
| | | | | PE + tape, | | | | | | | |
| | | | | HALT | | | | | | | |
| E | | | | Reversed, 1 m old, | | | | | | | |
| | | | | PE + tape, | | | | | | | |
| | | | | HALT | | | | | | | |
| F | | | | Sandwiched, 1 m old, | | | | | | | |
| | | | | PE + tape, | | | | | | | |
| | | | | HALT | | | | | | | |
| G | | | | Regular, 1 m old, | | | | | | | |
| | | | | Al, HALT | | | | | | | |
| H | | | | Reversed, 1 m old, | | | | | | | |
| | | | | Al, HALT | | | | | | | |
| I | | | | Sandwiched, 1 m old, | | | | | | | |
| | | | | Al, HALT | | | | | | | |
| J | | | | Regular, 1 m old, | | | | | | | |
| | | | | PE, RT | | | | | | | |
| K | | | | Reversed, 1 m old, | | | | | | | |
| | | | | PE, RT | | | | | | | |
| L | | | | Sandwiched, 1 m old, | | | | | | | |
| | | | | PE, RT | | | | | | | |
| M | | | | Regular, 1 m old, | | | | | | | |
| | | | | PE + tape, RT | | | | | | | |
| N | | | | Reversed, 1 m old, | | | | | | | |
| | | | | PE + tape, RT | | | | | | | |
| O | | | | Sandwiched, 1 m old, | | | | | | | |
| | | | | PE + tape, | | | | | | | |
| | | | | RT | | | | | | | |
| P | | | | Regular, 1 m old, | | | | | | | |
| | | | | Al, RT | | | | | | | |
| Q | | | | Reversed, 1 m old, | | | | | | | |
| | | | | Al, RT | | | | | | | |
| R | | | | Sandwiched, 1 m old, | | | | | | | |
| | | | | Al, RT | | | | | | | |
| S | | | | Reversed, | | | | | | | |
| | | | | “fresh” PC | | | | | | | |
| NC | N/A | N/A | N/A | NC | | N/A | 50 | | | | |

This is a yes/no experiment, thus counting is needed only for 0, 1 dilutions. Measure pH. To find out whether experimental error real degradation encouraged the failure of

Summary and conclusions

Efficacy of 1 m old assemblies was found to be dependent in the storage conditions. Assemblies that were stored under HALT of 40° C. and 80%RH were ineffective while assemblies stored under normal RT conditions were effective.

Notably, an earlier experiment (AWT037) did exhibit efficacy after 1 m of storage under HALT. A following trial examining efficacy of reversed assemblies (AWT059/60, AWT038-tₒ) results may him the correct trait.

A trial examining the influence of temperature alone was also initiated to explore whether the temperature or the humidity is the dominant degradation vector.

AWT054

In a non-limiting example, Sample 55 [AWT054] was tested regarding the efficacies of reversed and sandwiched assemblies under EPA #1 and EPA #2 conditions in 4 varying SC content, 20, 10, 7.5, and 5 ppm. All assemblies at all SC contents were effective in EPA #1, EPA #2 results were inconclusive.

Experiments Goals: Explore efficacy of reversed and sandwiched assemblies in varying SC contents.

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/18 + | Rev, 20 ppm, | See notes | 10 | 500 | R. Terrigena | 5 | 0.5, 4 hours | EPA#1: |
| | CG8-H | EtOAc | WE004 | EPA#1 | and | | | | | | RT |
| B | | (see also | | Rev, 10 ppm, | model | | | | | | EPA#2: |
| | | model | | EPA#1 | description | | | | | | 4° C. |
| C | | description) | | Rev, 7.5 ppm, | | | | | | | |
| | | | | EPA#1 | | | | | | | |
| D | | | | Rev, 5 ppm, | | | | | | | |
| | | | | EPA#1 | | | | | | | |
| E | | | | Sandw., 20 ppm, | | | | | | | |
| | | | | EPA#1 | | | | | | | |
| F | | | | Sandw., 10 ppm, | | | | | | | |
| | | | | EPA#1 | | | | | | | |
| G | | | | Sandw., 7.5 ppm, | | | | | | | |
| | | | | EPA#1 | | | | | | | |
| H | | | | Sandw., 5 ppm, | | | | | | | |
| | | | | EPA#1 | | | | | | | |
| I | | | | Rev, 20 ppm, | | | | | | | |
| | | | | EPA#2 | | | | | | | |
| J | | | | Rev, 10 ppm, | | | | | | | |
| | | | | EPA#2 | | | | | | | |
| K | | | | Rev, 7.5 ppm, | | | | | | | |
| | | | | EPA#2 | | | | | | | |
| L | | | | Rev, 5 ppm, | | | | | | | |
| | | | | EPA#2 | | | | | | | |
| M | | | | Sandw., 20 ppm, | | | | | | | |
| | | | | EPA#2 | | | | | | | |
| N | | | | Sandw., 10 ppm, | | | | | | | |
| | | | | EPA#2 | | | | | | | |
| O | | | | Sandw., 7.5 ppm, | | | | | | | |
| | | | | EPA#2 | | | | | | | |
| P | | | | Sandw., 5 ppm, | | | | | | | |
| | | | | EPA#2 | | | | | | | |
| NCQ | N/A | N/A | N/A | NC EPA #1 | | N/A | 50 | | | | |
| NCR | N/A | N/A | N/A | NC EPA #2 | | N/A | 50 | | | | |

Experiments Goals: Explore efficacy of reversed and sandwiched assemblies with varying assembly area by modification of the SC:Vinnol/EtOAc formulation wet thickness and SC content.

| | | | | | | SC | | | | | |
|---|---|---|---|---|---|---|---|---|---|---|---|
| | | | | | | Active | | | | | |
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE003/ | Reversed | TSB 1:500 | 10 | 500 | R. Terrigena | 5 | 0.5, 4 | RT |
| | CG8-H | EtOAc | 18 + | std.(120/200) | | | | | | hours | |
| B | | (see | WE004 | Sandwiched | | | | | | | |
| | | also model | | std. (200/120/200) | | | | | | | |
| C | | descrip- | | Reversed 12/200 | | | | | | | |
| D | | tion) | | Reversed 40/200 | | | | | | | |
| E | | | | Sandwiched 200/12/200 | | | | | | | |
| F | | | | Sandwiched 200/40/200 | | | | | | | |
| G | | | | Regular 200/40 | | | | | | | |
| H | | | WE003/ | Reversed | | | | | | | |
| | | | 18 + | std.(120/200), w/dil.SC | | | | | | | |
| I | | | WE019/ | Sandwiched | | | | | | | |
| | | | 01 | std. (200/120/200), | | | | | | | |
| | | | | w/dil.SC | | | | | | | |
| J | | | | Reversed 12/200, | | | | | | | |
| | | | | w/dil.SC | | | | | | | |
| K | | | | Reversed 40/200, | | | | | | | |
| | | | | dil.SC, w/dil.SC | | | | | | | |
| L | | | | Sandwiched 200/12/200, | | | | | | | |
| | | | | w/dil.SC | | | | | | | |
| M | | | | Sandwiched 200/40/200, | | | | | | | |
| | | | | w/dil.SC | | | | | | | |
| N | | | | Regular 200/12 | | | | | | | |
| NC | N/A | N/A | N/A | NC | | N/A | 50 | | | | |

This is a yes/no experiment, thus counting is needed only for 0, 1 dilutions. Measure pH.—Diluted SC formulation: WE019/001—SC:Vinnol/EtOAc (8%_{dry} SC)

Results: Table illustrating R. Terrigena viable counts

TABLE
ClOₓ analytic determination

| | | IX | 30 min | | | | | 4 h | | | | |
|---|---|---|---|---|---|---|---|---|---|---|---|---|
| | | content | | | | total | | | | | total | |
| # | model | [mg/ml] | Cl2 | ClO2 | ClO2− | ox' | pH | Cl2 | ClO2 | ClO2− | ox' | pH |
| A | Reversed | 0.031 | 0.98 | 0 | 4.992 | 4.992 | 4.55 | 0.21 | 0 | 3.444 | 3.444 | 4.23 |
| | std.(120/200) | | | | | | | | | | | |
| B | Sandwiched std. | 0.048 | 0 | 0.116 | 3.908 | 4.024 | 4.32 | 0 | 4.812 | 0 | 4.812 | 4.16 |
| | (200/120/200) | | | | | | | | | | | |
| C | Reversed | 0.037 | 0 | 0 | 0 | 0 | 4.56 | 0 | 0 | 0 | 0 | 4.47 |
| | 12/200 | | | | | | | | | | | |
| D | Reversed | 0.045 | 0 | 3.116 | 0 | 3.116 | 4.35 | 0 | 0 | 0 | 0 | 4.31 |
| | 40/200 | | | | | | | | | | | |
| E | Sandwiched | 0.065 | 0 | 0.052 | 4.368 | 4.42 | 4.43 | 0 | 5.152 | 0 | 5.152 | 4.33 |
| | 200/12/200 | | | | | | | | | | | |
| F | Sandwiched | 0.097 | 0 | 1.68 | 2.148 | 3.828 | 4.14 | 0 | 3.8 | 0 | 3.8 | 4.06 |
| | 200/40/200 | | | | | | | | | | | |
| H | Reversed | 0.165 | 0 | 0.872 | 2.172 | 3.044 | 4.1 | 0 | 2.82 | 2.428 | 5.248 | 4.04 |
| | std.(120/200), | | | | | | | | | | | |
| | w/dil.SC | | | | | | | | | | | |
| I | Sandwiched std. | 0.453 | 0 | 0.312 | 2.352 | 2.664 | 3.9 | 0 | 0.868 | 2.172 | 3.04 | 3.61 |
| | (200/120/200), | | | | | | | | | | | |
| | w/dil.SC | | | | | | | | | | | |
| J | Reversed | 0.27 | 0 | 0 | 0 | 0 | 4.21 | 0 | 0 | 0 | 0 | 4.2 |
| | 12/200, | | | | | | | | | | | |
| | w/dil.SC | | | | | | | | | | | |
| K | Reversed | 0.37 | 0 | 0 | 0 | 0 | 4.14 | 0 | 0 | 0 | 0 | 4.13 |
| | 40/200, dil.SC, | | | | | | | | | | | |
| | w/dil.SC | | | | | | | | | | | |
| L | Sandwiched | 0.55 | 0 | 0 | 0 | 0 | 3.95 | 0 | 0 | 0 | 0 | 3.91 |
| | 200/12/200, | | | | | | | | | | | |
| | w/dil.SC | | | | | | | | | | | |
| M | Sandwiched | 0.29 | 0 | 0 | 0 | 0 | 4.09 | 0 | 0 | 0 | 0 | 4.03 |
| | 200/40/200, | | | | | | | | | | | |
| | w/dil.SC | | | | | | | | | | | |

Summary and Conclusions

Reversed and sandwiched assemblies are effective vs. R. Terrigena after 30 min.

Dilution of the SC formulation or reduction of its wet thickness was carried out in order to examine the influence of the total IX content and the total assembly area. No indication of difference was observed within trial's resolution. it may be worthwhile repeating this case study with thinner IX layers to reduce the influence of the IX content. Clearly, the samples with the highest SC content yielded the lower medium pH.

Applying thin films of SC formulation yielded poor results. This may be due to the inconsistent thickness achieved, leading to incorrect calculations of the assembly required area. The fact that the Vinnol/EtOAc formulations are actually dispersions of particles with initial particle size distribution apparently exceeding the applied wet thickness may also encourage this phenomenon.

Assemblies with diluted SC formulations exhibited poor adhesion to the PET substrate resulting in full or partial detachment.

To conclude, the trial demonstrated the potential of utilization of formulations with lower SC content. Future experiments may explore wider span of compositions, variation of the wet thicknesses of both components layers and more precise application methods.

AWT058 (t₀), AWT066, AWT067, and AWT072 (t_{4w})

In a non-limiting example, Samples 57-60 [AWT058 (t₀), AWT066, AWT067 (t_{2w}), AWT072 (t_{4w})] were examined regarding the shelf-life of reversed and sandwiched assemblies. Assemblies were prepared with or without indicator reagents and with or without a top-protection layer of either Luvitec VA64 (16.67 wt % in 2-propanol) or Kollicoat Protect (10 wt % in deionized water). The assemblies were stored under HALT of 40° C. and 80% humidity, under 40° C. in a dry oven, and in RT. HALT-stored assemblies were ineffective after 2 w. 40° C. and RT stored assemblies were effective after 2 w both under EPA #1 and EPA #2 conditions. Analytic Cl0ₓ-species determination yielded the same conclusions.

AWT058 (tₒ)

Experiments Goals: Time zero trial of shelf life case study. Parameters: (1) geometry—Reversed/sandwiched, (2) indicator (yes/no), and (3) protection layer—Luvitec VA64/Kollicoat Protcct/w/o.

Experiments Content (AMA only)

| | | | | | | SC | | | Inocu- | | |
|---|---|---|---|---|---|---|---|---|---|---|---|
| | | Addi- | Formu- | | | Active | | | lation | Sam- | |
| Model | Active | tional | lation | | | conc. | Vol. | Micro- | [Log | pling | Temp, |
| No. | material | materials | number | Model description | Medium | [ppm] | [ml] | organism | cfu/ml] | times | C. |
| A | SC + | Vinnol/ | WE018/8 + | Davik III (10 ppm) | TSB | 10 unless | 500 | R. Terrigena | 5 | 0.5, 4 | EPA#1: |
| B | CG8-H | EtOAc | WE004/32 + | Davik I | 1:500 | specified | | | | hours | RT |
| C | | (see also | HB003/2 | Davik V | | otherwise | | | | | |
| D | | model | | Davik III + H3PO4 | | in the | | | | | |
| E | | description) | WE018/7 + | WE018 over Hycar | | model | | | | | |
| | | | WE021/2 | based SC(aq) | | desc. | | | | | |
| | | | | formulation | | | | | | | |
| | | | | (foamed) | | | | | | | |
| F | | | | WE018 over Hycar | | | | | | | |
| | | | | based SC(aq) | | | | | | | |
| | | | | formulation | | | | | | | |
| | | | | (defoamed) | | | | | | | |
| G | | | WE018/7 + | WE018 over | | | | | | | |
| | | | WE020/1 | EtOAc based | | | | | | | |
| | | | | SC(aq) | | | | | | | |
| | | | | formulation | | | | | | | |
| H | | | WE018/7 + | WE018 over | | | | | | | |
| | | | WE022/1 | BuOAc based | | | | | | | |
| | | | | SC(aq) | | | | | | | |
| | | | | formulation | | | | | | | |
| I | | | WE018/7 + | WE018 over | | | | | | | |
| | | | WE023/1 | EtBuOAc based | | | | | | | |
| | | | | SC(aq) | | | | | | | |
| | | | | formulation | | | | | | | |
| J | | | WE018/7 + | Rev, 10 ppm | | | | | 2 | | |
| K | | | WE004/31 + | Rev, 5 ppm | | | | | | | |
| L | | | WE003/16 | Sandw, 10 ppm | | | | | | | |
| M | | | | Sandw, 5 ppm | | | | | | | |
| NCN | N/A | N/A | N/A | NC 5-log | | N/A | 50 | | 5 | | |
| NCO | N/A | N/A | N/A | NC 2-log | | N/A | 50 | | 2 | | |

This is a yes/no experiment, thus counting is needed only for 0, 1 dilutions. Measure pH.

Results: Microbiology Results

| | | 0.5 h | | | | | 4 h | | | | |
|---|---|---|---|---|---|---|---|---|---|---|---|
| # | Sample | Cl2 | CDO | ClO2− | Total ox' | pH | Cl2 | CDO | ClO2− | Total ox' | pH |
| A | Reversed | 0 | 1.77 | 4.832 | 6.604 | 3.62 | 0 | 2.03 | 4.84 | 6.868 | 4.3 |
| B | Reversed | 1.51 | 3.42 | 4.808 | 8.224 | 3.99 | 0 | 3.26 | 3.45 | 6.716 | 4.2 |
| | w/VA64 | | | | | | | | | | |
| C | Reversed | 0 | 2.27 | 5.504 | 7.772 | 4.44 | 0 | 4.42 | 3.38 | 7.792 | 4.2 |
| | w/Kol.Protect | | | | | | | | | | |
| D | Reversed | 0 | 4.35 | 5.168 | 9.516 | 4.52 | 0 | 0.92 | 6.56 | 7.48 | 4.3 |
| | w/indicator | | | | | | | | | | |
| E | Reversed | 0 | 2.02 | 4.2 | 6.22 | 4.22 | 0 | 2.31 | 4.7 | 7.008 | 4.1 |
| | w/indicator | | | | | | | | | | |
| | w/VA64 | | | | | | | | | | |
| F | Reversed | 0 | 0.43 | 4.22 | 4.648 | 4.47 | 0 | 1.21 | 7.06 | 8.272 | 4.2 |
| | w/indicator | | | | | | | | | | |
| | w/Kol.protect | | | | | | | | | | |
| G | Sandwiched | 0.77 | 0 | 4.648 | 4.648 | 4.73 | 0 | 4.25 | 0 | 4.248 | 4.2 |
| | w/indicator | | | | | | | | | | |

Summary and Conclusions

All examined assemblies are effective after 30 min vs. R. Terrigena under EPA #1 and #2 conditions (excluding sample M which demonstrated inconclusive results). Samples are stored under RT, 40° C. and HALT of 40° C. and 80% RH and examined again after 1 month.

Both protection layers do not interfere with efficacy. It is important to point out that Kollicoat protect was applied as an aqueous solution of the copolymer (PVA-PEG/PVA).

The following experimental details are in connection with AWT066 and AWT067:

Experiments Goals

Shelf-life of reversed and sandwiched assemblies under HALT (40° C. and 80% RH) and (40° C.). The trial will examine efficacy under EPA #1 conditions. Successful specimens will also be tested against EPA #2 in AWT067.

TABLE 1
Experiments Content AWT066 (AMA only)

| | | | | | | SC | | | Inocu- | | |
|---|---|---|---|---|---|---|---|---|---|---|---|
| | | Addi- | Formu- | | | Active | | | lation | Sam- | |
| | Active | tional | lation | Model | | conc. | Vol. | Micro- | [Log | pling | |
| Model No. | material | materials | number | description | Medium | [ppm] | [ml] | organism | cfu/ml] | times | Temp, C. |
| A | SC + | Vinnol/ | WE003/18 + | R, HALT | TSB | 10 unless | 500 | R. Terrigena | 5 | 0.5, | EPA#1: |
| B | CG8-H | EtOAc | WE004 | S, HALT | 1:500 | specified | | | | 4 hours | RT |
| C | | (see also | | R w/VA64, HALT | | otherwise | | | | | |
| D | | model | | R w/Kollicoat, | | in the | | | | | |
| | | descrip- | | HALT | | model | | | | | |
| E | | tion) | | R w/indicator, | | desc. | | | | | |
| | | | | HALT | | | | | | | |
| F | | | | R w/indicator | | | | | | | |
| | | | | w/VA64, HALT | | | | | | | |
| G | | | | R w/indicator | | | | | | | |
| | | | | w/Kollicoat, | | | | | | | |
| | | | | HALT | | | | | | | |
| H | | | | S w/indicator, | | | | | | | |
| | | | | HALT | | | | | | | |
| I | | | | R, 40° C. | | | | | | | |
| J | | | | S, 40° C. | | | | | | | |
| K | | | | R w/VA64, | | | | | | | |
| | | | | 40 ° C. | | | | | | | |
| L | | | | R w/Kollicoat, | | | | | | | |
| | | | | 40° C. | | | | | | | |
| M | | | | R w/indicator, | | | | | | | |
| | | | | 40° C. | | | | | | | |
| N | | | | R w/indicator | | | | | | | |
| | | | | w/VA64, 40° C. | | | | | | | |
| O | | | | R w/indicator | | | | | | | |
| | | | | w/Kollicoat, 40° C. | | | | | | | |
| P | | | | S w/indicator, | | | | | | | |
| | | | | 40° C. | | | | | | | |
| Q | | | | R w/indicator, | | | | | | | |
| | | | | RT | | | | | | | |
| R | | | | R w/indicator | | | | | | | |
| | | | | w/Kollicoat, RT | | | | | | | |
| S | | | | PC, R | | | | | | | |
| NCO | N/A | N/A | N/A | NC | | N/A | 50 | | | | |

TABLE 2
Experiments Content AWT067 (AMA only)

| | | | | | | SC | | | Inocu- | | |
|---|---|---|---|---|---|---|---|---|---|---|---|
| | | | Formu- | | | Active | | | lation | Samp- | |
| Model | Active | Additional | lation | Model | | conc. | Vol. | Micro- | [Log | ling | Temp, |
| No. | material | materials | number | description | Medium | [ppm] | [ml] | organism | cfu/ml] | times | C. |
| A | SC + | Vinnol/EtOAc | WE003/18 + | R, RT | EPA #2 | 10 unless | 500 | R. Terrigena | 5 | 0.5, | EPA#1: |
| B | CG8-H | (see also model | WE004 | S, RT | | specified | | | | 4 hours | RT |
| C | | description ) | | R w/VA64, RT | | otherwise | | | | | EPA#2: |
| D | | | | R w/Kollicoat, RT | | in the | | | | | 4° C. |
| E | | | | R w/indicator, | | model | | | | | |
| | | | | RT | | desc. | | | | | |
| F | | | | R w/indicator | | | | | | | |
| | | | | w/VA64, RT | | | | | | | |
| G | | | | R w/indicator | | | | | | | |
| | | | | w/Kollicoat, RT | | | | | | | |
| H | | | | S w/indicator, RT | | | | | | | |
| I | | | | R, 40° C. | | | | | | | |
| J | | | | S, 40° C. | | | | | | | |
| K | | | | R w/VA64, 40° C. | | | | | | | |
| L | | | | R w/Kollicoat, 40° C. | | | | | | | |
| M | | | | R w/indicator, 40° C. | | | | | | | |
| N | | | | R w/indicator | | | | | | | |
| | | | | w/VA64, 40° C. | | | | | | | |
| O | | | | R w/indicator | | | | | | | |
| | | | | w/Kollicoat, 40° C. | | | | | | | |
| P | | | | S w/indicator, 40° C. | | | | | | | |
| Q | | | | WE027/1 50% + | TSB 1:500 | | | | | | |
| | | | | H3PO4 | | | | | | | |
| R | | | | WE027/1 50%, | TSB 1:500 | | | | | | |
| | | | | Rev | | | | | | | |
| NCS | N/A | N/A | N/A | NC EPA#1 | TSB 1:500 | N/A | 50 | | | | |
| NCT | N/A | N/A | N/A | NC EPA#2 | EPA #2 | N/A | 50 | | | | |

The above experiments, AWT066 and AWT067, are yes/no experiments, thus counting is needed only for 0,1 dilutions. Measurements of pH are taken. The experiments include a shaking step, shaking thoroughly after filling and once again in 15 minutes.

Results

TABLE 5
Analytic measurement ClOₓ-species

| | | 0.5 h | | | | | 4 h | | | | |
|---|---|---|---|---|---|---|---|---|---|---|---|
| | | | | | total | | | | | total | |
| # | Model | Cl2 | ClO2 | ClO2− | ox' | pH | Cl2 | ClO2 | ClO2− | ox' | pH |
| A | Reversed, | 0 | 0 | 0 | 0 | 5.35 | 0 | 0 | 0 | 0 | 4.96 |
| | HALT | | | | | | | | | | |
| B | Sandwiched, | 0 | 0 | 0 | 0 | 4.98 | 0 | 7.204 | 0 | 7.204 | 4.13 |
| | HALT | | | | | | | | | | |
| E | Reversed | 0 | 0 | 0 | 0 | 4.89 | 0 | 0 | 0 | 0 | 4.8 |
| | w/Indicator, | | | | | | | | | | |
| | HALT | | | | | | | | | | |
| F | Reversed | 0 | 0 | 0 | 0 | 4.77 | 0 | 0 | 0 | 0 | 4.65 |
| | w/Indicator, | | | | | | | | | | |
| | w/VA64, | | | | | | | | | | |
| | HALT | | | | | | | | | | |
| G | Reversed | 0 | 0 | 0 | 0 | 4.79 | 0 | 0 | 0 | 0 | 4.62 |
| | w/Indicator, | | | | | | | | | | |
| | w/Kollicoat, | | | | | | | | | | |
| | HALT | | | | | | | | | | |
| I | Reversed, | 0 | 3.256 | 0 | 3.256 | 4.85 | 0 | 2.712 | 3.26 | 5.972 | 4.4 |
| | 40° C. | | | | | | | | | | |
| J | Sandwiched, | 0.384 | 0 | 3.848 | 3.848 | 4.51 | 1.216 | 0 | 5.176 | 5.176 | 4.39 |
| | 40° C. | | | | | | | | | | |
| M | Reversed | 0 | 2.864 | 3.44 | 6.304 | 4.43 | 0 | 2.74 | 3.36 | 6.1 | 4.32 |
| | w/Indicator, | | | | | | | | | | |
| | 40° C. | | | | | | | | | | |
| N | Reversed | 0 | 1.704 | 6.848 | 8.552 | 4.66 | 0 | 2.568 | 7.736 | 10.304 | 4.26 |
| | w/Indicator, | | | | | | | | | | |
| | w/VA64, | | | | | | | | | | |
| | 40° C. | | | | | | | | | | |
| P | Reversed | | | | 0 | 4.53 | 0 | 2.9 | 0 | 2.9 | 4.38 |
| | w/Indicator, | | | | | | | | | | |
| | w/Kollicoat, | | | | | | | | | | |
| | 40° C. | | | | | | | | | | |
| Q | Reversed | 0 | 1.644 | 5.492 | 7.136 | 4.42 | 0 | 1.5 | 5.236 | 6.736 | 4.37 |
| | w/indicator, | | | | | | | | | | |
| | “fresh” | | | | | | | | | | |

Summary and Conclusions

2 week old assemblies were tested for their efficacy. Samples that were stored under RT and under 40° C. in a dry oven were effective both under EPA #1 and EPA #2 conditions. Samples that were stored under HALT of 40° C. were ineffective under EPA #1 (were not tested for EPA #2). Thus, it can be clearly deduced that humidity is primary degradation engine of the assembly. Application of top “protection” layer of Luvitec VA64 (PVP/PVAc, 16.67% in IPA) or Kollicoat Protect (PVA/PEG:PVA, 10% in DDW) did not seem to improve the humidity resistance of the assemblies (and nor did it have a negative impact). Hach measurements results come in agreement with the efficacy trial. Next steps: (1) test efficacy after longer durations (1 m, 2 m, . . . ), (2) explore new methods and materials for humidity protection (different polymers, bi-layer intermediate protection layer, etc.)

Temperature of 40° C. by itself does not seem to degrade efficacy potential of the assemblies, at least not in time scale of couple of weeks. Hence, it may be fruitful to examine again the concept of humidity resistant packaging or covering tape.

The fresh sample was not effective after 0.5 hours, but did exhibit positive ClOₓ-species readings. This may be due to preparation or sampling error.

Assemblies with SC formulation based on Joncryl DFC 3030 (50 wt % SC from SC_{(aq)}, 250 μm wet thickness), were not effective after 4 h both at reversed geometry and solely with H₃PO₄ addition. However, these same samples had shown partial efficacy after 4 h in FOM074 (vs. E. Coli, 10⁵ cfu/ml) as well as positive Hach readings.

In a non-limiting example, Sample 61 [AWT061] was tested regarding the feasibility of preparing the SC layer formulation with aqueous SC product (OxyChem Textone L or XL, 25 wt % and 31 wt %, respectively). Reversed assemblies were fabricated where the SC layer is prepared from either PE032 (Hycar 26288+SC+NH₃+KaMin 70C), or SC solution in Vinnol stock solution based in ethyl acetate, butyl acetate or their mixture (all 20 wt % Vinnol). All assemblies were ineffective. Analytic measurement did not reveal any ClOₓ species within the trial time scale.

AWT072

Experiments Goals:

Examine the efficacy of 1 m old assemblies stored under 40° C. in a “dry” oven.

TABLE 1
Experiments Content (AMA only)

| | | | | | | SC | | | Inocu- | | |
|---|---|---|---|---|---|---|---|---|---|---|---|
| | | | Formu- | | | Active | | | lation | Samp- | |
| Model | Active | Additional | lation | Model | | conc. | Vol. | Micro- | [Log | ling | Temp, |
| No. | material | materials | number | description | Medium | [ppm] | [ml] | organism | cfu/ml] | times | C. |
| A | SC + | Vinnol/ | WE003/18 + | R, GTW | GTW | 10 unless | 500 | E. Coli | 3 | 0.5, | GTW: |
| B | CG8-H | EtOAc | WE004 | S, GTW | | specified | | | | 4 hours | RT |
| C | | (see | | R w/VA64, GTW | | otherwise | | | | | CTW: |
| D | | also | | R w/Kollicoat, GTW | | in the | | | | | 4° C. |
| E | | model | | R w/indicator, GTW | | model | | | | | |
| F | | descrip- | | R w/indicator w/VA64, | | desc. | | | | | |
| | | tion) | | GTW | | | | | | | |
| G | | | | R w/indicator | | | | | | | |
| | | | | w/Kollicoat, GTW | | | | | | | |
| H | | | | S w/indicator, GTW | | | | | | | |
| I | | | | R, CTW | CTW | | | | | | |
| J | | | | S, CTW | | | | | | | |
| K | | | | R w/VA64, CTW | | | | | | | |
| L | | | | R w/Kollicoat, CTW C | | | | | | | |
| M | | | | R w/indicator, CTW | | | | | | | |
| N | | | | R w/indicator w/VA64, | | | | | | | |
| | | | | CTW C | | | | | | | |
| O | | | | R w/indicator | | | | | | | |
| | | | | w/Kollicoat, CTW | | | | | | | |
| P | | | | S w/indicator, CTW | | | | | | | |
| Q | | | | S, fresh, GTW | GTW | | | | | | |
| R | | | | S, fresh, CTW | CTW | | | | | | |
| NCS | N/A | N/A | N/A | NC GTW | GTW | N/A | 50 | | | | |
| NCT | N/A | N/A | N/A | NC CTW | CTW | N/A | 50 | | | | |

This is a yes/no experiment, thus counting is needed only for 0, 1 dilutions. All samples are to be sampled w/neutralizer (1:1). CTW: Humic acid, 15±5 mg/L; CaCO₃, 100±20 mg/L; NaCl, 1500±150 mg/L, Turbidity, not applicable at the moment. Measure pH. Shake thoroughly after filling and one again after 15′.

Results: E. coli viable counts

TABLE
ClO'x-species analytic measurement

| | | 0.5 h | | | | | 4 h | | | | |
|---|---|---|---|---|---|---|---|---|---|---|---|
| | | | | | total | | | | | total | |
| Serial | Model | Cl2 | ClO2 | ClO2− | ox′ | pH | Cl2 | ClO2 | ClO2− | ox' | pH |
| A | Reversed | 0 | 0.08 | 2.852 | 2.932 | 5.29 | 0 | 1.964 | 3.812 | 5.776 | 4.27 |
| B | Sandwiched | 0 | 3.428 | 0 | 3.428 | 4.46 | 1.304 | 0 | 5.588 | 5.588 | 4.08 |
| C | Reversed | 0 | 3.94 | 3.412 | 7.352 | 4.33 | 0 | 1.964 | 4.012 | 5.976 | 4.22 |
| | w/VA64 | | | | | | | | | | |
| D | Reversed | 0 | 3.612 | 2.964 | 6.576 | 4.35 | 0 | 3.272 | 3.368 | 6.64 | 4.25 |
| | w/Kollicoat | | | | | | | | | | |
| E | Reversed | 0 | 1.66 | 3.408 | 5.068 | 4.34 | 0 | 0.956 | 3.204 | 4.16 | 4.29 |
| | w/indicator | | | | | | | | | | |
| F | Reversed | 0 | 2.472 | 3.988 | 6.46 | 4.23 | 0 | 2.8 | 4.428 | 7.228 | 4.19 |
| | w/indicator | | | | | | | | | | |
| | w/VA64 | | | | | | | | | | |
| G | Reversed | 0 | 0.732 | 3.836 | 4.568 | 4.33 | 0 | 1.92 | 3.216 | 5.136 | 4.28 |
| | w/indicator | | | | | | | | | | |
| | w/Kollicoat | | | | | | | | | | |
| Q | Sandwiched, | 0 | 2.692 | 0 | 2.692 | 4.71 | 0 | 4.576 | 0 | 4.576 | 4.23 |
| | fresh | | | | | | | | | | |

Results and discussion: All assemblies were effective after 1 m of storage in 40° C. both in GTW and CTW (WHO protocol). Hence, temperature of 40° C. in the relatively dry atmosphere of the oven (˜30%) is not sufficient to promote degradation. I.e., humidity is the important source of degradation, not the temperature (at least below 40° C.).

Additional experiments: (1) examine the efficacy after longer storage periods. (2) Examine shelf life under different storage conditions (temperature and humidity).

AWT061 and AWT063

In a non-limiting example, Samples 62 and 63 [AWT061 and AWT063] examined efficacies of reversed and sandwiched assemblies of 2.5, 5 and 10 ppm against 10² cfu/ml of R. Terrigena (instead of 10⁵). 5 and 10 ppm assemblies were effective after 30 min. 10 ppm reversed assemblies and 5 and 10 ppm sandwiched assemblies were also effective after merely 15 min. 2.5 ppm assemblies were effective after the 4 h sampling only in the sandwiched geometry.

AWT061

Experimental Goals; (1) preliminary examination of Flexo-printed assemblies (Davik). (2) examination of aqueous SC based formulations, reversed geometry. 1^{st} aqueous SC formulation layer: (a) Hycar (water)-based, (b) EtOAc-based. (c) BuOAc-based. (d) Et/BuOAc-based, and (3) shallow inoculation case study (target: India tap water).

Experiments Content (AMA only)

| | | | | | | SC | | | Inocu- | | |
|---|---|---|---|---|---|---|---|---|---|---|---|
| | | | Formu- | | | Active | | | lation | Samp- | |
| Model | Active | Additional | lation | Model | | conc. | Vol. | Micro- | [Log | ling | Temp, |
| No. | material | materials | number | description | Medium | [ppm] | [ml] | organism | cfu/ml] | times | C. |
| A | SC + | Vinnol/ | WE018/8 + | Davik III (10 ppm) | TSB | 10 unless | 500 | R. Terrigena | 5 | 0.5, | EPA#1: |
| B | CG8-H | EtOAc (see | WE004/32 + | Davik I | 1:500 | specified | | | | 4 hours | RT |
| C | | also model | HB003/2 | Davik V | | otherwise | | | | | |
| D | | description) | | Davik III + H3PO4 | | in the | | | | | |
| E | | | WE018/7 + | WE018 over Hycar | | model | | | | | |
| | | | WE021/2 | based SC(aq) | | desc. | | | | | |
| | | | | formulation | | | | | | | |
| | | | | (foamed) | | | | | | | |
| F | | | | WE018 over Hycar | | | | | | | |
| | | | | based SC(aq) | | | | | | | |
| | | | | formulation | | | | | | | |
| | | | | (defoamed) | | | | | | | |
| G | | | WE018/7 + | WE018 over EtOAc | | | | | | | |
| | | | WE020/1 | based SC(aq) | | | | | | | |
| | | | | formulation | | | | | | | |
| H | | | WE018/7 + | WE018 over BuOAc | | | | | | | |
| | | | WE022/1 | based SC(aq) | | | | | | | |
| | | | | formulation | | | | | | | |
| I | | | WE018/7 + | WE018 over EtBuOAc | | | | | | | |
| | | | WE023/1 | based SC(aq) | | | | | | | |
| | | | | formulation | | | | | | | |
| J | | | WE018/7 + | Rev, 10 ppm | | | | | 2 | | |
| K | | | WE004/31 + | Rev, 5 ppm | | | | | | | |
| L | | | WE003/16 | Sandw, 10 ppm | | | | | | | |
| M | | | | Sandw, 5 ppm | | | | | | | |
| NCN | N/A | N/A | N/A | NC 5-log | | N/A | 50 | | 5 | | |
| NCO | N/A | N/A | N/A | NC 2-log | | N/A | 50 | | 2 | | |

This is a yes/no experiment, thus counting is needed only for 0, 1 dilutions. Measure pH.

Results: Table showing R. Terrigena viable counts

Hach's CIOₓ analytic measurement:

| | | 0.5 h | | | | | 4 h | | | | |
|---|---|---|---|---|---|---|---|---|---|---|---|
| | Model | | | | total | | | | | total | |
| # | description | Cl2 | ClO2 | ClO2− | ox' | pH | Cl2 | ClO2 | ClO2− | ox' | pH |
| A4 | Davik 10 ppm | 0 | 8.2 | 0 | 8.2 | 5.11 | 0 | 0 | 0 | 0 | 4.31 |
| A5 | | 0 | 0 | 0 | 0 | 5.24 | 0 | 0 | 0 | 0 | 4.37 |
| A6 | | 0 | 0 | 0 | 0 | 5.09 | 0 | 5.42 | 0 | 5.42 | 5.11 |
| E | WE018 over foamed | 0 | 0 | 0 | 0 | 4.43 | 0 | 0 | 0 | 0 | 4 |
| | Hycar-based SC(aq) | | | | | | | | | | |
| | formlation | | | | | | | | | | |
| F | WE018 over defoamed | 0 | 0 | 0 | 0 | 4.75 | 0 | 0 | 0 | 0 | 4.41 |
| | Hycar-based SC(aq) | | | | | | | | | | |
| | formlation | | | | | | | | | | |
| G | WE018 over | 0 | 0 | 0 | 0 | 4.21 | 0 | 0 | 0 | 0 | 3.94 |
| | Vinnol/EtOAc-based | | | | | | | | | | |
| | SC(aq) formulation | | | | | | | | | | |
| H | WE018 over | 0 | 0 | 0 | 0 | 4.6 | 0 | 0 | 0 | 0 | 3.95 |
| | Vinnol/BuOAc-based | | | | | | | | | | |
| | SC(aq) formulation | | | | | | | | | | |
| I | WE018 over | 0 | 0 | 0 | 0 | 4.35 | 0 | 0 | 0 | 0 | 3.43 |
| | Vinnol/EtBuOAc-based | | | | | | | | | | |
| | SC(aq) formulation | | | | | | | | | | |

Summary and Conclusions

Davik-printed assemblies were not effective vs. R. Terrigena (10⁵ cfu/ml) and did not demonstrated coherent positive ClOx's values in the Hach's analytic measurements. Since the pH was reduced both on the AMA and the analytical trial it is believed that lack of acidifier was not the cause of inefficacy. The inefficacy of sample D which was added with H₃PO₄ also supports this conclusion. It is therefore concluded that the lack of AM efficacy is due to an insufficient amount of CDO released into the medium. It may be due incorrect weighing and assembly active area calculation or inconsistent SC surface concentration. It is also possible that CDO could not have reached the medium due to formation of impermeable film or due to a rapid degradation. Next steps: 1. Re-weigh assemblies, repeat the trial. 2. Perform SEM analysis of the assemblies. 3 re-perform printing at Davik, make use of diluted SC formulation to avoid sticking.

Reversed assemblies were prepared with formulation bases on aqueous SC and either water-borne acrylic emulsion (Hycar 26288) or Vinnol H30/48M in ethyl or butyl acetate. All of these assemblies were ineffective and did not demonstrate any ClOx readings. The inefficacy may result from CDO consumption during preparation or from formation of impermeable matrix following the introduction of water. Furthermore, the medium in these bottles acquired a yellow-green hue. This may be derived from tartrazine leaching into the medium. This may be facilitated by insufficient drying of the SC layer followed by insufficient stability of the IX layer contents. Additional experiment: 1. Prepare similar assemblies with longer drying time and/or higher drying Tand repeat AMA trial.

All examined assemblies (reversed and sandwiched, 5 and 10 ppm) were effective against 10² cfu/ml of R. Terrigena after 30 min. Additional experiment: 1. Apply lower SC contents and shorter sampling times.

AWT063

The following experimenial details are in connection with AWT063:

Experiments Goals

Explore the AMA of reversed and sandwiched assemblies against shallow inoculation levels (10² cfu/ml) simulating tap water contamination in India.

Experiments Content (AMA only)

| | | | | | | SC | | | Inocu- | | |
|---|---|---|---|---|---|---|---|---|---|---|---|
| | | | Formu- | Model | | Active | | | lation | Samp- | |
| Model | Active | Additional | lation | descrip- | Med- | conc. | Vol. | Micro- | [Log | ling | Temp, |
| No. | material | materials | number | tion | ium | [ppm] | [ml] | organism | cfu/ml] | times | C. |
| A | SC + | Vinnol/ | WE018/9 + | R10 | TSB | 10 unless | 500 | R. Terrigena | 2 | 0.5, | EPA#1: |
| B | CG8-H | EtOAc | WE004/32 | R5 | 1:500 | specified | | | | 4 hours | RT |
| C | | (see | | S10 | | otherwise | | | | | |
| D | | also | | S5 | | in the | | | | | |
| E | | model | | R7.5 | | model | | | | | |
| F | | description) | | R2.5 | | desc. | | | | | |
| G | | | | S7.5 | | | | | | | |
| H | | | | S2.5 | | | | | | | |
| I | | | | R10 | | | | | 5 | | |
| J | | | | R5 | | | | | | | |
| K | | | | S10 | | | | | | | |
| L | | | | S5 | | | | | | | |
| M | | | | R7.5 | | | | | | | |
| N | | | | S7.5 | | | | | | | |
| NCO | N/A | N/A | N/A | NC 2-log | | N/A | 50 | | 2 | | |
| NCO | N/A | N/A | N/A | NC 5-log | | N/A | 50 | | 5 | | |

The above experiment is a yes/no experiment, thus counting is needed only for 0, 1 dilutions. Measurements of pH are taken. WE018 over WE021: apply WE02I, 60 μm, 1' @ 100° C.+60'@60° C. →apply WE018, 200 μm, 30' @60° C.

Results: Table-R. Terrigena viable counts

Sandwiched assemblies yielded total 5-log eradication after 30 min down to 5 ppm of SC. Reversed assemblies brought total eradication down to 7.5 ppm (5 ppm eradicated S-log only after 4 h).

The differences in efficacies is believed to be related to the IX content in contact with the SC. The IX content of the reversed assembly was 2.6·10⁻³ mg/ml/ppm_SC while the IX content of the sandwiched assembly was 3.6·10⁻³ mg/ml/ppm_SC. I.e., the IX content of the sandwiched assembly is 40% higher than that of the reversed assembly.

Additional experiments: 1. repeat the trial to obtain reproducibility and improved resolution (time and concentration). 2. Repeat the trial using neutralizer and/or filtration sampling technique.

In a non-limiting example, Samples 64 and 65 [AWT061 and AWT062] examined reversed assemblies prepared by flexo printing at Davik Sde-Boker. Flexo is a common printing technique utilizing a dosage roller (“anilxx”) dipped in the formulation fountain and pressed onto a second plate roller. The plate roller transfers the formulation or ink to the substrate. Assemblies were prepared on top of a thin background layer (formulation HB003, Vinnol/EtOAc KaMin 70° C.). Assemblies were ineffective unless an absurd amount of material was introduced. The samples were ineffective probably due to insufficient amount of material transferred to the sheet during the flexo printing. The relatively thin layers, ˜3 μm each, also made correct calculation of the active area required extremely hard. Analytic ClOₓ-species determination yielded the same conclusions.

AWT062

In a non-limiting example, Sample 66 [AWT062] also explored if the aspect ratio of the assembly (i.e., the ratio between the assembly's length and width) possess influence on its efficacy. The trial results demonstrated that the aspect ratio is insignificant, at least under the trial resolution and when the trial bottles are agitated.

Experiments Goals: (1) preliminary examination of Flexo-printed assemblies (@Davik), (2) examination of aqueous SC based formulations, reversed geometry. 1^{st} aqueous SC formulation layer: Hycar (water)-bascd, (3) examination of the influence of the assembly length/aspect ratio on the efficacy.

Experiments Content (AMA only)

| | | | | | | SC | | | Inocu- | | |
|---|---|---|---|---|---|---|---|---|---|---|---|
| | | | Formu- | Model | | Active | | | lation | Samp- | |
| Model | Active | Additional | lation | descrip- | Med- | conc. | Vol. | Micro- | [Log | ling | Temp, |
| No. | material | materials | number | tion | ium | [ppm] | [ml] | organism | cfu/ml] | times | C. |
| A | SC + | Vinnol/ | WE018/9 + | Davik by 0.2 mg/ml 10 ppm | TSB | 10 unless | 500 | R. | 5 | 0.5, | EPA#1: |
| B | CG8-H | EtOAc | WE004/32 + | Davik by 0.2 mg/ml 25 ppm | 1:500 | specified | | Terrigena | | 4 hours | RT |
| C | | (see also | HB003/2 | Davik by 0.05 mg/ml 10 ppm | | otherwise | | | | | |
| D | | model | | Davik by 0.05 mg/ml 25 ppm | | in the | | | | | |
| E | | description) | | Davik XX (0.005 mg/ml) 10 ppm | | model | | | | | |
| F | | | WE018/7 + | WE018 over WE021 | | desc. | | | | | |
| | | | WE021/2 | | | | | | | | |
| G | | | WE004/31 + | Reversed, 10 ppm | | | | | | | |
| H | | | WE018/9 | Sandwiched, 10 ppm | | | | | | | |
| I | | | | Reversed, long, 10 ppm | | | | | | | |
| J | | | | Sandwiched, long 10 ppm | | | | | | | |
| K | | | | Reversed, 5 ppm | | | | | | | |
| L | | | | Sandwiched, 5 ppm | | | | | | | |
| M | | | | Reversed, long, 5 ppm | | | | | | | |
| N | | | | Sandwiched, long, 5 ppm | | | | | | | |
| NCO | N/A | N/A | N/A | NC | | N/A | 50 | | | | |

This is a yes/no experiment, thus counting is needed only for 0, 1 dilutions. Measure pH. WE018 over WE021: apply WE021, 60 μm, 1′@100° C.+60′ @60° C. →apply WE018, 200 μm, 30′ @60° C.

Results: Table showing R. Terrigena viable counls:

TABLE
Hach's ClOx analytic measurement

| | | 0.5 h | | | | | 4 h | | | | |
|---|---|---|---|---|---|---|---|---|---|---|---|
| # | Model | Cl2 | CDO | ClO2− | Tot ClOx | pH | Cl2 | CDO | ClO2− | Tot ClOx | pH |
| A4 | Davik | 0 | 5 | 0 | 5.048 | 4.52 | 0 | 0 | 0 | 0 | 4.95 |
| A5 | 10 ppm | 0 | 0 | 0 | 0 | 4.82 | 0 | 0 | 0 | 0 | 4.95 |
| | [0.2 mg/ml] | | | | | | | | | | |
| C4 | Davik | 0 | 1 | 0 | 0.984 | 4.62 | 0 | 1.716 | 0 | 1.716 | 4.04 |
| C5 | 10 ppm | 0 | 1.1 | 0 | 1.104 | 4.6 | 0 | 2.08 | 0 | 2.08 | 4.53 |
| | [0.05 mg/ml] | | | | | | | | | | |
| E4 | Davik XX | 0 | 1.4 | 4.23 | 5.644 | 3.87 | 0 | 0.692 | 7.84 | 8.532 | 3.69 |

Summary and conclusions

Assemblies printed at Davik were not effective unless an extremely large area was inserted into the bottle. It may be derived from the inability of the flexo technique to transfer the relatively large grained SC to the assembly surface. This, together with the layers being thin and hard to weigh (and calculate correct SC surface concentrations) result in demand for large areas of printed sheet to obtain satisfying efficacy. Hach results support the AMA trial findings. Usage of large area also resulted in pigment leaching to the media and appearance of yellow-green hue in the medium.

Contamination control sample (i.e., sample immersed in non-inoculated medium to test for possible manufacture process microbial contaminations) was found to be negative for microbial growth.

Reversed assemblies prepared using SC solution in Hycar 26288 based formulation was once again ineffective (see AWT061). The water also developed a significant green hue. Similar samples are also investigated in a parallel technology trial (FOM073).

The aspect ratio (i.e., the ratio between the length and the width of the assembly) did not possess conclusive influence on the efficacy (at least within the trial resolution). It may be worthwhile to repeat this case study where the bottles are not agitated.

AWT064

In a non-limiting example, Sample 67 [AWT064] was examined regarding the efficacies of assemblies prepared with WE025, a formulation prepared with Hycar 26288 and SC solution (31%) in SC dry concentration of 10 wt %, 20 wt %, 50 wt % and 85 wt %. Each model was tested as is, with additional Vinnol/EtOAc layer and with WE018 layer on top (i.e., reversed assembly). None of the reversed assemblies yielded efficacy or detection of ClOₓ-species. SEM micrographs revealed that the SC in Hycar layer was devoured by the CG8-H in Vinnol layer, probably forcing SC annihilation upon preparation. The samples without the CG8-H layer (with H₃PO₄ addition) did show efficacy and ClOₓ-species detection, but full eradication was not achieved after 4 h. The release kinetics of the Hycar binder is significantly slower than that of the Vinnol.

Experiments Goals: Explore the effect of the SC volume concentration on release kinetics and efficacy of SC(aq)-bascd formulations with hycar 26288 or Kollicoat Protect as binders.

Experiments Content (AMA only)

| | | | | | | SC | | | Inocu- | | |
|---|---|---|---|---|---|---|---|---|---|---|---|
| | | | Formu- | | | Active | | | lation | Samp- | |
| Model | Active | Additional | lation | Model | | conc. | Vol. | Micro- | [Log | ling | Temp, |
| No. | material | materials | number | description | Medium | [ppm] | [ml] | organism | cfu/ml] | times | C. |
| A | SC + | Vinnol/ | WE018/9 + | Hycar, 10% SC, w/H3PO4 | TSB | 10 | 500 | R. | 5 | 0.5, | EPA#1: |
| B | CG8-H | EtOAc | WE004/31 | Hycar, 20% SC, w/H3PO4 | 1:500 | unless | | Terrigena | | 4 h | RT |
| C | | (see | | Hycar, 50% SC, w/H3PO4 | | specified | | | | | |
| D | | also | | Hycar, 85% SC, w/H3PO4 | | otherwise | | | | | |
| E | | model | | Kollicaot, 85% SC, w/H3PO4 | | in the | | | | | |
| F | | descrip- | | Hycar, 10% SC, w/vinnol | | model | | | | | |
| | | tion) | | top coat, w/H3PO4 | | desc. | | | | | |
| G | | | | Hycar, 20% SC, w/vinnol | | | | | | | |
| | | | | top coat, w/H3PO4 | | | | | | | |
| H | | | | Hycar, 50% SC, w/vinnol | | | | | | | |
| | | | | top coat, w/H3PO4 | | | | | | | |
| I | | | | Hycar, 85% SC, w/vinnol | | | | | | | |
| | | | | top coat, w/H3PO4 | | | | | | | |
| J | | | | Kollicaot, 85% SC, w/vinnol | | | | | | | |
| | | | | top coat, w/H3PO4 | | | | | | | |
| K | | | | Hycar, 10% SC, w/WE018, Rev | | | | | | | |
| L | | | | Hycar, 20% SC, w/WE018, Rev | | | | | | | |
| M | | | | Hycar, 50% SC, w/WE018, Rev | | | | | | | |
| N | | | | Hycar, 85% SC, w/WE018, Rev | | | | | | | |
| O | | | | Kollicaot, 85% SC, w/WE018, Rev | | | | | | | |
| P | | | | PC, Rev | | | | | | | |
| NCP | N/A | N/A | N/A | NC 2-log | | N/A | 50 | | | | |

This is a yes/no experiment, thus counting is needed only for 0, 1 dilutions. Measure pH. Thermal treatment of 1^{st} layer: ′@100° C.+30′@60° C. Shake bottles thoroughly immediately and 10′ after filling.

Results: Table illustrating R. Terrigena viable counts

TABLE
Hach ClOx's analytic measurement results

| | | 0.5 h | | | | | 4 h | | | | |
|---|---|---|---|---|---|---|---|---|---|---|---|
| # | Model | Cl2 | CDO | ClO2− | ClOx's | pH | Cl2 | CDO | ClO2− | ClOx's | pH |
| A | Hycar, 10% SC | 6.2 | 0 | 14.3 | 14.3 | 6.65 | 3.376 | 0 | 12.72 | 12.72 | 6.41 |
| B | Hycar, 20% SC | 0.076 | 0 | 5.136 | 5.136 | 6.06 | 0 | 0.272 | 4.708 | 4.98 | 6 |
| C | Hycar, 50% SC | 0.704 | 0 | 5.784 | 5.784 | 6.48 | 0 | 0.116 | 5.92 | 6.036 | 6.38 |
| D | Hycar, 85% SC | 0 | 0 | 4.856 | 4.856 | 6.66 | 0.248 | 0 | 5.264 | 5.264 | 6.39 |
| E | Kollicaot, 85% SC | 0 | 0.812 | 8.788 | 9.6 | 6.36 | 0 | 0.052 | 9.516 | 9.568 | 5.96 |
| K | Rev, Hycar, 10% SC | 0 | 0 | 0 | 0 | 4.22 | 0 | 0 | 0 | 0 | 3.81 |
| L | Rev, Hycar, 20% SC | 0 | 0 | 0 | 0 | 4.75 | 0 | 0 | 0 | 0 | 4.1 |
| M | Rev, Hycar, 50% SC | 0 | 0 | 0 | 0 | 4.86 | 0 | 2.364 | 1.948 | 4.312 | 4.09 |
| N | Rev, Hycar, 85% SC | 0 | 0 | 0 | 0 | 4.7 | 0 | 0 | 0 | 0 | 4.62 |
| O | Rev, Kollicaot, 85% SC | 0 | 0.336 | 6.304 | 6.64 | 4.45 | 0 | 0.52 | 6.248 | 6.768 | 4.34 |
| P | Rev, PC | 0 | 1.692 | 4.436 | 6.128 | 4.49 | 0 | 0.58 | 4.764 | 5.344 | 4.4 |

Summary and conclusions

Sheets prepared with SC(aq) in hycar formulations were slightly effective after 0.5 h when the medium was acidified using H₃PO₄. Total eradication was almost achieved after 4 h. This observation is also supported by the Hach results.

When reversed assemblies were prepared with the same sheets efficacy was not obtained. It may derive from mismatch in the interface forming an impermeable shield. This may impede the release of SC and protons as well as interfere with the formation of the “close contact geometry”.

The topography of the top WE018 layer was greatly influenced from the SC percentage of the bottom layer. The higher the SC content the rougher the top layer topography formed. See FIG. 1 for illustration. This may originate from the roughness of the SC bottom layer or alternatively from formation of gaseous CDO or trapped air forming bubbles.

Application of vinnol stock solution only over the SC layer has slightly impeded efficacy.

Utilization of SC layer composed of 85% SC in Kollicaot protect (10%) solution yielded slightly improved results. specifically, efficacy was also obtained in reversed geometry after 4 h. Nevertheless, IX layer lift-off was observed, probably due to dissolution of the SC layer based on SC impregnated in liquid water soluble matrix. See FIG. 20.

Additional experiments: 1. Examine efficacy after longer duration (technology trials). 2. Vary SC layer filler volume concentration by addition of clay instead of reducing solvent load (constant SC content). 3. Perform similar case study using SC(aq) in vinnol EtOAc, BuOAc, or their mixture.

AWT068

In an non-limiting example, sample 68 [AWT068] was examined regarding the efficacies of reversed and sandwiched assemblies in varying SC contents from 2.5 to 10 ppm vs. shallow inoculation of R. lerrigena (10² cfu/ml). reversed assemblies required 7.5 ppm of SC to exhibit total eradication after 0.5 h while sandwiched assemblies required only 5 ppm of SC. Reversed assemblies with 5 ppm of SC and sandwiched assemblies of 2.5 ppm was only partially effective afier 0.5 h and fully effective after 4 h.

Experiments Goals: (1) filtration sampling technique preliminary and (2) shallow-inoculation (2-log) w/SS+.

TABLE 1
Experiments Content (AMA only)

| | | | | | | SC Active | | | | | |
|---|---|---|---|---|---|---|---|---|---|---|---|
| Model | Active | Additional | Formulation | | | conc. | Vol. | | Inoculation | Sampling | Temp, |
| No. | material | materials | number | Model description | Medium | [ppm] | [ml] | Microorganism | [Log cfu/ml] | times | C. |
| A | SC + | Vinnol/ | WE018/9 + | 20 ppm filtration | TSB | 10 unless | 500 | R. Terrigena | 5 | 0.5, 4 hours | EPA#1: |
| B | CG8-H | EtOAc | WE004/32 | 20 ppm regular | 1:500 | specified | | | | | RT |
| C | | (see also | | 2.5 ppm filtration | | otherwise | | | | | |
| D | | model | | 2.5 ppm regular | | in the | | | | | |
| E | | description) | | R2.5 | | model | | | 2 | 0.25, 4 | |
| F | | | | R5 | | desc. | | | | | |
| G | | | | R7.5 | | | | | | | |
| H | | | | R10 | | | | | | | |
| I | | | | S2.5 | | | | | | | |
| J | | | | S5 | | | | | | | |
| K | | | | S7.5 | | | | | | | |
| L | | | | S10 | | | | | | | |
| NC | N/A | N/A | N/A | NC 5-log | | N/A | 50 | | 5 | 0.5, 4 | |
| | | | | filtration | | | | | | | |
| NC | N/A | N/A | N/A | NC 5-log regular | | N/A | 50 | | 5 | 0.5, 4 | |
| NC | N/A | N/A | N/A | NC 2-log | | N/A | 50 | | 2 | 0.25, 4 | |

Notes:

This is a yes/no experiment, thus counting is needed only for 0, 1 dilutions. All samples are to be measured w/neuiralizer.—Samples AᵢCᵢ, and NCMᵢ will be sampled using filtration. Measurements of pH are recorded. Shake thoroughly after filling and one again after 15′.

these values by dilution of the SC formulation or reducing the SC layer wet thickness. Next steps: repeat this trial with shorter sampling times, e.g., 15 min.

AWT069

In a non-limiting example, sample 69 [AWT069] was examined regarding the efficacies of reversed and sandwiched assemblies in varying SC contents from 2.5 to 10 ppm by utilizing the world health organization (WHO) protocol. Efficacy was examined against E. coli in two types of media, General test water (GTW: pH˜7, TOC˜1 mg/L, T˜20° C., TDS˜50-500 mg/L, alkalinity˜40 mg/L, typically replaced by TSB 1:500) and challenge test water (CTW: TOC˜30 mg/L, turbidity˜40 mg/L, T˜4° C., TDS˜1500 mg/L, alkalinity˜200 mg/L). In both GTW and CTW, reversed assemblies were fully effective in 0.5 h down to 7.5 ppm while sandwiched assemblies brought total eradication after 30 min down to 5 ppm.

Experiments Content (AMA Only):

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | |
| No. | material | materials | number | description | Medium | [ppm] | [ml] | organism | [Log cfu/ml] | times | Temp, C. |
| A | SC + | Vinnol/ | | R2.5-GTW | GTW: TSB | 10 unless | 500 | E. Coli | 3 | 0.5, 4 hours | GTW: RT |
| B | CG8-H | EtOAc | | R5-GTW | 1:500 | specified | | | | | CTW: 4° C. |
| C | | (see | | R7.5-GTW | CTW: see | otherwise | | | | | |
| D | | also | | R710-GTW | notes | in the | | | | | |
| E | | model | | S2.5-GTW | | model | | | | 0.25, 4 | |
| F | | description) | | S5-GTW | | desc. | | | | | |
| G | | | | S7.5-GTW | | | | | | | |
| H | | | | S10-GTW | | | | | | | |
| I | | | | R2.5-CTW | | | | | | | |
| J | | | | R5-CTW | | | | | | | |
| K | | | | R7.5-CTW | | | | | | | |
| L | | | | R710-CTW | | | | | | | |
| M | | | | S2.5-CTW | | | | | | | |
| N | | | | S5-CTW | | | | | | | |
| O | | | | S7.5-CTW | | | | | | | |
| P | | | | S10-CTW | | | | | | | |
| NC | N/A | N/A | N/A | NC GTW | | N/A | 50 | | | 0.5, 4 | |
| NC | N/A | N/A | N/A | NC CTW | | N/A | 50 | | | 0.25, 4 | |

This is a yes/no experiment, thus counting is needed only for 0, 1 dilutions. All samples are to be sampled w/neutralizer (1:1). CTW: Humic acid, 15±5 mg/L; CaCO3, 100±20 mg/L; NaCl, 1500±150 mg/L, Turbidity, not applicable at the moment. Measure pH. Shake thoroughly after filling and one again after 15′.

Results and discussion

This study aimed at setting a preliminary idea on the efficacy of reversed and sandwiched assemblies under the WHO protocol. Using GTW, reversed assemblies were effective down to 7.5 ppm and sandwiched assemblies were efficacious down to 5 ppm. Using CTW, sandwiched assemblies were effective down to 7.5 ppm.

AWT070

In a non-limiting example, sample 70 [AWT070] was examined regarding the efficacies of reversed and sandwiched assemblies vs. Clostridium Perfringens spores. 3-log reduction was obtained for 10 ppm assemblies after 4 h. as in sample 43.

Experiments Goals: Focus on formulation WK003+WE004, LbL, coated that showed efficacy. Different parameters that will be tested for impact on efficacy are: Repeat examination of benchmark assemblies for demonstration of reproducibility and positive control. Explore efficacy vs. Clostridium Perfringens.

Experiments content:

| Model | Active | Additional | Formulation | | | SC Active | Vol. | Micro- | Inoculation | Sampling | Temp, |
|---|---|---|---|---|---|---|---|---|---|---|---|
| No. | material | materials | number | Model description | Medium | conc. [ppm] | [ml] | organism | [Log cfu/ml] | times | C. |
| A | SC + | Vinnol/ | WE003/19 | Reversed, 20 ppm | TSB | See model | 500 | Clostridium | 4 | ½, 4 hours | RT |
| B | CG8-H | EtOAc | 0214 + | Reversed, 10 ppm | 1:500 | description | | Perfringens | | | |
| C | | | WE004/33 | Reversed, 7.5 ppm | | | | | | | |
| D | | | | Sandwiched, 20 ppm | | | | | | | |
| E | | | | Sandwiched, 10 ppm | | | | | | | |
| F | | | | Sandwiched, 7.5 ppm | | | | | | | |
| NC-J | N/A | N/A | N/A | NC | | N/A | 50 | | 4 | ½, 4 hours | RT |

This is a yes/no experiment, thus counting is needed only for 0, 1 dilutions. Measure ClOₓ concentrations and swelling at 1, 4 hours. If any of formulations are active will be tested for organoleptic attributes. Measure pH.

Microbiology Results: (Clostridium perfringensviable counts in test tubes)

Experiments Content

| Model | Active | Additional | Formulation | | Me- | SC Active | Vol. | Micro- | Inoculation | Sampling | Temp, |
|---|---|---|---|---|---|---|---|---|---|---|---|
| No. | material | materials | number | Model description | dium | conc. [ppm] | [ml] | organism | [Log cfu/ml] | times | C. |
| A | SC + | Vinnol/ | FLY098 | Sandwiched, 10 ppm | TSB | See model | 500 | R. Terrigena | 5 | ½, 4 hours | RT |
| B | CG8-H + | EtOAc | | Sandwiched, 7.5 ppm | 1:500 | description | | | | | |
| C | Kamin 70 | | | Sandwiched, 10 ppm | | | | | 3 | | |
| D | | | | Sandwiched, 7.5 ppm | | | | | | | |
| E | | | | Sandwiched, 10 ppm | | | | E. Coli | 5 | | |
| F | | | | Sandwiched, 7.5 ppm | | | | | | | |
| NC-R | N/A | N/A | N/A | NC | | N/A | 50 | R. Terrigena | 5/3 | ½, 4 hours | RT |
| NC-E | N/A | N/A | N/A | NC | | N/A | 50 | E. Coli | 5 | ½, 4 hours | RT |

This is a yes/no experiment, thus counting is needed only for 0, 1 dilutions. Measure ClOₓ concentrations and swelling at 0.5, 4 hours. If any of formulations are active will be tested for organoleptic attributes. Measure pH.

Microbiology Results: microorganisms viable counts in test tubes

See FIG. 21 for an illustration of microorganisms viable counts.

Microbiology commentS: both sandwiched 7.5&10 ppm had antimicrobial activity against E.coli & R. terrigena.

AWT073

In a non-limiting example, sample 72 [AWT073] was examined regarding the efficacies of reversed and sandwiched assemblies where KaMin 70° C. calcined clay was added to the CG8-H top layer in varying concentrations (0-20 wt%). All assemblies were found effective after 30 min in GTW. In CTW, addition of KaMin 70° C. had negative impact on the efficacy of reversed assemblies (impeded efficacy to bring total eradication after only 4 h). In sandwiched assemblies, addition of KaMin 70° C. did not possess any impact on the efficacy in CTW.

Experiments Goals: Examine the efficacy and organoleptic attributes of reversed and sandwiched assemblies with and without further addition of KaMin 70C. calcined clay to the CG8-H-bearing top layer.

TABLE 1
Experiments Content (AMA only)

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | Vinnol/ | WE018/9 (no | R, w/o clay | GTW | 10 unless | 500 | E. Coli | 3 | 0.5, 4 hours | GTW: |
| B | CG8-H | EtOAc | clay) | S, w/o clay | | specified | | | | | RT |
| C | | (see also | or WE032/1 | R, 10 wt % clay | | otherwise | | | | | CTW: |
| D | | model | (10 wt % clay) | S, 10 wt % clay | | in the | | | | | 4° C. |
| E | | description) | or WE033 (20 wt | R, 20 wt % clay | | model | | | | | |
| F | | | % clay) + | S, 20 wt % clay | | desc. | | | | | |
| G | | | WE004/32 | R, w/o clay | CTW | | | | | | |
| H | | | | S, w/o clay | | | | | | | |
| I | | | | R, 10 wt % clay | | | | | | | |
| J | | | | S, 10 wt % clay | | | | | | | |
| K | | | | R, 20 wt % clay | | | | | | | |
| L | | | | S, 20 wt % clay | | | | | | | |
| NCM | N/A | N/A | N/A | NC GTW | GTW | N/A | 50 | | | | |
| NCN | N/A | N/A | N/A | NC CTW | CTW | N/A | 50 | | | | |

Microbiology comments: bottles had antimicrobial activity against E. coli in both temperatures.

CIOₓ-species analytical measurement:

| | | 0.5 h | | | | | 4 h | | | | |
|---|---|---|---|---|---|---|---|---|---|---|---|
| | | | | | total | | | | | total | |
| Serial | Model | Cl2 | ClO2 | ClO2− | ox′ | pH | Cl2 | ClO2 | ClO2− | ox′ | pH |
| A | Reversed w/o | 0 | 1.772 | 2.652 | 4.424 | 4.54 | 0 | 1.136 | 3.456 | 4.592 | 4.51 |
| | Kamin | | | | | | | | | | |
| B | Sandwiched | 0 | 2.592 | 0 | 2.592 | 4.56 | 0 | 3.968 | 0 | 3.968 | 4.32 |
| | w/o Kamin | | | | | | | | | | |
| C | Reversed 10% | 0 | 5.588 | 3.952 | 9.54 | 4.58 | 0 | 0.2 | 5.728 | 5.928 | 4.5 |
| | Kamin | | | | | | | | | | |
| D | Sandwiched | 0 | 5.62 | 0 | 5.62 | 4.27 | 0 | 2.064 | 3.26 | 5.324 | 4.13 |
| | 10% Kamin | | | | | | | | | | |
| E | Reversed 20% | 0 | 0.556 | 5.524 | 6.08 | 4.34 | 0 | 0 | 5.676 | 5.676 | 4.33 |
| | Kamin | | | | | | | | | | |
| F | Sandwiched | 0 | 0 | 0 | 0 | 4.42 | 0 | 1.312 | 2.252 | 3.564 | 4.12 |
| | 20% Kamin | | | | | | | | | | |

In a non-limiting example, sample 74 [AWT074] was examined regarding the efficacies of reversed assembly prepared via screen printing technique. Assemblies were found to be effective and brought total eradication after 30 min in GTW and CTW.

Experimental goals: (1) examine the efficacy and organoleptic attributes of reversed and sandwiched assemblies prepared with Lucite Elvacite 4044 as binder, w/or w/o KaMin 70C addition and (2) examine the efficacy of reversed assemblies fabricated using screen printing technique.

Experiments Content: Table (AMA Only)

| | | | | | | SC | | | | | |
|---|---|---|---|---|---|---|---|---|---|---|---|
| | | | | | | Active | | | Inoculation | Sam- | |
| Model | Active | Additional | Formulation | | | conc. | Vol. | | [Log | pling | Temp, |
| No. | material | materials | number | Model description | Medium | [ppm] | [ml] | Microorganism | cfu/ml] | times | C. |
| A | SC + | Vinnol/ | Elavcite: | R, Elvacite, GTW | GTW | 10 | 500 | E. Coli | 3 | 0.5, 4 | GTW: |
| B | CG8-H | EtOAc | WE037/ | S, Elvacite, GTW | | unless | | | | hours | RT |
| C | | (see also | 1 + | R, Elvacite w/20 wt % | | specified | | | | | CTW: |
| | | model | WE038/ | KaMin 70 C., GTW | | otherwise | | | | | 4° C. |
| D | | description) | 1 + | S, Elvacite w/20 wt % | | in the | | | | | |
| | | | WE039/1 | KaMin 70 C., GTW | | model | | | | | |
| E | | | Vinnol: | Reversed PC, GTW | | desc. | | | | | |
| F | | | WE003/ | Sandwiched PC, GTW | | | | | | | |
| G | | | 140214 + | Reversed, Screen | | | | | | | |
| | | | WE004/ | printing, GTW | | | | | | | |
| H | | | 34 | R, Elvacite, CTW | CTW | | | | | | |
| I | | | Screen- | S, Elvacite, CTW | | | | | | | |
| J | | | printing: | R, Elvacite w/20 wt % | | | | | | | |
| | | | WE018/ | KaMin 70 C., CTW | | | | | | | |
| K | | | 8 + | S, Elvacite w/20 wt % | | | | | | | |
| | | | WE004/ | KaMin 70 C., CTW | | | | | | | |
| L | | | 35 | Reversed PC, CTW | | | | | | | |
| M | | | | Sandwiched PC, CTW | | | | | | | |
| N | | | | Reversed, Screen | | | | | | | |
| | | | | printing, CTW | | | | | | | |
| NCO | N/A | N/A | N/A | NC GTW | GTW | N/A | 50 | | | | |
| NCP | N/A | N/A | N/A | NC CTW | CTW | N/A | 50 | | | | |

This is a yes/no experiment, thus counting is needed only for 0, 1 dilutions. CG8-H dry percentage is held constant in all compositions (at each geometry). Elvacite stock solution: 30 wt % Elvacite 4044 in EtOAc. GTW: TSB 1:500 and CTW: Humic acid, 15±5 mg/L; CaCO₃, 100±20 mg/L; NaCl, 1500±150 mg/L, Turbidity, not applicable. Measure pH.

Observations: test bottles had antimicrobial activity against E.coli in both temperatures. The active material disintegrated from R. Elvacite w/20 wt% KaMin 70C, GTW (group C) & R, Elvacite, CTW (group J).

CIOₓ-species analytical measurement

| | | 0.5 h | | | | | 4 h | | | | |
|---|---|---|---|---|---|---|---|---|---|---|---|
| | | | | | total | | | | | total | |
| Serial | Model | Cl2 | ClO2 | ClO2− | ox′ | pH | Cl2 | ClO2 | ClO2− | ox′ | pH |
| A4 | Elvacite, | 0 | 0.384 | 3.884 | 4.268 | 4.24 | 0 | 1.4 | 4.252 | 5.652 | 4.12 |
| A5 | Reversed | 0 | 1.412 | 3.188 | 4.6 | 4.29 | 0 | 2.208 | 3.352 | 5.56 | 4.18 |
| B4 | Elvacite, | 0 | 0 | 2.908 | 2.908 | 4.3 | 0 | 3.204 | 3.984 | 7.188 | 3.93 |
| B5 | Sandwiched | 0 | 3.128 | 0 | 3.128 | 4.34 | 0 | 4.26 | 4.008 | 8.268 | 3.97 |
| C4 | Elvacite, | 0 | 0.68 | 4.708 | 5.388 | 4.15 | 0 | 0.28 | 4.532 | 4.812 | 4.1 |
| | Reversed, w/20_{wt} | | | | | | | | | | |
| | % KaMin 70 C. | | | | | | | | | | |
| D4 | Elvacite, | 0 | 1.652 | 3.136 | 4.788 | 4.07 | 0 | 1.568 | 3.464 | 5.032 | 4 |
| | sandwiched, | | | | | | | | | | |
| | w/20_{wt} % KaMin | | | | | | | | | | |
| | 70 C. | | | | | | | | | | |
| E4 | PC, reversed | 0 | 0 | 0 | 0 | 5.05 | 0 | 2.316 | 2.968 | 5.284 | 4.55 |
| F4 | PC, Sandwiched | 0 | 0 | 0 | 0 | 4.79 | 0 | 1.412 | 8.732 | 10.144 | 4.29 |
| G4 | Reversed, | 0 | 1.196 | 8.776 | 9.972 | 4.42 | 0 | 4.596 | 0 | 4.596 | 4.46 |
| G5 | screen-printed | 0 | 0 | 10.18 | 10.18 | 4.4 | 0 | 0.512 | 8.608 | 9.12 | 4.37 |

Summary and conclusions: (I) “homemade” screen-printed reversed assemblies were effective against 10³ cfu/ml of E. Coli in GTW and CTW. Screen-printed assemblies also exhibited superior CIOₓ-spccies release kinetics after 0.5 h relative to the other examined models (within this trial). Additional experiment: “mini-pilot” trial @ Ponger, (2) all specimens prepared with Elvacite were effective in GTW. The Elavicte reversed assemblies were ineffective in CTW after 0.5 h and partially effective after 4 h. KaMin 70C addition slightly improved the reversed geometry efficacy. Additional experiment: re-trial of Elvacite assemblies+organoleptic assessment+shelf-life examination; and (3) Elvacite prepared assemblies exhibited poor mechanical stability prior to the AMA trial and after their immersion in water. Elvacite formulation also demonstrated faster sedimentation profile than Vinnol-bascd formulations.

AWT075

In a non-limiting example, sample 75 [AWT075] was examined regarding the efficacies of reversed assemblies where the SC layer is prepared with Neocryl A-2092 (DSM) aqueous polymer emulsion and 31 wt % SC solution (Textone XL, OxyChem). Addition of KaMin 70° C. to the SC layer (0-20 wt%) was also examined. While all assemblies were effective in GTW, after 30 min, none was effective in CTW also after 4 h.

Experiments Goals: (1) self life time zero tested and (2) neocryl-base reversed assemblies.

TABLE
Experiments Content (AMA only)

| | | | | | | SC Active | | | | | |
|---|---|---|---|---|---|---|---|---|---|---|---|
| Model | Active | Additional | Formulation | | | conc. | Vol. | Micro- | Inoculation | Sampling | |
| No. | material | materials | number | Model description | Medium | [ppm] | [ml] | organism | [Log cfu/ml] | times | Temp, C. |
| A | SC + | Vinnol/ | WE018/9 + | R, GTW | GTW | 10 unless | 500 | E. Coli | 3 | 0.5, 4 | GTW: |
| B | CG8-H | EtOAc (see | WE04/36 | S, GTW | | specified | | | | hours | RT |
| C | | also model | | R + vinnol top-coat, | | otherwise | | | | | CTW: |
| | | description) | | GTW | | in the | | | | | 4° C. |
| D | | | | S + vinnol top-coat, | | model | | | | | |
| | | | | GTW | | desc. | | | | | |
| E | | | See AWT074 | S, Elvacite, GTW | | | | | | | |
| F | | | See AWT073 | S, w/KaMin, GTW | | | | | | | |
| G | | | WE030/2 or | R, Neocryl, GTW | | | | | | | |
| H | | | WE040/1 + | R, Neocryl w/10% | | | | | | | |
| | | | WE018/9 | Kamin, GTW | | | | | | | |
| I | | | | R, Neocryl w/20% | | | | | | | |
| | | | | Kamin, GTW | | | | | | | |
| J | | | WE018/9 + | R, CTW | CTW | | | | | | |
| K | | | WE04/36 | S, CTW | | | | | | | |
| L | | | | R + vinnol top-coat, | | | | | | | |
| | | | | CTW | | | | | | | |
| M | | | | S + vinnol top-coat, | | | | | | | |
| | | | | CTW | | | | | | | |
| N | | | See AWT074 | S, Elvacite, CTW | | | | | | | |
| O | | | See AWT073 | S, w/KaMin, CTW | | | | | | | |
| P | | | WE030/2 or | R, Neocryl, CTW | | | | | | | |
| Q | | | WE040/1 + | R, Neocryl w/10% | | | | | | | |
| | | | WE018/9 | Kamin, CTW | | | | | | | |
| R | | | | R, Neocryl w/20% | | | | | | | |
| | | | | Kamin, CTW | | | | | | | |
| NCS | N/A | N/A | N/A | NC GTW | GTW | N/A | 50 | | | | |
| NCT | N/A | N/A | N/A | NC CTW | CTW | N/A | 50 | | | | |

This is a yes/no experiment, thus counting is needed only for 0, 1 dilutions. All samples are to be sampled w/neutratizer (1:1). CTW: Humic acid, 15±5 mg/L; CaCO₃, 100±20 mg/L; NaCl, 1500±50 mg/L, Turbidity, not applicable at the moment. Measure pH. Shake thoroughly after filling and one again after 15′.

Observations: testbottles had antimicrobial activity against E. coli in both temperatures. There was a color change to green in R, Neocryl, GTW (group H) and R, Neocryl w/10% Kamin, GTW (group I).

ClOₓ-species analytical measurement

| | | 0.5 h | | | | | 4 h | | | | |
|---|---|---|---|---|---|---|---|---|---|---|---|
| | | | | | total | | | | | total | |
| Serial | Model | Cl2 | ClO2 | ClO2− | ox′ | pH | Cl2 | ClO2 | ClO2− | ox′ | pH |
| A | R | 0.044 | 0 | 2.764 | 2.764 | 4.7 | 0 | 1.468 | 2.3 | 3.768 | 4.31 |
| B | S | 0 | 3.448 | 0 | 3.448 | 4.38 | 0.296 | 0 | 4.88 | 4.88 | 4.24 |
| C | R + vinnol | 0 | 4.876 | 0 | 4.876 | 4.72 | 0 | 0.6 | 2.74 | 3.34 | 4.52 |
| | top-coat | | | | | | | | | | |
| D | S + vinnol | 0 | 0 | 0 | 0 | 4.8 | 0 | 5.616 | 0 | 5.616 | 4.11 |
| | top-coat | | | | | | | | | | |
| E | S, Elvacite | 0 | 1.108 | 2.412 | 3.52 | 4.27 | 0 | 1.172 | 2.936 | 4.108 | 4.05 |
| F | S, w/KaMin | 0 | 1.896 | 2.532 | 4.428 | 4.25 | 0 | 2.008 | 2.1 | 4.108 | 4.28 |
| G | R, Neocryl | 0 | 1.412 | 3.384 | 4.796 | 4.12 | 0 | 1.744 | 3.512 | 5.256 | 3.96 |
| H | R, Neocryl | 0 | 2.34 | 0 | 2.34 | 4.72 | 0 | 0.748 | 2.572 | 3.32 | 3.9 |
| | w/10% | | | | | | | | | | |
| | Kamin | | | | | | | | | | |
| I | R, Neocryl | 0 | 4.612 | 0 | 4.612 | 4.45 | 0 | 4.044 | 0 | 4.044 | 4.08 |
| | w/20% | | | | | | | | | | |
| | Kamin | | | | | | | | | | |

Summary and Conclusions

All examined assemblies were effective vs. E. Coli in GTW after 30 min, excluding the reversed assembly with Neocryl A-2092 based SC formulation with 20 wt % of KaMin 70C.

Neocryl-based assemblies did exhibit mild green coloration of the medium, probably following pigments' leaching.

The Neocryl based assemblies were not effective in CTW even after 4 h (an unclear insignificant <1-log inhibition can be observed). this may be due to insufficient acidification (i.e., CDO activation) or available precursor content following the formation of different morphology. Additional experiments: further explore Neocryl A-2092 (as well as different binders), Alternate PVC, fillers, thermal treatment, etc.

Reversed and sandwiched assemblies were deposited with additional top-coat layer of clear Vinnol stock solution (20 wt % in EtOAc, wet thickness=24 m) to explore whether it may serve as humidity degradation barrier. The Vinnol top-coat did not interfere with efficacy in GTW (total eradication after 30 min) but did impede eradication in CTW. While the sandwiched assemblies with the Vinnol top-coat where only fully effective after 4 h (or at least not after 30 min), the reversed assemblies with the Vinnol top-coat were not effective (slight and unclear inhibition). This might indicate that the Vinnol top-coat does inhibit some of the SC release, even in exposure for liquid water. Hach results support the AMA behavior. Next steps: further explore this issue pending successful shelf-life trial.

Standard indicator-integrated reversed assemblies' efficacy was validated. This production batch may be used for anti-protozoa trials.

AWT076

In a non-limiting example, sample 76 [AWT076] was examined regarding the efficacies of reversed and sandwiched assemblies with 10 or 7.5 ppm of SC. Utilization as Elvacite as the binder (see sample 73) and KaMin 70° C. in the top layer were also examined. All assemblies were found to be effective in CTW down to 7.5 ppm of SC and no clear advantage was found to either of them (efficacy-wise).

Experiments goals: (1) examine efficacy of different contents of CDO precursor (7.5 and 10 ppm of SC) in different geometries and systems in general and challenge test water (GTW and CTW, respectively): (a) Reversed, (b) Sandwiched, (c) Sandwiched w/10% KaMin 70C in top layer, and (d) Sandwiched, Elvacite-based.

Experiments Content (AMA only)

| | | | Formu- | | | SC Active | | | | Sam- | |
|---|---|---|---|---|---|---|---|---|---|---|---|
| Model | Active | Additional | lation | | Me- | conc. | Vol. | Micro- | Inoculation | pling | Temp, |
| No. | material | materials | number | Model description | dium | [ppm] | [ml] | organism | [Log cfu/ml] | times | C. |
| A | SC + | Vinnol/ | See | R, 10 ppm, GTW | GTW | 10/7.5 | 500 | E. Coll | 3 | 0.5, 4 hours | GTW: |
| B | CG8-H | EtOAc | AWT075 | S, 10 ppm, GTW | | unless | | | | | RT |
| C | | (see also | | S, Elvacite, 10 ppm, GTW | | specified | | | | | CTW: |
| D | | model | | S, w/Kamin, 10 ppm, GTW | | otherwise | | | | | 4° C. |
| E | | descrip- | | R, 7.5 ppm, GTW | | in the | | | | | |
| F | | tion) | | S, 7.5 ppm, GTW | | model | | | | | |
| G | | | | S, Elvacite, 7.5 ppm, GTW | | desc. | | | | | |
| H | | | | S, w/Kamin, 7.5 ppm, GTW | | | | | | | |
| J | | | | R, 10 ppm, CTW | CTW | | | | | | |
| K | | | | S, 10 ppm, CTW | | | | | | | |
| L | | | | S, Elvacite, 10 ppm, CTW | | | | | | | |
| M | | | | S, w/Kamin, 10 ppm, CTW | | | | | | | |
| N | | | | R, 7.5 ppm, CTW | | | | | | | |
| O | | | | S, 7.5 ppm, CTW | | | | | | | |
| P | | | | S, Elvacite, 7.5 ppm, CTW | | | | | | | |
| Q | | | | S, w/Kamin, 7.5 ppm, CTW | | | | | | | |
| NCS | N/A | N/A | N/A | NC GTW | GTW | N/A | 50 | | | | |
| NCT | N/A | N/A | N/A | NC CTW | CTW | N/A | 50 | | | | |

This is a yes/no experiment, thus counting is needed only for 0, 1 dilutions. All samples are to be sampled w/neutralizer (1:1). CTW: Humic acid. 15±5 mg/L; CaCO₃, 100±20 mg/L; NaCl, 1500±150 mg/L. Turbidity, not applicable.Measure pH. Shake thoroughly after filling and one again after 15′.

Microbiology Results: Table illustrating E. coli viable counts in test tubes

effective where the SC content (or alternatively the total filler weight content when KaMin 70° C. was added) was higher than 40 wt %.

AWT077

Experiments Goals: Examine efficacy of Elvacite based assemblies with solid or aqueous SC source.

TABLE
Experiments Content (AMA only)

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | ElVacite/Vinnol/ | WE018/9 + | Reversed | GTW | 10 unless | 500 | E. Coli | 3 | 0.5, 4 | GTW: |
| | CG8-H | EtOAc or | WE004/36 | Vinnol, GTW | | specified | | | | hours | RT |
| B | | (see also | WE037/2 + | Reversed, | | otherwise | | | | | CTW: |
| | | model | WE039/2 | Elvacite, 20 wt % | | in the | | | | | 4° C. |
| | | description) | | SC(s), GTW | | model | | | | | |
| C | | | WE037/2 + | Reversed, | | desc. | | | | | |
| | | | WE047/2 | Elvacite, 40 wt % | | | | | | | |
| | | | | SC(s), GTW | | | | | | | |
| D | | | WE037/2 + | Reversed, | | | | | | | |
| | | | WE048/2 | Elvacite, 20 wt % | | | | | | | |
| | | | | SC(aq), GTW | | | | | | | |
| E | | | WE037/2 + | Reversed, | | | | | | | |
| | | | WE049/2 | Elvacite, 40 wt % | | | | | | | |
| | | | | SC(aq), GTW | | | | | | | |
| F | | | WE037/2 + | Reversed, | | | | | | | |
| | | | WE050/2 | Elvacite, 20 wt % | | | | | | | |
| | | | | SC(aq) + 30% | | | | | | | |
| | | | | KaMin 70 C., GTW | | | | | | | |
| G | | | WE037/2 + | Sandwiched, | | | | | | | |
| | | | WE050/2 | Elvacite, 20 wt % | | | | | | | |
| | | | | SC(aq) + 30% | | | | | | | |
| | | | | KaMin 70 C., GTW | | | | | | | |
| H | | | WE018/9 + | Reversed, | CTW | | | | | | |
| | | | WE004/36 | Vinnol, CTW | | | | | | | |
| I | | | WE037/2 + | Reversed, | | | | | | | |
| | | | WE039/2 | Elvacite, 20 wt % | | | | | | | |
| | | | | SC(s), CTW | | | | | | | |
| J | | | WE037/2 + | Reversed, | | | | | | | |
| | | | WE047/2 | Elvacite, 40 wt % | | | | | | | |
| | | | | SC(s), CTW | | | | | | | |
| K | | | WE037/2 + | Reversed, | | | | | | | |
| | | | WE048/2 | Elvacite, 20 wt % | | | | | | | |
| | | | | SC(aq), CTW | | | | | | | |
| L | | | WE037/2 + | Reversed, | | | | | | | |
| | | | WE049/2 | Elvacite, 40 wt % | | | | | | | |
| | | | | SC(aq), CTW | | | | | | | |
| M | | | WE037/2 + | Reversed, | | | | | | | |
| | | | WE050/2 | Elvacite, 20 wt % | | | | | | | |
| | | | | SC(aq) + 30% | | | | | | | |
| | | | | KaMin 70 C., CTW | | | | | | | |
| N | | | WE037/2 + | Sandwiched, | | | | | | | |
| | | | WE050/2 | Elvacite, 20 wt % | | | | | | | |
| | | | | SC(aq) + 30% | | | | | | | |
| | | | | KaMin 70 C., CTW | | | | | | | |
| NCO | N/A | N/A | N/A | NC GTW | GTW | N/A | 50 | | | | |
| NCP | N/A | N/A | N/A | NC CTW | CTW | N/A | 50 | | | | |

This is a yes/no experiment, thus counting is needed only for 0, 1 dilutions. All samples are to be sampled w/neutralizer (1:1). CTW: Humic acid, 15±5 mg/L; CaCO₃, 100±20 mg/L; NaCl, 1500±150 mg/L, Turbidity, not applicable at the moment. Measure pH. Shake thoroughly after filling and one again after 15′.

In one embodiment of the present invention, bottles had antimicrobial activity after 0.5 hours against E. coli in both tempertures.

CIOₓ—specics analysis;

| | | 0.5 h | | | | | 4 h | | | | |
|---|---|---|---|---|---|---|---|---|---|---|---|
| | | | | | total | | | | | total | |
| Sample | Model | Cl2 | ClO2 | ClO2 | ox′ | pH | Cl2 | ClO2 | ClO2 | ox′ | pH |
| A | R, Vinnol | 0 | 2.688 | 0 | 2.688 | 4.43 | 0 | 1.356 | 2.104 | 3.46 | 4.35 |
| B | R, Elvacite, 20 wt % | 2.228 | 0 | 4.236 | 4.236 | 4.94 | 0 | 1.412 | 4.84 | 6.252 | 4.15 |
| | SC(s) | | | | | | | | | | |
| C | R, Elvacite, 40 wt % | 0 | 1.864 | 5.212 | 7.076 | 4.75 | 0 | 0.176 | 6.392 | 6.568 | 4.63 |
| | SC(s) | | | | | | | | | | |
| D | R, Elvacite, 20 wt % | 1.468 | 0 | 2.796 | 2.796 | 5.01 | 0.068 | 0 | 2.64 | 2.64 | 4.3 |
| | SC(aq) | | | | | | | | | | |
| E | R, Elvacite, 20 wt % | | | | 0 | | 0 | 1.472 | 2.732 | 4.204 | 4.27 |
| | SC(aq) | | | | | | | | | | |
| F | R, Elvacite, 20 wt % | 0 | 0 | 0 | 0 | 4.84 | 0 | 0.864 | 2.564 | 3.428 | 4.28 |
| | SC(aq) + 30 wt % | | | | | | | | | | |
| | KaMin 70 C. | | | | | | | | | | |
| G | S, Elvacite, 20 wt % | 0 | 0 | 0 | 0 | 5.03 | 0 | 0 | 0 | 0 | 4.36 |
| | SC(aq) + 30 wt % | | | | | | | | | | |
| | KaMin 70 C. | | | | | | | | | | |

All the examined models were effective vs. E. Coli (10³ cfu/ml) in CTW, including the specimens constructed with SC solution based formulations.

In CTW, Elvacite samples with 40 wt% solid SC and with 20 wt% aqueous SC (in the dry layer of relevant SC formulation) were ineffective, while the rest of the models were fully effective and exhibited total eradication alter 0.5 h.

Apparently, when one uses aqueous SC in organic solutions of polymers, the SC weight (or volume) percentage should rise above a certain threshold (either by elevating the SC concentration itself or by adding a different filler, e.g., KaMin 70C) to obtain efficacy. This may be related to film formation or morphological attributes of the resulting film.

Additional experiments: re-examine successful models (current and new batch of formulations and assemblies), explore same methodology with Vinnol (20 wt % models were unsuccessful in AWT061).

AWT078 and AWT080

In a non-limiting example, samples 80 and 81 [AWT078 and AWT080] were examined regarding the efficacies of reversed and sandwiched assemblies prepared with Vinnacoat LL8100 as the binder. 20 wt % dispersion of Vinnacoat was prepared using methyl ethyl ketone (MEK). Solid and aqueous SC were used as the CDO precursor sources. Samples prepared with solid SC were not effective in CTW (and only weakly effective in GTW after 30 min). Samples prepared with aqueous SC were effective in CTW where the total weight content of the fillers in the SC layer (either only SC or SC and KaMin 70C) was higher than 40 wt %.

AWT078

Experiments Goals: (1) Examine efficacy of Vinnacoat LL 8100 formulations. (2) Efficacy of Aquamira and Aquatabs in WHO protocol. (3) Preliminary inspection of the influence of application of a thin Vaseline layer on top of an assembly.

Experiments Content (AMA Only)

| | | | | | | SC Active | | | | | |
|---|---|---|---|---|---|---|---|---|---|---|---|
| Model | Active | Additional | Formulation | Model | | conc. | Vol. | | Inoculation | Sampling | Temp, |
| No. | material | materials | number | description | Medium | [ppm] | [ml] | Microorganism | [Log cfu/ml] | times | C. |
| A | SC + | Vinnol/ | WE018/9 + | S, Vinnol (PC), | GTW | 10unless | 500 | E. Coli | 3 | 0.5, 4 hours | GTW: |
| | CG8-H | EtOAc (see | WE004/36 | GTW | | specified | | | | | RT |
| B | (in | also model | WE052/1 + | R, 20 wt % | | otherwise | | | | | CTW: |
| | models) | description) | WE051/1 | SC(S), GTW | | in the | | | | | 4° C. |
| C | | | WE052/1 + | R, 20 wt % | | model | | | | | |
| | | | WE053/1 | SC(aq), GTW | | desc. | | | | | |
| D | | | WE052/1 + | R, 40 wt % | | | | | | | |
| | | | WE054/1 | SC(aq), GTW | | | | | | | |
| E | | | WE052/1 + | R, 20 wt % | | | | | | | |
| | | | WE055/1 | SC(q) + 30 wt | | | | | | | |
| | | | | % KaMin | | | | | | | |
| | | | | 70 C., GTW | | | | | | | |
| F | | | WE052/1 + | S, 20 wt % | | | | | | | |
| | | | WE055/1 | SC(aq) + 30 wt | | | | | | | |
| | | | | % KaMin | | | | | | | |
| | | | | 70 C., GTW | | | | | | | |
| G | | | N/A | Aquamira, | | | | | | | |
| | | | | GTW | | | | | | | |
| H | | | N/A | Aquatabs, | | | | | | | |
| | | | | GTW | | | | | | | |
| I | | | WE018/9 + | Vaseline top- | | | | | | | |
| | | | WE004/36 | coat, CTW | | | | | | | |
| J | | | WE018/9 + | R, Vinnol (PC), | CTW | | | | | | |
| | | | WE004/36 | CTW | | | | | | | |
| K | | | WE052/1 + | R, 20 wt % | | | | | | | |
| | | | WE051/1 | SC(S), CTW | | | | | | | |
| L | | | WE052/1 + | R, 20 wt % | | | | | | | |
| | | | WE053/1 | SC(aq), CTW | | | | | | | |
| M | | | WE052/1 + | R, 40 wt % | | | | | | | |
| | | | WE054/1 | SC(aq), CTW | | | | | | | |
| N | | | WE052/1 + | R, 20 wt % | | | | | | | |
| | | | WE055/1 | SC(aq) + 30 wt | | | | | | | |
| | | | | % KaMin | | | | | | | |
| | | | | 70 C., CTW | | | | | | | |
| O | | | WE052/1 + | S, 20 wt % | | | | | | | |
| | | | WE055/1 | SC(aq) + 30 wt | | | | | | | |
| | | | | wt % KaMin | | | | | | | |
| | | | | 70 C., CTW | | | | | | | |
| P | | | N/A | Aquamira, | | | | | | | |
| | | | | CTW | | | | | | | |
| Q | | | N/A | Aquatabs, | | | | | | | |
| | | | | CTW | | | | | | | |
| R | | | WE018/9 + | Vaseline top- | | | | | | | |
| | | | W0004/36 | coat, CTW | | | | | | | |
| NCS | N/A | N/A | N/A | NC GTW | GTW | N/A | 50 | | | | |
| NCT | N/A | N/A | N/A | NC CTW | CTW | N/A | 50 | | | | |

This is a yes/no experiment, thus counting is needed only for 0,1 dilutions. All samples are to be sampled w/neutralizer (1:1). CTW: Humic acid, 15±5 mg/L; CaCO₃, 100±20 mg/L; NaCl, 1500±150 mg/L, Turbidity, not applicable at the moment. Measure pH. Shake thoroughly after filling and one again after 15′.

Results: Table Showing E.coli viable counts

TABLE
ClOx-species determination:

| | | 0.5 h | | | | | 4 h | | | | |
|---|---|---|---|---|---|---|---|---|---|---|---|
| | | | | | total | | | | | total | |
| Sample | Model | Cl2 | ClO2 | ClO2− | ox′ | pH | Cl2 | ClO2 | ClO2− | ox′ | pH |
| A | S, Vinnol | 0 | 4.264 | 0 | 4.26 | 4.6 | 0 | 3.528 | 0 | 3.53 | 4.02 |
| B | R, Vinnacoat, 20 wt | 2.28 | 2.012 | 7.42 | 9.43 | 5.55 | 0 | 2.256 | 0 | 2.26 | 4.49 |
| | % SC(s) | | | | | | | | | | |
| C | R, Vinnacoat, 20 wt | 0 | 1.632 | 7.112 | 8.74 | 6.43 | 0 | 2.256 | 0 | 2.26 | 4.52 |
| | % SC(aq) | | | | | | | | | | |
| D | R, Vinnacoat, 40 wt | 0 | 6.332 | 0 | 6.33 | 5.39 | 0 | 2.64 | 0 | 2.64 | 4.6 |
| | % SC(aq) | | | | | | | | | | |
| E | R, Vinnacoat, 20 wt | 0 | 0 | 0 | 0 | 6.36 | 0 | 2.596 | 0 | 2.6 | 4.28 |
| | % SC(aq) + | | | | | | | | | | |
| | 30 wt % KaMin | | | | | | | | | | |
| | 70 C. | | | | | | | | | | |
| F | S. Vinnacoat, 20 wt | 0 | 2.264 | 0 | 2.26 | 5.98 | 0 | 2.476 | 0 | 2.48 | 4.29 |
| | % SC(aq) + | | | | | | | | | | |
| | 30 wt % KaMin | | | | | | | | | | |
| | 70 C | | | | | | | | | | |
| I1 | S, Vinnol | 0 | 6.596 | 0 | 6.6 | 5.45 | 0 | 2.564 | 0 | 2.56 | 4.52 |
| I2 | w/Vaseline top- | 0 | 0 | 0 | 0 | 5.14 | 0 | 2.1 | 0 | 2.1 | 4.93 |
| I3 | coat | 0 | 0 | 0 | 0 | 6.18 | 0 | 0 | 0 | 0 | 4.66 |

Vinnacoat Case Study:

Vinnacoat LL 8100 is a binder resin composed of styrene-olefin (ca. 50 wt % ea. With ˜1 wt % carboxylic acid) copolymers mixture. It is dispersed (20 wt %) in Methyl Ethyl Ketone (MEK) to form a stable white dispersion. the obtained films are highly flexible (compared to Vinnol H30/48M and Elvacite 4044).

As in Elvacite 4044 (see AWT077), efficacy was only obtained with aqueous SC when the solid content of the fillers, either SC alone or together with KaMin 70C) was above 40 wt %. This phenomenon may be related to the obtained morphology of the SC layer.

Assemblies prepared with solid SC were ineffective, probably due to inhomogeneous dispersion of the powder.

Vaseline Case Study:

applying a thin Vaseline layer on top of a sandwiched assembly was carried out to examine possible method for blocking humidity and extending shelf life.

The Vaseline-applied samples were only effective after 4 h, and not after 30 min, clearly indicating on slower release profile.

As can be seen in FIG. 24, while the bare assembly is all blue, indicating on complete consumption of the CDO in the assembly, the vaselined assembly still exhibit dark green area in its center. The latter indicates that not all of the SC within the assembly was leached and or activated. It also indicates that the Vaseline blocked perpendicular migration of SC/CDO through the face of the assembly, and that only parallel lateral diffusion and migration were possible through the assembly sliced edges (hence the core-shell look of the vaselined assembly).

Aquamira was only effective in GTW, Cl₂ tabs were effective (2.5 ppm) also in CTW.

AWT079

In a non-limiting example, sample 82 [AWT079] was examined regarding ihe efficacies of reversed and sandwiched assemblies with Vinnol H30/48M and aqueous SC. None of the prepared assemblies were effective in GTW or CTW regardless of the filler weight content in the SC layer (see, e.g., samples 77-81).

Experiments Goals: efficacy of assemblies with aqueous SC formulations based on Vinnol and Elvacite.

Experiments Content (AMA only):

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | VInnol/ | WE018/9 + | R, Vinnol, 40 wt % | GTW | 10unless | 500 | E. Coli | 3 | 0.5, | GTW: |
| | CG8-H | EtOAc | WE004/36 | SC(s), GTW | | specified | | | | 4 hours | RT |
| B | (in | (see also | WE018/9 + | R, Vinnol, 20 wt % | | otherwise | | | | | CTW: |
| | models) | model | WE020/2 | SC(aq), GTW | | in the | | | | | 4° C. |
| C | | description) | WE018/9 + | R, Vinnol, 40 wt % | | model | | | | | |
| | | | WE056/1 | SC(aq), GTW | | desc. | | | | | |
| D | | | WE018/9 + | R, Vinnol, 20 wt % | | | | | | | |
| | | | WE057/1 | SC(aq) + 30 wt % | | | | | | | |
| | | | | KaMin 70 C., GTW | | | | | | | |
| E | | | See | R, Elvacite, 20 wt % | | | | | | | |
| | | | AWT077 | SC(aq), GTW | | | | | | | |
| F | | | | R, Elvacite, 40 wt % | | | | | | | |
| | | | | SC(aq), GTW | | | | | | | |
| G | | | | R, Elvacite, 20 wt % | | | | | | | |
| | | | | SC(aq) + 30 wt % | | | | | | | |
| | | | | KaMin 70 C., GTW | | | | | | | |
| H | | | WE018/9 + | R, Vinnol, 40 wt % | CTW | | | | | | |
| | | | WE004/36 | SC(s), CTW | | | | | | | |
| I | | | WE018/9 + | R, Vinnol, 20 wt % | | | | | | | |
| | | | WE020/2 | SC(aq), CTW | | | | | | | |
| J | | | WE018/9 + | R, Vinnol, 40 wt % | | | | | | | |
| | | | WE056/1 | SC(aq), CTW | | | | | | | |
| K | | | WE018/9 + | R, Vinnol, 20 wt % | | | | | | | |
| | | | WE057/1 | SC(aq) + 30 wt % | | | | | | | |
| | | | | KaMin 70 C., CTW | | | | | | | |
| L | | | See | R, Elvacite, 20 wt % | | | | | | | |
| | | | AWT077 | SC(aq), CTW | | | | | | | |
| M | | | | R, Elvacite, 40 wt % | | | | | | | |
| | | | | SC(aq), CTW | | | | | | | |
| N | | | | R, Elvacite, 20 wt % | | | | | | | |
| | | | | SC(aq) + 30 wt % | | | | | | | |
| | | | | KaMin 70 C., CTW | | | | | | | |
| NCO | N/A | N/A | N/A | NC GTW | GTW | N/A | 50 | | | | |
| NCP | N/A | N/A | N/A | NC CTW | CTW | N/A | 50 | | | | |

This is a yes/no experiment, thus counting is needed only for 0, 1 dilutions. GTW: TSB 1:500. CTW: Humic acid, 15±5 mg/L; CaCO₃, 100±20 mg/L; NaCl, 1500±150 mg/L. Turbidity, not applicable. Measure pH. Shake thoroughly after filling and one again after 15′.

Microbiology results: E. coli viable counk in test tubes:

and 20 wt % SC+30 wt % KaMin 70C. Assemblies prepared with formulation of 20 wt % SC were ineffective only in GTW and not in CTW. Thus, it is indicated that a certain threshold of fillers weight or volume percentage exists, allowing rapid release and efficacy on short times.

Assemblies prepared with Vinnol formulations with aqueous SC were not effective in both media at any fillers weight percentage. Apparently, the Vinnol system is not suited to aqueous SC utilization and formation of effective morphologies and films.

Additional experiments: validation of Elvacite results, organoleptic trial, additional binders, lowering Elvacite formulations viscosity.

AWT080

Experiments goals: 1) reexamine efficacy of Vinnacoat LL 8100 assemblies and 2) reexamine efficacy of Elvacite 4044 assemblies.

Experiments Content (AMA Only):

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/ | WE052/1 + | R, Vinnacoat, | GTW | 10unless | 500 | E. Coli | 3 | 0.5, | GTW: |
| | CG8-H | EtOAc (see | WE053/1 | 20 wt % | | specified | | | | 4 hours | RT |
| | (in models) | also model | | SC(aq), GTW | | otherwise | | | | | CTW: |
| B | | description) | WE052/1 + | R, Vinnacoat, | | in the | | | | | 4° C. |
| | | | WE054/1 | 40 wt % | | model | | | | | |
| | | | | SC(aq), GTW | | desc. | | | | | |
| C | | | WE052/1 + | R, Vinnacoat, | | | | | | | |
| | | | WE055/1 | 20 wt % | | | | | | | |
| | | | | SC(aq) + 30 | | | | | | | |
| | | | | wt % KaMin | | | | | | | |
| | | | | 70 C., GTW | | | | | | | |
| D | | | WE058/1 + | R, Elvacite, | | | | | | | |
| | | | WE048/2 | 20 wt % | | | | | | | |
| | | | | SC(aq), GTW | | | | | | | |
| E | | | WE058/1 + | R, Elvacite, | | | | | | | |
| | | | WE049/2 | 40 wt % | | | | | | | |
| | | | | SC(aq), GTW | | | | | | | |
| F | | | WE058/1 + | R, Elvacite, | | | | | | | |
| | | | WE050/2 | 20 wt % | | | | | | | |
| | | | | SC(aq) + 30 | | | | | | | |
| | | | | wt % KaMin | | | | | | | |
| | | | | 70 C., GTW | | | | | | | |
| G | | | WE018/8 + | R, Vinnol, | | | | | | | |
| | | | WE004/36 | GTW | | | | | | | |
| H | | | WE052/1 + | R, Vinnacoat, | CTW | | | | | | |
| | | | WE053/1 | 20 wt % | | | | | | | |
| | | | | SC(aq), CTW | | | | | | | |
| I | | | WE052/1 + | R, Vinnacoat, | | | | | | | |
| | | | WE054/1 | 40 wt % | | | | | | | |
| | | | | SC(aq), CTW | | | | | | | |
| J | | | WE052/1 + | R, Vinnacoat, | | | | | | | |
| | | | WE055/1 | 20 wt % | | | | | | | |
| | | | | SC(aq) + 30 | | | | | | | |
| | | | | wt % KaMin | | | | | | | |
| | | | | 70 C., CTW | | | | | | | |
| K | | | WE058/1 + | R, Elvacite, | | | | | | | |
| | | | WE048/2 | 20 wt % | | | | | | | |
| | | | | SC(aq), CTW | | | | | | | |
| L | | | WE058/1 + | R, Elvacite, | | | | | | | |
| | | | WE049/2 | 40 wt % | | | | | | | |
| | | | | SC(aq), CTW | | | | | | | |
| M | | | WE058/1 + | R, Elvacite, | | | | | | | |
| | | | WE050/2 | 20 wt % | | | | | | | |
| | | | | SC(aq) + 30 | | | | | | | |
| | | | | wt % KaMin | | | | | | | |
| | | | | 70 C., CTW | | | | | | | |
| N | | | WE018/8 + | R, Vinnol, | | | | | | | |
| | | | WE004/36 | CTW | | | | | | | |
| NCO | N/A | N/A | N/A | NC GTW | GTW | N/A | 50 | | | | |
| NCP | N/A | N/A | N/A | NC CTW | CTW | N/A | 50 | | | | |

This is a yes/no experiment, thus counting is needed only for 0,1 dilutions. All GTW: TSB, 1:500; CTW: Humic acid, 15±≡mg/L; CaCO₃, 100±20 mg/L; NaCl, 1500±150 mg/L, Turbidity, not applicable. Measure pH. Shake thoroughly upon filling and one again after 15′.

Microbiology Results: E.coli viable counts in test tubes:

Microbiology comments: in some embodiments of the present invention, bottles except R. Vinnacoat, 20 wt% SC(aq), CTW & R. Elvacite, 20 wt% SC(aq), CTW had antimicrobial activity after 0.5 hour. There was a color change in group D-R, Elvacite, 20 wt% SC(aq), GTW.

Hach results:

| | | 0.5 h | | | | | 4 h | | | | |
|---|---|---|---|---|---|---|---|---|---|---|---|
| Sample | Model | Cl2 | ClO2 | ClO2 | total ox | pH | Cl2 | ClO2 | ClO2 | total ox′ | pH |
| A | R, Vinnacoat, | 0 | 0 | 0 | 0 | 6.32 | 0 | 2.128 | 0 | 2.128 | 4.62 |
| | 20 wt % | | | | | | | | | | |
| | SC(aq) | | | | | | | | | | |
| B | R, Vinnacoat, | 0 | 0 | 0 | 0 | 5.54 | 0 | 2.228 | 0 | 2.228 | 4.71 |
| | 40 wt % | | | | | | | | | | |
| | SC(aq) | | | | | | | | | | |
| C | R, Vinnacoat, | 0 | 0 | 0 | 0 | 4.73 | 0 | 3.148 | 0 | 3.148 | 4.34 |
| | 20 wt % | | | | | | | | | | |
| | SC(aq) + 30 | | | | | | | | | | |
| | wt % KaMin | | | | | | | | | | |
| | 70 C. | | | | | | | | | | |
| D | R, Elvacite, 20 | 0 | 0 | 0 | 0 | 4.54 | 0 | 1.868 | 3.516 | 5.384 | 4.24 |
| | wt % SC(aq) | | | | | | | | | | |
| E | R, Elvacite, 40 | 0 | 0 | 0 | 0 | 4.79 | 0 | 0.792 | 4.152 | 4.944 | 4.32 |
| | wt % SC(aq) | | | | | | | | | | |
| F | R, Elvacite, 20 | 0 | 0 | 0 | 0 | 4.72 | 0 | 1.156 | 3.404 | 4.56 | 4.17 |
| | wt % SC(aq) + | | | | | | | | | | |
| | 30 wt % | | | | | | | | | | |
| | KaMin 70 C. | | | | | | | | | | |
| G | R, Vinnol, 40 | 0 | 0 | 0 | 0 | 4.69 | 0 | 1.176 | 2.704 | 3.88 | 4.36 |
| | wt % SC(s) | | | | | | | | | | |

Discussion: Both Elvacite- and Vinnacoat-based assemblies were effective vs. 10³ cfu/ml of E. Coli in GTW and CTW when the total filler dry content was higher than 40 wt %. It was demonstrated either by applying a layer of 40 wt % of SC or of 20 wt % of SC and 30 wt % of KaMin 70C. Samples with 20 wt % of SC were ineffective.

The Elvacite samples were also integrated with indicator pigments and were found effective.

Additional experiments: 1. Reduce Elvacite IX formulation viscosity. 2. Replace Vinnacoat stock solution dispersing agent (currently MEK, which is hard to fully get rid of). 3. Additional binders.

AWT078 (t₀), AWT81-82 (t_{2w})

In a non-limiting example, samples 83-85 [AWT078 (t₀), 81-82 (t_{2w})] was examined regarding the efficacies of reversed and sandwiched assemblies after aging in two environments, 30° C. and 70 rh %, and 40° C. and 50 rh %. All standard reversed and sandwiched assemblies prepared with either Vinnol or Elvacite were effective after 2 w under both storage conditions. Application of additional Vinnol layer (w/o additional fillers) on top of the assembly held negative impact on the assembly efficacy (also on t₀).

AWT081

Experimental Goals: Shelf life time: time=2 weeks.

Experiments Content (AMA Only):

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | VInnol/ | WE018/9 + | R, GTW | GTW | 10 unless | 500 | E. Coli | 3 | 0.5, | GTW: |
| B | CG8-H | EtOAc (see | WE04/36 | S, GTW | | specified | | | | 4 hours | RT |
| C | | also model | | R + vinnol top- | | otherwise | | | | | CTW: |
| | | description) | | coat, GTW | | in the | | | | | 4° C. |
| D | | | | S + vinnol top- | | model | | | | | |
| | | | | coat, GTW | | desc. | | | | | |
| E | | | See AWT074 | S, Elvacite, GTW | | | | | | | |
| F | | | See AWT073 | S, w/KaMin, GTW | | | | | | | |
| G | | | WE018/9 + | R, Fresh, GTW | | | | | | | |
| H | | | WE04/36 | R, | | | | | | | |
| | | | | w/Vaseline/BuOAc | | | | | | | |
| | | | | coat, GTW | | | | | | | |
| I | | | WE018/9 + | R, CTW | CTW | | | | | | |
| J | | | WE04/36 | S, CTW | | | | | | | |
| K | | | | R + vinnol top- | | | | | | | |
| | | | | coat, CTW | | | | | | | |
| L | | | | S + vinnol top- | | | | | | | |
| | | | | coat, CTW | | | | | | | |
| M | | | See AWT074 | S, Elvacite, CTW | | | | | | | |
| N | | | See AWT073 | S, w/KaMin, CTW | | | | | | | |
| O | | | WE018/9 + | R, Fresh, CTW | | | | | | | |
| P | | | WE04/36 | R, | | | | | | | |
| | | | | w/Vaseline/BuOAc | | | | | | | |
| | | | | coat, CTW | | | | | | | |
| NCQ | N/A | N/A | N/A | NC GTW | GTW | N/A | 50 | | | | |
| NCr | N/A | N/A | N/A | NC CTW | CTW | N/A | 50 | | | | |

This is a yes/no experiment, thus counting is needed only for 0,1 dilutions. CTW: Humic acid, 15±5 mg/L; CaCO₃, 100±20 mg/L; NaCl, 1500±150 mg/L, Turbidity, not applicable. Measure pH. Shake thoroughly after filling and one again after 15′.

In some embodiments, bottles had antimicrobial activity after 0.5 hours.

Hach results:

| | | 0.5 h | | | | | 4 h | | | | |
|---|---|---|---|---|---|---|---|---|---|---|---|
| Sample | Model | Cl2 | ClO2 | ClO2 | total ox | pH | Cl2 | ClO2 | ClO2 | total ox′ | pH |
| A | R | 0 | 2.756 | 0 | 2.756 | 4.81 | 0 | 1.252 | 2.364 | 3.616 | 4.5 |
| B | S | 0 | 2.192 | 2.18 | 4.372 | 4.43 | 0 | 3.024 | 0 | 3.024 | 4.39 |
| C | R + vinnol top-coat | 0 | 0 | 0 | 0 | 4.96 | 0 | 2.712 | 0 | 2.712 | 4.56 |
| D | S + vinnol top-coat | 0 | 2.6 | 0 | 2.6 | 4.56 | 0 | 2.876 | 0 | 2.876 | 4.34 |
| E | S, Elvacite | 0 | 2.104 | 0 | 2.104 | 4.48 | 0 | 3.04 | 0 | 3.04 | 4.19 |
| F | S, w/KaMin | 0 | 2.316 | 0 | 2.316 | 4.58 | 0 | 3.172 | 0 | 3.172 | 4.37 |
| G | R, Fresh | 0 | 3.636 | 2.812 | 6.448 | 4.81 | 0 | 0.292 | 4.788 | 5.08 | 4.71 |
| H | R, | 0 | 2.656 | 0 | 2.656 | 5.2 | 0 | 0.468 | 3.016 | 3.484 | 4.57 |
| | w/Vaseline/BuOAc | | | | | | | | | | |
| | coat | | | | | | | | | | |

Discussion: All examined models were effective after 30 min in GTW and CTW, excluding the reversed assembly with the Vinnol top-coat which was not effective after 30 min in CTW (the same as in the tₒ trial). Hence, 2 w in HALT of 40° C. and 50 rh% do not yield in detectible efficacy (or analytic) degradation.

Application of a diluted Vaseline top coat (30 wt%, in BuOAc) impeded the efficacy. Total eradication was observed only after 4 h.

Additional experiments: 30/70 2 w trial (AWT082), 1 m trial, shelf-life of SC(aq)-based assemblies.

AWT082

Experimentals Goals: examine shelf life by trial: time=2 weeks, 30° C./70 rh%.

TABLE
Experiments Content (AMA only)

| | | | | | | SC Active | | | | | |
|---|---|---|---|---|---|---|---|---|---|---|---|
| Modal | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | VInnol/EtOAc (see | WE018/9 + | R, GTW | GTW | 10 unless | 500 | E. Coli | 3 | 0.5, | GTW: |
| B | CG8-H | also model | WE04/36 | S, GTW | | specified | | | | 4 hours | RT |
| C | | description) | | R + vinnol | | otherwise | | | | | CTW: |
| | | | | top-coat, | | in the | | | | | 4° C. |
| | | | | GTW | | model | | | | | |
| D | | | | S + vinnol | | desc. | | | | | |
| | | | | top-coat, | | | | | | | |
| | | | | GTW | | | | | | | |
| E | | | See AWT074 | S, Elvacite, | | | | | | | |
| | | | | GTW | | | | | | | |
| F | | | See AWT073 | S, w/KaMin, | | | | | | | |
| | | | | GTW | | | | | | | |
| G | | | WE018/9 + | R, Fresh, | | | | | | | |
| | | | WE04/36 | GTW | | | | | | | |
| H | | | | TBD | | | | | | | |
| I | | | WE018/9 + | R, CTW | CTW | | | | | | |
| J | | | WE04/36 | S, CTW | | | | | | | |
| K | | | | R + vinnol | | | | | | | |
| | | | | top-coat, | | | | | | | |
| | | | | CTW | | | | | | | |
| L | | | | S + vinnol | | | | | | | |
| | | | | top-coat, | | | | | | | |
| | | | | CTW | | | | | | | |
| M | | | See AWT074 | S, Elvacite, | | | | | | | |
| | | | | CTW | | | | | | | |
| N | | | See AWT073 | S, w/KaMin, | | | | | | | |
| | | | | CTW | | | | | | | |
| O | | | WE018/9 + | R, Fresh, | | | | | | | |
| | | | WE04/36 | CTW | | | | | | | |
| P | | | | TBD | | | | | | | |
| NCQ | N/A | N/A | N/A | NC GTW | GTW | N/A | 50 | | | | |
| NCR | N/A | N/A | N/A | NC CTW | CTW | N/A | 50 | | | | |

This is a yes/no experiment, thus counting is needed only for 0, 1 dilutions. CTW: Humic acid, 15±5 mg/L; CaCO₃, 100±20 mg/L; NaCl, 1500±150 mg/L, Turbidity, not applicable. Measure pH. Shake thoroughly after filling and one again after 15′.

In some embodiments of the present invention, bottles had antimicrobial activity after 0.5 hours.

Hach results:

| | | 0.5 h | | | | | 4 h | | | | |
|---|---|---|---|---|---|---|---|---|---|---|---|
| Sample | Model | Cl2 | ClO2 | ClO2 | total ox′ | pH | Cl2 | ClO2 | ClO2 | total ox′ | pH |
| A | R | 0.944 | 2.172 | 3.96 | 6.132 | 4.94 | 0.872 | 0 | 4.196 | 4.196 | 4.54 |
| B | S | 0 | 6.124 | 0 | 6.124 | 4.51 | 0 | 2.12 | 2.124 | 4.244 | 4.39 |
| C | R + vinnol | 0 | 0 | 0 | 0 | 5.05 | 0 | 2.624 | 0 | 2.624 | 4.58 |
| | top-coat | | | | | | | | | | |
| D | S + vinnol | 0 | 0 | 0 | 0 | 4.68 | 0.388 | 0 | 3.584 | 3.584 | 4.41 |
| | top-coat | | | | | | | | | | |
| E | S, Elvacite | 0 | 4.22 | 0 | 4.22 | 4.43 | 0 | 2.66 | 2.104 | 4.764 | 4.14 |
| F | S, | 0 | 0 | 0 | 0 | 4.71 | 0 | 2.856 | 0 | 2.856 | 4.47 |
| | w/KaMin | | | | | | | | | | |
| G | R, Fresh | 0 | 0 | 0 | 0 | 4.89 | 0 | 0.82 | 3.152 | 3.972 | 4.7 |
| H | R, | 0 | 0 | 0 | 0 | 4.99 | 0 | 0.556 | 4.38 | 4.936 | 4.66 |
| | w/Paraffin | | | | | | | | | | |
| | oil | | | | | | | | | | |

Discussion and Outlook:

Most of the AWT assemblies were effective in CTW in 30 min after 2 w in HALT of 30° C. and 70 rh %. Additional Vinnol layer (filler-free) yielded impeded efficacy (also in time zero). Furthermore, Vinnol-top-coated sheets exhibited apparent degradation of its color (green to blue), indicating on CDO release.

Application of paraffin oil did not impact efficacy. Its potential as humidity barrier are further examined.

Additional experiments: 1 m shelf-life trial (also with 40° C./50 rh %), shelf life of assemblies of different binder-systems (Elvacite, Vinacoat).

AWT083, AWT084, AWT085, and AWT087

In a non-limiting example, samples 86-89 [AWT083-5 and AWT087] were examined regarding the efficacies of reversed and sandwiched assemblies prepared with Vinnacoat LL8100 dispersed in EtOAc (instead of MEK). While Vinnacoat/EtOAc were typically effective in GTW, it were significantly less effective than Vinnacoat/MEK assemblies in CTW (not effective after 30 min to not at all. See samples 80-81 for MEK-dispersed reference). Variation of the fillers weight contents, 50-60 wt % in the CG8-H layer and/or 40-60 wt % in the SC layer, either by SC alone or also with KaMin 70° C. (20-30 wt % SC and 30 wt % KaMin 70C) was not successful.

AWT083

Experimental Goals: Examination of Vinnacoat LL8100 assemblies with EtOAc as the dispersing agent and SC(aq) as the precursor source.

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | VInnol/EtOAc | WE060/1 + | R, 40% SC(aq), GTW | GTW | 10 unless | 500 | E. Coli | 3 | 0.5, | GTW: |
| B | CG8-H | (see also model | WE061/1 | S, 40% SC(aq), GTW | | specified | | | | 4 hours | RT |
| C | | description) | WE060/1 + | R, 20% SC(aq) + | | otherwise | | | | | CTW: |
| | | | WE062/1 | 30% KaMin 70 C., | | in the | | | | | 4° C. |
| | | | | GTW | | model | | | | | |
| D | | | | S, 20% SC(aq) + | | desc. | | | | | |
| | | | | 30% KaMin 70 C., | | | | | | | |
| | | | | GTW | | | | | | | |
| E | | | WE052/1 + | R, 40% SC(aq), 1 | | | | | | | |
| | | | WE054/1 | w old (MEK), | | | | | | | |
| | | | | GTW | | | | | | | |
| F | | | WE052/1 + | R, 20% SC(aq) + | | | | | | | |
| | | | WE055/1 | 30% KaMin 70 C., | | | | | | | |
| | | | | 1 w old (MEK), GTW | | | | | | | |
| G | | | WE018/9 + | R, Vinnol, GTW | | | | | | | |
| | | | WE004/36 | | | | | | | | |
| H | | | WE060/1 + | R, 40% SC(aq), | CTW | | | | | | |
| | | | WE061/1 | CTW | | | | | | | |
| I | | | | S, 40% SC(aq), | | | | | | | |
| | | | | CTW | | | | | | | |
| J | | | WE060/1 + | R, 20% SC(aq) + | | | | | | | |
| | | | WE062/1 | 30% KaMin 70 C., | | | | | | | |
| | | | | CTW | | | | | | | |
| K | | | | S, 20% SC(aq) + | | | | | | | |
| | | | | 30% KaMin 70 C., | | | | | | | |
| | | | | CTW | | | | | | | |
| L | | | WE052/1 + | R, 40% SC(aq), 1 | | | | | | | |
| | | | WE054/1 | w old (MEK), | | | | | | | |
| | | | | CTW | | | | | | | |
| M | | | WE052/1 + | R, 20% SC(aq) + | | | | | | | |
| | | | WE055/1 | 30% KaMin 70 C., | | | | | | | |
| | | | | 1 w old (MEK), aw | | | | | | | |
| N | | | WE018/9 + | R, Vinnol, CTW | | | | | | | |
| | | | WE004/36 | | | | | | | | |
| NCO | N/A | N/A | N/A | NC GTW | GTW | N/A | 50 | | | | |
| NCP | N/A | N/A | N/A | NC CTW | CTW | N/A | 50 | | | | |

This is a yes/no experiment, thus counting is needed only for 0,1 dilutions. CTW: Humic acid, 15±5 mg/L; CaCO₃, 100±20 mg/L; NaCl, 1500±150 mg/L, Turbidity, not applicable at the moment. Measure pH. Shake thoroughly after filling and one again after 15′.

Hach results:

| | | 0.5 h | | | | | 4 h | | | | |
|---|---|---|---|---|---|---|---|---|---|---|---|
| Sample | Model | Cl2 | ClO2 | ClO2 | total ox′ | pH | Cl2 | ClO2 | ClO2 | total ox′ | pH |
| A | R[1], Vinnacoat/EtOAc | 0 | 0.608 | 0 | 0.608 | 6.74 | 0.62 | 0 | 4.068 | 4.068 | 4.71 |
| | (20/80) 40 wt % SC(ac), | | | | | | | | | | |
| | GTW | | | | | | | | | | |
| B | S[2], Vinnacoat/EtOAc | 0 | 0 | 0 | 0 | 5.08 | 0 | 0 | 0 | 0 | 4.58 |
| | (20/80) 40 wt % SC(ac), | | | | | | | | | | |
| | GTW | | | | | | | | | | |
| C | R, Vinnacoat/EtOAc | 0 | 4.728 | 0 | 4.728 | 4.83 | 0 | 3.916 | 0 | 3.916 | 4.49 |
| | (20/80) 20 wt % SC(ac) + | | | | | | | | | | |
| | 30 wt % KaMin 70 C., GTW | | | | | | | | | | |
| D | S, Vinnacoat/EtOAc | 0 | 0 | 0 | 0 | 4.85 | 0.98 | 0 | 4.096 | 4.096 | 4.46 |
| | (20/80) 20 wt % SC(ac) + | | | | | | | | | | |
| | 30 wt % KaMin 70 C., GTW | | | | | | | | | | |
| E | R, Vinnacoat/MEK | 0 | 3.912 | 0 | 3.912 | 4.76 | 0.488 | 0 | 3.164 | 3.164 | 4.53 |
| | (20/80) 40 wt % SC(ac), | | | | | | | | | | |
| | GTW | | | | | | | | | | |
| F | R, Vinnacoat/EtOAc | 0 | 2.332 | 0 | 2.332 | 4.54 | 0 | 2.46 | 0 | 2.46 | 4.2 |
| | (20/80) 20 wt % SC(ac) + | | | | | | | | | | |
| | 30 wt % KaMin 70, GTW | | | | | | | | | | |
| G | R, Vinnol, 40 wt % SC(s), | 0 | 0.116 | 4.188 | 4.304 | 4.67 | 0 | 0.452 | 5.436 | 5.888 | 4.48 |
| | GTW | | | | | | | | | | |

Dispersing Vinnacoat LL8100 in EtOAc did not yield effective samples as with using MEK as dispersant. The films obtained exhibited slower release kinetics and impeded efficacy (only after 0.5 h). Possible reasons for this behavior are permeable film formation, insufficient stability of the resin dispersion in EtOaC or its formulations, etc. This gap may be mitigated by using higher filler volume content (to-be-examined in AWT084 and preliminarily/qualitatively in the diapers project).

Additional experiments: elevate volume fraction of fillers, other solvents (e.g., acetone, diethyl ether), elevating drying temperature of MEK-dispersed formulations.

AWT084

Experiment goals: (1) CG8-H batch validation. (2) Vinnacoat/EtOAc 60 wt% of IX, and (3) Paraffin oils utilization at tₒ.

TABLE
Experiments Conetent (AMA only)

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | VInnol/ | WE018/10 + | R, GTW | GTW | 10 unless | 500 | E. Coli | 3 | 0.5, | GTW: |
| B | CG8-H | EtOAc | WE004/37 | S, GTW | | specified | | | | 4 hours | RT |
| C | | (see also | HP003/1 + | R, | | otherwise | | | | | CTW: |
| | | model | WE061/1 | vinnacoat/EtOAc | | in the | | | | | 4° C. |
| | | description) | | 60 wt %, GTW | | model | | | | | |
| D | | | WE018/10 + | R w/paraffin 1, | | desc. | | | | | |
| | | | WE004/37 | GTW | | | | | | | |
| E | | | | R w/paraffin 2, | | | | | | | |
| | | | | GTW | | | | | | | |
| F | | | | Paraffin oil | | | | | | | |
| | | | | only, GTW | | | | | | | |
| G | | | WE018/10 + | R, CTW | CTW | | | | | | |
| H | | | WE004/37 | S, CTW | | | | | | | |
| I | | | HP003/1 + | R, | | | | | | | |
| | | | WE061/1 | vinnacoat/EtOAc | | | | | | | |
| | | | | 60 wt %, CTW | | | | | | | |
| J | | | WE018/10 + | R w/paraffin 1, | | | | | | | |
| | | | WE004/37 | CTW | | | | | | | |
| K | | | | R w/paraffin 2, | | | | | | | |
| | | | | CTW | | | | | | | |
| L | | | | Paraffin oil | | | | | | | |
| | | | | only, CTW | | | | | | | |
| NCM | N/A | N/A | N/A | NC GTW | GTW | N/A | 50 | | | | |
| NCN | N/A | N/A | N/A | NC CTW | CTW | N/A | 50 | | | | |

This is a yes/no experiment, thus counting is needed only for 0, 1 dilutions. CTW: Humic acid, 15±5 mg/L; CaCO₃, 100±20 mg/L; NaCl, 1500±150 mg/L, Turbidity, not applicable at the moment. Measure pH. Shake thoroughly after filling and one again after 15′.

Microbiology Results:

| | | 0.5 h | | | | | 4 h | | | | |
|---|---|---|---|---|---|---|---|---|---|---|---|
| Sample | Model | Cl2 | ClO2 | ClO2— | total ox′ | pH | Cl2 | ClO2 | ClO2— | total ox′ | pH |
| A | R, Vinnol | 0 | 0 | 0 | 0 | 5.08 | 0 | 2.7 | 0 | 2.7 | 4.62 |
| B | S, Vinnol | 0 | 0 | 0 | 0 | 5 | 0 | 3.116 | 0 | 3.12 | 4.5 |
| C | R, | 0 | 4.088 | 0 | 4.09 | 5.58 | 0 | 3.652 | 0 | 3.65 | 4.28 |
| | Vinnacoat/EtoAc, | | | | | | | | | | |
| | 40 wt % SC/60 | | | | | | | | | | |
| | wt % IX | | | | | | | | | | |
| D | R w/paraffin oil 1 | 2.024 | 0 | 3.852 | 3.85 | 5.69 | 0 | 2.252 | 0 | 2.25 | 4.7 |
| E | R w/paraffin oil 2 | 0 | 0 | 0 | 0 | 5.63 | 0 | 4.596 | 0 | 4.6 | 4.69 |
| F | Paraffin oil | 0 | 0 | 0 | 0 | 5.81 | 0 | 0 | 0 | 0 | 5.06 |

Vinnacoat/EtOAc formulation assembly with 60 wt % of CG8-H in the dry layer yielded fully effective behavior in 30 min only in GTW. Assemblies were effective in CTW only after 4 h.

However, the fact that none of the examined assemblies was effective after 30 min in (including the usually effective R and S Vinnol assemblies) CTW raises some doubt about the validity of this part of the trial. Hence, a wider trial exploring the efficacy of Vinnacoat/EtOAc formulation with different filler's weight/volume fractions (in both layers) was performed (AWT085).

Application of paraffin oils on top of the assemblies resulted in impeded efficacy. Total eradication was obtained only after 4 h.

AWT085

Experiments Goals: Vinnacoat/EtOAc based formulations w/SC(aq) enable efficacy by elevating the filler weight/volume concentration.

TABLE
Experiments Content (AMA only)

| | | | | | | SC Active | | | | | |
|---|---|---|---|---|---|---|---|---|---|---|---|
| Model | Active | Additional | Formulation | | | conc. | Vol. | Micro- | Inoculation | Sampling | Temp, |
| No. | material | materials | number | Model description | Medium | [ppm] | [ml] | organism | [Log cfu/ml] | times | C. |
| A | SC + | VInnol/EtOAc | WE060/2 + | R, 50 wt % CG8-H, | GTW | 10 unless | 500 | E. Coli | 3 | 0.5, | GTW: |
| | CG8-H | (see also model | WE064/1 | 60 wt % SC, GTW | | specified | | | | 4 hours | RT |
| B | | description) | WE060/2 + | R, 50 wt % CG8-H, | | otherwise | | | | | CTW: |
| | | | WE065/1 | 30 wt % SC + | | in the | | | | | 4° C. |
| | | | | 30 wt % KaMin | | model | | | | | |
| | | | | 70 C., GTW | | desc. | | | | | |
| C | | | WE063/1 + | R, 60 wt % CG8-H, | | | | | | | |
| | | | WE064/1 | 60 wt % SC, GTW | | | | | | | |
| D | | | WE063/1 + | R, 60 wt % CG8-H, | | | | | | | |
| | | | WE065/1 | 30 wt % SC + | | | | | | | |
| | | | | 30 wt % KaMin | | | | | | | |
| | | | | 70 C., GTW | | | | | | | |
| E | | | WE063/1 + | R, 60 wt % CG8-H, | | | | | | | |
| | | | WE061/1 | 40 wt % SC, GTW | | | | | | | |
| F | | | WE063/1 + | R, 60 wt % CG8-H, | | | | | | | |
| | | | WE062/1 | 20 wt % SC + | | | | | | | |
| | | | | 30 wt % KaMin | | | | | | | |
| | | | | 70 C., GTW | | | | | | | |
| G | | | WE060/2 + | R, 50 wt % CG8-H, | | | | | | | |
| | | | WE062/1 | 20 wt % SC + | | | | | | | |
| | | | | 30 wt % KaMin | | | | | | | |
| | | | | 70 C., GTW | | | | | | | |
| H | | | WE060/2 + | R, 50 wt % CG8-H, | CTW | | | | | | |
| | | | WE064/1 | 60 wt % SC, CTW | | | | | | | |
| I | | | WE060/2 + | R, 50 wt % CG8-H, | | | | | | | |
| | | | WE065/1 | 30 wt % SC + | | | | | | | |
| | | | | 30 wt % KaMin | | | | | | | |
| | | | | 70 C., CTW | | | | | | | |
| J | | | WE063/1 + | R, 60 wt % CG8-H, | | | | | | | |
| | | | WE064/1 | 60 wt % SC, CTW | | | | | | | |
| K | | | WE063/1 + | R, 60 wt % CG8-H, | | | | | | | |
| | | | WE065/1 | 30 wt % SC + | | | | | | | |
| | | | | 30 wt % KaMin | | | | | | | |
| | | | | 70 C., CTW | | | | | | | |
| L | | | WE063/1 + | R, 60 wt % CG8-H, | | | | | | | |
| | | | WE061/1 | 40 wt % SC, CTW | | | | | | | |
| M | | | WE063/1 + | R, 60 wt % CG8-H, | | | | | | | |
| | | | WE062/1 | 20 wt % SC + | | | | | | | |
| | | | | 30 wt % KaMin | | | | | | | |
| | | | | 70 C., cTW | | | | | | | |
| N | | | WE060/2 + | R, 50 wt % CG8-H, | | | | | | | |
| | | | WE062/1 | 20 wt % SC + | | | | | | | |
| | | | | 30 wt % KaMin | | | | | | | |
| | | | | 70 C., CTW | | | | | | | |
| NCO | N/A | N/A | N/A | NC GTW | GTW | N/A | 50 | | | | |
| NCP | N/A | N/A | N/A | NC CTW | CTW | N/A | 50 | | | | |

This is a yes/no experiment, thus counting is needed only for 0,1 dilutions. CTW: Humic acid, 15±5 mg/L; CaCO₃, 100±20 mg/L; NaCl, 1500±150 mg/L, Turbidity, not applicable at the moment. Measure pH. Shake thoroughly after filling and one again after 15′.

Results: Table showing E.coli viable counts.

Table showing Hach results:

| | | 0.5 h | | | | | 4 h | | | | |
|---|---|---|---|---|---|---|---|---|---|---|---|
| Sample | Model | Cl2 | ClO2 | ClO2— | total ox′ | pH | Cl2 | ClO2 | ClO2— | total ox′ | pH |
| A | R, 50 wt % | 0 | 0 | 0 | 0 | 5.36 | 0 | 4.584 | 0 | 4.584 | 5.00 |
| | CG8-H, 60 | | | | | | | | | | |
| | wt % SC, | | | | | | | | | | |
| | GTW | | | | | | | | | | |
| B | R, 50 wt % | 0 | 0 | 0 | 0 | 5.21 | 0.524 | 2.12 | 4.216 | 6.336 | 4.53 |
| | CG8-H, 30 | | | | | | | | | | |
| | wt % SC + | | | | | | | | | | |
| | 30 wt % | | | | | | | | | | |
| | KaMin 70 C., | | | | | | | | | | |
| | GTW | | | | | | | | | | |
| C | R, 60 wt % | 0 | 0 | 0 | 0 | 4.76 | 0 | 3.684 | 0 | 3.684 | 4.65 |
| | CG8-H, 60 | | | | | | | | | | |
| | wt % SC, | | | | | | | | | | |
| | GTW | | | | | | | | | | |
| D | R, 60 wt % | 0 | 0 | 0 | 0 | 5.07 | 0 | 3.616 | 2.86 | 6.476 | 4.37 |
| | CG8-H, 30 | | | | | | | | | | |
| | wt % SC + | | | | | | | | | | |
| | 30 wt % | | | | | | | | | | |
| | KaMin 70 C., | | | | | | | | | | |
| | GTW | | | | | | | | | | |
| E | R, 60 wt % | 0 | 0 | 0 | 0 | 5.28 | 0 | 2.284 | 0 | 2.284 | 4.63 |
| | CG8-H, 40 | | | | | | | | | | |
| | wt % SC, | | | | | | | | | | |
| | GTW | | | | | | | | | | |
| F | R, 60 wt % | 0 | 0 | 0 | 0 | 4.60 | 0 | 2.468 | 0 | 2.468 | 4.49 |
| | CG8-H, 20 | | | | | | | | | | |
| | wt % SC + | | | | | | | | | | |
| | 30 wt % | | | | | | | | | | |
| | KaMin 70 C., | | | | | | | | | | |
| | GTW | | | | | | | | | | |
| G | R, 50 wt % | 0 | 0 | 0 | 0 | 5.32 | 0.504 | 0 | 3.12 | 3.12 | 4.89 |
| | CG8-H, 20 | | | | | | | | | | |
| | wt % SC + | | | | | | | | | | |
| | 30 wt % | | | | | | | | | | |
| | KaMin 70 C., | | | | | | | | | | |
| | GTW | | | | | | | | | | |

### Discussion

While all of the Vinnacoat LL8100 in EtOAc based samples were effective in GTW after 30 min, none was effective in CTW at the same time. This comes in agreement with the previous AWT084. This may be related to slow CDO release kinetics of the Vinnacoat film (when it is dispersed in EtOAc), as indicated by the Hach results. The apparent slow release kinetics may be insufficient to allo AMA in the challenging CTW environment. However, since the trial is lacking a true positive control sample, some of the samples will be reexamined in a different trial (AWT087).

No clear dependence of the efficacy or CDO kinetics in the solid content of the filler in each layer was observed. Nonetheless, samples with 60 wt % of CG8-H and 30 wt % KaMin 70C in the SC layer (either 20 wt % or 30 wt % of SC) were effective after 4 h in CTW. Those samples will be examined further. One must also remember that Vinnacoat/MEK samples were effective also after 30 min in CTW.

AWT087

Experiments Goals: Vinnacoat/EtOAc based formulations w/SC(aq) enable efficacy by elevating the filler weight/volume concentration.

TABLE
Experiments Content (AMA only)

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | Temp, |
| No. | material | materials | number | description | Medium | [ppm] | [ml] | organism | [Log cfu/ml] | times | C. |
| A | SC + | see model | WE063/1 + | R, | GTW | 10 unless | 500 | E. Coli | 3 | 0.5, | GTW: |
| | CG8-H | description | WE065/1 | Vinnacoat/EtOAc, | | specified | | (ATCC | | 4 hours | RT |
| | | | | 60 wt % CG8- | | otherwise | | 11229) | | | CTW: |
| | | | | H, 30 wt % SC + | | in the | | | | | 4° C. |
| | | | | 30 wt % KaMin | | model | | | | | |
| | | | | 70 C., GTW | | desc. | | | | | |
| B | | | WE063/1 + | R, | | | | | | | |
| | | | WE062/1 | Vinnacoat/EtOAc, | | | | | | | |
| | | | | 60 wt % CG8- | | | | | | | |
| | | | | H, 20 wt % SC + | | | | | | | |
| | | | | 30 wt % KaMin | | | | | | | |
| | | | | 70 C., GTW | | | | | | | |
| C | | | WE052/2 + | R, | | | | | | | |
| | | | WE054/2 | Vinnacoat/MEK, | | | | | | | |
| | | | | 50 wt % CG8-H, | | | | | | | |
| | | | | 40 wt % SC, 80° C. | | | | | | | |
| | | | | GTW | | | | | | | |
| D | | | WE052/1 + | R, | | | | | | | |
| | | | WE054/1 | Vinnacoat/MEK, | | | | | | | |
| | | | | 50 wt % CG8-H, | | | | | | | |
| | | | | 40 wt % SC, 60° | | | | | | | |
| | | | | C., 2 w aged, | | | | | | | |
| | | | | GTW | | | | | | | |
| E | | | WE018/9 + | R, Vinnol/EtOAc, | | | | | | | |
| | | | WE004/36 | 40 wt % SC(s), | | | | | | | |
| | | | | GTW | | | | | | | |
| F | | | | S, Vinnol/EtOAc, | | | | | | | |
| | | | | 40 wt % SC(s), | | | | | | | |
| | | | | GTW | | | | | | | |
| G | | | WE063/1 + | R, 60 wt % CG8- | CTW | | | | | | |
| | | | WE065/1 | H, 30 wt % SC + | | | | | | | |
| | | | | 30 wt % KaMin | | | | | | | |
| | | | | 70 C., CTW | | | | | | | |
| H | | | WE063/1 + | R, 60 wt % CG8- | | | | | | | |
| | | | WE062/1 | H, 20 wt % SC + | | | | | | | |
| | | | | 30 wt % KaMin | | | | | | | |
| | | | | 70 C., CTW | | | | | | | |
| I | | | WE052/2 + | R, | | | | | | | |
| | | | WE054/2 | Vinnacoat/MEK, | | | | | | | |
| | | | | 50 wt % CG8-H, | | | | | | | |
| | | | | 40 wt % SC, 80° C. | | | | | | | |
| | | | | CTW | | | | | | | |
| J | | | WE052/1 + | R, | | | | | | | |
| | | | WE054/1 | Vinnacoat/MEK, | | | | | | | |
| | | | | 50 wt % CG8-H, | | | | | | | |
| | | | | 40 wt % SC, 60° | | | | | | | |
| | | | | C., 2 w aged, | | | | | | | |
| | | | | CTW | | | | | | | |
| K | | | WE018/9 + | R, Vinnol/EtOAc, | | | | | | | |
| | | | WE004/36 | 40 wt % SC(s), | | | | | | | |
| | | | | CTW | | | | | | | |
| L | | | | S, Vinnol/EtOAc, | | | | | | | |
| | | | | 40 wt % SC(s), | | | | | | | |
| | | | | CTW | | | | | | | |
| NCM | N/A | N/A | N/A | NC GTW | GTW | N/A | 50 | | | | |
| NCN | N/A | N/A | N/A | NC CTW | CTW | N/A | 50 | | | | |

This is a yes/no experiment, thus counting is needed only for 0,1 dilutions. CTW: Humic acid, 15±5 mg/L; CaCO₃, 100±20 mg/L; NaCl, 1500±150 mg/L, Turbidity, not applicable at the moment. Measure pH. Shake thoroughly after filling and one again after 15′.

Microbiology Results: Table showing E.coli viable counts.

Table Showing Hach Results:

| | | 0.5 h | | | | | 4 h | | | | |
|---|---|---|---|---|---|---|---|---|---|---|---|
| Sample | Model | Cl2 | ClO2 | ClO2— | total ox′ | pH | Cl2 | ClO2 | ClO2— | total ox′ | pH |
| A | R, Vinnacoat/EtOAc, | 0 | 0.248 | 2.392 | 2.64 | 4.72 | 0 | 2.06 | 2.604 | 4.664 | 4.25 |
| | 60 wt % CG8-H, 30 | | | | | | | | | | |
| | wt % SC + 30 wt % | | | | | | | | | | |
| | KaMin 70 C., GTW | | | | | | | | | | |
| B | R, Vinnacoat/EtOAc, | 0 | 0 | 0 | 0 | 5.05 | 0 | 2.248 | 0 | 2.248 | 4.49 |
| | 60 wt % CG8-H, 20 | | | | | | | | | | |
| | wt % SC + 30 wt % | | | | | | | | | | |
| | KaMin 70 C., GTW | | | | | | | | | | |
| C | R, Vinnacoat/MEK, 50 | 0 | 0 | 0 | 0 | 4.87 | 0 | 2.176 | 0 | 2.176 | 4.53 |
| | wt % CG8-H, 40 wt % | | | | | | | | | | |
| | SC, 80° C. GTW | | | | | | | | | | |
| D | R, Vinnacoat/MEK, 50 | 0 | 0 | 0 | 0 | 5.47 | 0 | 4.304 | 0 | 4.304 | 4.77 |
| | wt % CG8-H, 40 wt % | | | | | | | | | | |
| | SC, 60° C., 2 w aged, | | | | | | | | | | |
| | GTW | | | | | | | | | | |
| E | R, Vinnol/EtOAc, 40 | 0 | 2.22 | 0 | 2.22 | 4.82 | 0 | 3.708 | 0 | 3.708 | 4.42 |
| | wt % SC(s), GTW | | | | | | | | | | |
| F | S, Vinnol/EtOAc, 40 | 0 | 0 | 0 | 0 | 4.78 | 0 | 3.42 | 0 | 3.42 | 4.44 |
| | wt % SC(s), GTW | | | | | | | | | | |

### Discussion

MEK is clearly superior to EtOAc, efficacy-wise, as dispersant to Vinnacoat LL8100. This is indicated by the improved efficacy of the MEK-dispersed samples (dried at 60° C., >2 w aged) over the EtOAC-dispersed samples in CTW (at least in the current formulations).

Drying MEK-dispersed Vinnacoat formulation yield less effective inserts. Furthermore, the elevation of drying temperature is not effective in reducing the MEK residues odor in the dry films.

Additional experiment: find alternative dispersants Vinnacoat other than MEK and EtOAc, explore other binder systems.

AWT086

In a non-limiting example, samples 90-91 [AWT086] were examined regarding the efficacies of reversed and sandwiched assemblies. As-prepared (“fresh”) assemblies with SC contents of 5, 7.5 or 10 ppm were examined parallel to 5 months old assemblies that were stored in RT (see samples 17 and 18 for time zero reference). Aged reversed assemblies with alkalized SC formulation (WE007) yielded total eradication after 30 min. i.e. 5 months of shelf-life. The as-prepared assemblies were effective down to 7.5 ppm after 30 min and down to 5 ppm after 4 h (only partially effective after 30 min).

Experiments Goals: (1) Shelf life of reversed and sandwiched assemblies, 5 m in RT. (2) Utilization of the WHO-dictated E. coli strain. ATCC 11229.

Table: Experiments Content (AMA only)

| | | | | | | SC Active | | | | | |
|---|---|---|---|---|---|---|---|---|---|---|---|
| Model | Active | Additional | Formulation | Model | | conc. | Vol. | Micro- | Inoculation | Sampling | |
| No. | material | materials | number | description | Medium | [ppm] | [ml] | organism | [Log cfu/ml] | times | Temp, C. |
| A | SC + | Vinnol/ | WE018/9 + | R, fresh, 10 ppm, | GTW | 10 unless | 500 | E. Coli | 3 | 0.5, 4 hours | GTW: |
| | CG8-H | EtOAc | WE004/36 | GTW | | specified | | (ATCC | | | RT |
| B | | (see also | | S, fresh, 10 ppm, | | otherwise | | 11229) | | | CTW: |
| | | model | | GTW | | in the | | | | | 4° C. |
| C | | description) | | R, fresh, 7.5 ppm, | | model | | | | | |
| | | | | GTW | | desc. | | | | | |
| D | | | | S, fresh, 7.5 ppm, | | | | | | | |
| | | | | GTW | | | | | | | |
| E | | | | S, fresh, 5 ppm, | | | | | | | |
| | | | | GTW | | | | | | | |
| F | | | See AWT018 | R, 5 m old, GTW | | | | | | | |
| G | | | | S, 5 m old, GTW | | | | | | | |
| H | | | See AWT019 | R, alk., 5 m old, | | | | | | | |
| | | | | GTW | | | | | | | |
| I | | | | S, alk., 5 m old, | | | | | | | |
| | | | | GTW | | | | | | | |
| J | | | WE018/9 + | R, fresh, 10 ppm, | CTW | | | | | | |
| | | | WE004/36 | CTW | | | | | | | |
| K | | | | S, fresh, 10 ppm, | | | | | | | |
| | | | | CTW | | | | | | | |
| L | | | | R, fresh, 7.5 ppm, | | | | | | | |
| | | | | CTW | | | | | | | |
| M | | | | S, fresh, 7.5 ppm, | | | | | | | |
| | | | | CTW | | | | | | | |
| N | | | | S, fresh, 5 ppm, | | | | | | | |
| | | | | CTW | | | | | | | |
| O | | | See AWT018 | R, 5 m old, CTW | | | | | | | |
| P | | | | S, 5 m old, CTW | | | | | | | |
| Q | | | See AWT019 | R, alk., 5 m old, | | | | | | | |
| | | | | CTW | | | | | | | |
| R | | | | S, alk., 5 m old, | | | | | | | |
| | | | | CTW | | | | | | | |
| NCS | N/A | N/A | N/A | NC GTW | GTW | N/A | 50 | | | | |
| NCT | N/A | N/A | N/A | NC CTW | CTW | N/A | 50 | | | | |

This is a yes/no experiment, thus counting is needed only for 0, 1 dilutions, CTW: Humic acid, 15±5 mg/L; CaCO₃, 100±20 mg/L; NaCl, 1500±150 mg/L, Turbidity, not applicable. Measure pH. Shake thoroughly after filling and one again after 15′.

Results: E. coli viable counts

Hach Results:

| | | 0.5 h | | | | | 4 h | | | | |
|---|---|---|---|---|---|---|---|---|---|---|---|
| | | | | | total | | | | | total | |
| Sample | Model | Cl2 | ClO2 | ClO2− | ox′ | pH | Cl2 | ClO2 | ClO2− | ox′ | pH |
| A | R, fresh, 10 ppm | 0.044 | 0 | 3.724 | 3.724 | 4.58 | 1.412 | 1 | 2.392 | 3.392 | 4.46 |
| B | S, fresh, 10 ppm | 0 | 2.708 | 0 | 2.708 | 4.7 | 0.96 | 0 | 6.176 | 6.176 | 4.5 |
| C | R, fresh, 7.5 ppm | 0.312 | 0 | 3.52 | 3.52 | 4.72 | 0 | 0.48 | 3.388 | 3.868 | 4.56 |
| D | S, fresh, 7.5 ppm | 0 | 1.972 | 0 | 1.972 | 4.87 | 0.196 | 0 | 3.864 | 3.864 | 4.55 |
| E | S, fresh, 5 ppm | 0 | 0 | 0 | 0 | 5.32 | 0 | 0.124 | 2.508 | 2.632 | 4.67 |
| F | R, 5 m old | 0 | 0 | 0 | 0 | 5.15 | 0 | 2.984 | 0 | 2.984 | 4.49 |
| G | S, 5 m old | 0 | 2.408 | 0 | 2.408 | 4.98 | 0 | 2.66 | 0 | 2.66 | 4.6 |
| H | R, alk., 5 m old | 0 | 0 | 0 | 0 | 5.17 | 0 | 0 | 0 | 0 | 4.54 |
| I | S, alk., 5 m old | 0 | 0 | 0 | 0 | 4.95 | 0 | 0 | 0 | 0 | 4.42 |

Discussion:

Shelf life trial: 5 m old reversed and sandwiched assemblies were partially effective. While in GTW both reversed assemblies (alkalized and not) were effective after 0.5 h, as well as the alkalized sandwiched, on CTW the picture was different. Only the alkalized reversed exhibited almost full efficacy after 30 min and full after 4 h. the superiority of the reversed sample may be regarded to the smaller IX content, resulting in less uptake of humidity from the ambient during storage. Hach results are inconclusive, and do not come in agreement with the AMA results.

Additional experimentation: resume shelf-life trials at various environment conditions alongside the RT storage trials, explore humidity protection solutions.

“New” E. coli results: both reversed and sandwiched assemblies were effective vs. E. coli ATCC 11229 (the strain dictated by the WHO protocol). Both configurations were effective after 0.5 h down to 7.5 ppm. Sandwiched assembly of 5 ppm was partially effective after 30 min and fully effective after 4 h.

AWTan005

In a non-limiting example, sample 92 [AWTan005] was examined regarding the efficiency of ClOx-species neutralizing sheets. The neutralizing sheets were prepared with a 33.3 wt % solution of sodium thiosulfate (Na₂S₂O₃) in either Vinnol/EtOAc or Neocryl A-2092 as binders, yielding a 5 wt % formulation. Dry 50 ppm of thiosulfate sheets were prepared and inserted into 500 ml bottles that were previously inserted with 10 ppm SC active sheets. Both type of sheets succeeded in reducing the CDO, ClO₂⁻, and free Cl₂ residues to zero in 15 min (first time point measured). Bottles that were not added with a neutralizing sheet yielded positive readings for ClOx-species.

Hach trial results, showing the feasibility of neutralizing ClOx-species using Na₂S₂O₃ sheets.

Tests: (1)No neutralizer; (2) 5 ml of neutralizer (SS+ 0.3 wt % Na2S2O3 and 0.85 wt % NaCl) per 200 ml of treated water, (3)_50 ppm of Na2S2O3 in vinnol/EtOAc sheet (prepared from 50 wt % aqueous Na2S2O3 sol). 120 mic wet thickness, dried for 30′ at 60° C., and 50 ppm of Na2S2O3 in Neocryl A-2092 sheet (prepared from 50 wt % aqueous Na2S2O3 sol). 120 mic wet thickness, dried for 60′ at 60° C.

500 ml bottles were filled with DDW. 10 ppm sandwiches sheet were placed in the water. After 4 h the bottles were added with the neutralizing model and measure for their ClOx-species content after 15′ and 60′.

| | additives | dil | Time | | | | total | | dil | Time | | | | total | |
|---|---|---|---|---|---|---|---|---|---|---|---|---|---|---|---|
| Serial # | [neutralizer] | [°] | [min] | Cl2 | ClO2 | ClO2− | ox′ | pH | [°] | [min] | Cl2 | ClO2 | ClO2− | ox′ | pH |
| A | no | 4 | 0 | 0 | 2.82 | 1.132 | 3.952 | 4.53 | | | | | | 0 | |
| B | 5 ml SS+ | no | 30 | 0 | 0 | 0 | 0 | 4.28 | | | | | | 0 | |
| C | 50 ppm in | 4 | 15 | 0 | 0 | 0 | 0 | 4.55 | 4 | 60 | 0 | 0 | 0 | 0 | 4.6 |
| | Vinnol | | | | | | | | | | | | | | |
| D | 50 ppm in | 4 | 15 | 0 | 0 | 0 | 0 | 4.49 | 4 | 60 | 0 | 0 | 0 | 0 | 4.5 |
| | Vinnol | | | | | | | | | | | | | | |
| E | 50 ppm in | 4 | 15 | 0 | 0 | 0 | 0 | 4.44 | 4 | 60 | 0 | 0 | 0 | 0 | 4.47 |
| | Vinnol | | | | | | | | | | | | | | |
| F | 50 ppm in | 4 | 15 | 0 | 0 | 0 | 0 | 4.23 | 4 | 60 | 0 | 0 | 0 | 0 | 4.16 |
| | Neocryl | | | | | | | | | | | | | | |
| G | 50 ppm in | 4 | 15 | 0 | 0 | 0 | 0 | 4.23 | 4 | 60 | 0 | 0 | 0 | 0 | 4.17 |
| | Neocryl | | | | | | | | | | | | | | |
| H | 50 ppm in | 4 | 15 | 0 | 0 | 0 | 0 | 4.23 | 4 | 60 | 0 | 0 | 0 | 0 | 4.16 |
| | Neocryl | | | | | | | | | | | | | | |

As can be seen in the table above, the neutralizing sheets completely neutralized the ClOx-species content of the media after 15 min.

Additional experiments: (1) shorter time points, (2) smaller neutralizer concentrations, (3) SS+ in a sheet (i.e., add also NaCl), (4) different neutralizers (Ferrous salts, phenols, WBAIX), and (5) organoleptic assessment.

Protocol for Evaluation of the Antibacterial “Killing” Effectiveness of the Present Technology in PET Drinking Water Bottles:

Preparation of Bacteria:

1.1 Subculturing of Micro-Organisms:

E. coli (strain #11229) and Raoultalla terrigena (strain #33257) were purchased from ATCC (Manassas, Va. 20108 USA) and stored at 4° C. or −20° C. prior to use. Three (3) days before each anti-microbial activity test (AMAT), bacteria were T-streaked using sterile circular loops on commercially-derived, Difco™ Tryptic Soy Agar (cat #236950, Becton Dickinson, New Jersey, USA) and grown at 30° C. for 48 hours. Sixteen (16) hours prior to AMAT, single colonies were picked with sterile pipette tips, inoculated into 3 ml pre-warmed Bacto™ Tryptic Soy Broth (cat #211825, Becton Dickinson, New Jersey, USA) media within 15 ml “v-shaped” sterile polystyrene tubes. Tubes were grown overnight at 30° C. with shaking of 200 rpm. Resultant bacterial densities (cfu's per ml) for e. coli and Raoultalla terrigena were assumed as being 10⁹/ml. These densities were based on previously accumulated data generated.

1.2 Preparation of Serial Dilutions of Micro-Organisms for Efficacy Experiments:

From the overnight cultures as described in 1.1, serial 10 fold dilutions of bacterial densities were prepared. To that end, 0.118 ml of the overnight e. coli and Raoultalla terrigena cultures were added separately to 1.062 ml sterile PBS in sterile eppendorf tubes to generate the respective 10′ dilutions. After thorough mixing, 0.118 ml from the 10¹ fold dilution tubes were diluted into 1.062 PBS to create the 10² fold dilution. Consequently, for both bacteria, densities available for use were 10⁹ (i.e. neat), 10⁸ and 10⁷ cfu's per ml.

2. AMAT Using the Present Invention's Coating Technology: Bacterial Spiking of Water, Preparation of “Negative Control Bottles” (NCB's) and “Active Bottles” and Bacterial Sampling:

2.1 Preparation of Water for Inoculation:

In 5 l sterile glass bottles, 2 liters of filtered water (Ionex 1000) were autoclaved and allowed to cool overnight to room temperature. This water is termed General Test Water (GTW). Challenge Test Water (CTW) was prepared in order to simulate WHO specifications. To that end, Humic acid (15 mg/l), NaCl (1.5 g/l) and CaCO₃ (100 mg/l) were added to GTW to create CTW and these bottles swirled thoroughly to ensure dissolution of these additives.

2.1 Spiking of Prepared Bacteria into Water:

Following the preparation of GTW and CTW and just prior to inoculation, TSB was added to achieve a final dilution of 1:500 dilution. Thereafter, e. coli or Raoultalla terrigena were added (i.e. “t=0”) to achieve final densities of 10³ cfu's per ml or 10⁵ cfu's per ml for both types of water. For the lowest inoculum, 0.2 ml bacteria from the diluted 10⁷ cfu's per ml vial (see 1.2) was added whilst the highest inoculum was achieved by adding 0.2 ml from the overnight bacterial cultures (10⁹ cfu's per ml). Once the inocula were added and the 5 l bottles mixed by gentle swirling for 5-10 few seconds, two separate samples were withdrawn (0.118 ml and 1 ml) per water types and taken for bacterial enumeration as described in section 3.

2.2 Preparation of “NCB's” Bottles (i.e. without the Present Invention's Antimicrobial Coating):

Immediately following 2.1, 3×50 ml aliquots from each of the two types of inoculated water (GTW and CTW) were removed and transferred to 3×50 ml sterile blue capped tubes (Miniplast, Ein Shemer, Israel) devoid of the present invention's anti-microbial coating. These NCB's served as the reference bottles to which the anti-microbial efficacy of AB's could be compared throughout the duration of the experiment. These 50 ml tubes were maintained at the desired temperature (eg. 4° C. or 25° C.) throughout the study.

2.3 Preparation of “AB's” (i.e. with the Present Invention's Antimicrobial Coating):

Immediately following 2.2, PET bottles for the different experimental group (n=3) containing the present invention's anti-microbial counting, were filled with 500 mls inoculated GTW and CTW.

As soon as the AB's were prepared, this was considered to be t=0 for the AMAT of the present invention's anti-microbial coating technology. The bottles were maintained at the desired temperature (eg. 4° C. or 25° C.) throughout the duration of the study.

3. Harvesting and Processing of Samples for the Presence and Enumeration of Bacterial Counts:

3.1 Time 0 Harvesting to Quantitate Initial Bacterial Inoculum:

For e. coli and Raoultalla terrigena bacteria, the 0.118 ml sample was withdrawn (see 2.1) from 3 separately inoculated 2 l volumes of water (GTW and CTW) and diluted down to a 1:10³ dilution (for the 10³ cfu's per ml final inoculum) or 1:10⁵ (for the 10⁵ cfu's per ml final inoculum). For both bacteria, in sterile eppendorf tubes, successive 10 fold dilutions were made by adding the 0.118 ml sample to 1.062 ml sterile PBS and further diluting this 10 fold dilution sample in an identical manner. For the 10³ cfu's per ml inoculum, successive 10 fold dilutions ranged from 10¹ (i.e. neat) to 10³ fold. For the 10⁵ cfu's per ml inoculum, successive 10 fold dilutions ranged from 10¹ (i.e. neat) to 10⁵ fold. The additional 1 ml “neat” sample withdrawn from the 2 l inoculated water was taken directly for bacterial growth as in section 3.3.1.

3.2 30 Min Harvesting of Bacterial Samples:

3.2.1 NCB's:

From the 3 separate NCB's prepared in section 2.2, 1 ml samples were withdrawn from both GTW and CTW and diluted as described in 3.1 according to the final density of bacterial inoculum (10³ or 10⁵ cfu's per ml).

3.2.2 AB's:

For all AB's per experimental groups, 1.3 mls were removed and transferred directly to sterile Eppendorf tubes for processing (see section 3.3).

3.3 Four Hr Harvesting of Bacterial Samples:

3.3.1 NCB's:

From the 3 separate NCB's prepared in section 2.2, 1 ml samples were withdrawn and diluted as described in 3.1 according to the final density of bacterial inoculum (10³ or 10⁵ cfu's per ml).

3.3.2 AB's:

For all AB's per experimental groups, 1.3 mls were removed and transferred directly to sterile Eppendorf tubes for processing (see section 3.3).

3.3 Plating of Bacterial Samples

3.3.1 NCB's at Time=0 for Quantitation of Bacterial Inoculum:

The 1 ml “neat” sample as well as 1 ml diluted samples 10¹-10³ (for 10³ cfu's per ml) of 10¹-10⁵ (for 10⁵ cfu's per ml final inoculum) were taken for bacterial growth using the pour plate technology. Following the addition of the bacterial samples to sterile petri dishes, 12 ml molten Tryptic Soy Agar (45° C.) was added and dishes immediately mixed in a circular rotation to ensure optimal mixing of the inoculum. Plates were allowed to solidify at room temperature for 1 hr and transferred overnight to a 30° C. bacterial incubator to allow growth of colonies.

3.3.2 NCB's at Time=30 Min and 4 hr Following Initiation of Experiment:

From each of the 50 ml NCB's, 1 ml “neat” samples as well as 1 ml samples at successive dilutions of 10¹-10⁵ (depending on the starting inoculum density, see 3.3.1) were taken for the pour plate methodology as described in 3.3.1.

3.3.3 AB's at Time=30 Min and 4 hr Following Initiation of Experiment:

From the 1.3 ml “neat” samples removed from the AB's (see 3.2.2), 0.1 and 1 ml aliquots were added to separate sterile petri dishes and taken for the pour plate methodology as described in 3.3.1.

Enumeration of Bacterial Counts:

For all samples, only those specific plates which harbored 25-250 cfu's were counted using an electronic registered colony counter fitted with a magnifying glass (MRC, Holon, Israel). Cfu's >250 per plate were recorded as TNTC (too numerous to count). Plates that demonstrated zero cfu's were recorded as <1. At each time point which generally consisted of triplicate enumerations derived from 3 separate bottles, the actual cfu's per ml were calculated by multiplying the actual number of colonies with the corresponding dilution factor. Thereafter, an average bacterial density per time point was recorded.

In the case of evidence of the appearance of fungal or molds by visual scrutinization, these were recorded according to the specific bottles and not taken for enumeration.

Preparation of Reports and Data Analysis:

For all experiments, reports are generated which consist of an outline of the experimental design (i.e. description of experimental AB groups), experimental purpose and calculated cfu's per ml (in triplicate) for NCB's and AB's. An average cfu/ml is then calculated for all experimental groups. Samples where cfu's are >250 were recorded as TNTC and samples in which cfu's were not determined noted as “nd”.

Evaluation of Efficacy of the Present Invention's Anti-Microbial Coating:

The anti-microbial efficacy of the present technology's coating was assessed by comparing the average cfu's per ml in AB's versus the corresponding cfu's ml in NCB's at 30 min and 4 hr post-inoculation. Positive anti-microbial effects were underscored by: a) demonstration that the present invention's coating reduced bacterial burden; b) positive efficacy met the originally-defined experimental aim and pre-determined target in reducing bacterial burden. A >2 fold log decrease in bacterial burden in AB's as compared to the control NCB's at the same time point were considered as significant.

Present Invention Efficacy Results Summation:

| | | | | | [symbol] | Production | | [symbol] after [symbol] | | | |
|---|---|---|---|---|---|---|---|---|---|---|---|
| | | | | | Challenge | after [symbol] ( [symbol] | | ( [symbol] | | | |
| | | Organism | | | [symbol] | challenge | | challange | | Number | |
| Organism | | ATCC | | [symbol] | ( [symbol] | volume/ [symbol] | | volume/ [symbol] | | of [symbol] | Location of [symbol] |
| Type | Organism | (MTO [symbol] ) | [symbol] | ATCC | [symbol] | percentage [symbol] ) | | percentage) | | [symbol] | [symbol] |
| Bacteria | E. coli | 11235 | N/A | N/A | 5/3 | 5/3 | 59.9% | 3/2 | 99.9% | >10 | [symbol] |
| | | 15322 | N/A | N/A | 5/3 | 5/3 | 59.9% | 3/2 | 59.9% | >10 | [symbol] |
| | [symbol] | 25297 | N/A | N/A | 7/7 | 5/3 | 99.9% | 5/3 | 79.1% | >10 | [symbol] |
| | [symbol] | | | | | | | | | | |
| | [symbol] | 9027 | N/A | N/A | 5 | 5 | 99.9% | 5 | 89.9% | 2 | [symbol] |
| | [symbol] | | | | | | | | | | |
| | [symbol] | 32352 | N/A | N/A | 4 | 2 | <50% | 4 | 58% | 2 | [symbol] |
| | | | | | | | | | | | [symbol] |
| | [symbol] | 13124 | N/A | N/A | 4 | 5 | 80% | 4 | 99% | 1 | [symbol] |
| | [symbol] | | | | | | | | | | [symbol] |
| | [symbol] | (420) | N/A | N/A | 4 | | | | | [symbol] | [symbol] |
| | [symbol] | | | | | | | | | | |
| | [symbol] | (3204) | N/A | N/A | 4 | | | | | [symbol] | [symbol] |
| | [symbol] | | N/A | N/A | 4 | | | | | [symbol] | |
| | [symbol] | | | | | | | | | | |
| | [symbol] | | N/A | N/A | 4 | | | | | [symbol] | |
| | [symbol] | | N/A | N/A | 4 | | | | | [symbol] | [symbol] |
| | [symbol] | | | | | | | | | | |
| [symbol] | [symbol] | [symbol] | E. coli | 12587 | 5 | 6 | 59.9% | 4 | 99.99% | 3 | The [symbol] |
| | | | | | | | | | | | [symbol] |
| | | | | | | | | | | | [symbol] |
| | [symbol] | [symbol] | | | 4 | 23 | 58% | 2.5 | 62% | 1 | [symbol] |
| | [symbol] | [symbol] | | | 4 | 23 | 58% | 2.5 | 62% | 1 | [symbol] |
| | [symbol] | [symbol] | E. coli | [symbol] | 8 | | | | | [symbol] | |
| [symbol] | [symbol] | N/A | N/A | N/A | 4 | <2 | <10% | 2 | <50% | 1 | [symbol] |
| | [symbol] | | | | | | | | | | |
| | [symbol] | N/A | N/A | N/A | 4 | | | | | [symbol] | |

¹Inoculation and sampling was conducted according to the WHO [symbol] .Testing.Protocol [symbol] .
²According to WHO method of calculating log removal [symbol] inactivation in terms of percentage (100 · 10 [symbol] where x is the number of log removal)
³Commercial lab located in [symbol] , [symbol]
⁴Multinational lab with a branch in india.
⁵Commercial lab located in R [symbol] , least.
⁶National lab part of the Ministry of Health.
⁷Commercial lab specializing in testing microbiological water purifier efficacy according to USEPA protocols. [symbol]
indicates data missing or illegible when filed

Test conditions: all tests were conducted in a medium volume of 500 mL. Test medium was prepared according to WHO harmonized testing protocol general test water (GTW):

| Constituent | Specification | Adjustment Materials (CAS#³) |
|---|---|---|
| Chlorine¹ mg/L) | <0.1 | None |
| pH | 7.0 ± 0.5 | Inorganic acid or base |
| | | Hydrochloric acid (7647-01-0) |
| | | Sodium hydroxide (1310-73-2) |
| TOC (mg/L) | 1.05 ± 0.95 mg/L | Tannic acid |
| | | (1401-55-4, Supplier: Alfa Aesar) |
| Turbidity (NTU) | <1 NTU | No adjustment |
| Temp (° C.) | 20 ± 3° C. | Not applicable |
| TDS (mg/L) | 275 ± 225 mg/L | Sea Salts, Sigma Chemical Company |
| | | (7732-18-5) |
| Alkalinity 2 (mg/L as CaCO3) | 80 ± 120 mg/L | Sodium bicarbonate (144-55-8) |

Absence Protocol for Measurement of ClO₂ (Chlorine Dioxide) Gas Release in Confined Volume):

Objective: Test the kinetics of ClO₂ (CDO, Chlorine dioxide) gas release, from a sheet of the present invention, to a confined volume which in conditions of 88% RH and ambient temperature.

Materials: 20 ml glass petri dish, Syringe, Silicone grease, Designated CDO measurement unit—sensor+rubber-sealed plastic box, Tested sheet, Optional—temperature/humidity sensor.

Protocol: (1) preparation. (A) Cut the sheet in appropriate size. Default size should contain 10 mg sodium chlorite precursor. At all times CDO accumulation in the box should not exceed 100 ppm since the sensor loses reliability above this threshold. (B) Weigh the sheet before the test. (C) While the box is open, place the sheet facing up in the bottom of the box. (D) For exact humidity and temperature measurement, place a humidity sensor inside the box. (Alternatively, one can assume RT and 88% RH instead of using this device). (E) Apply silicone grease in the interface between the sensor's fitting part and the cover of the box (around the area of the hole). (F) Place the petri dish inside the box and fill it with 10 ml of boiling water (a kettle may be of use). This is the water reservoir for humidity control in the box—reaches 88% RH 10 min after closing the box. (G) Close the cover of the box and make sure the sensor is well placed over the cover—gently rotate the sensor 20° to each side in order to spread the grease and assure appropriate sealing. See FIG. 23 for the CDO measurement array.

(2) CDO measurement: (A) Track CDO ppm reading in the display every several minutes (also temperature and humidity if device is used), according to the CDO release profile—fast release will require short intervals between readings, slower release may allow longer intervals. (B) When CDO concentration in the box starts to decrease after reaching the maximum concentration measured, test can be stopped.

(3) After the measurement: (A) Unplug the CDO sensor display from the electrical outlet. (B) TO PERFORM ONLY INSIDE A VENTED HOOD—open the cover of the box. (C) Weigh the sheet after the test (for water uptake calculations). (D) Discard the water. Clean the petri dish and the box with appropriate detergent to get rid of any CDO residues.

### Additional Non-Limiting Examples

Characterization of Various Inserts of the Water Project:

Objectives: To characterize the kinetics of ClO₂ (CDO, Chlorine dioxide) gas release from various inserts to a confined volume at 88 rh % and ambient temperature.

Rationale: Since liquid phase in-situ continuous ClOₓ-species measurement is not currently applicable, the kinetics in the gas phase may also provide meaningful insights.

Methods: Measurement of CDO burst in the gas phase: (1) Default size of the insert (active sheet) should contain 10 mg sodium chlorite precursor. (2) The concentration of CDO (in ppm units) is recorded every several minutes, according to the CDO release profile—fast release required short intervals between readings, slower release allowed longer intervals. (3) When CDO concentration starts to decrease after reaching the maximum concentration measured, the test is terminated. (4) Further details of the measurement is given in protocol OCTP004 for measurement of Cl_{O2} (Chlorine dioxide) gas release in confined volume.

The tested inserts are detailed in the table below:

| | CG8-H | SC | | Solvent/ | | SC precursor | SC conc. | [symbol] (KaMin) |
|---|---|---|---|---|---|---|---|---|
| Model # | Formulation | Formulation | Binder | Dispersant | Geometry | Form | [wt %] | conc. [wt %] |
| 1 | WE018 | WE004 | Vinnol (20 wt %) | EtOAc | Reverse | Solid | 40 | 0 |
| 2 | WE018 | WE004 | | | [symbol] | Solid | 40 | 0 |
| 3 | WE018 | WE004 | | | Regular | Solid | 40 | 0 |
| 4 | WE037 | WE038 | Elvacite (20 wt %) | EtOAc | Reverse | Solid | 20 | 0 |
| 5 | WE058 | WE048 | | | Reverse | Solution | 20 | 0 |
| 6 | WE058 | WE049 | | | Reverse | Solution | 40 | 0 |
| 7 | WE058 | WE050 | | | Reverse | Solution | 20 | 30 |
| 8 | WE052 | WE053 | Vinnacoat (20 wt %) | MEK | Reverse | Solution | 20 | 0 |
| 9 | WE052 | WE054 | | | Reverse | Solution | 40 | 0 |
| 10 | WE052 | WE055 | | | Reverse | Solution | 20 | 30 |
| 11 | WE075 | WE078 | Elvacite/Vinnacoat | EtOAc | Reverse | Solution | 40 | 0 |
| 12 | WE075 | WE079 | (80/20) | | Reverse | Solution | 20 | 30 |
| 13 | WE076 | WE080 | Elvacite/Vinnacoat | EtOAc | Reverse | Solution | 40 | 0 |
| 14 | WE076 | WE081 | (50/50) | | Reverse | Solution | 20 | 30 |
| 15 | WE077 | WE082 | Elvacite/Vinnacoat | EtOAc | Reverse | Solution | 40 | 0 |
| 16 | WE077 | WE083 | (20/80) | | Reverse | Solution | 20 | 30 |

Comments: The SC layer and CG8-H layer were 120 μm and 200 μm respectively. [symbol]
indicates data missing or illegible when filed

See FIGS. 22 A-D for additional illustrative examples.

Applications:

In some embodiments, the composition of the present invention can be included in/on, for example, but not limited to, an absorbent pad and/or a package insert. In some embodiments, the absorbent pad can be selected from the group consisting of: an absorbent pad used in the packaging for meat/poultry/fish, an absorbent pad used in the packaging for fruits/berries/vegetables, a feminine hygiene pad, diapers and incontinent products, non-woven materials, disposable absorbent cutting services, absorbent tray liners, bandages, drapes, mats, surface liners, absorbent pouches, or any combination thereof. In some embodiments, the composition of the present invention can be included in/on, for example, a package insert, e.g., but not limited to, an insert made of flexible plastic for purifying water, an insert made from wood for purifying water, a sticker, a plastic film and/or bag, an insert made of flexible plastic for use with consumer goods (e.g., but not limited to creams and/or gels), coated plastic sheets and paper sheets, or any combination thereof. In some embodiments, the package insert can be in the form of a sticker(s), where the package insert can be used in packages containing eggs.

Diapers

In some embodiments of the present invention, the present invention is a diaper including the following materials: i) a top layer, where the top layer is a non-woven polyester fabric, an optional layer comprising ii) a non-woven polyester fabric, vinyl chloride/vinyl acetate copolymer, iii) an acquisition layer, where the acquisition layer is woven/non-woven polyester/polyethylene fabric, iv) a fluff and/or a super absorbent polymer including cellulose fibers and sodium polyacrylate, v) at least one bottom layer, where the at least one bottom layer is polyethylene, and vi) an active layer, where the active layer has a reversed configuration of 2×15×0.3 cm, where the active layer includes polyvinyl chloride to function as a substrate, a vinyl chloride/vinyl acetate copolymer, sulfonated polystyrene, and sodium chlorite.

In some embodiments of the present invention, an active component for the diaper is prepared as follows: i) a Poly vinyl chloride thin sheet is corona treated and cleaned with 99% EtOH; ii) the sheet, comprising a first layer, is coated with a formulation containing sodium chlorite, poly vinyl chloride-co-vinyl acetate and ethyl acetate, using a 120 μm bar; iii) the coated layer is dried in a dry oven at 60° C. for 30 minutes to yield a layer containing 50% wt. sodium chlorite/50% wt. polymer (e.g., but not limited to, polyvinyl chloride-co-vinyl acetate); iv) a second layer is placed on top of the first layer, and the layer is coated with a formulation containing cation-exchange resin (CG8-H), poly vinyl chloride-co-vinyl acetate and ethyl acetate, using a 20004 bar; and v) the coated layer is dried in a dry oven at 60° C. for 30 minutes to yield a layer containing 50% wt. cation-exchange resin/50% wt. polymer.

In some embodiments of the present invention, the active layer contains two conjunct overlays: 1) a sodium chlorite salt and 2) a cation exchange resin. In some embodiments of the present invention, as water enters the diaper, the water is absorbed in the fluff (e.g., chopped cellulose fibers) and/or super absorbent polymer, creating a moist environment in the proximity of the adjacent active layer.

In some embodiments of the present invention, water then penetrates the porous layer through crevices, creating a chemical reaction between protons from the cation exchange resin (referred to as “activator”) and chlorite ions from the sodium chlorite salt (=precursor), to produce the chlorine dioxide radical (referred to as an “odor control substance”/“anti-microbial agent’).

In some embodiments of the present invention, the CDO radical reacts with the microorganisms and eliminates odor forming substances (“odor reduction”) and microorganisms present in the absorbed fluids.

In some embodiments of the composition of the present invention, the composition maintains odor reduction characteristics, e.g., but not limited to, having between a 0.1%-20% decrease (e.g., but not limited to, 1%, 2%, 3%, 4%, 5%, 6%, 7%, 8%, 9%, 10%, etc.) in odor reduction capabilities and/or anti-microbial activity after 6 months of storage in an original retail packaging (at a temperature of 40 degrees C., 50% RH).

In some embodiments, some embodiments of the composition of the present invention can include a coating including at least one active agent and at least one chlorite salt, where the coating is applied on a non-woven fabric in at least one shape (e.g., but not limited to, at least one circle, at least one strip/rectangle (continuous or non-continuous), a plurality of strips/lines, etc.), for example, but not limited to, between 0.5-3 cm wide (e.g., but not limited

present invention has an active agent of about 150-200 mg per non-woven fabric. In some embodiments, the composition of the present invention has an active agent of about 175-200 mg per non-woven fabric. In some embodiments, the composition of the present invention has an active agent of about 0.2-175 mg per non-woven fabric. In some embodiments, the composition of the present invention has an active agent of about 0.2-150 mg per non-woven fabric. In some embodiments, the composition of the present invention has an active agent of about 0.2-125 mg per non-woven fabric. In some embodiments, the composition of the present invention has an active agent of about 0.2-100 mg per non-woven fabric. In some embodiments, the composition of the present invention has an active agent of about 0.2-75 mg per non-woven fabric. In some embodiments, the composition of the present invention has an active agent of about 0.2-50 mg per non-woven fabric. In some embodiments, the composition of the present invention has an active agent of about 0.2-25 mg per non-woven fabric. In some embodiments, the composition of the present invention has an active agent of about 0.2-10 mg per non-woven fabric. In some embodiments, the composition of the present invention has an active agent of about 1-175 mg per non-woven fabric. In some embodiments, the composition of the present invention has an active agent of about 10-150 mg per non-woven fabric. In some embodiments, the composition of the present invention has an active agent of about 25-125 mg per non-woven fabric. In some embodiments, the composition of the present invention has an active agent of about 50-100 mg per non-woven fabric.

In some embodiments, the composition of the present invention has an active agent of about 60-80 mg per non-woven fabric. In some embodiments, the composition of the present invention has an active agent of about 60-70 mg per non-woven fabric. In some embodiments, the composition of the present invention has an active agent of about 70-80 mg

post-contact with urine, 1.7 after 2 hours post-contact with urine, 1.6 after 4 hours post-contact with urine, and 1.8 after 6 hours post-contact with urine. FIG. 8C shows anti-microbial activity, where both E. coli and Pseudomonas aeruginosa were reduced in number when placed in an environment with either coated Cyprus or coated Italian site material containing the composition of the present invention. FIG. 8D shows anti-microbial activity, where Klebsiella and Staphylococcus aureus were reduced in number when placed in an environment with either coated Cyprus or coated Italian site material containing the composition of the present invention.

Meat Wrap

In some embodiments of the present invention, the following materials are used in meat wrap: includes a) peripheral active component on external extended pad (e.g., 4×1.5×1 cm² of 120/200 m “reversed” configuration active sheets are attached to the perimeter of a standard poultry polystyrene tray) and/or b) active component on interior active pad (e.g., 2000 mg of MP-SC015 active formula (e.g., Hycar 26288/calcined clay (e.g., Kamin70C)/sodium chlorite) are dispersed over the perforated side (bottom side) of a standard poultry soaker pad). In some embodiments, the meat wrap further includes c) plastic separators (sterile PP parts which prevent overwrapping sheet collapsing over the poultry slice when trays are stacked one on top of the other in storage.) and/or d) sealed PE bag, where the PE bag is hermetically sealed using a designated bag-sealer.

In some embodiments of the present invention, the active components are prepared in the following manner: a) a PET thin sheet is corona treated (i.e., exposed to a high-voltage electric corona for elevation of the sheet surface energy and subsequent adhesion enhancement)) and cleaned with ethanol (>99%); b) the sheet (first layer) is coated with a formulation containing sodium chlorite, poly vinyl chloride-co-vinyl acetate and ethyl acetate, using a 120 micron bar; c) the coated layer is dried in a dry oven at 60° C. for 30 minutes to yield a layer containing 50% weight sodium chlorite/50% weight polymer; d) as a second layer, on top of the first layer, the sheet is coated with a formulation containing cation-exchange resin (CG8-H), poly vinyl chloride-co-vinyl acetate and ethyl acetate, using a 200 μm bar; and e) the coated layer is dried in a dry oven at 60° C. for 30 minutes to yield a layer containing 50 wt % cation-exchange resin/50 wt % polymer.

Active component on interior active pad: i) 2000 mg of MP-SC015 active formula containing “Hycar 26288” acrylic emulsion “Kamin70C” granular calcined clay and granular sodium chlorite are dispersed over the perforated side (bottom side) of a standard poultry soaker pad; and ii) the coated pad is then dried in a dry oven at 60° C. for 30 minutes to yield a layer containing 12% wt. sodium chlorite/24% wt. clay/64% wt. polymer. FIG. 10 illustrates the structure of the active pad, as displayed by cross-section.

In summary, the operation mechanism is a follows: a) a fresh moist poultry meat is packaged and stored in hermetically sealed bags, humid environment (>90% RH) is formed in the sealed tray atmosphere; b) as the tray enters a refrigerated storage condition, low temperature and humidity induce condensation of water droplets on exposed surfaces, in particular on the active sheet surface; c) humidity including condensed water droplets penetrates the porous layer structure and activates the formation and release of CDO to the tray atmosphere in a form of a “surge,” which means that all potential CDO yield is released to the tray atmosphere in 1-2 hours; and d) CDO molecules reach the poultry surfaces and react with bacteria by oxidation, showing antimicrobial activity in the form of growth inhibition (inhibition of spoilage relative to untreated reference meat).

Fruit and/or Vegetable Packaging

The present invention can also be utilized as fruit and/or vegetable packaging. The present invention can generate CDO in a packaging used for at least one fruit and/or vegetable. In an embodiment, the present invention increases shelf life and decreases decay of at least one fruit and/or at least one vegetable. In another embodiment, the present invention prevents and/or reduces microbial gastrointestinal infections (food safety). The present invention sufficiently decreases the rate of microbial growth on and/or in a fruit and/or vegetable. The present invention sufficiently decreases the number of microbes in at least one microbial population on at least one fruit and/or vegetable. In some embodiments, microbes are at least one bacterium, fungus, protozoan, and/or mold.

Milk

The ingredients regarding a milk-treating structure are a) 1 L carton and b) an active layer “sandwiched” configuration—vinyl chloride/vinyl acetate copolymer, sulfonated polystyrene, sodium chlorite, and fillers.

The active ingredient component is activated by the following steps: a) “sandwiched” configuration of active component, being an active film, is applied to the carton by one of the following methods: 1) spraying, 2) flexo/gravure printing, or 3) draw down on PET and its adhering to the carton. PET (100 microns) should be treated with corona and cleaned with 99% ethanol before formulation is applied; b) as a first layer, the carton/sheet is coated with a formulation containing cation-exchange resin (CG8-H), poly vinyl chloride-co-vinyl acetate and ethyl acetate, using a 200 micron bar. % NVS in dry film is 50% IX resin/50% binder; c) film drying at dry oven, 60° C. for 30 minutes; d) as a second layer, on top of the first layer, the carton/sheet is coated with a formulation containing granular sodium chlorite, poly vinyl chloride-co-vinyl acetate and ethyl acetate, using a 120 micron bar, % NVS in dry film—40% NaClO2/50% binder; e) film drying at dry oven, 60° C. for 30 minutes; f) as a third layer, the carton/sheet is coated with a formulation containing grinded cation-exchange resin (CG8-H), poly vinyl chloride-co-vinyl acetate and ethyl acetate, using a 200 micron bar; g) the formulation of the third layer may be different from the first layer (ratio between IX resin and binder, fillers may be added); and h) film drying at dry oven, 60° C. for 30 minutes.

In another embodiment, the active layer contains 3 conjunct overlays—one contains a sodium chlorite salt and 2 other contain a cation exchange resin. As milk is filled in the carton, water penetrates the porous active layer through crevices and voids, creating a chemical reaction between protons released from the cation exchange resin, the activator, and chlorite ions dissolved from the salt, the precursor, to produce chlorine dioxide radical (“antimicrobial agent”). The CDO radical reacts and eradicates the microorganisms present in the milk.

Sterilization System

Similar to the Velcorin™ DT Touch sterilization system, prior to the filling process, the active CDO solution and system scheme is utilized using CDO inserts, applied on site and supplied to the water system. The active CDO solution is ready to use and is delivered by an exterior bottle prepared before use and supplied to a spraying system.

Further examples utilizing spray machines for the sterilization processes are, e.g., Seridox-VP CD Sterilizers, the PET-Ascept process and filler (Krones), a bactericidal spray machine (Gongda Machine Co.). In some embodiments, a spray machine introduces droplets of chemicals or steam to a bottle/container, thereby sterilizing the bottle container by killing bacteria, virus, protozoa and/or fungus.

Examples of methods and/or uses of CDO are found in food safety (e.g., sterilization of surfaces and/or containers prior to, during, or after filling with food/beverage to kill bacteria, viruses, protozoa, and/or fungus), animal science (e.g., decontamination of rooms or facilities (including, but not limited to, HVAC ductwork, and equipment), isolators, pass-through chambers, biological safety cabinets), pharmaceutical and medical devices (e.g., decontamination), hospitals (including, but not limited to, rooms, clothing, curtains, bathrooms, furniture, equipment, and ambulances), decontamination of water resources (reservoirs, well water, or any other water delivered to consumers.). Additional examples of methods and/or uses of CDO are treatment of wounds, treatment of infections, treatment of oral infections (e.g., cavities and gums) or any soft tissue infection, treatment of biofilms, treatment of tissue grafts, and treatment of soft tissues.

Additional methods to produce CDO are the Direct Acid System generator, aqueous chlorine-chlorite generator, recycled aqueous chlorine or “French Loop” generator, gaseous chlorine-chlorite generator, gaseous chlorine-solids chlorite matrix generator, electrochemical generator, and acid/peroxide/chloride generator.

### Example: The Following is a Non-Limiting Example Showing, in One Embodiment, the Present Invention Used as a Water Purification Insert

Water uptake is a measurement of the amount of water absorbed into a model of known size in a known period of time.

### Example: The Following is a Non-Limiting Example Showing, in One Embodiment, an Evaluation of the Microbiocidal Efficacy of the Reverse Sodium Chlorite Antimicrobial Coatings

The of the reverse sodium chlorite antimicrobial coatings were tested for efficacy. Two different concentrations of the coatings were tested, a 10 ppm coating and a 25 ppm coating. The coatings were tested in two water types: General Test Water 1 (GTW 1) and General Test Water 3 (GTW 3). GTW 1 provides a clean water test system while GTW 3 provides a test system that stresses the disinfectant by having a high pH, high total organic carbon concentration (TOC), high total dissolved solids, a high turbidity and a low temperature. The coating concentrations were challenged in both water types with a Polio and Rotavirus mix and as a separate challenge, Cryptosporidium parvum. Prior to performing the efficacy test a neutralization test was performed to ensure that the antimicrobial activity could be effectively neutralized and that the antimicrobial/neutralizer combination did not have a toxic effect on the cell lines used for the assay.

The 25 ppm coatings were used for the neutralization study described in the protocol below. Twenty 10 ppm and twenty 25 ppm coatings were used in the efficacy study.

Results and Discussion:

Description of Challenge Organisms—C. parvum oocysts were acquired from Bunch Grass Farm, Deary, Id. (Lot#4-14) and were checked for viability potential of greater than 50% by excystation. Total excystation evaluation revealed a rate of 98.4%.

Poliovirus vaccine strain, ATCC VR-59 and Rotavirus SA-11, ATCC VR-899, were propagated using the Buffalo Green Monkey Kidney cell line, designated BV-BGMK. This cell line is sensitive to both Poliovirus and Rotavirus.

Toxicity/Neutralization Test—The toxicity and neutralization tests were carried out for virus and crypto. General test water (GTW) 1 and 3 was made to the specifications outlined in the protocol from a base water of de-chlorinated Benicia tap water. Final water qualities are detailed in Tables 1 and 2 below.

TABLE 5
General Test Water For Virus Challenge Test

| | Chlorine | | Turbidity | TOC | Temp | TDS |
|---|---|---|---|---|---|---|
| Water Type | (mg/L) | pH | (NTU) | (mg/L) | (° C.) | (mg/L) |
| 1 | ND | 7.46 | 0.21 | 1.7 | 20.4 | 190 |
| 3 | ND | 9.01 | 62 | 12 | 4.3 | 1470 |

[symbol]

Non Detect [symbol]
indicates data missing or illegible when filed

TABLE 6
General Test Water For Crypto Challenge Test

| | Chlorine | | Turbidity | TOC | Temp | TDS |
|---|---|---|---|---|---|---|
| Water Type | (mg/L) | pH | (NTU) | (mg/L) | (° C.) | (mg/L) |
| 1 | ND | 7.57 | 0.23 | 1.4 | 18.5 | 203 |
| 3 | ND | 8.93 | 32 | 11 | 6.8 | 1370 |

[symbol]

Non Detect [symbol]
indicates data missing or illegible when filed

The reduction results for the virus challenge are presented in Tables 7-10. The 25 ppm coating showed a greater than 6 log reduction in GTW 1 when allowed to react for as little as 30 minutes, but was unable to demonstrate a similar reduction when tested in GTW 3, even when allowed to react for 4 hours. The 10 ppm coating was only able to achieve a 2.5 log reduction in GTW 1, regardless of the reaction time, and when applied to GTW 3, the reduction demonstrated was even less.

TABLE 7
Virus Results for 10 ppm Treatment in GTW 1

| Untreated | 30 mins. | Log Reduction | 4 hrs. | Log Reduction |
|---|---|---|---|---|
| (PFU/mL) | (PFU/mL) | @ 30 mins. | (PFU/mL) | @ 4 hrs. |
| 9.8 × 10⁴ | 3.1 × 10² | 2.5 | 3.3 × 10² | 2.5 |

TABLE 8
Virus Results for 25 ppm Treatment in GTW 1

| Untreated | 30 mins. | Log Reduction | 4 hrs. | Log Reduction |
|---|---|---|---|---|
| (PFU/mL) | (PFU/mL) | @ 30 mins. | (PFU/mL) | @ 4 hrs. |
| 9.8 × 10⁴ | <0.1 | >6.0 | <0.1 | >6.0 |

TABLE 9
Virus Results for 10 ppm Treatment in GTW 3

| Untreated | 30 mins. | Log Reduction | 4 hrs. | Log Reduction |
|---|---|---|---|---|
| (PFU/mL) | (PFU/mL) | @ 30 mins. | (PFU/mL) | @ 4 hrs. |
| 2.3 × 10⁴ | 7.4 × 10³ | 0.5 | 3.6 × 10³ | 0.8 |

TABLE 10
Virus Results for 25 ppm Treatment in GTW 3

| Untreated | 30 mins. | Log Reduction | 4 hrs. | Log Reduction |
|---|---|---|---|---|
| (PFU/mL) | (PFU/mL) | @ 30 mins. | (PFU/mL) | @ 4 hrs. |
| 2.3 × 10⁴ | 3.3 × 10³ | 0.8 | 1.2 × 10³ | 1.3 |

Tables 11 and 12 present the results of the Crytosporidium challenge. The 25 ppm coating was able to demonstrate a 3 log reduction of C. parvum in GTW 1 when allowed to react for 4 hours but was not able to demonstrate the same when applied to GTW 3. The 10 ppm coating was only able to show a 2 log reduction when allowed to react for 4 hours in GTW 1.

TABLE 11
C. parvum Results for General Test Water 1

| Treatment | Log Reduction |
|---|---|
| 10 ppm, 30 mins | <2 |
| 10 ppm, 4 hrs | 2 |
| 25 ppm, 30 mins | <2 |
| 25 ppm, 4 hrs | 3 |

TABLE 12
C. parvum Results for General Test Water 3

| Treatment | Log Reduction |
|---|---|
| 10 ppm, 30 mins | <2 |
| 10 ppm, 4 hrs | <2 |
| 25 ppm, 30 mins | <2 |
| 25 ppm, 4 hrs | <2 |

Protocol for Evaluating the Microbiocidial Efficacy of Antimicrobial the Coatings Water Treatment System:

This screening protocol is designed to demonstrate the efficacy of the anti-microbial coatings, a water treatment product. This protocol calls for the challenge of a protozoan (Cryptosporidium parvum) and human enteric viruses (Poliovirus and Rotavirus) exposed for 30 minutes and 4 hours to two preparations of the test product in water of various quality. The reduction in microbial numbers over the test time period is recorded.

Media/Reagents: Record Sample Manufacturer Name, product name and other appropriate information. Different production doses of the test material as used in practice is used. Their identity as to lot number and any other identifying numbers or letters is recorded and the materials handled as called for in current Good Laboratory Practice as described in FIFRA 40 CFR 160(GLP).

Stock Cultures:

Poliovirus 1, LSc (ATCC VR-59); BGMK host cell line.

Rotavirus SA-11 (ATCC VR-899); BGMK host cell line.

Cryptosporidium parvum (Bunch Grass Farms; Deary, Id.)

HCT-8 host cell line

Sporo-Gio staining reagent (Waterborne, Inc., New Orleans, La.)

Neutralizing Fluid—The neutralizing fluid used to stop the disinfection action of the germicide must be specific to the chemical nature of the disinfectant used in the product. The efficacy of this fluid must be tested using the test protocol in Attachment A. The active agent in the coatings is chlorine dioxide based, thus the neutralizing fluid contains sodium thiosulfate. The composition is as follows:

250 g/L Na₂S₂0₄.5H₂0 (This concentration may vary depending upon the results of the neutralization tests) at pH 7.4; sterilize by autoclaving at 121° C. for 15 min.

General Test Water 1 (GTW 1):

Dechlorinated City of Benicia tap water that meets the following quality measures pH 7.5±1.0; TOC 0.1-5.0 mg/L; Turbidity≦5 NTU; Temperature 20° C.±5.0° C.; TDS 50-500 mg/L.

General Test Water 3 (GTW3). GTW 3 will have the following characteristics:

Dechlorinated City of Benicia tap water; pH 9.0±0.2; TOC≧10 mg/L (adjusted with Humic Acid); Turbidity≧30 NTU (adjusted ISO 1203-1 A2 Fine); Temperature 4° C.±1° C.; TDS 1350-1650 mg/L, adjusted with Sea Salts.

Equipment: Sterile culture tubes and plates, Glass culture slides, Dilution blanks containing PBS having various volumes, pH meter, Vortex mixer, Sterile pipettes, Nalgene bottle and cap (sterilized by autoclaving), top loading balance, Timer, Incubators: 37° C., Gas burner, Steam autoclave, Epifluorescence Microscope.

Detailed Procedure:

Procedure for determining the efficacy of neutralization (See Neutralization Test Protocol). Challenge viruses will be maintained at −80° C. and will be propagated following the protocol in the standardized procedure detailed below. Challenge Cryptosporidium parvum oocysts will be procured from Bunch Grass Farms (Deary, Id.). Testing procedure-for each type of insert (run in duplicate). Challenge viruses. A minimum of three mLs of a 107 pfu/mL suspension of both viruses will be added to 6 liters of General Test Water in a 10 liter carboy with a stir bar and mixed for approximately 10 mins Collect a 25 mL untreated sample. Take 1 (one) insert out of the bag. Reseal bag to avoid contact of other inserts with moisture. Put 1 (one) insert inside 500 ml Nalgene bottle. Fill 500 ml of test medium (mixed already with ingredients and inoculated) into bottle. Close cap of bottle. Shake thoroughly for 10 seconds. Leave to stand for 15 minutes at room temperature for GTW 1 and 4° C. for GTW 3. After 15 minutes, shake thoroughly for 10 seconds. Leave to stand for 15 minutes (total time of 30 minutes) at room temperature for GTW 1 and 4° C. for GTW 3, depending upon test segment. Sample bottle according to protocol. At the end of the exposure period, the coated material will be aseptically removed from the beakers and 3 mL of a 25% sodium thiosulfate solution will be added to stop all further antimicrobial action. Repeat steps (filling 500 mL test medium through removable of coated material from beakers and halt antimicrobial activity of this material) with the viruses for 4 hours (240 min). Submit samples for virus assay.

Challenge protozoa. Add sufficient volume of the stock C. parvum to achieve a final concentration of 10⁶-10⁷ oocysts in 6 L of General Test Water 1 and 3 in a 10 L carboy. Repeat steps (filling with 500 mL test medium through submitting samples for assay) with the protozoa. Submit samples for oocyst infectivityassay.

Quality Control:

Good Laboratory Practice (GLP) as described in FIFRA 40 CFR 160 will be followed. All viral and protozoan cultures will be checked for purity and infectivity. Appropriate negative controls will be included in all phases of the testing. Calculation: (Log₁₀ of Untreated Target Organism)−(Log₁₀ Treated Target Organism)=Log₁₀ Reduction in Numbers.

Data reporting: For viruses the average pfu/mL is reported and the percent and Log₁₀ reduction will be calculated. For protozoans, the most probable number associated with the infectivity slide format and Log₁₀ reduction will be calculated.

Protocol for Testing the Neutralization of Disinfection Action:

When testing for disinfection efficacy, it is essential that the biocidal activity of the active ingredient be neutralized at the end of a given time period. The ability of a selected neutralizing agent to achieve this goal must be determined for each disinfectant and for each challenge microorganism. In addition, it must also be demonstrated that the combination of the neutralizer with the disinfectant material will not be toxic to the cell lines used in the assays. The ability to neutralize the active ingredient is to be determined using the protocol described below.

A 0.15% final sodium thiosulfate concentration is sufficient in the neutralized solution. This study will confirm that a final concentration of 0.15% sodium thiosulfate will neutralize the active ingredient(s) and that the combination of active ingredient(s) with sodium thiosulfate will be non-toxic to the cell lines. (See, ASTM Standards on Materials and Environmental Microbiology, 2^{nd} Edition, Sec E1054-91, 1993, which is herein incorporated by reference in its entirety.)

Reagents:

Stock Cultures: BGMK cell line, Poliovirus type 1 LSc (vaccine strain), HCT-8 cell line, Cryptosporidium parvum, 25% solution of sterile sodium thiosulfate, antimicrobial coated material, General Test Water 1, Dechlorinated City of Benicia tap water that meets the following quality measures: pH 7.5±1.0, TOC 0.1-5.0 mg/L, Turbidity≦5 NTU, Temperature 20° C.±5.0° C., and TDS 50-500 mg/L.

General Test Water 3 (GTW3). GTW 3 will have the following characteristics: Dechlorinated City of Benicia tap water, pH 9.0±0.2 TOC≧10 mg/L (adjusted with Humic Acid), Turbidity≧30 NTU (adjusted ISO 1203-1 A2 Fine), Temperature 4° C.±1° C., and TDS 1350-1650 mg/L, adjusted with Sea Salts.

Equipment: Sterile culture tubes and plates, Glass culture slides, Dilution blanks containing PBS, various volumes, pH meter, Vortex mixer, Sterile pipettes, Beakers, various sizes, Top loading balance, Timer, Incubators: 37° C., Gas burner, Steam autoclave, and Epifluorescence Microscope.

Procedure:

A sample of each of the submitted production lots of the coated material is evaluated. The un-seeded test material consists of one 500 mL volume of GTW 1 and GTW 3 combined with the neutralizing agent (3 mL of 25% sodium thiosulfate). The coated material is added to 500 mL of unseeded GTW 1 and GTW 3. At the end of the mixing period incubate at 4° C. for 4 hours (the maximum dose of the study). At the end of the incubation period aseptically remove the coated material, add 3 mL of 25% sodium thiosulfate and submit to tissue culture assay to check for toxicity to the cell lines. Known concentrations of virus or C. parvum will be added to the neutralized solution and assayed to demonstrate that the neutralized solution does not negatively impact the assays and create false negative outcomes. Control samples are processed in the same manner as above.

Quality Control: Good Laboratory Practice (GLP) as described in FIFRA 40 CFR 160 will be followed. Appropriate negative controls will be included in all phases of the testing regimen.

Calculations: The number of virus present in the control sample and the test samples will be determined using standard plate counting procedures. For protozoa, the number added will be determined by microscopy.

Data Reporting: The data will be reported as colony forming units (cfu) per mL. Neutralization will be considered effective if there is <one log 10 difference between the test results and the control results.

### Example: Release Kinetics

Experiments testing release kinetics of the composition of the present invention were performed at 25° C. and 95-100% relative humidity (RH). Hydrophobic polymer (V) dividing layer(s) and top coats on silk printed (SPL) stickers were applied for testing and evaluating the effect of V top coats and dividing layers on the CLO₂ generated kinetic profiles.

Stickers (NaClO2 (S. C.) (AG192/007) and proton donor (H) (AG009/002) formulations) were prepared as described herein. A V formulation (AG016/02) was prepared and used for all top coats and dividing layers. Stickers of the structures seen below were silk printed according to the procedures described herein, employing the coarse screen for all V layers. All layers were dried at 60° C. for 30 min before the next layer was printed. Table AA below shows the structure of the stickers tested:

TABLE AA

| Model No. | Class | structure |
|---|---|---|
| 1 | Control | S.C/H/H |
| 2 | One V layer | S.C/V/H/H |
| 3 | | S.C/H/V/H |
| 4 | | S.C/H/H/V |
| 5 | Two V layers | S.C/V/H/V/H |
| 6 | | S.C/H/V/H/V |
| 7 | | S.C/V/H/H/V |
| 8 | Three V layers | S.C/V/H/V/H/V |

Gas-Phase Measurement Protocol:

Methods

Hydrophilic Layers in the Coating System

NaClO2 (S.C.) (AG192/007) and proton donor CG8-H (H) (AG009/002) formulations were prepared according to previous procedures and used for all stickers configurations.

The hydrophilic formulations are comprised of: (1) Hycar 26288 which is carboxylate acrylic copolymer latex combined with Sodium chlorite and (2) Elvacit 2046, which is a high molecular weight iso-butyl/n-butyl methacrylate polymer combined with the proton donor—acid component. These binders, combined with the highly hygroscopic components, form the hydrophilic films that generate the driving force and process of the water absorption and by that enable the chemical reaction for generating CDO in the sticker coating.

Hydrophobic Layer in the Coating System

A Vinnacoat LL 8100 (V) formulation (AG016/02) was prepared and used for all hydrophobic top coats and dividing layers. Vinnacoat LL 8100 is a hydrophobic styrene-olefin copolymer with carboxyl functional groups. The formulation, when dried, results in an exceptionally hydrophobic film that decelerates the water absorption rate in to the coating system. As a result, it modifies the kinetics of CDO generating chemical reaction in the sticker

Stickers of the structures seen below (models 1-8)were silk printed according to the procedures herein, employing a 90° oriented Teflon screen (pw47/435, 200 mesh, 47% open area) for the S.C layer and a 45° polyester screen (12/300, 300 mesh, 39% open area) for the V and H layers.

All layers were dried at 60° C. for 30 min before the next layer was printed.

Sticker Models structure and dimensions

The structure and dimensions of the sticker models are show in Table CC below:

TABLE CC

| | | | S.C | V1 | CG8H(1) | V2 | CG8H (2) | V3 | |
|---|---|---|---|---|---|---|---|---|---|
| | Model | Model | thickness | thickness | thickness | thickness | thickness | thickness | Overall |
| Model | class | description | (Mm) | (Mm) | (Mm) | (Mm) | (Mm) | (Mm) | thickness |
| 1 | control | S.C/H/H | 28.9 | 0 | 22.9 | 0 | 33.5 | 0 | 85.3 |
| 2 | One V | S.C/V/H/H | 28.9 | 16.7 | 20.3 | 0 | 27.5 | 0 | 93.4 |
| 3 | layer | S.C/H/V/H | 28.9 | 0 | 22.9 | 10.9 | 25.7 | 0 | 88.4 |
| 4 | | S.C/H/H/V | 28.9 | 0 | 22.9 | 0 | 33.5 | 8.7 | 94.1 |
| 5 | Two V | S.C/V/H/H/V | 28.9 | 16.7 | 20.3 | 0 | 27.5 | 8.3 | 101.8 |
| 6 | layers | S.C/V/H/V/H | 28.9 | 16.7 | 20.3 | 9.05 | 40.7 | 0 | 115.7 |
| 7 | | S.C/H/V/H/V | 28.9 | 0 | 22.9 | 10.9 | 25.7 | 14.5 | 103.0 |
| 8 | Three V | S.C/V/H/V/H/V | 28.9 | 16.7 | 20.3 | 9.05 | 40.7 | 6.8 | 122.5 |
| | layers | | | | | | | | |
| Average | | | 28.9 | 16.7 | 21.6 | 9.975 | 31.8 | 9.6 | |
| thickness: | | | | | | | | | |

Sticker Model Dimension Ranges

Below, in Table DD, are exemplary ranges of layer thickness. These ranges are an example of possible layer thicknesses and are not a limiting criterion for the proper operation of the sticker.

TABLE DD

| S.C | CG8-H | V | Overall thickness |
|---|---|---|---|
| 5-60_{(Mm)} | 15-100_{(Mm)} | 1-50_{(Mm)} | 20-210_{(Mm)} |

A chlorine dioxide measurement chamber was constructed using a “Duran” glass reactor (2.0 L, model DN120) equipped with a chlorine dioxide sensor (ATi, 0-100 ppm) and a CPU fan used to circulate the air in the chamber.

Measurements:
1. The bottom part of the apparatus was filled with 10 ml of deionized water.
2. Lid closed tight and the apparatus was placed in a preheated oven at 25° C. Circulation fan was turned on.
3. The apparatus was left to equilibrate for ˜30 min.
4. A small piece (˜1×2 cm) of double sided tape was attached to the back of the sticker to be tested.
5. Sample septum was opened and the sticker attached to the plastic holder so it was aligned with the holder and centered.
6. Apparatus neck was wiped with a Chemwipe to ensure it is dry.
7. Sample septum inserted with the sticker facing the center of the apparatus.
8. t₀ voltage was recorded (39.5 mV).

FIG. 25 shows the results of testing the above models. A summary of experimental results is show in Table EE below.

TABLE EE

| | | | Time to | Retention | Exposure |
|---|---|---|---|---|---|
| Model | | Max ClO₂ | max | time | time |
| No. | Model | (ppm) | (hr.) | (hr.) | (hr.) |
| 1 | S.C/H/H | 45.813 | 1.15 | 0.033 | 9.3 |
| 2 | S.C/V/H/H | 19.563 | 4.183 | 0.1 | 20.61 |
| 3 | S.C/H/V/H | 27.066 | 1.983 | 0.033 | 21.58 |
| 4 | S.C/H/H/V | 21.688 | 5.817 | 0.167 | 21.33 |
| 5 | S.C/V/H/V/H | 5.938 | 14.37 | 0.633 | 50.02 |
| 6 | S.C/H/V/H/V | 20.125 | 3.25 | 0.1 | 21.37 |
| 7 | S.C/V/H/H/V | 11.688 | 13.283 | 1.65 | 36.85 |
| 8 | S.C/V/H/V/H/V | 1.2 | 44 | 10 | 55.38 |

As used herein, the “retention time” refers to the time difference from the insertion of the sticker in to the measurement chamber (t₀), to the time when there was the initial concentration measurement of chlorine dioxide in the chamber.

As used herein, the “exposure time” refers to the time from the beginning of chlorine dioxide generation to a zero reading of the sensor.

Results: One V Layer

As shown in Table BB above, all V dividing layers and top coats slowed down and extended the time duration of ClO₂ generation from the sticker. Additionally, all single V layer models provided immediated ClO₂ generation when exposed to high humidity (about 95%), with the highest retention time (about 6 minutes) associated with a V top-coat. Model 4 provided a “delayed release” kinetic profile, with a Cmax of 21 ppm attained at 5.8 hours. All three models (e.g., models 2, 3, and 4) including a single V layer demonstrated the presence of ClO₂ for about 20 hours, which is more than twice the release time that was measured for model 1, which does not include a V layer.

Results: Two V Layers

Also shown in Table BB above, models 5 and 7 containing two V layers resulted in providing an extended concentration peak, indicating that the ClO₂ in the composition maintained a relatively constant concentration for an extended period of time. Thus, ClO₂ was generated at a rate of increased consistency, indicating a slow release and longer lasting profile (a “delayed” release). Model 7 provided a higher maximum concentration than model 5 and longer retention time. Model 5 provided results of a typical slow release profile, resulting in a short retention time combined with a long duration of generating ClO₂, achieving a maximum concentration of 5.9 ppm and maintaining a concentration of 4-6 ppm for more than 10 hours. The profile of model 6 was surprisingly rapid, with a Cmax of about 20 ppm after 3.25 hours.

Results: Three V Layers

Model 8 contained three V layers, which, upon testing, provided a slow and reduced ClO₂ generation with a retention time of about 10 hours and a Cmax of 1.2 ppm after 44 hours. Model 8 was releasing ClO₂ at low concentrations for longer than 72 hours.

Discussion

The models 2-8, which contained V dividing layers and coats, provided surprisingly different results when tested. Models 4 and 7 provided a “delayed” kinetic profile. Models having a top H layer (i.e., 1, 2, 3, 5) demonstrated a reduced retention time relative to the other models tested in these experiments. Models which had a V layer between the S.C. (salt) and H (hydrophobic) layers (i.e., models 2, 5, 6, 7) demonstrated both a small inhibitory effect, by observation of longer retention time, and a strong slowing effect, demonstrated by a longer time to attain ClO₂ Cmax. Models including a V layer between the two H layer (i.e., models 3, 5, and 8) demonstrated a moderate effect on the release profiles, slowing the reaction to provide an extended ClO₂ generation time and extended time to reach Cmax. Sticker models 1-8 provided different ClO₂ release profiles/release kinetics, displaying release profiles from about 5 hours to more than 3 days. ClO₂ concentrations ranged from exhibiting a Cmax of about 1 ppm (model 8) to 40 ppm (model 1).

Experimental Results: Gas Phase Measurements of Models 1, 4, and 7 in 250 Gram Clamshell Containers

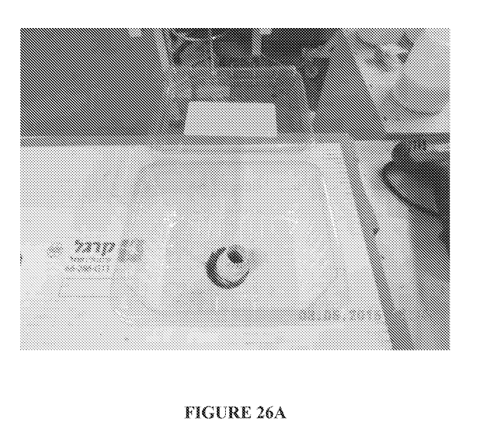

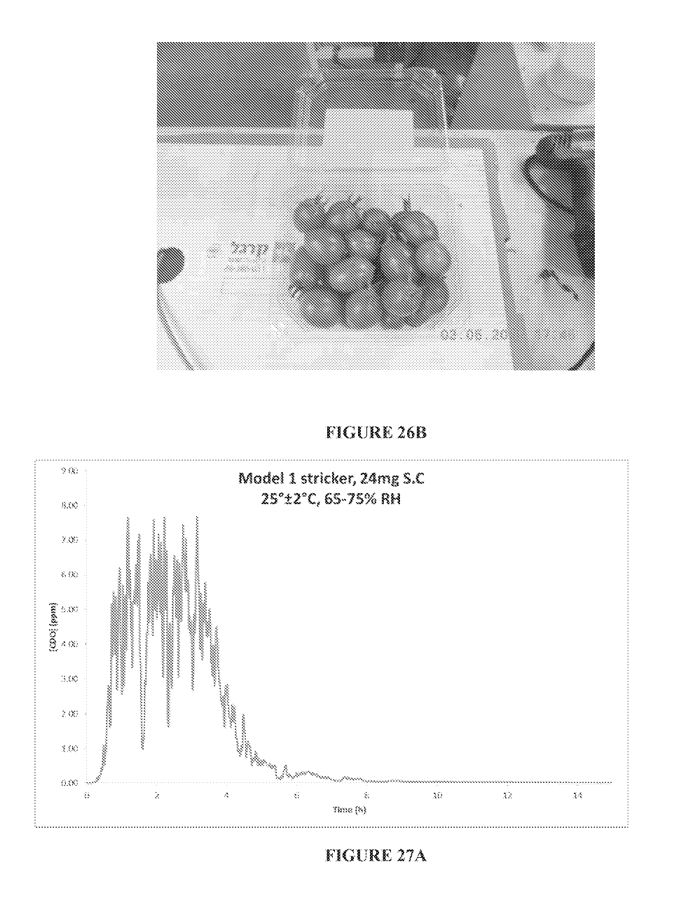

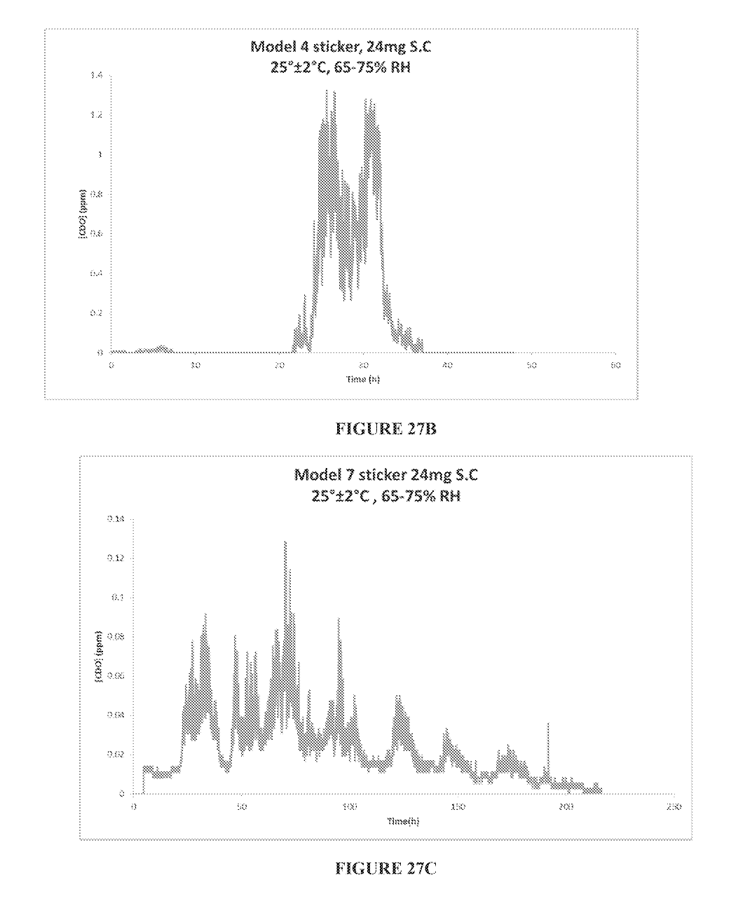

Model 1, 4 &7 stickers were analyzed by chlorine dioxide release experiments, using in a 250 gram(g) clamshell container of grape tomatoes (size: 12.5×8.5×8.5 cm). The stickers were placed on the lid of the clamshell (3 stickers, 24 mg SC). Measurements were taken using an Ati chlorine dioxide sensor (0-5 ppm) connected to a datalogger. Voltage readings were converted to ppm values based on ATi chlorine dioxide transmitter part-per-million readings which were used to prepare a Voltage-ppm calibration curve. The chlorine dioxide sensor's head was inserted through a hole at the bottom of the clamshell so that the sensor's opening was approximately flush with the center of the clamshell. The apparatus is shown in FIG. 26A and FIG. 26B and results are shown in FIG. 27A-C.

Distribution of Active Material in a Fluid

In some embodiments, the system includes a floating device that is configured to:

1) 1) Keep active material on upper part of the medium;

2) If active material is more dense than medium, prevent the active material from sinking to the bottom;

3) allow the micro-organisms to sink to the bottom of the bottle and settle there;

4) Locate active material and active surface at the top layers and surface of a liquid in a container;

5) Enable simple, fast and safe removal of an active insert in a container with a liquid; and/or

6) Enable an active insert with a simple removal for various/changing amounts of liquid in a container (the longer wave fom1ing insert that can also be a buoy).

In some embodiments, the floating device ailovvs for Brownian motion of active material so as to results in good distribution in the medium. In some embodiments, the floating material is configured so as to result in release from upper part of medium and results in better distribution than release from lower or middle part, because of gravity. In some embodiments, the floating device is configured for better distribution that results in higher possibility of “meeting” micro-organism and reaction.

In some embodiments, the floating material may include, but is not limited to foamed polystyrene, polypropylene, rubber, a polymer that can be foamed-using air bubbles, and any sufficiently buoyant material for supporting an active material.

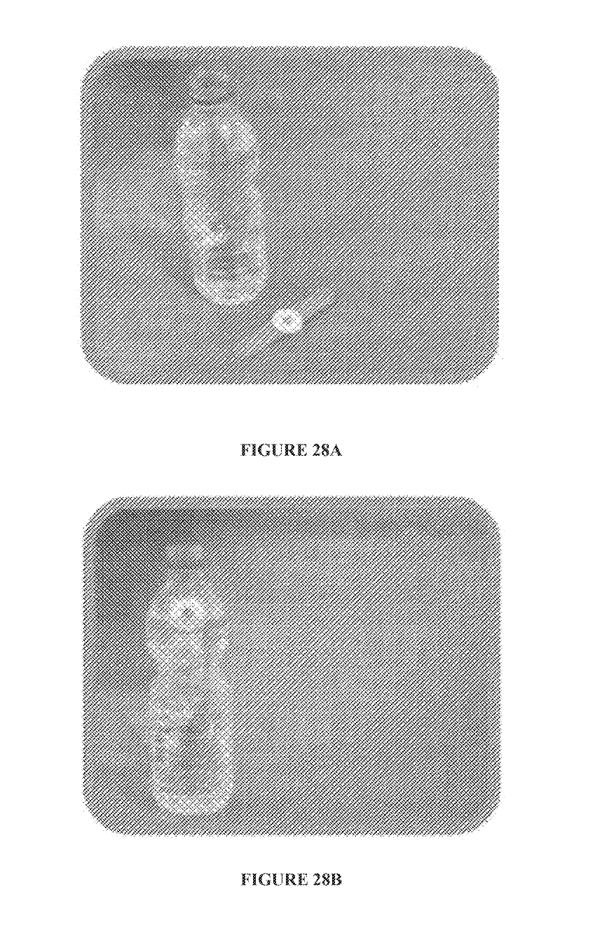

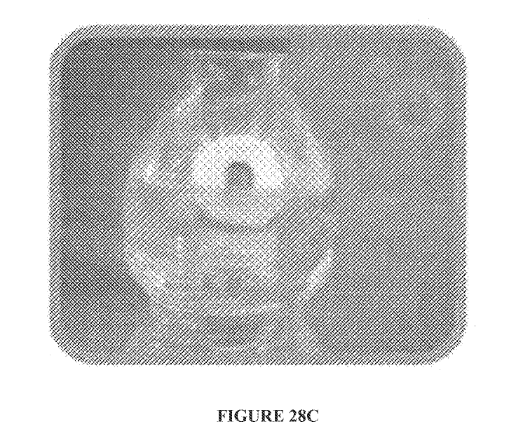

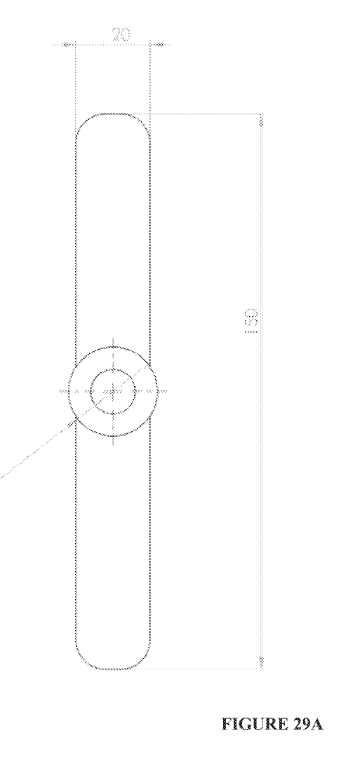

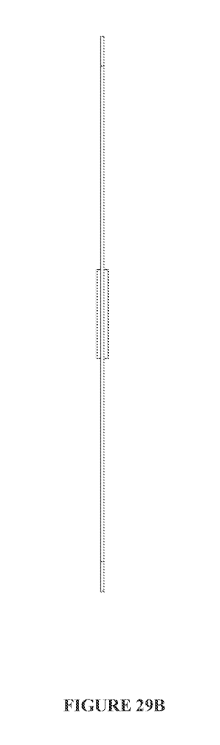

FIGS. 28A-28C show the action of an embodiment of the floating device. FIGS. 29A and 29B show an embodiment of the floating device.

In some embodiments, the present invention provides for a composition, including: a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the composition is contacted with an aqueous liquid, the sufficient first amount of the first active agent dispersion and the sufficient second amount of the at least one chlorite salt dispersion results in a generation of chlorine dioxide radicals at a rate ranging from 0.001 mg/min-0.02 mg/min. In some embodiments, the at least one chlorite salt dispersion is selected from the group consisting of: sodium chlorite, potassium chlorite, barium chlorite, calcium chlorite, magnesium chlorite, and any combination thereof. In some embodiments, the sufficient first amount of the first active agent dispersion is in a first layer, and the sufficient second amount of the at least one chlorite salt dispersion is in a second layer. In some embodiments, the active agent dispersion and the at least one chlorite salt dispersion are configured in the composition to define a plurality of cavities. In some embodiments, each cavity of the plurality of cavities measures between 0.5-50 micrometers in length. In some embodiments, the first active agent dispersion has a pKa of 0.1-1.5. In some embodiments, the composition is configured to allow for a water uptake measurement ranging from 10-90% over 1 hour. In some embodiments, the composition further includes: a substrate component in contact with of the first active agent dispersion or the at least one chlorite salt dispersion, where the substrate component includes polyethylene terephthalate, high-density polyethylene, low-density polyethylene, polypropylene, polystyrene, polyamide, polyvinylchloride, or any combination thereof. In some embodiments, the composition further includes: a protection component configured to reduce a reaction between the first active agent dispersion and the at least one chlorite salt dispersion, where the protection component includes an acrylic dispersion, a styrene acrylate dispersion, a poly urathene, an epoxy co-polymer, a cellulose, a polymer or copolymer dispersion, or any combination thereof, and where the protection component is in contact with at least the first active agent dispersion or the at least one chlorite salt dispersion. In some embodiments, the composition further includes: a neutralizing agent selected from the group consisting of: sodium thiosulfate, ferrous chloride, ferrous sulfate, vitamin E, and any combination thereof. In some embodiments, the composition further includes: a second active agent dispersion having a pKa of 0.1-2.0, where the at least one chlorite salt dispersion is in contact with the first active agent dispersion and the second active agent dispersion. In some embodiments, the sufficient second amount of the second active agent dispersion has a pKa of 0.1-1.5. In some embodiments, the sufficient first amount of the first active agent dispersion is in a first layer, where the sufficient second amount of the at least one chlorite salt dispersion is in a second layer, where the sufficient second amount of the second active agent dispersion is in a third layer, and where the second layer is positioned between the first layer and the third layer. In some embodiments, the composition further includes: a stabilizing agent selected from the group consisting of: ammonia, methylamine, sodium hydroxide, sodium bicarbonate, Purolite A200-MBOH, Dow FPA-55, a basic zeolite, and any combination thereof.

In some embodiments, the present invention provides for a composition, including: a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the sufficient second amount of the at least one chlorite salt dispersion is tested in the composition with the sufficient first amount of the first active agent dispersion, the sufficient first amount of the first active agent dispersion and the sufficient second amount of the at least one chlorite salt dispersion are contacted with an aqueous liquid, and chlorine dioxide radicals are generated at a rate ranging from 0.001 mg/min-0.02 mg/min so as to result in a microbial reduction of between 2 log CFU/mL and 10 log CFU/mL from between 1 and 60 minutes.

In some embodiments, the present invention provides for a composition including a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the composition is contacted with an aqueous liquid, the sufficient first amount of the first active agent dispersion and the sufficient second amount of the at least one chlorite salt dispersion results in a generation of chlorine dioxide radicals at a Cmax ranging from 15 ppm-25 ppm from between 4 hours-6 hours.

In some embodiments, the first active agent dispersion has a pKa of 0.1-1.0. In some embodiments, the first active agent dispersion has a pKa of 0.1-0.5. In some embodiments, the first active agent dispersion has a pKa of 0.5-2.0. In some embodiments, the first active agent dispersion has a pKa of 1.0-2.0. In some embodiments, the first active agent dispersion has a pKa of 1.5-2.0. In some embodiments, the first active agent dispersion has a pKa of 1.0-1.5.

In some embodiments, the plurality of particles has a median diameter of between 1-1000 micrometers. In some embodiments, the plurality of particles has a median diameter of between 10-1000 micrometers. In some embodiments, the plurality of particles has a median diameter of between 100-1000 micrometers. In some embodiments, the plurality of particles has a median diameter of between 500-1000 micrometers. In some embodiments, the plurality of particles has a median diameter of between 0.1-500 micrometers. In some embodiments, the plurality of particles has a median diameter of between 0.1-100 micrometers. In some embodiments, the plurality of particles has a median diameter of between 0.1-10 micrometers. In some embodiments, the plurality of particles has a median diameter of between 0.1-1 micrometers. In some embodiments, the plurality of particles has a median diameter of between 1-500 micrometers. In some embodiments, the plurality of particles has a median diameter of between 10-100 micrometers.

In some embodiments, the present invention provides for a composition, including: a sufficient first amount of a first active agent dispersion; where the first active agent dispersion has a pKa of 0.1-2.0, where the first active agent dispersion is selected from the group consisting of: an acid cation exchange resin, an acidic zeolite, an acidic clay, an organic acid, an inorganic acid, and any combination thereof, and where the first active agent dispersion includes a plurality of particles; where the plurality of particles has a median diameter of between 0.5-1000 micrometers; and a sufficient second amount of at least one chlorite salt dispersion; where, when the composition is contacted with an aqueous liquid, the sufficient first amount of the first active agent dispersion and the sufficient second amount of the at least one chlorite salt dispersion results in a generation of chlorine dioxide radicals at a Cmax ranging from 5 ppm-15 ppm from between 10 hours-20 hours. In some embodiments, the present invention is a product, including an absorbant pad, where the absorbent pad includes the composition of claim 1. In some embodiments, the present invention is a product, including a package insert, where the package insert includes the composition of claim 1.

In some embodiments, the acid cation exchange resin can include a sulfonic or phosphoric functional group(s) in a hydrogen form or a sodium form (e.g., but not limited to, ResinTech CG8-H, Dow Amberlyst 15, Dow FPC-23H, Purolite NRW1160, Purolite C-100, Sulfonated styrene-ethylene-butylene-styrene polymers (e.g., but not limited to, Kraton Nexar MD-9200)), or any combination thereof. In some embodiments, the acidic zeolite can include, but is not limited to, zeolyst zeolite Y CBV 720, zeolyst zeolite beta CP811C-300, zeolyst mordenite CBV 10A, or any combination thereof. In some embodiments, the organic acid can include oxalic acid, phosphoric acid, sulfonic acid (e.g., but not limited to, p-Toluenesulfonic acid, aminomethylphosphoric acid), or any combination thereof. In some embodiments, the inorganic acid can include, but is not limited to, sodium hydrogen sulfate, sulfuric acid, phosphoric acid, iodic acid, or any combination thereof.

In some embodiments, the substrate component can be cellulose, acrylics, polyvinyl chloride, wood, glass, or metal.

In some embodiments, the protection component can be an acrylic dispersion (e.g., but not limited to, Lubrizol hycar 26288, celanese vinamul 3171, Lucite elvacite 2044, Lucite elvacite 2046, Lucite elvacite 4044, or any combination thereof), a styrene acrylate dispersion (e.g., but not limited to, DSM neocryl A-2092 or A-1095, BASF ioncryl DFC 3030, or any combination thereof), a polyurathene (e.g., but not limited to, DSM neorez), an epoxy co-polymer, a cellulose (e.g., but not limited to, ethyl cellulose, methyl cellulose, carboxymethyl cellulose, or any combination thereof; e.g., but not limited to, Dow ethocel, Dow methocel, Dow walocel, Ashland Aqualon, or any combination thereof), a polymer or copolymer dispersion (e.g., but not limited to, Polyvinylchloride, Polyvinyl acetate and their co-polymers (e.g., Wacker Vinnol H30/48M, DOW VAGH); polyvinyl butyral (e.g., but not limited to, Kuraray mowital, Eastman butvar, or any combination thereof), polyvinyl alcohol (e.g., but not limited to, kuraray mowiol and excelval, or any combination thereof), styrene-olefin co-polymers (e.g., but not limited to, wacker vinnacoat LL 8100), vinyl acetate ethylene co-polymers (e.g., but not limited to, wacker vinnapas EP 8010, vinavil EVA 202, or any combination thereof), polyvinyl pyrrolidone, polyethylene glycol and co-polymers (e.g., but not limited to, BASF kollidon, BASF kollicoat, or any combination thereof), or any combination thereof.

In some embodiments, a stabilizing agent can include: ammonia solution (e.g., but not limited to, 25% ammonium hydroxide); organic bases (e.g., but not limited to, methylamine, triethanolamine, monoethanolamine, AMP (2-amino-1-methyl-1-3 propandiol), DMAMP, and T(HM)AM)); inorganic bases, e.g., sodium hydroxide, potassium hydroxide, sodium bicarbonate, or any combination thereof; anion exchange resins, e.g., purolite A-300 MBOH, Dow FPA-55, or any combination thereof; basic zeolite, e.g., 4A, 13x or any combination thereof. In some embodiments, a weak base can range in pKa from between 7.01 and 11 (e.g., but not limited to, 7.01, 8, 9, 10, 11). In some embodiments, a strong base having a pKa over 11, e.g., from between 11 and 14 (e.g., but not limited to, 11, 12, 13, 14).

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A composition, comprising:
a sufficient first amount of a first active agent dispersion;
wherein the first active agent dispersion has a pKa of 0.1-2.0,
wherein the first active agent dispersion is selected from the group consisting of:
an acid cation exchange resin, an acidic zeolite, an acidic clay, an organic acid, an inorganic acid, and any combination thereof, and
wherein the first active agent dispersion comprises a plurality of particles;
wherein the plurality of particles has a median diameter of between 0.5-1000 micrometers; and
a sufficient second amount of at least one chlorite salt dispersion;
wherein, when the composition is contacted with an aqueous liquid, the sufficient first amount of the first active agent dispersion and the sufficient second amount of the at least one chlorite salt dispersion results in a generation of chlorine dioxide radicals at a rate ranging from 0.001 mg/min-0.02 mg/min.

2. The composition of claim 1,
wherein the at least one chlorite salt dispersion is selected from the group consisting of: sodium chlorite, potassium chlorite, barium chlorite, calcium chlorite, magnesium chlorite, and any combination thereof.

3. The composition of claim 1,
wherein the sufficient first amount of the first active agent dispersion is in a first layer, and
wherein the sufficient second amount of the at least one chlorite salt dispersion is in a second layer.

4. The composition of claim 1, wherein the active agent dispersion and the at least one chlorite salt dispersion are configured in the composition to define a plurality of cavities.

5. The composition of claim 4, wherein each cavity of the plurality of cavities measures between 0.5-50 micrometers in length.

6. The composition of claim 1, wherein the first active agent dispersion has a pKa of 0.1-1.5.

7. The composition of claim 1,
wherein the composition is configured to allow for a water uptake measurement ranging from 10-90% over 1 hour.

8. The composition of claim 1, further comprising:
a substrate component in contact with of the first active agent dispersion or the at least one chlorite salt dispersion,
wherein the substrate component comprises polyethylene terephthalate, high-density polyethylene, low-density polyethylene, polypropylene, polystyrene, polyamide, polyvinylchloride, or any combination thereof.

9. The composition of claim 1,
further comprising a protection component configured to reduce a reaction between the first active agent dispersion and the at least one chlorite salt dispersion,
wherein the protection component comprises an acrylic dispersion, a styrene acrylate dispersion, a polyurathene, an epoxy co-polymer, a cellulose, a polymer or copolymer dispersion, or any combination thereof, and
wherein the protection component is in contact with at least the first active agent dispersion or the at least one chlorite salt dispersion.

10. The composition of claim 1,
further comprising a neutralizing agent selected from the group consisting of: sodium thiosulfate, ferrous chloride, ferrous sulfate, vitamin E, and any combination thereof.

11. The composition of claim 1,
further comprising a second active agent dispersion having a pKa of 0.1-2.0,
wherein the at least one chlorite salt dispersion is in contact with the first active agent dispersion and the second active agent dispersion.

12. The composition of claim 11, wherein the sufficient second amount of the second active agent dispersion has a pKa of 0.1-1.5.

13. The composition of claim 12,
wherein the sufficient first amount of the first active agent dispersion is in a first layer,
wherein the sufficient second amount of the at least one chlorite salt dispersion is in a second layer,
wherein the sufficient second amount of the second active agent dispersion is in a third layer, and
wherein the second layer is positioned between the first layer and the third layer.

14. The composition of claim 1,
further comprising a stabilizing agent selected from the group consisting of: ammonia, methylamine, sodium hydroxide, sodium bicarbonate, Purolite A200-MBOH, Dow FPA-55, a basic zeolite, and any combination thereof.

15. A composition, comprising:
a sufficient first amount of a first active agent dispersion;
wherein the first active agent dispersion has a pKa of 0.1-2.0,
wherein the first active agent dispersion is selected from the group consisting of: an acid cation exchange resin, an acidic zeolite, an acidic clay, an organic acid, an inorganic acid, and any combination thereof, and
wherein the first active agent dispersion comprises a plurality of particles;
wherein the plurality of particles has a median diameter of between 0.5-1000 micrometers; and
a sufficient second amount of at least one chlorite salt dispersion;
wherein, when the sufficient second amount of the at least one chlorite salt dispersion is tested in the composition with the sufficient first amount of the first active agent dispersion, the sufficient first amount of the first active agent dispersion and the sufficient second amount of the at least one chlorite salt dispersion are contacted with an aqueous liquid, and chlorine dioxide radicals are generated at a rate ranging from 0.001 mg/min-0.02 mg/min so as to result in a microbial reduction of between 2 log CFU/mL and 10 log CFU/mL from between 1 and 60 minutes.

16. A composition, comprising:
a sufficient first amount of a first active agent dispersion;
wherein the first active agent dispersion has a pKa of 0.1-2.0,
wherein the first active agent dispersion is selected from the group consisting of: an acid cation exchange resin, an acidic zeolite, an acidic clay, an organic acid, an inorganic acid, and any combination thereof, and
wherein the first active agent dispersion comprises a plurality of particles;
wherein the plurality of particles has a median diameter of between 0.5-1000 micrometers; and
a sufficient second amount of at least one chlorite salt dispersion;
wherein, when the composition is contacted with an aqueous liquid, the sufficient first amount of the first active agent dispersion and the sufficient second amount of the at least one chlorite salt dispersion results in a generation of chlorine dioxide radicals at a Cmax ranging from 15 ppm-25 ppm from between 4 hours-6 hours.

17. A composition, comprising:
a sufficient first amount of a first active agent dispersion;
wherein the first active agent dispersion has a pKa of 0.1-2.0,
wherein the first active agent dispersion is selected from the group consisting of: an acid cation exchange resin, an acidic zeolite, an acidic clay, an organic acid, an inorganic acid, and any combination thereof, and
wherein the first active agent dispersion comprises a plurality of particles;
wherein the plurality of particles has a median diameter of between 0.5-1000 micrometers; and
a sufficient second amount of at least one chlorite salt dispersion;
wherein, when the composition is contacted with an aqueous liquid, the sufficient first amount of the first active agent dispersion and the sufficient second amount of the at least one chlorite salt dispersion results in a generation of chlorine dioxide radicals at a Cmax ranging from 5 ppm-15 ppm from between 10 hours-20 hours.

18. A product, comprising an absorbent pad,
wherein the absorbent pad comprises the composition of claim 1.

19. A product, comprising a package insert,
wherein the package insert comprises the composition of claim 1.
